(19) United States
(12) Patent Application Publication
(10) Pub. No.: US 2026/0237456 A1
(43) Pub. Date: Aug. 13, 2026

(54) METHODS FOR PREDICTING RESPONSE TO A THERAPY FOR A DISORDER THROUGH CORE MICROBIOME GUILDS

(71) Applicants: RUTGERS, THE STATE UNIVERSITY OF NEW JERSEY, New Brunswick, NJ (US); SHANGHAI JIAO TONG UNIVERSITY, Shanghai (CN)

(72) Inventors: Liping ZHAO, Princeton, NJ (US); Guojun WU, North Brunswick, NJ (US); Chenhong ZHANG, Shanghai (CN)

(21) Appl. No.: 19/477,366

(22) PCT Filed: Apr. 25, 2024
(86) PCT No.: PCT/US2024/026282

§ 371 Date: Oct. 21, 2025

Related U.S. Application Data
(60) Provisional application No. 63/498,177, filed on Apr. 25, 2023.
(60) Provisional application No. 63/595,189, filed on Nov. 1, 2023.

(51) Int. Cl. G16B 20/00; A61K 35/00; A61K 35/741; A61K 35/744; A61K 35/745; A61K 35/747; G16B 30/20; G16B 40/20; G16B 40/30

(52) U.S. Cl. CPC G16B 20/00; A61K 35/741; A61K 35/744; A61K 35/745; A61K 35/747; G16B 30/20; G16B 40/20; G16B 40/30; A61K 2035/115

(57) ABSTRACT

Methods and systems for predicting a subject's response to a therapy by obtaining a first plurality of nucleic acid sequences for genomic DNA from a sample from the gut of a subject. Determine, from the nucleic acid sequences, a plurality of genomic abundance values for a plurality of gut bacteria. Apply a model to the plurality of genomic abundance values, thereby obtaining the prediction of a subject's response to a therapy as an output of the model.

### CROSS REFERENCE TO RELATED APPLICATIONS

This application claims priority to U.S. Provisional Patent Application No. 63/498,177, filed Apr. 25, 2023, and U.S. Provisional Patent Application No. 63/595,189, filed Nov. 1, 2023, the contents of which are hereby incorporated by reference herein, in their entireties, for all purposes.

### BRIEF DESCRIPTION OF THE SEQUENCE LISTING

This submission incorporates by reference the “Sequence Listing XML” file named ST26_126146_5001_WO.XML containing SEQ ID NOs: 1-99534, created on Apr. 24, 2024, and having a size of 2,491,699 kilobytes, in accordance with 37 CFR §§ 1.831 through 1.835, submitted on a read only optical disc (DVD) as an XML file via mail on Apr. 25, 2024. The Sequence Listing XML is hereby incorporated by reference herein in its entirety.

### BACKGROUND

The human gut microbiome, emblematic of a complex adaptive system (CAS), hosts trillions of microorganisms, embodying a rich array of phylogenetic diversity. This sophisticated ecosystem not only sustains active interaction with its host environment but also showcases dynamic adaptability, thereby playing a pivotal role in the maintenance of health and modulation of disease susceptibility. Amidst the staggering microbial diversity resident within the human gut, the concept of a ‘core microbiome’ has gained considerable traction. This core is hypothesized to incorporate microbes that ubiquitously colonize healthy individuals, thus contributing significantly to the preservation of homeostasis in nutrition, metabolism, immunity, and behavior. The integral role of this core microbiome is akin to that of an essential organ, underscoring its criticality in overall health management.

Historically, the demarcation of the core microbiome has predominantly rested upon the evaluation of the presence or absence, supplemented by the quantification of the abundance or prevalence of specific taxa or genes/pathways within a cohort of healthy individuals. While these methodologies have undoubtedly provided significant insights into the structural configuration and potential functional traits of the microbiome, they may inadequately represent the vital ecological interactions that underscore the stability and resilience of this intricate system. This oversight is particularly relevant when considering the critical role these interactions play in the inception, progression, and remission of various disease states.

As a CAS, the microbiome adheres to the modular design principle. Integral components of a CAS are organized into modules, which interconnect to establish a network. Within the gut ecosystem, individual microbes are integrated into a modular structure referred to as guilds. Each guild, despite comprising microorganisms of diverse taxonomic backgrounds, functions as a coherent functional unit or module within the microbiome's CAS. Members of a guild display cooperative behavior through co-abundance, and different guilds may engage in cooperative or competitive interactions to shape an ecological network. Consequently, the characterization of the core microbiome in terms of guilds emerges as a promising and intriguing approach.

### SUMMARY

Throughout their co-evolution, gut microbiota has established a vital role in sustaining human health. Identifying core microbiome constituents that reliably confer essential health benefits, however, remains a significant challenge. It was posited that these core members should sustain their ecological interactions, cooperative or competitive, in spite of changing environmental conditions. Drawing from a high-fiber intervention trial in type 2 diabetes patients and 26 diverse case-control datasets, 284 high-quality metagenome-assembled genomes consistently forming stable pairs across individuals amidst dietary shifts or disease progression were identified. These genomes correspond to two guilds, encompassing the most resilient and highly interconnected bacteria, which collectively correlate with an expansive range of health conditions. One guild's genomes were gene-rich for plant polysaccharide degradation and butyrate production, while the other was typified by a high prevalence of genes linked to virulence and antibiotic resistance. Utilizing these genomes as a reference, Random Forest models adeptly differentiated between cases and controls across 15 distinct diseases and forecasted patients' responses to immunotherapy. Therefore, this core microbiome signature has potential as a unifying therapeutic target for enhancing health.

Individual microbial cells are considered to be the fundamental components or agents of the CAS, representing the principal ecologically meaningful structural and functional units within the gut ecosystem. In some embodiments, the use of high-quality metagenome-assembled genomes (HQMAGs) are used as a surrogate to profile these microbial cells, thus providing a more comprehensive and realistic depiction of the microbiome compared to gene-, pathway-, or taxon-centric approaches. This perspective encompasses the complete genetic potential and ecological identity of a microbe, reinforcing the essential ecological axiom that organisms (or more accurately, cells) interact with each other and their environment, rather than genes/pathways or taxa.

To identify the core constituents of the gut microbiome, a genome-centric, reference-free approach that emphasizes the stability of ecological interactions was used. This methodology involved detecting stable relationships among HQMAGs across varying conditions, with environmental perturbations to the gut ecosystem being introduced via dietary interventions or disease progression. These stable relationships can unveil the core members of the microbiome. This aligns with a foundational principle of systems biology, whereby relationship stability often signifies pivotal system components. In the context of the gut microbiome, these core components are likely to execute essential functions contributing to system resilience and host health, demanding their persistent presence and predictable interaction patterns. Therefore, uncovering these stable relationships could disclose these critical microbial components, potentially exposing the backbone of the ecological network conserved within the gut microbiome, across individuals, populations, or health states.

A robust seesaw-like network comprising two competing bacterial guilds was identified. This network was discerned by searching for stable genome pairs across co-abundance networks among individuals pre- and post-high fiber intervention (the QD trial, FIG. 1A), or between healthy and diseased cohorts. This seesaw-like network embodies both cooperative and competitive interactions, potentially indicating a key feature of a stable microbiome structure. The HQMAGs identified within this novel core microbiome demonstrated correlations with various clinical parameters in patients with type 2 diabetes mellitus (T2DM) undergoing a high fiber intervention. Moreover, a universal machine learning model, premised on these HQMAGs in the seesaw-networked core microbiome, successfully differentiated cases from controls in 26 independent datasets spanning 15 different diseases. Furthermore, these HQMAGs supported a machine learning model for predicting personalized treatment responses to immunotherapy in patients with cancer or autoimmune diseases. The disclosure introduces a novel conceptual and analytical paradigm for studying the core gut microbiome. This paradigm provides enhanced health maintenance strategies and disease management, enabling personalized interventions that accommodate the intricate interplay of microbial relationships within the gut ecosystem.

Given the background above, a genome-centric MWAS was adopted in which high-quality draft genomes assembled from metagenomic datasets (metagenome-assembled genomes, MAGs) are used as the basic building blocks of the gut ecosystem and the most important microbiome features for correlation analysis with disease phenotypes. MAGs again are not independent microbiome features. They have ecological interactions such as competition or cooperation with each other and organize themselves into a higher-level structure called “guilds” [5]. Each guild is potentially a functional unit in the gut ecosystem and its members may have widely diverse taxonomic background but show co-abundant behavior. Guilds have been shown to be positively or negatively correlated with disease phenotypes [17]. Thus, MAGs and their guild-level aggregation are ecologically meaningful features for identifying microbiome signatures associated with human diseases.

Dysbiosis in the gut microbiome has been linked with an increased risk for a wide range of human diseases [1, 2]. To date, much effort has concentrated on identifying gene- or taxon-based microbial signatures as disease biomarkers. However, such signatures remain controversial [3, 4] and overlook the fact that gut bacterial strains are not independent but rather form coherent functional groups (a.k.a. “guilds”) to interact with each other and affect host health [5]. Therefore, embodiments may propose to search for strain-level microbiome signature in the form of robust guilds, through which the gut microbiome provides stable health-relevant functions to the host. Here embodiments may show that two competing bacterial guilds are organized as two ends of a robustly stable seesaw-like network and their abundance are correlated with a wide range of chronic diseases. 141 out of a total of 1,845 metagenome-assembled genomes (MAGs) formed the two competing guilds given their stable ecological relationships while experiencing profound structural changes in the gut microbiome during a 3-month high fiber intervention and 1-year follow-up in patients with type 2 diabetes (T2DM). The 50 genomes in Guild 1 harbored more genes for plant polysaccharide degradation and butyrate production, while the 91 genomes in Guild 2 included almost all the virulence or antibiotic resistance gene carriers predicted from the 1,845 MAGs. Random Forest regression model showed that the abundance distribution of the 141 genomes were associated with 41 out of 43 bio-clinical parameters. With these 141 MAGs as reference genomes, such a seesaw network was not only detectable but also conducive to machine learning models for predictive classification between case and control of 9 diseases including T2DM, atherosclerosis, hypertension, liver cirrhosis, inflammatory bowel diseases, colorectal cancer, ankylosing spondylitis, schizophrenia, and Parkinson's disease in 12 independent metagenomic datasets from 1,874 participants across ethnicity and geography. The two seesaw networked guilds may work as a core microbiome and their balance can be modulated for disease risk management.

In one aspect, the disclosure provides a pharmaceutical composition comprising a first gut microorganism selected from those microorganisms listed in FIG. 13A-13XX. In some embodiments, the composition further includes a pharmaceutically acceptable excipient.

In one aspect, the disclosure provides a method for treating a subject in need thereof, the method comprising administering to the subject a therapeutically effective amount of a pharmaceutical composition as described herein. In some embodiments, the administering is by fecal microbiome transplantation. In some embodiments, the administering is by direct transplantation into the gut of the subject. In some embodiments, the administering is by oral ingestion.

In one aspect, the present disclosure provides methods, and systems for training a model for predicting a subject's response to a therapy. The method includes, at a computer system having at least one processor, and memory storing one or more programs for execution by the one or more processors, obtaining, in electronic form, for each respective training subject in a plurality of training subjects, wherein each respective training subject in the plurality of training subjects has received a therapy for a disorder: (i) a corresponding plurality of genomic abundance values for the respective training subject at a time prior to receiving the therapy, wherein the corresponding plurality of genomic abundance values comprising, for each respective gut microorganism in a plurality of gut microorganisms, a corresponding value for the abundance of the genome of the respective gut microorganism in a corresponding biological sample from the gut of the respective training subject, and (ii) an indication of the respective training subject's response to the therapy of the respective training subject. The method also includes inputting, for each respective training subject in the plurality of training subjects, information about the respective training subject into a model comprising a plurality of parameters, wherein the model applies the plurality of parameters to the information through at least 10,000 computations to obtain a corresponding output for the respective training subject from the model, wherein the corresponding output comprises a prediction of the respective training subject's response to the therapy, the information about the respective training subject comprises the corresponding genomic abundance value for each respective gut microorganism in the plurality of gut microorganisms, and the plurality of gut microorganisms are selected from Table 1, Table 2, or FIG. 13A-13XX. The method also includes adjusting the plurality of parameters based on, for each respective training subject in the first plurality of training subjects, one or more differences between (i) the corresponding output from the model and (ii) the corresponding indication of the respective training subject's response to the therapy.

Accordingly, another aspect of the present disclosure provides methods, and systems for using a model for predicting a subject's response to a therapy. The method includes, at a computer system having one or more processors, and memory storing one or more programs for execution by the one or more processors, obtaining, in electronic form, a plurality of genomic abundance values comprising, for each respective gut microorganism in a plurality of gut microorganisms selected from Table 1, Table 2, or FIG. 13A-13XX, a corresponding abundance value for the genome of the respective species of gut microorganisms, in the plurality of gut microorganisms, in a biological sample from the subject. The method also includes inputting the plurality of genomic abundance values into a model comprising a plurality of parameters, wherein the model applies the plurality of parameters to the plurality of genomic abundance values through at least 10,000 computations to generate as output from the model a prediction of the subject's response to the therapy.

As disclosed herein, any embodiment disclosed herein when applicable can be applied to any other aspect.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

Accordingly, one aspect of the invention provides a method of training a model for predicting subject response to a therapy at a computer system having one or more processors, and memory storing one or more programs for execution by the one or more processors.

In some embodiments, the method includes obtaining, in electronic form, for each respective training subject in a plurality of training subjects, wherein each respective training subject in the plurality of training subjects has received a therapy for a disorder, (i) a corresponding plurality of genomic abundance values for the respective training subject at a time prior to receiving the therapy, wherein the corresponding plurality of genomic abundance values comprise, for each respective gut microorganism in a plurality of gut microorganisms, a corresponding value for the abundance of the genome of the respective gut microorganism in a corresponding biological sample from the gut of the respective training subject, (ii) an indication of the respective training subject's response to the therapy of the respective training subject.

In some such embodiments, the method includes sequencing, for each respective subject in the plurality of training subjects, genomic DNA from the corresponding biological sample from the gut of the respective training subject, thereby obtain the corresponding plurality of at least 100,000 nucleic acid sequences.

In some such embodiments, the method includes obtaining, for each respective training subject in the plurality of training subjects, in electronic form, a corresponding plurality of at least 100,000 nucleic acid sequences for genomic DNA from the corresponding biological sample from the gut of the respective training subject.

In some such embodiments, the method includes determining, for each respective gut microorganism in the plurality of gut microorganisms, the corresponding value for the abundance of the genome of the respective gut microorganism from the corresponding plurality of at least 100,000 nucleic acid sequences.

In some such embodiments, the method includes, for each respective training subject in the plurality of training subjects, assembling, in electronic form, a corresponding plurality of gut microorganism genomes by metagenomic de novo sequence assembly from the corresponding plurality of at least 100,000 nucleic acid sequences, and calculating, for each respective gut microorganism in the plurality of gut microorganisms, the corresponding value for the abundance of the genome of the respective gut microorganism based on the prevalence of respective nucleic acid sequences, in the plurality of at least 100,000 nucleic acid sequences, used to assemble a respective gut microorganism genome in the plurality of gut microorganism genomes corresponding to the respective gut microorganism.

In some such embodiments, the method includes, for each respective subject in the plurality of training subjects, assigning each respective nucleic acid sequence in the corresponding plurality of at least 100,000 sequences to a respective gut microorganism in the plurality of gut microorganisms, thereby generating, for each respective gut microorganism in the plurality of gut microorganism, a corresponding count of respective nucleic acid sequences in the corresponding plurality of nucleic acid sequences assigned to the respective gut microorganism, and determining, for each respective gut microorganism in the plurality of gut microorganisms, the corresponding genomic abundance value for the respective gut microorganism based on the corresponding count of respective nucleic acid sequences assigned to the respective gut microorganism.

In some such embodiments, the plurality of gut microorganisms comprises at least 20 gut microorganisms selected from Table 1, Table 2, or FIG. 13A-13XX.

In some such embodiments, the plurality of gut microorganisms comprises at least 20 microorganisms selected from those microorganisms in Table 1, Table 2, or FIG. 13A-13XX having a connectivity of at least 2.

In some such embodiments, the biological sample from the gut of the respective subject is a fecal sample from the respective training subject.

In some such embodiments, the therapy is a biological therapy, an immunotherapy, a chemotherapy, a radiation therapy, a gene therapy, a hormone therapy, a photodynamic therapy, a targeted therapy, small molecules, antibodies, polynucleotide, natural compound, immune modulator, bone marrow therapy, stem cell therapy, surgery therapy, induction therapy, maintenance therapy, or a combination thereof.

In some such embodiments, the disorder is selected from the group consisting of type-2 diabetes, hypertension, schizophrenia, atherosclerotic cardiovascular disease (ACVD), liver cirrhosis (LC), inflammatory bowel diseases (IBD), colorectal cancer (CRC), ankylosing spondylitis (AS), and Parkinson's disease (PD), inflammatory bowel disease (IBD, rheumatoid arthritis (RA), or advanced melanoma and B cell lymphoma.

In some such embodiments, the disorder is cancer.

In some embodiments, the method includes inputting, for each respective training subject in the plurality of training subjects, information about the respective training subject into a model comprising a plurality of parameters, wherein the model applies the plurality of parameters to the information through at least 10,000 computations to obtain a corresponding output for the respective training subject from the model, wherein the corresponding output comprises a prediction of the respective training subject's response to the therapy, and the information about the respective training subject comprises the corresponding genomic abundance value for each respective gut microorganism in the plurality of gut microorganisms, and the plurality of gut microorganisms are selected from Table 1, Table 2, or FIG. 13A-13XX.

In some such embodiments, the prediction of the respective training subject's response is a class output of a respective response, in a plurality of possible responses, of the respective training subject.

In some such embodiments, the prediction of the respective training subject's response is a probability output for the respective training subject's response.

In some such embodiments, the model is a neural network algorithm, a support vector machine algorithm, a Naïve Bayes algorithm, a nearest neighbor algorithm, a boosted trees algorithm, a Random Forest algorithm, a convolutional neural network algorithm, a decision tree algorithm, a regression algorithm, or a clustering algorithm.

In some such embodiments, the plurality of parameters is at least 1000, at least 10,000, at least 15,000, at least 50,000, at least 100,000, at least 250,000, at least 500,000, or at least 1,000,000 parameters.

In some such embodiments, the model applies the plurality of parameters to the information through at least 25,000, at least 50,000, at least 100,000, at least 250,000, at least 500,000, or at least 1,000,000 computations to obtain a corresponding output for the respective training subject from the model.

In some embodiments, the method includes adjusting the plurality of parameters based on, for each respective training subject in the plurality of training subjects, one or more differences between (i) the corresponding output from the model and (ii) the corresponding indication of the respective training subject's response to the therapy.

Another aspect of the present disclosure provides a method of using a model for predicting a subject's response to a therapy for a disorder at a computer system having one or more processors, and memory storing one or more programs for execution by the one or more processors.

In some embodiments, the method includes obtaining, in electronic form, a plurality of genomic abundance values comprising, for each respective gut microorganism in a plurality of gut microorganisms selected from Table 1, Table 2, or FIG. 13A-13XX, a corresponding abundance value for the genome of the respective species of gut bacteria, in the plurality of gut microorganisms, in a biological sample from the subject.

In some such embodiments, the method includes sequencing genomic DNA from the biological sample from the gut of the subject, thereby obtaining the plurality of at least 100,000 nucleic acid sequences.

In some such embodiments, the method includes obtaining, in electronic form, a plurality of at least 100,000 nucleic acid sequences for genomic DNA from the biological sample from the gut of the subject.

In some such embodiments, the meth od includes determining, for each respective gut microorganism in the plurality of gut microorganisms, the corresponding value for the abundance of the genome of the respective gut microorganism from the plurality of at least 100,000 nucleic acid sequences.

In some such embodiments, the method includes assembling, in electronic form, a corresponding plurality of gut microorganism genomes by metagenomic de novo sequence assembly from the plurality of at least 100,000 nucleic acid sequences, and calculating, for each respective gut microorganism in the plurality of gut microorganisms, the corresponding value for the abundance of the genome of the respective gut microorganism based on the prevalence of respective nucleic acid sequences, in the plurality of at least 100,000 nucleic acid sequences, used to assemble a respective gut microorganism genome in the plurality of gut microorganism genomes corresponding to the respective gut microorganism.

In some such embodiments, the method includes assigning each respective nucleic acid sequence in the plurality of at least 100,000 sequences to a respective gut microorganism in the plurality of gut microorganisms, thereby generating, for each respective gut microorganism in the plurality of gut microorganism, a corresponding count of respective nucleic acid sequences in the plurality of nucleic acid sequences assigned to the respective gut microorganism, and determining, for each respective gut microorganism in the plurality of gut microorganisms, the corresponding genomic abundance value for the respective gut microorganism based on the corresponding count of respective nucleic acid sequences assigned to the respective gut microorganism.

In some such embodiments, the plurality of gut microorganisms comprises at least 20 microorganisms selected from those microorganisms in Table 1, Table 2, or FIG. 13A-13XX having a connectivity of at least 2.

In some such embodiments, the biological sample from the gut of the subject is a fecal sample.

In some such embodiments, the therapy is a biological therapy, an immunotherapy, a chemotherapy, a radiation therapy, a gene therapy, a hormone therapy, a photodynamic therapy, a targeted therapy, small molecules, antibodies, polynucleotide, natural compound, immune modulator, bone marrow therapy, stem cell therapy, surgery therapy, induction therapy, maintenance therapy, or a combination thereof.

In some such embodiments, the disorder is selected from the group consisting of type-2 diabetes, hypertension, schizophrenia, atherosclerotic cardiovascular disease (ACVD), liver cirrhosis (LC), inflammatory bowel diseases (IBD), colorectal cancer (CRC), ankylosing spondylitis (AS), and Parkinson's disease (PD), inflammatory bowel disease (IBD), rheumatoid arthritis (RA), advanced melanoma and B cell lymphoma.

In some such embodiments, the disorder is cancer.

In some embodiments, the method includes inputting the plurality of genomic abundance values into a model comprising a plurality of parameters, wherein the model applies the plurality of parameters to the plurality of genomic abundance values through at least 10,000 computations to generate as output from the model a prediction of the subject's response to the therapy.

In some such embodiments, the prediction of the subject's response is a class output of a respective response, in a plurality of possible responses, of the respective subject.

In some such embodiments, the prediction of the subject's response of the subject is a probability output for the respective subject's response.

In some such embodiments, the model is a neural network algorithm, a support vector machine algorithm, a Naïve Bayes algorithm, a nearest neighbor algorithm, a boosted trees algorithm, a Random Forest algorithm, a convolutional neural network algorithm, a decision tree algorithm, a regression algorithm, or a clustering algorithm.

In some such embodiments, the plurality of parameters is at least 1000, at least 10,000, at least 15,000, at least 50,000, at least 100,000, at least 250,000, at least 500,000, or at least 1,000,000 parameters.

In some such embodiments, the model applies the plurality of parameters to the information through at least 25,000, at least 50,000, at least 100,000, at least 250,000, at least 500,000, or at least 1,000,000 computations to obtain a corresponding output for the respective subject from the model.

In some embodiments, the method includes treating the subject by: i) when the prediction of the subject's response to the therapy satisfies a threshold likelihood that the subject will respond favorably to the therapy, administer the therapy to the subject; ii) when the prediction of the subject's response to the therapy does not satisfy the threshold likelihood that the subject will respond favorably to the therapy, administer one or more of the plurality of gut microorganisms to the subject.

Another aspect of the present disclosure provides a computer system. The computer system comprises one or more processors and a non-transitory computer-readable medium including computer-executable instructions that, when executed by the one or more processors, cause the processors to perform the method described herein.

Another aspect of the present disclosure provides a non-transitory computer readable storage medium. The non-transitory computer readable storage medium stores instructions, which when executed by a computer system, cause the computer system to perform any of the methods described herein.

### BRIEF DESCRIPTION OF THE DRAWINGS

FIG. 1 illustrates a block diagram of an example computing device, in accordance with some embodiments of the present disclosure.

FIGS. 2A, 2B, 2C, and 2D collectively provide a flow chart of processes and features for training a model for predicting a subject's response to a therapy for a disorder, in accordance with some embodiments of the present disclosure.

FIGS. 3A, 3B, and 3C collectively provide a flow chart of processes and features for predicting a subject's response to a therapy for a disorder, in accordance with some embodiments of the present disclosure.

FIGS. 4A, 4B, 4C, 4D, 4E, 4F, 4G, and 4H collectively illustrate reversible alterations in the gut microbiota induced by a high-fiber diet are associated with corresponding shifts in metabolic phenotypes in patients with Type 2 Diabetes Mellitus (T2DM). (A) Study design of the QD trial. During the Run-in period, written informed consent, questionnaire of personal information and HbA1c-based screening were conducted. After Run-in, medical checkup and sample collection were conducted at baseline (M0), three months (M3) after on the high fiber intervention (W) or usual diet (U) and one year (M15) after the high fiber intervention stopped. (B) Changes of fiber intake. (C) Global changes of the gut microbiome as shown by the principal coordinate analysis based on the Bray-Curtis distance with the 1845 HQMAGs and (D) Average Bray-Curtis distance between the groups. PERMANOVA test (9,999 permutations) was performed to compare the groups. * P<0.05 and *** P<0.001. The color intensity of the square showed the magnitude of average Bray-Curtis distance. (E) Change of HbA1c, (F) The percentage of participants who achieved adequate glycemic control, (G) Fasting blood glucose, and (H) The glucose area under the curve (AUC) in a meal tolerance test (MTT). For (E), (G) and (H), data shown as percent changes from baseline (±S.E.M). Friedman test followed by Nemenyi post-hoc test was used for comparison in the same group, compact letters reflect significance (P<0.05). n=67 in W group and n=28 in U group. Mann-Whitney test (two-sided) was used for comparison between W and U at the same time point, * P<0.05, ** P<0.01 and *** P<0.001. n=74 in W (M0) (For panel H, n=72), n=74 in W (M3), n=67 in W (M15), n=36 in U (M0), n=36 in U (M3) and n=28 in U (M15).

FIGS. 5A and 5B collectively illustrate that despite substantial global changes in the gut microbiota induced by the high-fiber intervention, two competing bacterial guilds, which are associated with HbA1c levels, form a robust seesaw-like network within the ecosystem. (A) The distribution of different types of correlations of the genome pairs during the trial. The 3 letters show the correlations (N for negative, P for positive and U for un-correlated) of the genome pairs at M0, M3 and M15 subsequently. Stable correlations, NNN and PPP, were highlighted. (B) Correlations between genome clusters and HbA1c using linear mixed effect model by MaAslin2 package. Abundance was log transformed. Subject was used as random effect. N=67. * BH adjusted P<0.05, *** BH adjusted P<0.001.

FIGS. 6A, 6B, 6C1, 6C2, 6D, 6E1, 6E2, 6E3, 6E4, 6E5, 6E6, 6E7, 6E8, 6E9, 6E10, and 6E11 collectively illustrate Genomes within the two competing guilds predict metabolic health outcomes in T2DM patients of the QD trial, and distinguish cases from controls across seven diseases in eleven independent case-control metagenomic datasets (Case-Control Dataset Collection I). (A) Change of the mean range-scaled robust centered-log-ratio (rclr) transformed abundances of Guild 1, Guild 2, and their ratio across the trial in the W group. Friedman test followed by Nemenyi test was used to analyze the difference between time points. Compact letters reflect the significance at P<0.05. (B) Prediction of clinical parameters with the genomes in the two competing guilds. Linear mixed effect models were trained based on the mean range-scaled rclr abundances of Guild 1 and Guild 2, and clinical parameters at M0 and M3 with subject as random effect. The mean range-scaled rclr abundances of Guild 1 and Guild 2 at M15 were used to predict clinical parameters based on the trained model. The bar plot shows the Pearson's correlations coefficient between the predicted clinical parameters and measured values at M15. The asterisk before the parameter's name shows the significance of the Pearson's correlations. P values were adjusted by Benjamini & Hochberg's method. * adjusted P<0.05, ** adjusted P<0.01 and *** adjusted P<0.001. BMI, body mass index; SBP, systolic blood pressure; DBP, diastolic blood pressure; WC, waist circumference; HP, hip circumference; TNF-α, tumor necrosis factor-α; WBC, white blood cell count; CRP, C-reactive protein; LBP, lipopolysaccharide-binding protein; TC, total cholesterol; TG, triglyceride; Lpa, lipoprotein a; HDL, high-density lipoprotein; APOA, apolipoprotein A; LDL, low-density lipoprotein; APOB, apolipoprotein B; GFR (MDRR), glomerular filtration rate; CysC, Cystatin C; ACR, urinary microalbumin to creatinine ratio; IMT, intima-media thickness; DAN, diabetic autonomic neuropathy score; MHR, mean heart rate; SDNN, standard deviation of NN intervals; SDANN, standard deviation of the average NN intervals calculated over 5 minutes; SDNNIndex, mean of standard deviation of NN intervals for 5-minute segments; rMSSD, root-mean-square of the differences of successive NN intervals; pNN50, percentage of the interval differences of successive NN intervals greater than 50 ms; TP, total power; VLF, very low frequency power; LF, low frequency power; HF, high frequency power; DPN, diabetic peripheral neuropathy score. (C) Differences in genetic capacity of carbohydrate substrate utilization (CAZy), short-chain fatty acid production (SCFA), antibiotic resistance genes (ARG) and virulence factor genes (VF). The heatmaps show the proportion (CAZy) or gene copy numbers (SCFA, ARG and VF) of each category in each genome. For carbohydrate substrate utilization, CAZy genes were predicted in each genome. The proportion of CAZy genes for a particular substrate was calculated as the number of the CAZy genes involved in its utilization divided by the total number of the CAZy genes. Arabinoxylan-related CAZy families: CE1, CE2, CE4, CE6, CE7, GH10, GH11, GH115, GH43, GH51, GH67, GH3 and GH5; cellulose-related: GH1, GH44, GH48, GH8, GH9, GH3 and GH5; inulin-related: GH32 and GH91; mucin-related families: GH1, GH2, GH3, GH4, GH18, GH19, GH20, GH29, GH33, GH38, GH58, GH79, GH84, GH85, GH88, GH89, GH92, GH95, GH98, GH99, GH101, GH105, GH109, GH110, GH113, PL6, PL8, PL12, PL13 and PL21; pectin-related: CE12, CE8, GH28, PL1 and PL9; starch-related: GH13, GH31 and GH97. For short chain fatty acid production, FTHFS: formate-tetrahydrofolate ligase for acetate production; ScpC: propionyl-CoA succinate-CoA transferase and Pct: propionate-CoA transferase for propionate production; But: Butyryl-coenzyme A (butyryl-CoA): acetate CoA transferase, Buk: butyrate kinase, 4Hbt: butyryl-CoA: 4-hydroxybutyrate CoA transferase, Ato: butyryl-CoA: acetoacetate CoA transferase (AtoA: alpha subunit, AtoD: beta subunit) for butyrate production. Mann-Whitney test (two-sided) was used to analyze the difference between Guild 1 and Guild 2. #P<0.1, * P<0.05, ** P<0.01 and *** P<0.001. Number of genomes in Guild 1 (green bar): n=50, in Guild 2 (purple bar): n=91. (D) Datasets from 11 different datasets on 7 diseases including type 2 diabetes (T2D), liver cirrhosis (LC), ankylosing spondylitis (AS), atherosclerotic cardiovascular disease (ACVD), schizophrenia (SCZ), colorectal cancer (CRC), inflammatory bowel disease (IBD) dataset were collected as Case-Control Dataset Collection I. Sample size of each case-control dataset is showed in red and green number in the left panel respectively. For each dataset in the collection, metagenomic reads were recruited to the 141 genomes of the QD two competing guilds to estimate genome abundance in each sample (E) Based on the abundance matrix of the 141 genomes, a Random Forest classification model with leave-one-out cross validation was trained to classify case and control subjects in each dataset. ROC curves and the area under the curves (AUC) were shown here.

FIGS. 7A and 7B collectively illustrate genomes forming the two competing guilds, as identified from a case-control dataset specific to one disease, demonstrate significant effectiveness in classifying cases from controls across independent datasets on different diseases within the Case-Control Dataset Collection I. (A) Identification of the seesaw networked two competing guilds in Case-Control Dataset Collection I. Sample size of each case-control study is showed in red and green number in the left panel respectively. Case-Control Dataset Collection I has 11 published metagenomic case-control datasets on 7 diseases including type 2 diabetes (T2D), liver cirrhosis (LC), ankylosing spondylitis (AS), atherosclerotic cardiovascular disease (ACVD), schizophrenia (SCZ), colorectal cancer (CRC), inflammatory bowel disease (IBD) dataset. Datasets from 3 studies were combined to analyze CRC. Datasets from 2 studies were combined to analyze IBD. The percentage of correlations followed the pattern in the seesaw networked two competing guilds (i.e., positive edges within each guild, negative edges between the 2 guilds) was in yellow, and the ratio of correlations that were negative within each guild and positive between the guilds was in black of the 100% stacked bar. (B) The two competing guilds found in one dataset was used as predictors to classify case and control in the same dataset and in all the other 10 datasets. Random Forest classification model with leave-one-out cross validation was applied in each dataset on each set of the two competing guilds. The area under the ROC curve (AUC) values were showed in the heatmap.

FIGS. 8A, 8B1, 8B2, 8B3, 8B4, 8B5, 8B6, 8B7, 8B8, 8B9, 8B10, 8B11, 8B12, 8B13, 8B13, 8B14, 8B15, 8B16, 8C1, and 8C2 collectively illustrate the combined core genomes, drawn from all identified competing guilds, effectively differentiate cases from controls across a broader range of diseases, and predict treatment outcomes in independent datasets. (A) Identification of the combined genomes and the combined core genomes from the 8 sets of the two competing guilds, which were generated from QD dataset and Case-Control Dataset Collection I. All the HQMAGs in each set of the two competing guilds were dereplicated based on the cutoff of 99% average nucleotide identity (ANI) between two genomes. 788 non-redundant HQMAGs were obtained as the combined genomes of all the 8 sets of the two competing guilds. Random forest classification model with leave-one-out cross validation was constructed based on the 788 HQMAGs in each dataset. The HQMAGs were ranked based on their importance across all the models. From the least important HQMAGs (biggest importance rank), subsequently removing one HQMAGs to do random forest classification model in each dataset. In each dataset, rank the HQMAG number based on the area under the ROC curve (AUC) values. The scatter plot shows the relationship between HQMAG number and model performance. The y axis is the sum of rank based on AUC values (the smaller the value, the better the performance). 302 HQMAG reached best performance. After excluding 18 HQMAGs that exhibited inconsistent C1A and C1B assignments across the datasets, a total of 284 HQMAG were kept from the 302 HQMAG as the Combined Core genomes of all the 8 sets of the two competing guilds. (B) The Combined Core were used as predictors in Case-Control Dataset Collection II which has 15 published metagenomic case-control datasets on 10 diseases including 1 dataset on ankylosing spondylitis (AS #2): Case n=85, Control n=55, 1 on autism spectrum disorder (ASD): Case n=64, Control n=64, 1 on Behcet's disease (BD): Case n=24, Control n=52, 1 on COVID-19: Case n=47, Control n=19, 3 on colorectal cancer (CRC): CRC #4 Case n=40, Control n=40; CRC #5 Case n=61, Control n=52; CRC #6 Case n=52, Control n=52; 1 on Graves' disease (GD), Case n=88, Control n=62, 2 on hypertension (HT): HT #1 Case n=60, Control n=56, HT #2 Case n=99, Control n=41; 1 on multiple sclerosis (MS): Case n=24, Control n=24, 3 on pancreatic cancer (PC): PC #1 Case n=43, Control n=235, PC #2 Case n=57, Control n=50, PC #3 Case n=44, Control n=32; and 1 on Parkinson's disease (PD): Case n=39, Control n=40. Random Forest classification model with leave-one-out cross validation was applied in each dataset. (C) The Combined Core genomes were used as predictors in Treatment Dataset Collection to predict responder (R) and non-responder (NR) under a treatment. For inflammatory bowel disease (IBD), 14-week remission was used to determine R and NR of patients with IBD to anti-cytokine or anti-antigen treatment. IBD anti-cytokine: R n=29, NR n=18; IBD_anti-integrin #1, R n=27, NR n=40; IBD_anti-integrin #2: R n=29, NR n=53. For rheumatoid arthritis (RA), responder to methotrexate (MTX) was defined a priori as any patient with new-onset RA with an improvement in the Disease Activity Score in 28 joints (DAS28) of >1.8 by month 4 after initiation of MTX monotherapy. R n=19, NR n=28. For advanced melanoma, progression-free survival was used to determined R and NR to immune checkpoint inhibitor (ICI) treatment. AM_ICI #1: R n=4, NR n=7, AM_ICI #2: Rn=10, NR n=8, AM_ICI #3: R n=12, NR n=13, AM_ICI #4: R n=25, NR n=30, AM_ICI #5: R n=26, NR n=28. For B cell lymphoma, tumor response to CAR-T cell immunotherapy was classified as either complete remission or non-complete remission (partial remission, stable disease, progressive disease or death) at 180 days after CAR-T cell infusion by the treating physician. Model was trained on German cohort and validated by US cohort. Germany: R n=21, NR n=29; US: R n=21, NR n=24.

FIGS. 9A, 9B, and 9C collectively illustrate the discriminative power of the combined core genomes from all the 8 sets of the two competing guilds in classifying healthy individuals vs. patients across colorectal cancer (CRC), inflammatory bowel diseases (IBD), and Pancreatic Cancer (PC) datasets in the Case-Control Dataset Collection I and II. A prediction matrix was shown for the classification of cases and controls based on the combined core genomes from all eight sets of the two competing guilds within each dataset (diagonal values), across pairs of datasets (one dataset used for model training and the other for testing), and in a leave-one-dataset-out setting (training the model on all but one dataset and testing on the left-out dataset). Random Forest classification model with leave-one-out cross validation was applied. The area under the ROC curve (AUC) values were shown in the matrix. (A) CRC. #1: case n=74, control n=54; #2: case n=46, control n=63; #3: case n=22, control n=60; #4: case n=40, control n=40; #5: case n=61, control n=52; #6: case n=52, control n=52. (B) IBD. #1: case n=80 control n=26; #2: case n=121, control n=34; #3: case n=43, control n=22; (C) PC. #1: case n=43 control n=235; #2: case n=57 control n=50; #3: case n=44 control n=32.

FIGS. 10A1, 10A2, 10B1, 10B2, 10C1, 10C2, 10D1, and 10D2 collectively illustrate the combined core of the two competing guilds supports the prediction of therapeutic effects in the Treatment Dataset Collection for inflammatory bowel diseases, rheumatoid arthritis, advanced melanoma, and B cell lymphoma. The abundance of the combined core genomes (284 HQMAGs) in the pre-treatment samples were used as predictors in Random Forest classification models to predict responder (R) and non-responder (NR) under treatment. Area under the ROC curve (AUC) and AUC values were showed in the panels. (A) 14-week remission was used to determine R and NR. IBD_anti-cytokine, R n=29, NR n=18; IBD_anti-integrin #1, R n=27, NR n=40; IBD_anti-integrin #2, R n=29, NR n=53. (B) Responder to MTX was defined a priori as any patient with new-onset RA with an improvement in the Disease Activity Score in 28 joints (DAS28) (25) of >1.8 by month 4 after initiation of MTX monotherapy. R n=19, NR n=28. (C) Overall response Rate (ORR, left matrix) and progression-free survival (PFS12, right matrix) was used to determined R and NR, respectively. Prediction matrix for microbiome-based prediction of response assessed via ORR (left matrix) and PFS12 (right matrix) within each cohort (values on the diagonal), across pairs of cohorts (one cohort used to train the model and the other for testing) and in the leave-one-cohort-out setting (training the model on all but one cohort and testing on the left-out cohort). ORR: R n=94, NR n=71; PFS12: R n=77, NR n=86. (D) Tumor response to CAR-T cell immunotherapy was classified as either complete remission or non-complete remission (partial remission, stable disease, progressive disease or death) at 180 days after CAR-T cell infusion by the treating physician. Model was trained on #1 (German cohort) and validated by #2 (US cohort). #1: R n=21, NR n=29; #2: R n=21, NR n=24.

FIGS. 11A, 11A2, 11B1, 11B2, 11C1, 11C2, 11D1, and 11D2 collectively illustrate the Combined Core genomes of the two competing guilds provide a universal model for distinguishing between cases and controls across a variety of diseases (Case-Control Dataset Collection I and II). (A) All control and case samples from Case-Control Dataset Collection I and II, encompassing a total of 26 datasets on 15 different diseases, were combined and randomly allocated, with 80% used for training a Random Forest classification model and 20% for testing. (B) ROC curve and the area under the ROC curve (AUC). (C) The density plot of the probability score of between case and control. The probability score was generated from the Random Forest classification model and showed the probability of one sample to be predicted as case. (D) Box plot of the probability score between control and case samples. Mann-Whitney test was applied. *** P<0.001. Training: control n=1,285, case n=1424; testing: control n=319, case n=356.

FIGS. 12A, 12B, 12C, 12D, 12E, 12F, 12G, 12H, and 12I collectively illustrate the corresponding contigs, referenced by SEQ IDs, obtained for each of the 788 genomes.

FIGS. 13A, 13B, 13C, 13D, 13E, 13F, 13G, 13H, 13I, 13J, 13K, 13L, 13M, 13N, 130, 13P, 13Q, 13R, 13S, 13T, 13U, 13V, 13W, 13X, 13Y, 13Z, 13AA, 13BB, 13CC, 13DD, 13EE, 13FF, 13GG, 13HH, 13II, 13JJ, 13KK, 13LL, 13MM, 13NN, 1300, 13PP, 13QQ, 13RR, 13SS, 13TT, 13UU, 13VV, 13WW and 13XX collectively illustrate the Taxonomy Assignment of 788 combined microbiome.

FIGS. 14A and 14B collectively illustrate genome pairwise ANI comparison. FIG. 14A depicts all genome pairwise ANI comparison among the 788 combined pool of genomes. FIG. 14B depicts the pairwise ANI comparison between Guild 1 genomes and Guild 2 genomes.

FIGS. 15A and 15B collectively illustrate the capacity of the combined pool to classify case and control across different studies. The eight sets of signature microbiome obtained from QD and various diseases cases: T2D, LC, SCZ, IBD, AS, ACVD, CRC were pooled together as a combined microbiome signature. FIG. 15A shows the comparison of classification performance of the combined pool with each of the individual signature microbiome based on AUC values. FIG. 15B shows the significance of intra-group comparison. Friedman test followed by Dunn's post hoc was performed for the analysis (#BH adjusted P<0.1, * BH adjusted P<0.05).

FIGS. 16A, 16B and 16C collectively illustrate the rank of the classification performance of the microbiome signature. The nine sets of microbiome signature obtained from combined pool, QD or various diseases cases: T2D, LC, SCZ, IBD, AS, ACVD, CRC were ranked according to their performance in classifying case and control across 11 datasets. All the ranking numbers assigned to each set of signature microbiome are plotted FIG. 16A. FIG. 16B shows the significance of intra-group comparison. FIG. 16C shows the sum of the ranks for each set of microbiome signatures. Kruskal-Wallis test followed by Dunn's post hoc was performed for the analysis (#BH adjusted P<0.1, * BH adjusted P<0.05). The microbiome signature obtained from the combined pool has the best performance to classify the healthy subjects vs. patients across different datasets.

FIG. 17 illustrates the selection of the combined core pool. Random Forest classification based on a combined 788 genomes are performed for each dataset. Each of the 788 genome was ranked based on its importance. A summed rank was obtained by adding up the value of ranks across 11 datasets all 788 genomes are ranked again based on the summed value. The most important genome across 11 dataset gets the lowest summed rank value. Starting from the least important genome, every genome one by one was removed from each dataset based on order of importance. The classification performance (AUCs) was calculated for the remaining numbers of genomes after each removal by Random Forest model and all the genome numbers are ranked based on AUC values. The rank values for each genome number across 11 datasets was summed. The sum of ranks for each genome number across 11 datasets was plotted. 302 genomes achieved lowest summed AUC ranks. After removing 18 genomes which exhibit inconsistent C1A and C1B assignment, 284 genomes remained as the combined core pool.

FIGS. 18A, 18B, 18C, 18D, 18E, 18F, 18G, 18H, 18I, and 18J collectively illustrate the classification capacity of the two competing guilds identified from QD, various types of diseases, combined pool, and combined core pool. Microbiome signature comprising the genomes of two competing guilds were obtained from various disease: T2D (FIG. 18A), LC (FIG. 18B), AS (FIG. 18C), CRC (FIG. 18D), IBD (FIG. 18E), QD (FIG. 18F), AVCD (FIG. 18G), SCZ (FIG. 18H), combined pool (FIG. 18I), and combined core pool (FIG. 18J). The identified microbiome signature for each condition was utilized to classify control and patients in each dataset using Random Forest classifiers. FIG. 31 shows all microbiome signature have the capacity to classify case and control across different studies.

FIG. 19 illustrates combined case and control sampales from the 25 datasets that corresponded to 15 various diseases (type-2 diabetes (T2D), hypertension (HT), schizophrenia (SCZ), atherosclerotic cardiovascular disease (ACVD), liver cirrhosis (LC), inflammatory bowel diseases (IBD), colorectal cancer (CRC), ankylosing spondylitis (AS), Parkinson's disease (PD), Multiple Sclerosis (MS), Gaucher disease type II (GDII), COVID-19 (COV), Behcet's disease (BD), autism spectrum disorder (ASD), and pancreatic cancer (PC).

FIGS. 20A1, 20A2, 20A3, 20B1, 20B2, and 20B3 collectively illustrate the Universal Random Forest classification model for case vs control based on the abundance of the 284 core genomes. A: 80% training: Control, n=1285; Case, n=1424, 10-fold CV (A1: The area under the ROC curve (AUC) of the Random Forest classifier; A2: Score density for case and control; A3: Probability score for case and control); B: 20% testing: Control, n=319; Case, n=356 (B1: The area under the ROC curve (AUC) of the Random Forest classifier; B2: Score density for case and control; B3: probability score for case and control).

FIGS. 21A and 21B collectively illustrate the repeated training of Universal Random Forest classification model for case vs control with randomly selected number of genomes. (A) Each data point represents average AUC for a Random Forest model trained ten times using a different set of randomly selected genomes at a total number of X (as indicated by the X-axis) determined against the training set. (B) Each data point represents average AUC for a Random Forest model trained ten times using a different set of randomly selected genomes at a total number of X (as indicated by the X-axis) determined against a testing set.

Like reference numerals refer to corresponding parts throughout the several views of the drawings.

### DETAILED DESCRIPTION

The methods and systems described herein facilitate prediction of a subject's response to a therapy for a disorder based on the constitution of the subject's microbiome.

### Definitions

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “comprising,” or any variation thereof, when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As used herein, the term “measure of central tendency” refers to a central or representative value for a distribution of values. Non-limiting examples of measures of central tendency include an arithmetic mean, weighted mean, midrange, midhinge, trimean, geometric mean, geometric median, Winsorized mean, median, and mode of the distribution of values.

As used herein, the term “subject” refers to any living or non-living organism including, but not limited to, a human (e.g., a male human, female human, fetus, pregnant female, child, or the like), a non-human mammal, or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a woman, or a child).

As used herein, the term “administering” with respect to the methods of the invention, means a method for therapeutically or prophylactically preventing, treating or ameliorating a syndrome, disorder or disease as described herein. Such methods include administering an effective amount of said therapeutic agent at different times during the course of a therapy or concurrently in a combination form. The methods of the invention are to be understood as embracing all known therapeutic treatment regimens.

As used herein, the term “cancer,” “cancerous tissue,” or “tumor” refers to an abnormal mass of tissue in which the growth of the mass surpasses, and is not coordinated with, the growth of normal tissue, including both solid masses (e.g., as in a solid tumor) or fluid masses (e.g., as in a hematological cancer). A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue. Accordingly, a “tumor sample” refers to a biological sample obtained or derived from a tumor of a subject, as described herein.

Non-limiting examples of cancer types include ovarian cancer, cervical cancer, uveal melanoma, colorectal cancer, chromophobe renal cell carcinoma, liver cancer, endocrine tumor, oropharyngeal cancer, retinoblastoma, biliary cancer, adrenal cancer, neural cancer, neuroblastoma, basal cell carcinoma, brain cancer, breast cancer, non-clear cell renal cell carcinoma, glioblastoma, glioma, kidney cancer, gastrointestinal stromal tumor, medulloblastoma, bladder cancer, gastric cancer, bone cancer, non-small cell lung cancer, thymoma, prostate cancer, clear cell renal cell carcinoma, skin cancer, thyroid cancer, sarcoma, testicular cancer, head and neck cancer (e.g., head and neck squamous cell carcinoma), meningioma, peritoneal cancer, endometrial cancer, pancreatic cancer, mesothelioma, esophageal cancer, small cell lung cancer, Her2 negative breast cancer, ovarian serous carcinoma, HR+ breast cancer, uterine serous carcinoma, uterine corpus endometrial carcinoma, gastroesophageal junction adenocarcinoma, gallbladder cancer, chordoma, and papillary renal cell carcinoma.

As used herein, the terms “cancer state” or “cancer condition” refer to a characteristic of a cancer patient's condition, e.g., a diagnostic status, a type of cancer, a location of cancer, a primary origin of a cancer, a cancer stage, a cancer prognosis, and/or one or more additional characteristics of a cancer (e.g., tumor characteristics such as morphology, heterogeneity, size, etc.). In some embodiments, one or more additional personal characteristics of the subject are used further describe the cancer state or cancer condition of the subject, e.g., age, gender, weight, race, personal habits (e.g., smoking, drinking, diet), other pertinent medical conditions (e.g., high blood pressure, dry skin, other diseases), current medications, allergies, pertinent medical history, current side effects of cancer treatments and other medications, etc.

As used herein, the term “treat”, “treating”, “treatment”, or “therapy”, refers to both therapeutic treatment and prophylactic or preventative measures, wherein the object is to prevent or slow down (lessen) the targeted pathologic condition or disorder. Those in need of treatment include those diagnosed with the disorder as well as those prone to have the disorder (e.g., a genetic predisposition) or those in whom the disorder is to be prevented. The terms “prevent,” “preventing,” and “prevention” refer to reducing the likelihood of the onset (or recurrence) of a disease, disorder, condition, or associated symptom(s). The term means obtaining beneficial or desired results, for example, clinical results. Beneficial or desired results can include, but are not limited to, alleviation of one or more symptoms. The term “alleviation”, for example in reference to a symptom of a condition, as used herein, refers to reducing at least one of the frequency and amplitude of a symptom of a condition in a patient.

As used herein, the “response”, as used herein, refers to the response to a biological drug, chemical drug, or physical therapy of the subject suffering from a pathology which is treatable with said biological drug, chemical drug, or physical therapy. Standard criteria may vary from disease to disease.

As used herein, “immunotherapies” are all therapies that either directly or indirectly modify the immune response or the immune system of a patient. For immunotherapeutic strategies, it has been found that the detection of a strong immune response at the tumor site was a reliable marker for a plurality of cancers, like colon cancers as well as rectum cancers, this association of a pre-existing immune response with a better therapeutic efficacy was assumed. Immune response encompasses any form of immune response of said patient through direct or indirect, or both, action towards said cancer or tumor sites. The immune response means the immune response of the host cancer patient in reaction to the tumor and encompasses the presence of, the number of, or alternatively the activity of, cells and related signaling molecules involved in the immune response of the host which includes: all cytokines, chemokines, growth factors, stem cell growth factors. In some embodiments, the immune response encompasses a multitude of different cellular subtypes, such as T cell lineage, the B cell lineage, the natural killer cells, macrophages, dendritic cells, myelo-derived suppressor cells, lytic dendritic cells, fibroblasts, endothelial cells, as well as an enormous number of signaling molecules (cytokines, chemokines, other signaling molecules).

As used herein, “immunotherapeutic agent,” refers to a compound, composition or treatment that indirectly or directly enhances, stimulates, or augments the body's immune response against cancer cells and/or that lessens the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies, and non-cytokine adjuvants. Alternatively the immunotherapeutic treatment may consist of administering the patient with an amount of immune cells (T cells, NK, cells, dendritic cells, B cells . . . ).

Immunotherapeutic agents can be non-specific, i.e. boost the immune system generally so that it becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly augment the immune system. Non-specific immunotherapeutic agents have been used alone as the main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors.

Interferons (IFNs) contemplated by the present invention include the common types of IFNs, IFN-alpha (IFN-a), IFN-beta (IFN-beta) and IFN-gamma (IFN-y). IFNs can act directly on cancer cells, for example, by slowing their growth, promoting their development into cells with more normal behavior and/or increasing their production of antigens thus making the cancer cells easier for the immune system to recognize and destroy. IFNs can also act indirectly on cancer cells, for example, by slowing down angiogenesis, boosting the immune system and/or stimulating natural killer (NK) cells, T cells and macrophages. Recombinant IFN-alpa is available commercially as Roferon (Roche Pharmaceuticals) and Intron A (Schering Corporation). The use of IFN-alpha, alone or in combination with other immunotherapeutics or with chemotherapeutics, has shown efficacy in the treatment of various cancers including melanoma (including metastatic melanoma), renal cancer (including metastatic renal cancer), breast cancer, prostate cancer, and cervical cancer (including metastatic cervical cancer).

Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Interleukins, alone or in combination with other immunotherapeutics or with chemotherapeutics, have shown efficacy in the treatment of various cancers including renal cancer (including metastatic renal cancer), melanoma (including metastatic melanoma), ovarian cancer (including recurrent ovarian cancer), cervical cancer (including metastatic cervical cancer), breast cancer, colorectal cancer, lung cancer, brain cancer, and prostate cancer. Interleukins have also shown good activity in combination with IFN-α in the treatment of various cancers (Negrier et al., Ann Oncol. 2002 13(9):1460-8; Touranietal, J Clin Oncol. 2003 21(21):398794).

Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in patients undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Colony stimulating factors have shown efficacy in the treatment of cancer, including melanoma, colorectal cancer (including metastatic colorectal cancer), and lung cancer.

Non-cytokine adjuvants suitable for use in the combinations of the present invention include, but are not limited to, Levamisole, alum hydroxide (alum), bacillus Calmette-Guerin (ACG), incomplete Freund's Adjuvant (IFA), QS-21, DETOX, Keyhole limpet hemocyanin (KLH) and dinitrophenyl (DNP). Non-cytokine adjuvants in combination with other immuno- and/or chemotherapeutics have demonstrated efficacy against various cancers including, for example, colon cancer and colorectal cancer (Levimasole); melanoma (BCG and QS-21); renal cancer and bladder cancer (BCG).

In addition to having specific or non-specific targets, immunotherapeutic agents can be active, i.e. stimulate the body's own immune response, or they can be passive, i.e. comprise immune system components that were generated external to the body.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Monoclonal antibodies are used in the treatment of a wide range of cancers including breast cancer (including advanced metastatic breast cancer), colorectal cancer (including advanced and/or metastatic colorectal cancer), ovarian cancer, lung cancer, prostate cancer, cervical cancer, melanoma and brain tumours.

Other examples include antibodies specific a co-stimulatory molecule. Co-stimulatory molecules include, for example B7-1/CD80, CD28, B7-2/CD86, CTLA-4, B7-H1/PD-L1, Gi24/Dies 1/VISTA, B7-H2, ICOS, B7-H3 PD-1, B7-H4, PD-L2/B7-DC, B7-H6, PDCD6, BTLA, 4-1 BB/TNFRSF9/CD137, CD40 Ligand/TNFSF5, 4-1BB Ligand/TNFSF9 GITR/TNFRSF18, HVEM/TNFRSF14, CD27/TNFRSF7, LIGHT/TNFSF14, CD27 Ligand/TNFSF7, OX40/TNFRSF4, CD30/TNFRSF8, 0X40 Ligand/TNFSF4, CD30 Ligand/TNFSF8, TACI/TNFRSF13B, CD40/TNFRSF5, 2B4/CD244/SLAMF4 CD84/SLAMF5, BLAME/SLAMF8, CD229/SLAMF3, CD2CRACC/SLAMF7, CD2F-10/SLAMF9 NTB-A/SLAMF6, CD48/SLAMF2, SLAM/CD 150, CD58/LFA-3, CD2 Ikaros, CD53 Integrin alpha 4/CD49d, CD82/Kai-1 Integrin alpha 4 beta 1, CD90/Thyl Integrin alpha 4 beta 7/LPAM-1, CD96 LAG-3, CD160 LMIR1/CD300A, CRTAM TCL1A, DAP 12 TCL1B, Dectin-1/CLEC7A TIM-1/KIM-1/HAVCR, DPPIV/CD26 TIM-4, EphB6 TSLP, HLA Class I TSLP R, HLA-DR.

In particular, the antibody is selected from the group consisting of anti-CTLA4 antibodies (e.g. Ipilimumab), anti-PD1 antibodies, anti-PDL1 antibodies, anti-TIMP3 antibodies, anti-LAG3 antibodies, anti-B7H3 antibodies, anti-B7H4 antibodies anti-TREM antibodies, anti-BTLA antibodies, anti-LIGHT antibodies or anti-B7H6 antibodies.

Monoclonal antibodies can be used alone or in combination with other immunotherapeutic agents or chemotherapeutic agents.

Active specific immunotherapy typically involves the use of cancer vaccines. Cancer vaccines have been developed that comprise whole cancer cells, parts of cancer cells or one or more antigens derived from cancer cells. Cancer vaccines, alone or in combination with one or more immuno- or chemotherapeutic agents are being investigated in the treatment of several types of cancer including melanoma, renal cancer, ovarian cancer, breast cancer, colorectal cancer, and lung cancer. Non-specific immunotherapeutics are useful in combination with cancer vaccines in order to enhance the body's immune response.

The immunotherapeutic treatment may consist of an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the patient's circulating lymphocytes, or tumor infiltrated lymphocytes, are isolated in vitro, activated by lymphokines such as IL-2 or transuded with genes for tumor necrosis, and readministered (Rosenberg et al., 1988; 1989). The activated lymphocytes are most preferably the patient's own cells that were earlier isolated from a blood or tumor sample and activated (or “expanded”) in vitro. This form of immunotherapy has produced several cases of regression of melanoma and renal carcinoma.

As used herein, the term “genomic abundance value” refers to an absolute or relative amount of a microorganism's genome in a biological sample from the gut of a subject. A genomic abundance value can be expressed different units, including copy number, molarity, mass (e.g., normalized against the size of the genome), unique sequence reads (e.g., normalized against the size of the genome), a percentage of any of the former metrics relative to the total amount of the metric across all genomes in the sample, a percentage of any of the former metrics relative to the total amount of the metric across a plurality of genomes in the sample, etc. In some embodiments, a genomic abundance value is normalized against a total genomic abundance in the sample. In some embodiments, a genomic abundance value is normalized against a genomic abundance value for a control genome in the sample. In some embodiments, the values for a plurality of genomic abundance values in a sample are standardized, normalized, and/or scaled. Examples of methods for normalizing genomic abundance values are described, for example, in Lin, H., Peddada, S. D., Analysis of microbial compositions: a review of normalization and differential abundance analysis, Biofilms Microbiomes, 6(60) (2020) and Lutz K. C., et al., A Survey of Statistical Methods for Microbiome Data Analysis, Frontiers in Applied Mathematics and Statistics, 8 (2022) the contents of which are incorporated herein by reference in their entireties. Methods for measuring genomic abundance values are known in the art. For example, metagenomic sequencing can be used to largely reconstruct microbial genomes from next generation sequencing of genomic DNA in biological samples, such as biological samples from the gut of a subject. For a review of metagenomic sequence see, for example, Quince C, et al., Shotgun metagenomics, from sampling to analysis, Nat Biotechnol, 35 (9): 833-44 (2017), the content of which is incorporated herein by reference in its entirety. Genomic abundance may also be determined by quantification of the copy number of a ribosomal gene, for example the 16S rRNA gene. Examples of rRNA quantification are described in Manzari C., et al., Accurate quantification of bacterial abundance in metagenomic DNAs accounting for variable DNA integrity levels, Microb Genom., 6(10):mgen000417 (2020) and Barlow, J. T., et al., A quantitative sequencing framework for absolute abundance measurements of mucosal and lumenal microbial communities, Nat Commun., 11:2590 (2020), the contents of which are incorporated herein by reference in their entireties.

As used herein, the term “relative abundance” refers to a ratio of a first amount of a compound measured in a sample, e.g., a genome for a first microorganism, to a second amount of a compound measured in a second sample. In some embodiments, relative abundance refers to a ratio of an amount of a compound, e.g., a genome for a first microorganism, to a total amount of compounds, e,g., the total amount of microorganism genomes or the total amount of a plurality of genomes, in the same sample. In other embodiments, relative abundance refers to a ratio of an amount of a compound, e.g., a genome for a first microorganism, in a first sample to an amount of the compound of the compound in a second sample. For instance, a ratio of a normalized amount of a genome for a first microorganism in a first sample to a normalized amount of the genome for the first microorganism in a second and/or reference sample.

As used herein, the terms “sequencing,” “sequence determination,” and the like refer to any biochemical processes that may be used to determine the order of biological macromolecules such as nucleic acids or proteins. For example, sequencing data can include all or a portion of the nucleotide bases in a nucleic acid molecule such as an mRNA transcript or a genomic locus.

As used herein, the term “sequence reads” or “reads” refers to nucleotide sequences produced by any nucleic acid sequencing process described herein or known in the art. Reads can be generated from one end of nucleic acid fragments (“single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore® sequencing, for example, can provide sequence reads that can vary in size from tens to hundreds to thousands of base pairs. Illumina® parallel sequencing, for example, can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “read segment” refers to any form of nucleotide sequence read including the raw sequence reads obtained directly from a nucleic acid sequencing technique or from a sequence derived therefrom, e.g., an aligned sequence read, a collapsed sequence read, or a stitched sequence read.

As used herein, the term “read count” refers to the total number of nucleic acid reads generated, which may or may not be equivalent to the number of nucleic acid molecules generated, during a nucleic acid sequencing reaction.

As used herein, the term “read-depth,” “sequencing depth,” or “depth” can refer to a total number of unique nucleic acid fragments encompassing a particular locus or region of the genome of a microorganism that are sequenced in a particular sequencing reaction. Sequencing depth can be expressed as “Yx”, e.g., 50×, 100×, etc., where “Y” refers to the number of unique nucleic acid fragments encompassing a particular locus that are sequenced in a sequencing reaction. In such a case, Y is necessarily an integer, because it represents the actual sequencing depth for a particular locus. Alternatively, read-depth, sequencing depth, or depth can refer to a measure of central tendency (e.g., a mean or mode) of the number of unique nucleic acid fragments that encompass one of a plurality of loci or regions of the genome of a microorganism that are sequenced in a particular sequencing reaction. For example, in some embodiments, sequencing depth refers to the average depth of every locus across a targeted sequencing panel, an exome, or an entire genome for the microorganism. In such case, Y may be expressed as a fraction or a decimal, because it refers to an average coverage across a plurality of loci. When a mean depth is recited, the actual depth for any particular locus may be different than the overall recited depth. Metrics can be determined that provide a range of sequencing depths in which a defined percentage of the total number of loci fall. For instance, a range of sequencing depths within which 90% or 95%, or 99% of the loci fall. As understood by the skilled artisan, different sequencing technologies provide different sequencing depths. For instance, low-pass whole genome sequencing can refer to technologies that provide a sequencing depth of less than 5×, less than 4×, less than 3×, or less than 2×, e.g., from about 0.5× to about 3×.

As used herein, the term “sequencing breadth” refers to what fraction of a particular microorganism genome has been sequenced. Sequencing breadth can be expressed as a fraction, a decimal, or a percentage, and is generally calculated as (the number of loci analyzed/the total number of loci in the genome). The denominator of the fraction can be a repeat-masked genome, and thus 100% can correspond to all of the reference genome minus the masked parts. A repeat-masked genome can refer to a genome in which sequence repeats are masked (e.g., sequence reads align to unmasked portions of the genome). In some embodiments, any part of a genome can be masked and, thus, sequencing breadth can be evaluated for any desired portion of a genome.

As used herein, the terms “sequence ratio” and “coverage ratio” interchangeably refer to any measurement of a number of units of a genomic sequence in a first one or more biological samples (e.g., a test and/or tumor sample) compared to the number of units of the respective genomic sequence in a second one or more biological samples (e.g., a reference and/or control sample). In some embodiments, a sequence ratio is a copy ratio, a log 2-transformed copy ratio (e.g., log 2 copy ratio), a coverage ratio, a base fraction, an allele fraction (e.g., a variant allele fraction), and/or a tumor ploidy. In some embodiments sequence ratio is a logN-transformed copy ratio, where N is any real number greater than 1.

As used herein, the term “sequencing probe” refers to a molecule that binds to a nucleic acid with affinity that is based on the expected nucleotide sequence of the RNA or DNA present at that locus.

As used herein, the term “targeted panel” or “targeted gene panel” refers to a combination of probes for sequencing (e.g., by next-generation sequencing) nucleic acids present in a biological sample from a subject (e.g., a tumor sample, liquid biopsy sample, germline tissue sample, white blood cell sample, or tumor or tissue organoid sample), selected to map to one or more loci of interest in a genome.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having a particular biological characteristic.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having a particular biological characteristic.

As used interchangeably herein, the term “classifier” or “model” refers to a machine learning model or algorithm.

In some embodiments, a model includes an unsupervised learning algorithm. One example of an unsupervised learning algorithm is cluster analysis. In some embodiments, a model includes supervised machine learning. Nonlimiting examples of supervised learning algorithms include, but are not limited to, logistic regression, neural networks, support vector machines, Naïve Bayes algorithms, nearest neighbor algorithms, random forest algorithms, decision tree algorithms, boosted trees algorithms, multinomial logistic regression algorithms, linear models, linear regression, Gradient Boosting, mixture models, hidden Markov models, Gaussian NB algorithms, linear discriminant analysis, diffusion models, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level model).

Neural networks. In some embodiments, the model is a neural network (e.g., a convolutional neural network and/or a residual neural network). Neural network algorithms, also known as artificial neural networks (ANNs), include convolutional and/or residual neural network algorithms (deep learning algorithms). In some embodiments, neural networks are machine learning algorithms that are trained to map an input dataset to an output dataset, where the neural network includes an interconnected group of nodes organized into multiple layers of nodes. For example, in some embodiments, the neural network architecture includes at least an input layer, one or more hidden layers, and an output layer. In some embodiments, the neural network includes any total number of layers, and any number of hidden layers, where the hidden layers function as trainable feature extractors that allow mapping of a set of input data to an output value or set of output values. In some embodiments, a deep learning algorithm is a neural network including a plurality of hidden layers, e.g., two or more hidden layers. In some instances, each layer of the neural network includes a number of nodes (or “neurons”). In some embodiments, a node receives input that comes either directly from the input data or the output of nodes in previous layers, and performs a specific operation, e.g., a summation operation. In some embodiments, a connection from an input to a node is associated with a parameter (e.g., a weight and/or weighting factor). In some embodiments, the node sums up the products of all pairs of inputs, xi, and their associated parameters. In some embodiments, the weighted sum is offset with a bias, b. In some embodiments, the output of a node or neuron is gated using a threshold or activation function, f, which, in some instances, is a linear or non-linear function. In some embodiments, the activation function is, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

In some implementations, the weighting factors, bias values, and threshold values, or other computational parameters of the neural network, are “taught” or “learned” in a training phase using one or more sets of training data. For example, in some implementations, the parameters are trained using the input data from a training dataset and a gradient descent or backward propagation method so that the output value(s) that the ANN computes are consistent with the examples included in the training dataset. In some embodiments, the parameters are obtained from a back propagation neural network training process.

Any of a variety of neural networks are suitable for use in accordance with the present disclosure. Examples include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. In some implementations, convolutional and/or residual neural networks are used, in accordance with the present disclosure.

For instance, a deep neural network model includes an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 50 parameters, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the model is a support vector machine (SVM). SVM algorithms suitable for use as models are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For certain cases in which no linear separation is possible, SVMs work in combination with the technique of ‘kernels’, which automatically realizes a non-linear mapping to a feature space. The hyper-plane found by the SVM in feature space corresponds, in some instances, to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms. In some embodiments, the model is a Naïve Bayes algorithm. Naïve Bayes models suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naïve Bayes model is any model in a family of “probabilistic models” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a model is a nearest neighbor algorithm. In some implementations, nearest neighbor models are memory-based and include no model to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. In some embodiments, Euclidean distance in feature space is used to determine distance as d₍ᵢ₎=∥x₍ᵢ₎−x₍₀₎∥. Typically, when the nearest neighbor algorithm is used, the abundance data used to compute the linear discriminant is standardized to have mean zero and variance 1. In some embodiments, the nearest neighbor rule is refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor model is such that a computer is used to solve the model for a given input because it cannot be mentally performed.

Random forest, decision tree, and boosted tree algorithms. In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. For example, one specific algorithm is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

Regression. In some embodiments, the model uses a regression algorithm. In some embodiments, a regression algorithm is any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the model makes use of a regression model disclosed in Hastie et al., 2001, The Elements of Statistical Learning, Springer-Verlag, New York. In some embodiments, the logistic regression model includes at least 10, at least 20, at least 50, at least 100, or at least 1000 parameters (e.g., weights) and requires a computer to calculate because it cannot be mentally solved.

Linear discriminant analysis algorithms. In some embodiments, linear discriminant analysis (LDA), normal discriminant analysis (NDA), or discriminant function analysis is a generalization of Fisher's linear discriminant, a method used in statistics, pattern recognition, and machine learning to find a linear combination of features that characterizes or separates two or more classes of objects or events. In some embodiments, the resulting combination is used as the model (linear model) in some embodiments of the present disclosure.

Mixture model and Hidden Markov model. In some embodiments, the model is a mixture model, such as that described in McLachlan et al., Bioinformatics 18(3):413-422, 2002. In some embodiments, in particular, those embodiments including a temporal component, the model is a hidden Markov model such as described by Schliep et al., 2003, Bioinformatics 19(1):1255-i263.

Clustering. In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. As an illustrative example, in some embodiments, the clustering problem is described as one of finding natural groupings in a dataset. To identify natural groupings, two issues are addressed. First, a way to measure similarity (or dissimilarity) between two samples is determined. This metric (e.g., similarity measure) is used to ensure that the samples in one cluster are more like one another than they are to samples in other clusters. Second, a mechanism for partitioning the data into clusters using the similarity measure is determined. One way to begin a clustering investigation is to define a distance function and to compute the matrix of distances between all pairs of samples in the training set. If distance is a good measure of similarity, then the distance between reference entities in the same cluster is significantly less than the distance between the reference entities in different clusters. However, in some implementations, clustering does not use a distance metric. For example, in some embodiments, a nonmetric similarity function s(x, x′) is used to compare two vectors x and x′. In some such embodiments, s(x, x′) is a symmetric function whose value is large when x and x′ are somehow “similar.” Once a method for measuring “similarity” or “dissimilarity” between points in a dataset has been selected, clustering uses a criterion function that measures the clustering quality of any partition of the data. Partitions of the dataset that extremize the criterion function are used to cluster the data. Particular exemplary clustering techniques contemplated for use in the present disclosure include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering includes unsupervised clustering (e.g., with no preconceived number of clusters and/or no predetermination of cluster assignments).

Ensembles of models and boosting. In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n>40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

As used herein, the term “untrained model” (e.g., “untrained classifier” and/or “untrained neural network”) refers to a machine learning model or algorithm, such as a classifier or a neural network, that has not been trained on a target dataset. In some embodiments, “training a model” (e.g., “training a neural network”) refers to the process of training an untrained or partially trained model (e.g., “an untrained or partially trained neural network”). Moreover, it will be appreciated that the term “untrained model” does not exclude the possibility that transfer learning techniques are used in such training of the untrained or partially trained model. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained model described above is provided with additional data over and beyond that of the primary training dataset. Typically, this additional data is in the form of parameters (e.g., coefficients, weights, and/or hyperparameters) that were learned from another, auxiliary training dataset. Moreover, while a description of a single auxiliary training dataset has been disclosed, it will be appreciated that there is no limit on the number of auxiliary training datasets that can be used to complement the primary training dataset in training the untrained model in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning is used, in some such embodiments. For instance, consider the case where there is a first auxiliary training dataset and a second auxiliary training dataset in addition to the primary training dataset. In such a case, the parameters learned from the first auxiliary training dataset (by application of a first model to the first auxiliary training dataset) are applied to the second auxiliary training dataset using transfer learning techniques (e.g., a second model that is the same or different from the first model), which in turn results in a trained intermediate model whose parameters are then applied to the primary training dataset and this, in conjunction with the primary training dataset itself, is applied to the untrained model. Alternatively, in another example embodiment, a first set of parameters learned from the first auxiliary training dataset (by application of a first model to the first auxiliary training dataset) and a second set of parameters learned from the second auxiliary training dataset (by application of a second model that is the same or different from the first model to the second auxiliary training dataset) are each individually applied to a separate instance of the primary training dataset (e.g., by separate independent matrix multiplications) and both such applications of the parameters to separate instances of the primary training dataset in conjunction with the primary training dataset itself (or some reduced form of the primary training dataset such as principal components or regression coefficients learned from the primary training set) are then applied to the untrained model in order to train the untrained model.

As used herein, the term “AUC” refers to the Area Under the Curve, for example, of a ROC Curve. That value can assess the merit of a test on a given sample population with a value of 1 representing a good test ranging down to 0.5 which means the test is providing a random response in classifying test subjects. Since the range of the AUC is only 0.5 to 1.0, a small change in AUC has greater significance than a similar change in a metric that ranges for 0 to 1 or 0 to 100%. When the % change in the AUC is given, it will be calculated based on the fact that the full range of the metric is 0.5 to 1.0. A variety of statistics packages can calculate AUC for an ROC curve. AUC can be used to compare the accuracy of the classification algorithm across the complete data range. Classification algorithms with greater AUC have, by definition, a greater capacity to classify unknowns correctly between the two groups of interest (disease and no disease, responder and non-responder).

As used herein, the term “instruction” refers to an order given to a computer processor by a computer program. On a digital computer, each instruction is a sequence of Os and Is that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal instruction set computers (MISC), Very long instruction word (VLIW), Explicitly parallel instruction computing (EPIC), and One instruction set computer (OISC).

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Example System Embodiments

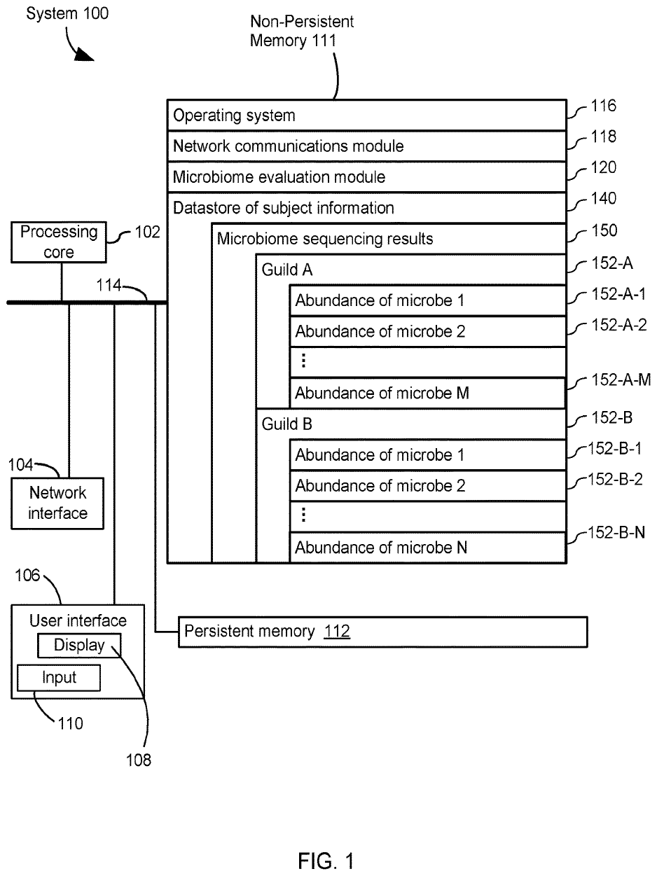

Now that an overview of some aspects of the present disclosure and some definitions used in the present disclosure have been provided, details of an exemplary system are described in conjunction with FIG. 1. FIG. 1 is a block diagram illustrating a system 100 in accordance with some implementations. The system 100 in some implementations includes one or more processing units CPU(s) 102 (also referred to as processors), one or more network interfaces 104, a user interface 106 including (optionally) a display 108 and an input system 110, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the system 100 with other devices and/or a communication network 104;
a microbiome evaluation module 140 for determining a disease state, in a plurality of disease states, of a subject based on the constitution of the subject's microbiome; and
a datastore of subject information 140 based on microbiome sequencing results 150, including abundance values 152 for microbes in each of guilds 152-A and 152-B as described herein.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

Although FIG. 1 depicts a “system 100,” the figure is intended more as a functional description of the various features that may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules instead may be stored in persistent memory 112.

### 1. Methods of Training a Model for Predicting Subject Response to a Therapy for a Disorder

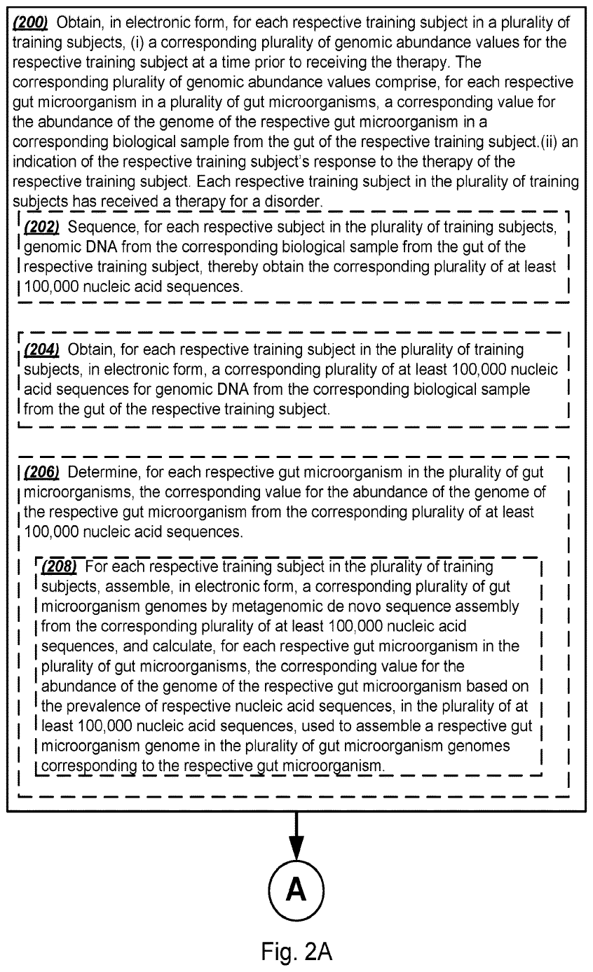

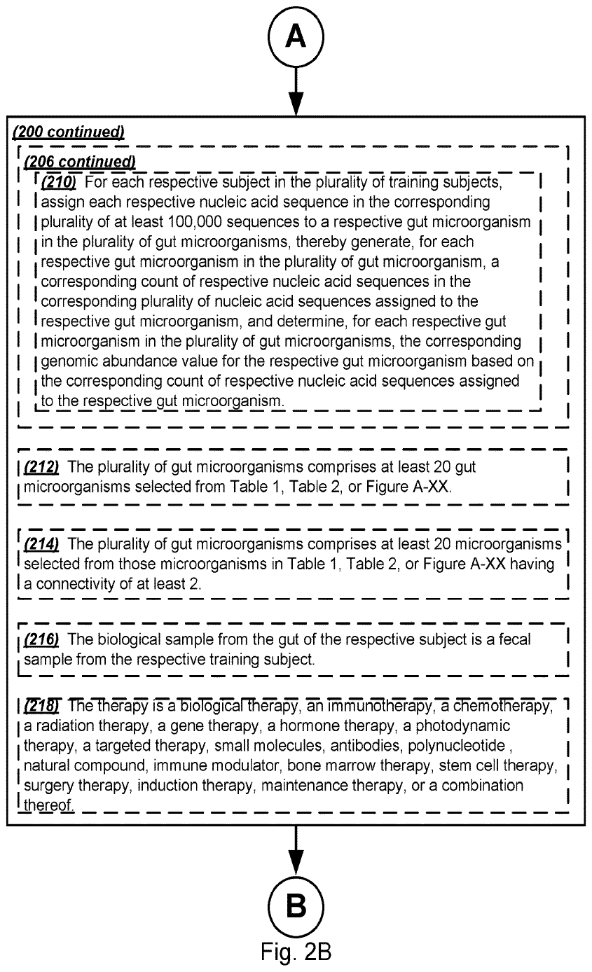

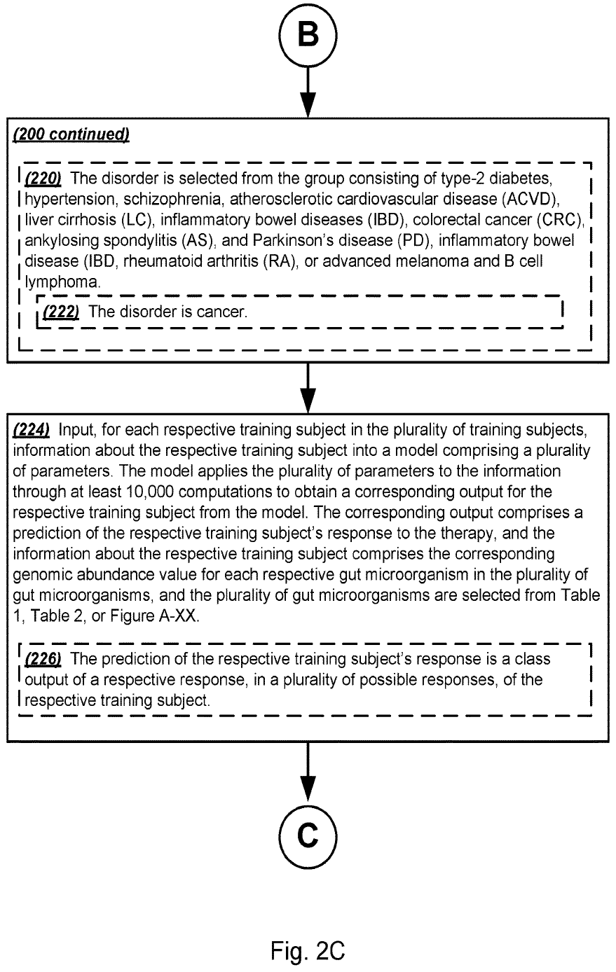

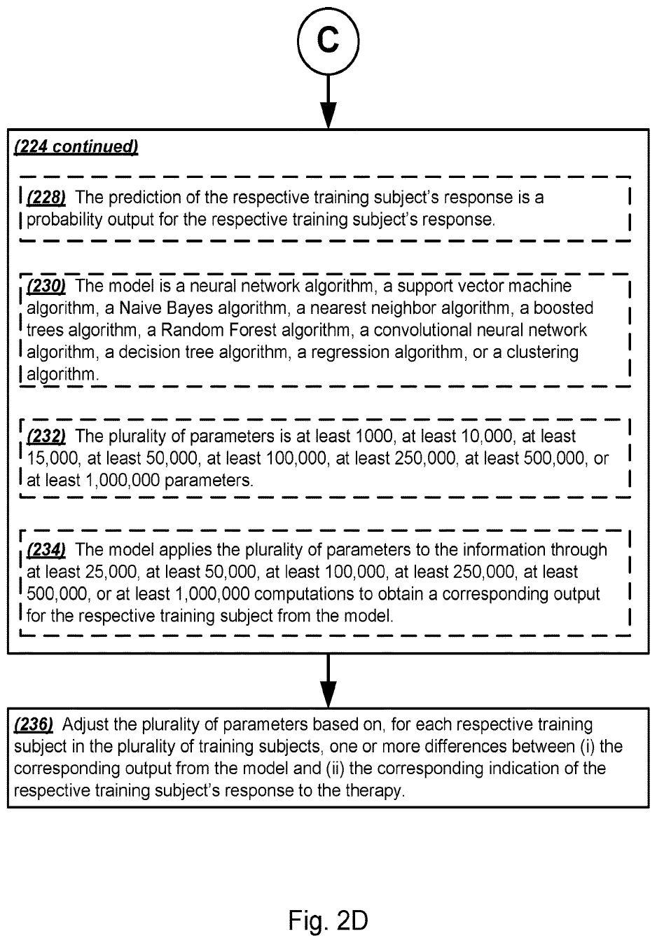

FIG. 2 is a schematic diagram of a method of training a model for predicting a subject's response to a therapy for a disorder as discussed below. The method may be implemented using a computer system (e.g., the computer system 100 shown and described above in reference to FIG. 1).

Referring to block 200, in some embodiments, the methods including obtaining, in electronic form, for each respective training subject in a plurality of training subjects, (i) a corresponding plurality of genomic abundance values for the respective training subject at a time prior to receiving the therapy, wherein the corresponding plurality of genomic abundance values comprise, for each respective gut microorganism in a plurality of gut microorganisms, a corresponding value for the abundance of the genome of the respective gut microorganism in a corresponding biological sample from the gut of the respective training subject, (ii) an indication of the respective training subject's response to the therapy of the respective training subject. Each respective training subject in the plurality of training subjects has received a therapy for a disorder.

In some embodiments, the plurality of training subjects comprises at least 50, at least 100, at least 200, at least 500, at least 1000, at least 5000, at least 10,000, at least 20,000, at least 50,000, at least 100,000, at least 500,000, or at least 1,000,000 subjects. In some embodiments, the plurality of training subjects comprises no more than 1,000,000, no more than 500,000, no more than 100,000, no more than 50,000, no more than 20,000, no more than 10,000, no more than 1000 subjects, no more than 500 subjects, no more than 100 subjects, or no more than 50 subjects. In some embodiments, the plurality of training subjects consists of from 50 to 100, from 50 to 200, from 50 to 500, from 100 to 500, from 200 to 500, from 200 to 1000, from 500 to 1000, from 200 to 5,000, from 1000 to 10,000, from 5000 from 200,00, from 10,000 to 50,000, from 20,000 to 100,000, or from 500,000 to 1,000,000. In some embodiments, the plurality of training subjects falls within another range starting no lower than 50 subjects and ending no higher than 100,000,000 subjects. In some embodiments, the plurality of subjects shares similar health status (such as physical or mental conditions, medical history, gene carrier, or medication use).

In some embodiment, a corresponding biological sample from the gut of the respective training subject was taken prior to a treatment or a therapy. In some embodiments, the biological sample is taken no more than 15 minutes, 30 minutes, 1 hour, 2 hours, 3 hours, 4 hours, 5 hours, 6 hours, 12 hours, or 24 hours prior to a treatment or a therapy. In some embodiments, the biological sample is taken 1 day, 2, days, 3 days, 4 days, 5 days, 6 days, 1 week, 2 weeks, 3 weeks, 4 weeks, or more prior to a treatment or a therapy. In some embodiments, the biological sample is taken about any of 1 month, 2 months, 3 months, 4 months, 5 months, 6 months, or more prior to a treatment or a therapy.

In some embodiments, sample data (including plasma, stool specimens) and corresponding clinical information (including gender/age/body fat count/underlying disease/histopathological characteristics, etc.) were collected for each training subject prior to receiving a therapy. Individual biological samples were subjected to full microbiome analysis. In some embodiments, the sample is a tissue biopsy, an intestinal, or mucosal sample. See, for example, Tang Q, Jet al., Current Sampling Methods for Gut Microbiota: A Call for More Precise Devices, Front Cell Infect Microbiol., 10:151 (2020), the content of which is incorporated herein by reference in its entirety. In some embodiment, the biological sample from the gut of the respective subject is a fecal sample from the respective training subject.

In some of the embodiments, the corresponding value for the abundance of the genome is a value representative of the absolute abundance of a microorganism genome. In some of the embodiments, the corresponding value for the abundance of the genome is a value representative of a normalized abundance value, or a relative abundance value (e.g., an abundance of one microorganism normalized against the abundance of total microbiome of interest). In some of the embodiments, corresponding value for the abundance of the genome is a value representative of an averaged abundance value (e.g., average of abundances obtained at different time points or from different biological samples from the patients, or average of abundances obtained using different probes, etc.), or a combination of any of above. The corresponding value for the abundance of the genome is measured by any technique known in the art. In some embodiments, the genomic abundance value for the genome is measured by quantitative PCR (qPCR), such as bacterial 16S rRNA qPCR, RT-PCR, or qRT-PCR, for quantifying the abundance of region of interests in the genome, e.g., as described in U.S. Pat. No. 11,427,865, the disclosure of which is hereby incorporated by reference in its entirety. In some embodiments, the genomic abundance value is measured by targeted sequencing (e.g., 16S rRNA sequencing, or any other suitable biomarker), partial genome sequencing or whole genome sequencing, thereby quantifying the number of reads of the targeted regions in a microorganism genome to determine the abundance of the genome, e.g., as disclosed in U.S. Patent Application Publication No. 2021/0403986 or U.S. Pat. No. 11,332,783, the disclosures of which are hereby incorporated by reference in their entireties. In some embodiments, deep sequencing is employed to determine the abundance of targeted sequences, e.g., as disclosed in U.S. Patent Application Publication No. 2018/0237863, the disclosure of which is incorporated herein by reference in its entirety. In some embodiments, the sequencing depth is at least 2×, at least 3×, at least 4×, at least 5×, at least 6×, at least 7×, at least 8×, at least 9×, at least 10×, at least 11×, at least 12×, at least 13×, at least 14×, at least 15×, at least 16×, at least 17×, at least 18×, at least 19×, at least 20×, at least 21×, at least 22×, at least 23×, at least 24×, at least 25×, at least 26×, at least 27×, at least 28×, at least 29×, at least 30×, at least 31×, at least 32×, at least 33×, at least 34×, at least 35×, at least 36×, at least 37×, at least 38×, at least 39×, at least 40×, at least 41×, at least 42×, at least 43×, at least 44×, at least 45×, at least 46×, at least 47×, at least 48×, at least 49×, at least 50×, at least 51×, at least 52×, at least 53×, at least 54×, at least 55×, at least 56×, at least 57×, at least 58×, at least 59×, at least 60×, at least 70×, at least 80×, at least 90×, at least 100×, at least 110×, at least 120×, at least 130×, at least 150×, at least 200×, at least 300×, at least 400×, at least 500×, at least 750×, at least 1000×, or more. In some embodiments, shotgun metagenomic sequencing is employed to provide sequence reads for genomes in a sample, e.g., as described in U.S. Pat. No. 11,028,449, the content of which is incorporated herein by reference in its entirety.

In some embodiments, the indication of subject's response is characterized by clinical outcome measures include, but are not limited to, complete remission, partial remission, non-remission, survival, development of adverse events, or any combination thereof. In some embodiments, one responder has complete remission in response to the treatment, and the non-responders has non-remission or partial remission in response to the treatment. In some embodiment, training subjects were subjected to routine clinical examinations, laboratory analyses, and computed tomography. Tumor responses were evaluated using RECIST criteria. In some embodiment, complete response was defined as complete radiographic disappearance of measurable or evaluable disease or stable, minimal radiographic findings; partial response was defined as reduction of the longest dimension of measurable disease by at least 50%; stable disease was defined as reduction of the longest dimension by less than 25%; Progressive disease was defined as growth of the tumor by more than 25% in the longest dimension or development of new lesions. In some embodiment, overall response rate was defined as the sum of the complete and partial response rates and the tumor control rate was defined as the sum of overall response rates with stable disease rates.

In some embodiments, the indication of subject's response is characterized by the actual treatment efficacy of an therapy, including progression-free survival (PFS), the duration of the progression free survival under treatment, total Survival (OS), response to therapy (RT), overall response rate (ORR), sustained clinical effect (DCB), Disease Activity Score, or any combination thereof, or any other method for evaluating the progression or prognosis of a disease or disorder known in the art.

In some embodiments, “progression free survival” (PFS) has its art-understood meaning relating to the length of time during and after the treatment of a disease, such as cancer, that a patient lives with the disease but it does not get worse. In some embodiments, measuring the progression-free survival is utilized as an assessment of how well a new treatment works. In some embodiments, PFS is determined in a randomized clinical trial; in some such embodiments, PFS refers to time from randomization until objective tumor progression and/or death.

In some embodiments, ORR may be defined as the proportion of patients in whom partial (PR) or complete (CR) responses are identified as a best overall response (BOR) according to some metric, such as Response Evaluation Criteria in Solid Tumors (RECIST 1.1). Stable disease (SD) was categorized as non-response together with progressive disease (PD). In some embodiments, ORR has its art-understood meaning referring to the proportion of patients with tumor size reduction of a predefined amount and for a minimum time period. In some embodiments, response duration usually measured from the time of initial response until documented tumor progression. In some embodiments, ORR involves the sum of partial responses plus complete responses.

In some embodiments, “clinical effect” refers to a clinical benefit. In some embodiments, such a clinical benefit is or comprises reduction in tumor size, increase in progression free survival, increase in overall survival, decrease in overall tumor burden, decrease in the symptoms caused by tumor growth such as pain, organ failure, bleeding, damage to the skeletal system, and other related sequelae of metastatic cancer and combinations thereof. In some embodiments, the clinical effect is a “sustained clinical effect” (DCB) that is maintained for a relevant period of time. In some embodiments, the relevant period of time is at least 1 month, 2 months, 3 months, 4 months, 5 months, 6 months, 7 months, 8 months, 9 months, 10 months, 11 months, 1 year, 2 years, 3 years, 4 years, 5 years, or longer.

In some embodiments, the subject's response is measured by Disease Activity Score (DAS) (see, e.g., Van der Heijde D. M. et al., J Rheumatol, 1993, 20(3):579-81; Prevoo M. L. et al, Arthritis Rheum, 1995, 38:44-8). The DAS system represents both current state of disease activity and change. The DAS scoring system uses a weighted mathematical formula, derived from clinical trials in RA. For example, the DAS 28 is 0.56(T28)+0.28 (SW28)+0.70 (Ln ESR)+0.014 GH wherein T represents tender joint number, SW is swollen joint number, ESR is erythrocyte sedimentation rate, and GH is global health. Various values of the DAS represent high or low disease activity as well as remission, and the change and endpoint score result in a categorization of the patient by degree of response (none, moderate, good).

In some embodiments, the indication of the subject's response is measured by the level of the immune response or immune parameters of a cancer-bearing patient resulting from an immunotherapy. In some embodiments, the immune response or immune parameters are characterized by expression level of various biological markers of the host immune response in conjunction with the occurrence of a cancer at a given stage of cancer development (i.e., treatment efficacy). In some embodiments, the expression level of a biological marker is compared with a reference value for the same biological marker, and when required with reference values. The reference value for the same biological marker is thus predetermined and is already known to be indicative of a reference value that is pertinent for discriminating between a low level and a high level of the immune response of a patient with cancer, for said biological marker. Said predetermined reference value for said biological marker is correlated with a responder to treatment in a cancer patient, or conversely is correlated with non-responder to treatment in a cancer patient.

In some embodiments, a change of a combination of biological markers are quantified. In some embodiments, a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more distinct biological markers are quantified.

In certain embodiments, biological markers are quantified with immunohistochemical techniques. Example biological markers include 18s, ACE, ACTB, AGTR1, AGTR2, APC, APOA1, ARF1, AXIN1, BAX, BCL2, BCL2L1, CXCR5, BMP2, BRCA1, BTLA, C3, CASP3, CASp9, CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCL5, CCL7, CCL8, CCNB1, CCND1, CCNE1, CCR1, CCR10, CCR2, CCR3, CCR4, CCR5, CCR6, CCR7, CCR8, CCR9, CCRL2, CD154, CD19, CD1a, CD2, CD226, CD244, PDCD1LG1, CD28, CD34, CD36, CD38, CD3E, CD3G, CD3Z, CD4, CD40LG, CD5, CD54, CD6, CD68, CD69, CLIP, CD80, CD83, SLAMF5, CD86, CD8A, CDH1, CDH7, CDK2, CDK4, CDKN1A, CDKN1B, CDKN2A, CDKN2B, CEACAM1, COL4A5, CREBBP, CRLF2, CSF1, CSF2, CSF3, CTLA4, CTNN81, CTSC, CX3CL1, CX3CR1, CXCL1, CXCL10, CXCL11, CXCL12, CXCL13, CXCL14, CXCL16, CXCL2, CXCL3, CXCL5, CXCL6, CXCL9, CXCR3, CXCR4, CXCR6, CYP1A2, CYP7A1, DCC, DCN, DEFA6, DICER1, DKK1, Dok-1, Dok-2, DOK6, DVL1, E2F4, EBI3, ECE1, ECGF1, EDN1, EGF, EGFR, EIF4E, CD105, ENPEP, ERBB2, EREG, FCGR3A, CGR3B, FN1, FOXP3, FYN, FZD1, GAPD, GLI2, GNLY, GOLPH4, GRB2, GSK3B, GSTP1, GUSB, GZMA, GZMH, GZMK, HLA-B, HLA-C, HLA-, MA, HLA-DMB, HLA-DOA, HLA-DOB, HLA-DPA1, HLA-DQA2, HLA-DRA, HLX1, HMOX1, HRAS, HSPB3, HUWE1, ICAM1, ICAM-2, ICOS, ID1, ifna1, ifna17, ifna2, ifna5, ifna6, ifna8, IFNAR1, IFNAR2, IFNG, IFNGR1, IFNGR2, IGF1, IHH, IKBKB, IL10, IL12A, IL12B, IL12RB1, IL12RB2, IL13, IL13RA2, IL15, IL15RA, IL17, IL17R, IL17RB, IL18, IL1A, IL1B, IL1R1, IL2, IL21, IL21R, IL23A, IL23R, IL24, IL27, IL2RA, IL2RB, IL2RG, IL3, IL31RA, IL4, IL4RA, IL5, IL6, IL7, IL7RA, IL8, CXCR1, CXCR2, IL9, IL9R, IRF1, ISGF3G, ITGA4, ITGA7, integrin, alpha E (antigen CD103, human mucosal lymphocyte, antigen 1; alpha polypeptide), Gene hCG33203, ITGB3, JAK2, JAK3, KLRB1, KLRC4, KLRF1, KLRG1, KRAS, LAG3, LAIR2, LEF1, LGALS9, LILRB3, LRP2, LTA, SLAMF3, MADCAM1, MADH3, MADH7, MAF, MAP2K1, MDM2, MICA, MICB, MKI67, MMP12, MMP9, MTA1, MTSS1, MYC, MYD88, MYH6, NCAM1, NFATC1, NKG7, NLK, NOS2A, P2X7, PDCD1, PECAM-, CXCL4, PGK1, PIAS1, PIAS2, PIAS3, PIAS4, PLAT, PML, PP1A, CXCL7, PPP2CA, PRF1, PROM1, PSMB5, PTCH, PTGS2, PTP4A3, PTPN6, PTPRC, RAB23, RAC/RHO, RAC2, RAF, RB1, RBL1, REN, Drosha, SELE, SELL, SELP, SERPINE1, SFRP1, SIRP beta 1, SKI, SLAMF1, SLAMF6, SLAMF7, SLAMF8, SMAD2, SMAD4, SMO, SMOH, SMURF1, SOCS1, SOCS2, SOCS3, SOCS4, SOCS5, SOCS6, SOCS7, SOD1, SOD2, SOD3, SOS1, SOX17, CD43, ST14, STAM, STAT1, STAT2, STAT3, STAT4, STAT5A, STAT5B, STAT6, STK36, TAP1, TAP2, TBX21, TCF7, TERT, TFRC, TGFA, TGFB1, TGFBR1, TGFBR2, TIMP3, TLR1, TLRO1, TLR2, TLR3, TLR4, TLR5, TLR6, TLR7, TLR8, TLR9, TNF, TNFRSF10A, TNFRSF11A, TNFRSF18, TNFRSF1A, TNFRSF1B, OX-40, TNFRSF5, TNFRSF6, TNFRSF7, TNFRSF8, TNFRSF9, TNFSF10, TNFSF6, TOB1, TP53, TSLP, VCAM1, VEGF, WIF1, WNT1, WNT4, XCL1, XCR1, ZAP70 and ZIC2.

In some embodiment, a treatment regime or a therapy can be administered via any common route so long as the target tissue or cell is available via that route. This includes, but is not limited to, intravenous, catheterization, orthotopic, intradermal, subcutaneous, intramuscular, intraperitoneal intertumoral, oral, nasal, buccal, rectal, vaginal, or topical administration. Selection of therapeutic agents and dosage regimes may depend on various factors, such as the drug combination employed, the particular disease being treated, and the condition and prior history of the patient.

Referring to block 202, in some embodiments, the methods include sequencing, for each respective subject in the plurality of training subjects, genomic DNA from the corresponding biological sample from the gut of the respective training subject, thereby obtain the corresponding plurality of nucleic acid sequences. In some embodiments, the corresponding plurality of nucleic acid sequences comprises at least 10,000, at least 100,000, at least 250,000, at least 500,000, at least 1,000,000, at least 2,500,000, at least 5,000,000, at least 10,000,000 or at least 50,000,000 nucleic acid sequences. In some embodiments, the corresponding plurality of nucleic acid sequences comprises no more than 250,000,000, no more than 100,000,000, no more than 50,000,000, no more than 25,000,000, no more than 10,000,000, no more than 5,000,000, no more than 1,000,000, no more than 100,000 nucleic acid sequences. In some embodiments, the corresponding plurality of nucleic acid sequences consists of from 100,000 to 1,000,000, from 200,000 to 5,000,000, from 500,000 from 10,000,000, from 1,000,000 to 20,000,000, from 5,000,000 to 50,000,000, from 10,000,000 to 100,000,000, or from 50,000,000 to 250,000,000 nucleic acid sequences. In some embodiments, the corresponding plurality of nucleic acid sequences falls within another range starting no lower than 1000 nucleic acid sequences and ending no higher than 250,000,000 nucleic acid sequences.

In some embodiments, the corresponding plurality of nucleic acid sequences are obtained through metagenomic sequencing, e.g., as disclosed in U.S. Patent Application Publication No. 2016/0239602 or U.S. Pat. No. 11,495,326, the contents of which are incorporated herein by reference in their entireties. In some embodiments, metagenomes sequencing further comprise generating the plurality of metagenomic fragment reads. In some embodiments, metagenomic sequencing further comprise fragmenting microbial genomes into random fragments of targeted sizes. The resulting fragments can vary in size. In one embodiment, fragments of approximately 500 nucleotides can be obtained. In some embodiments, fragments of from 100-2000 nucleotides, e.g., 200-800, 100-900, 100-1000, 300-800, 400-900 nucleotides can be obtained. In some embodiments, the method may further comprise extracting the metagenomic fragments from the corresponding biological sample. In some embodiments, metagenomes sequencing further comprise sequencing the fragments using high throughput sequencing methods to generate a plurality of sequencing reads.

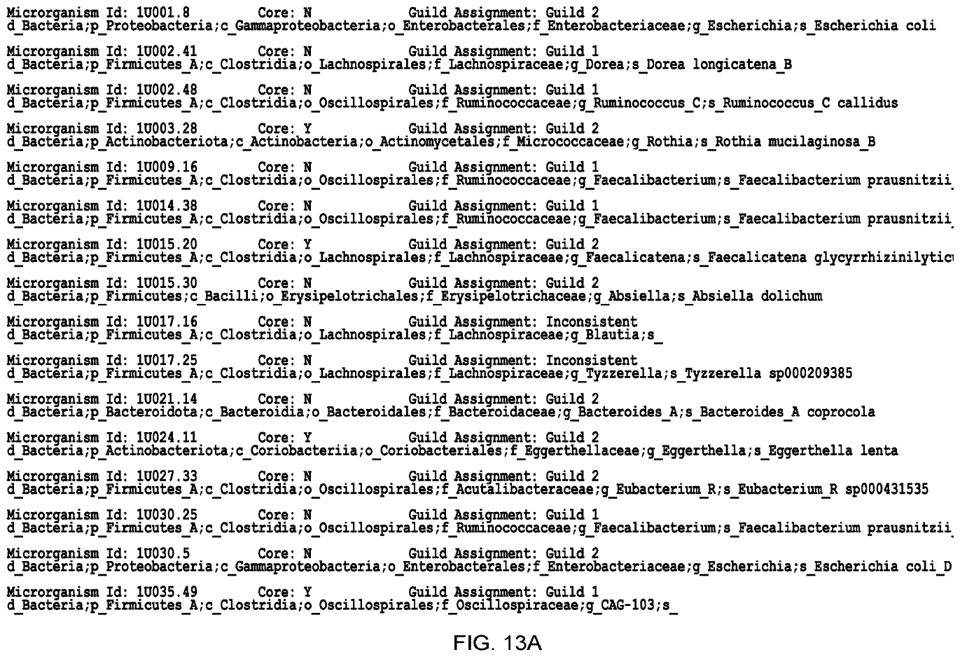

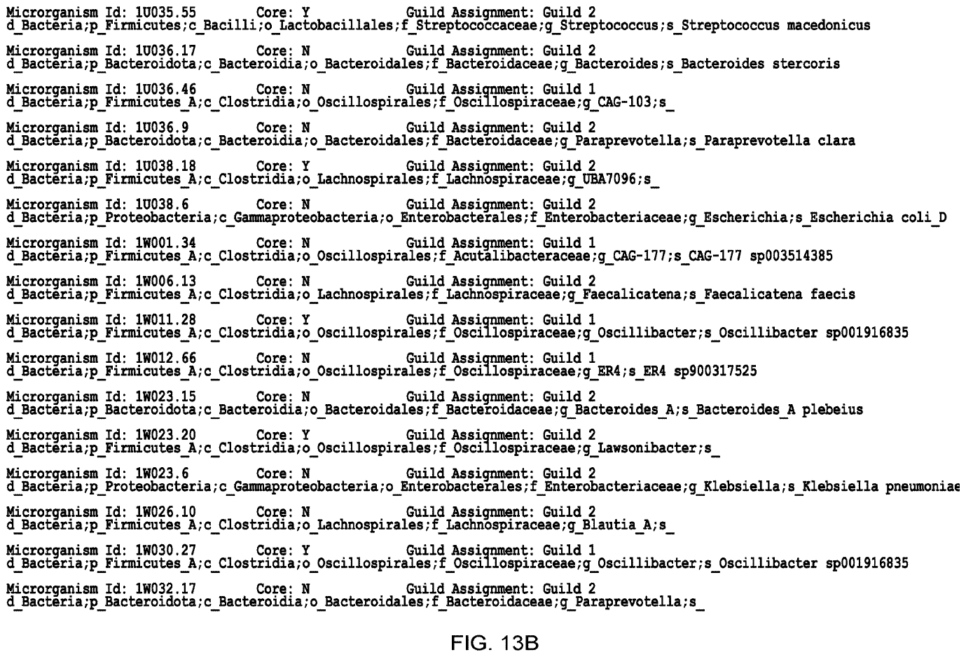

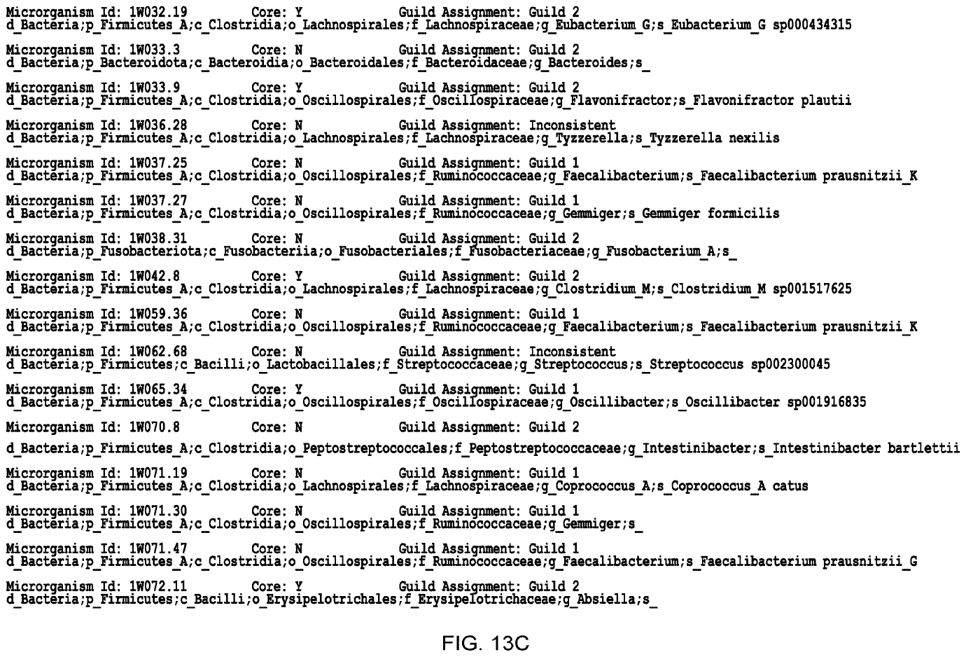

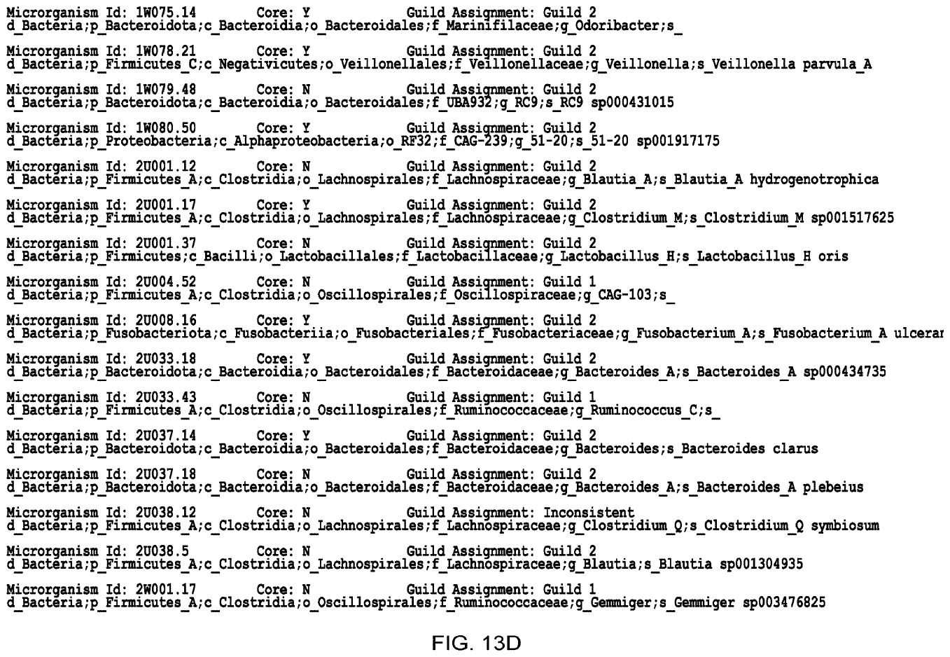

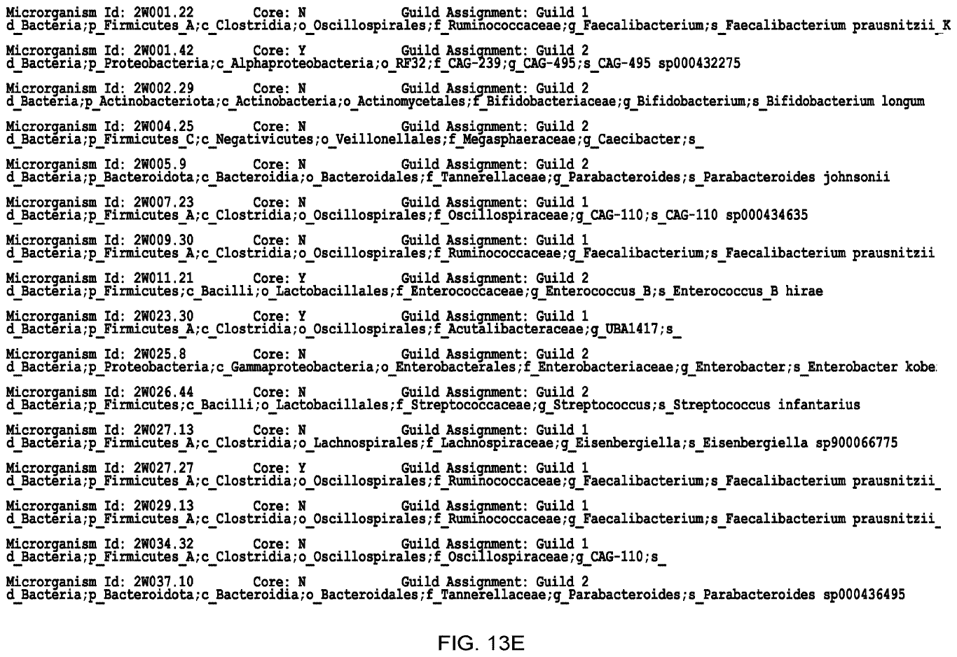

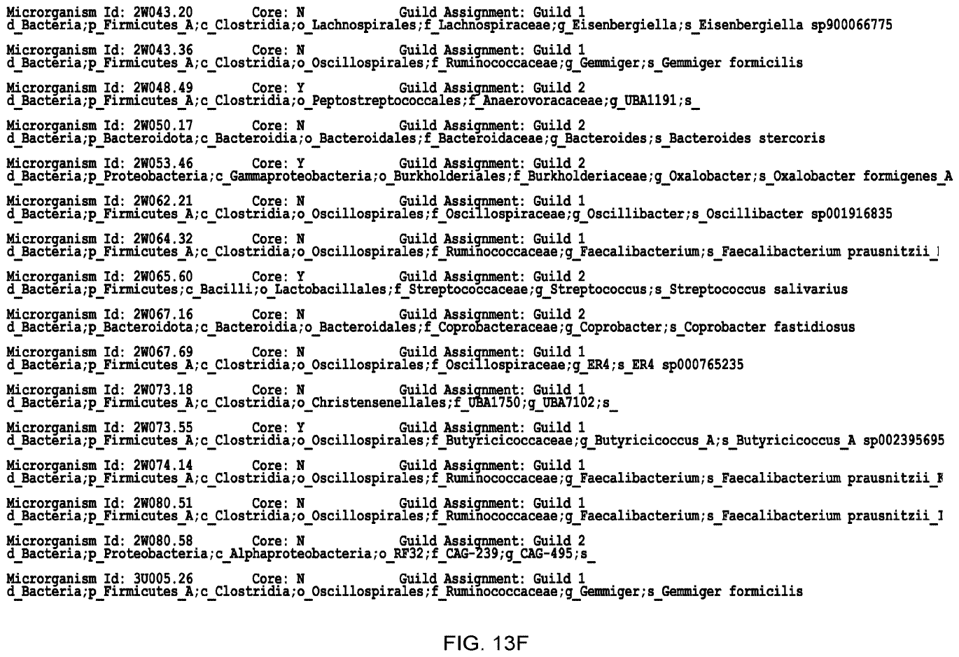

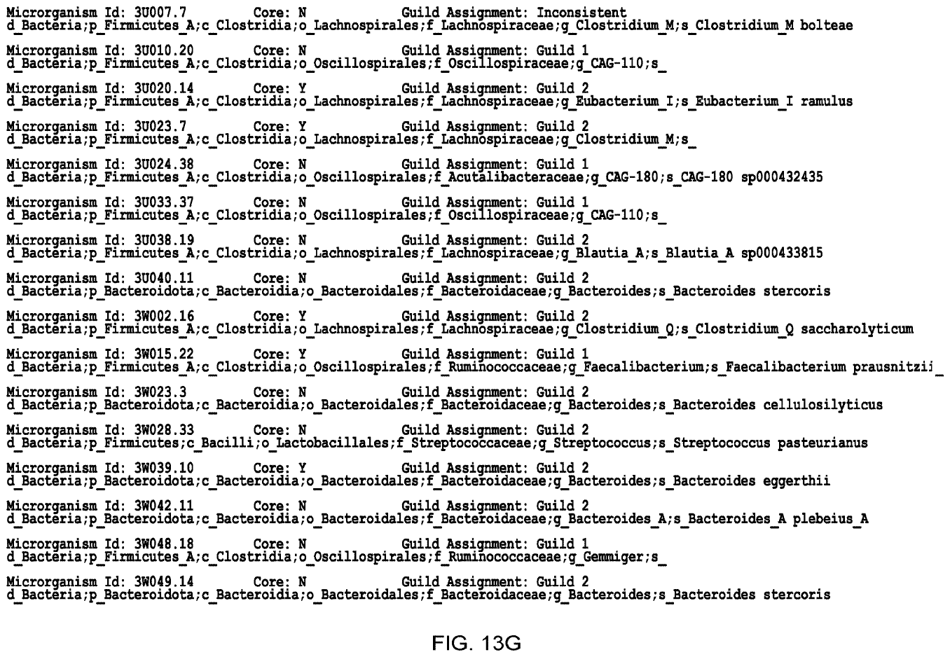

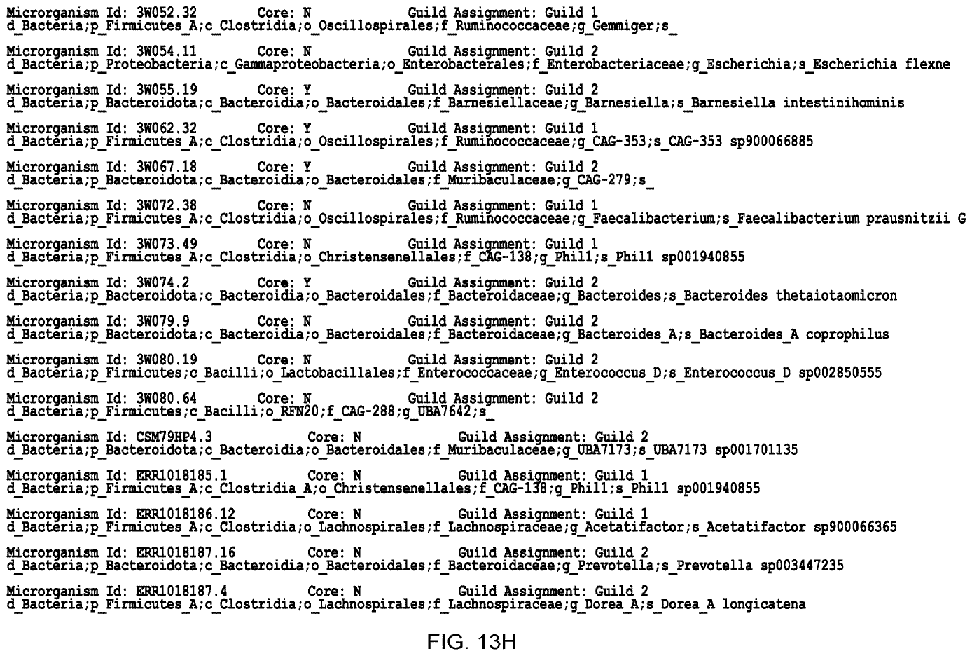

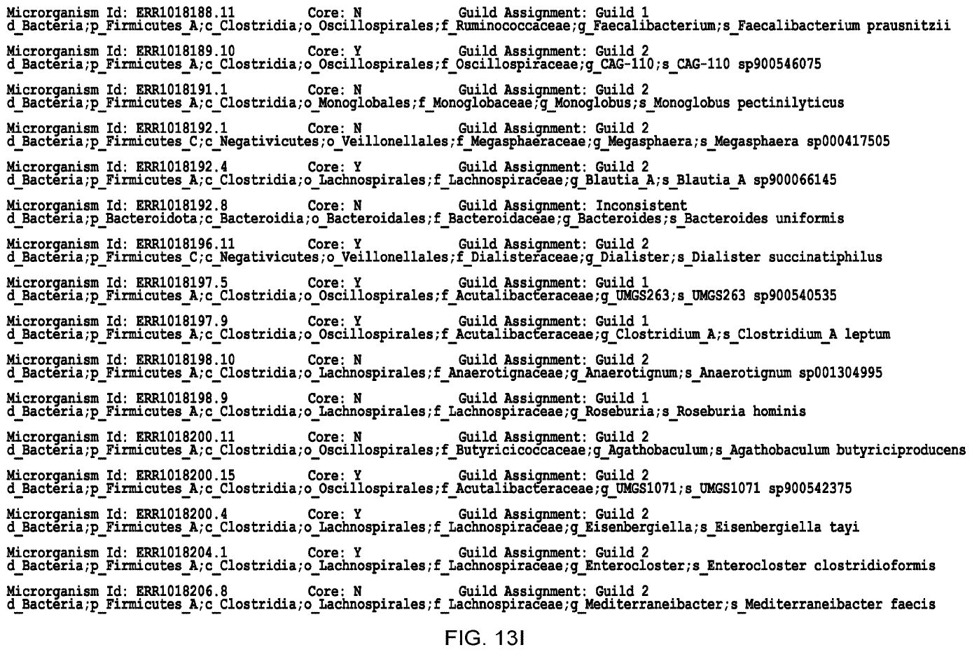

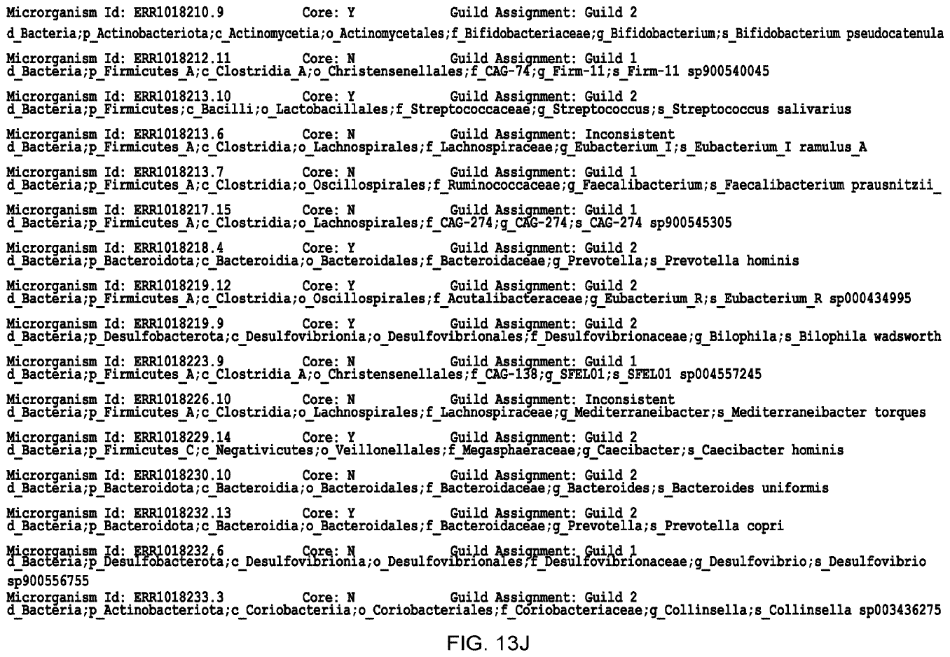

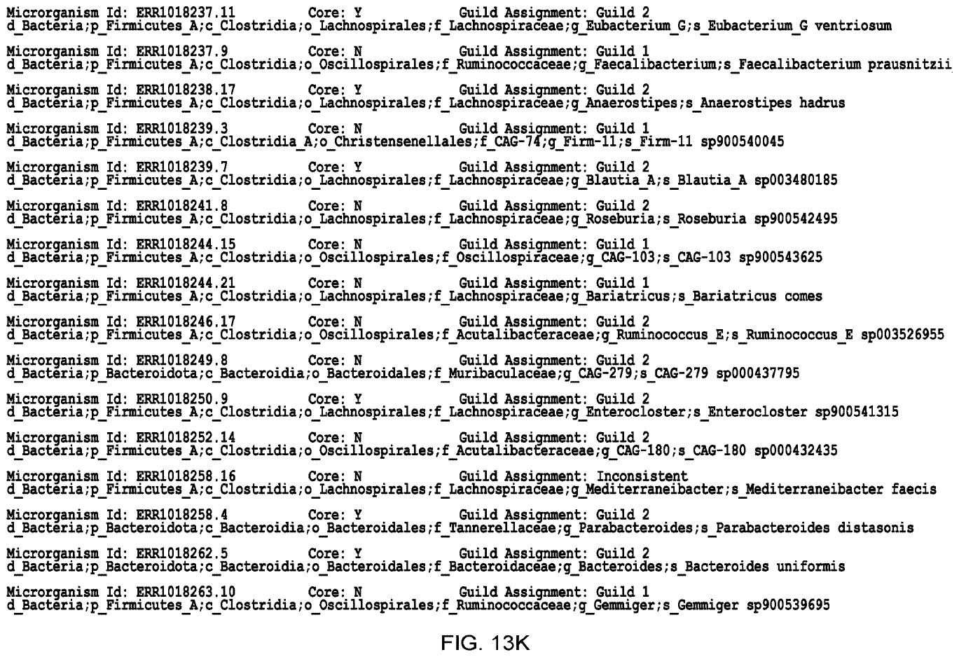

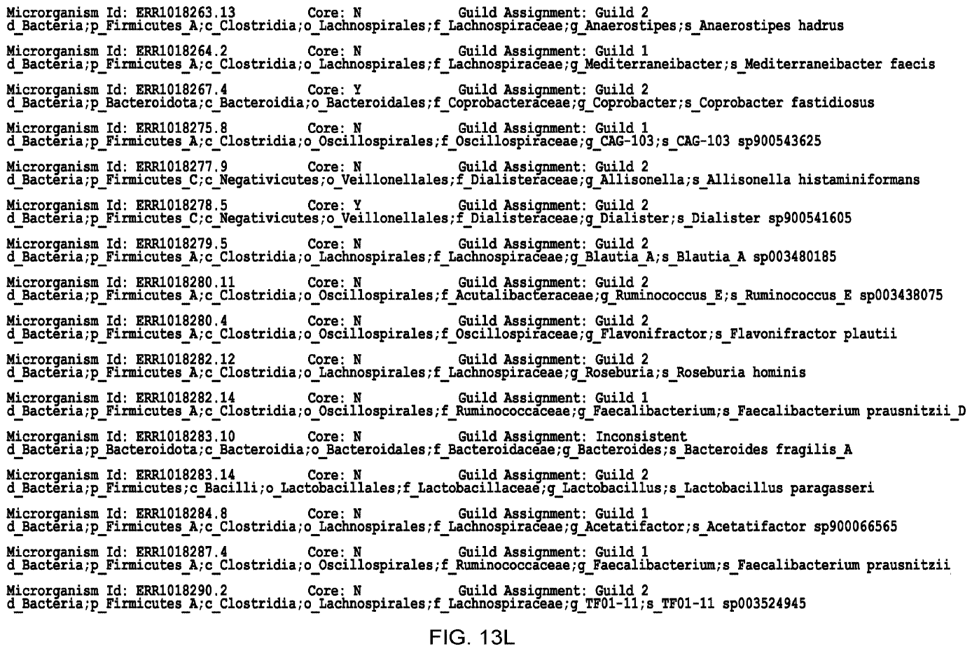

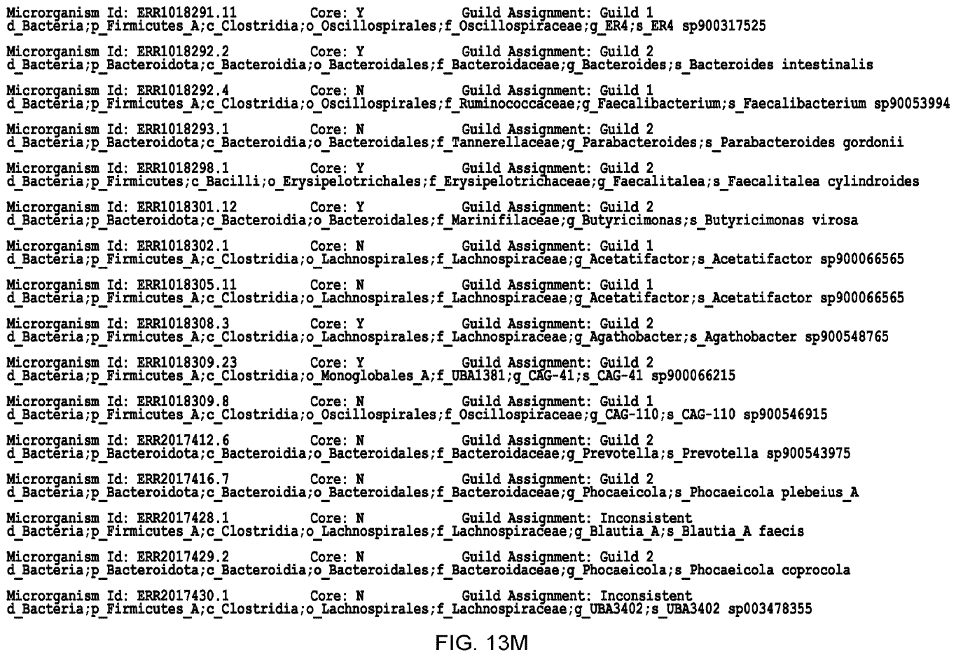

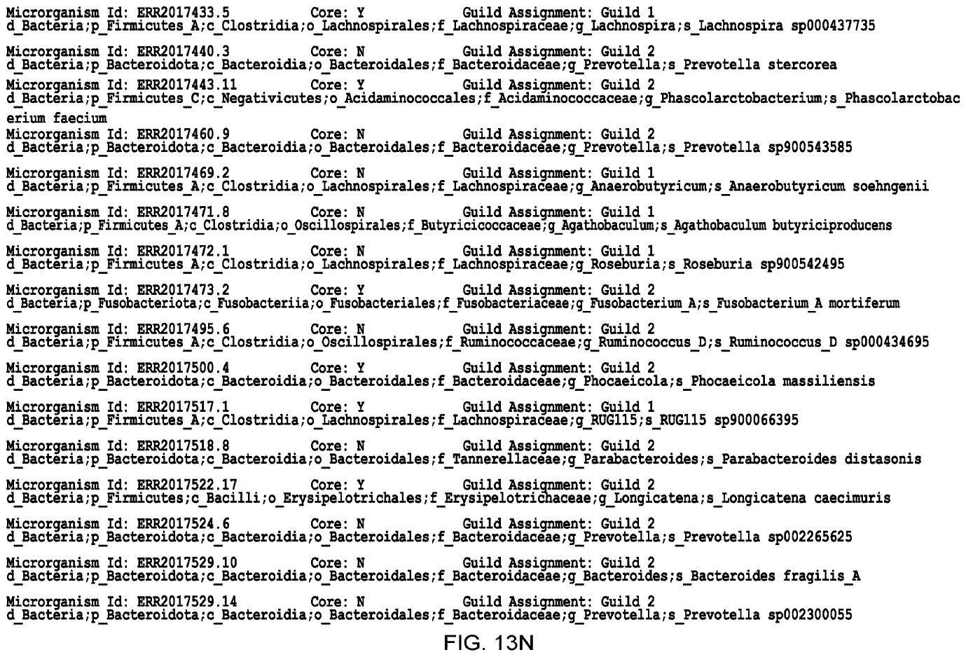

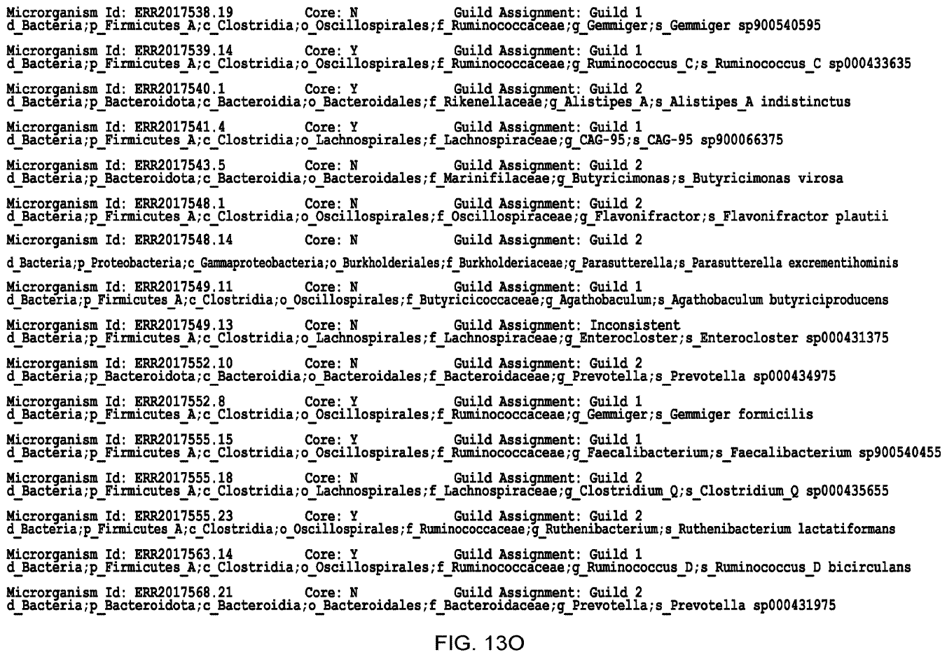

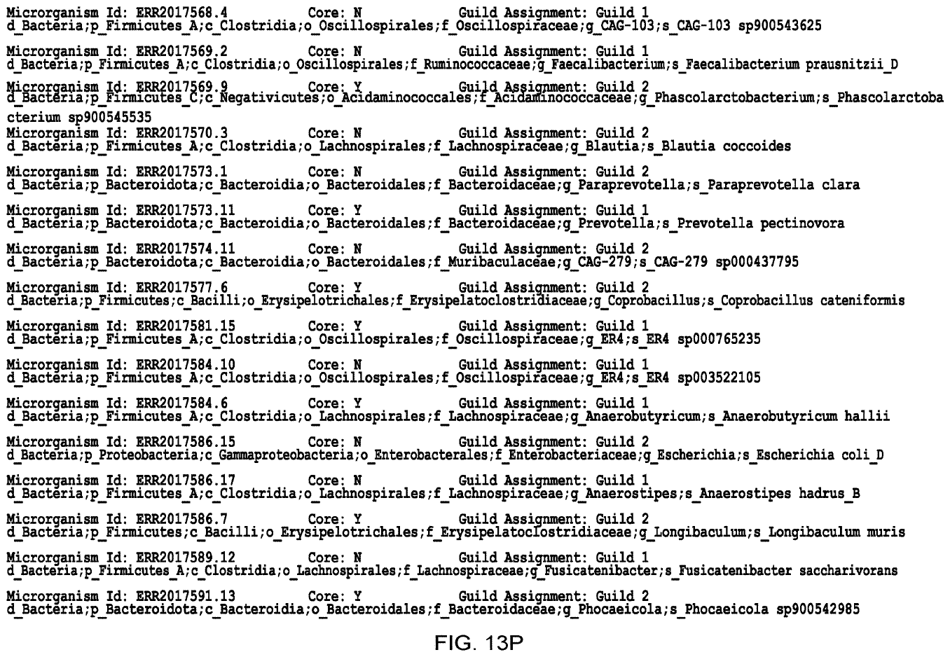

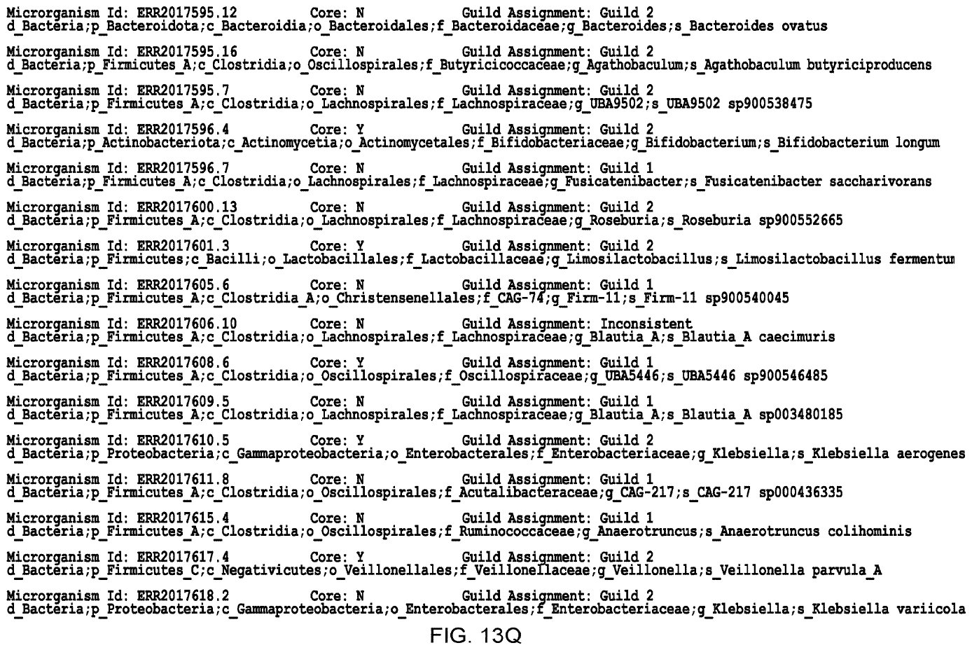

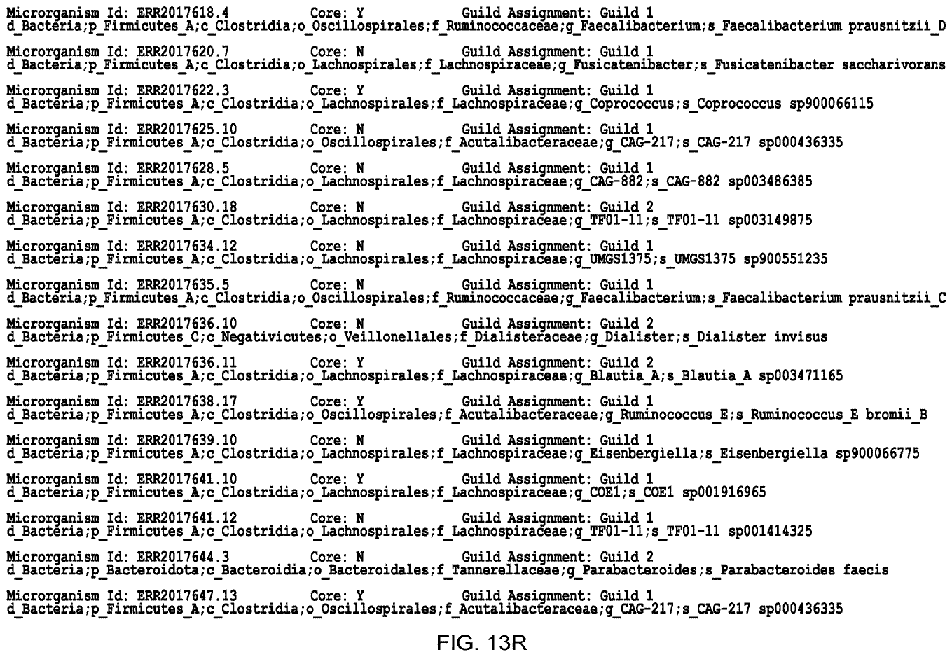

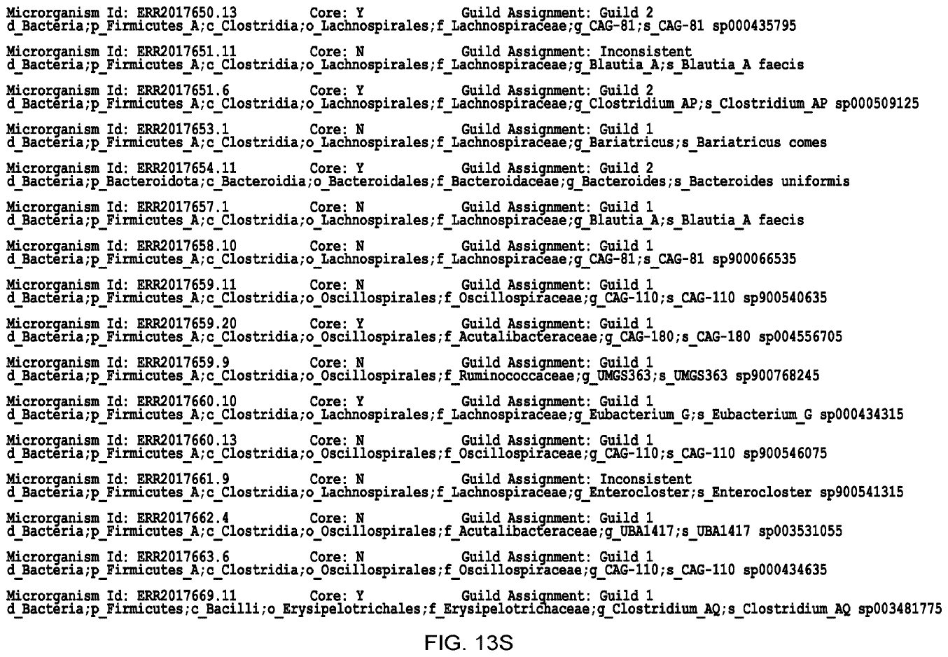

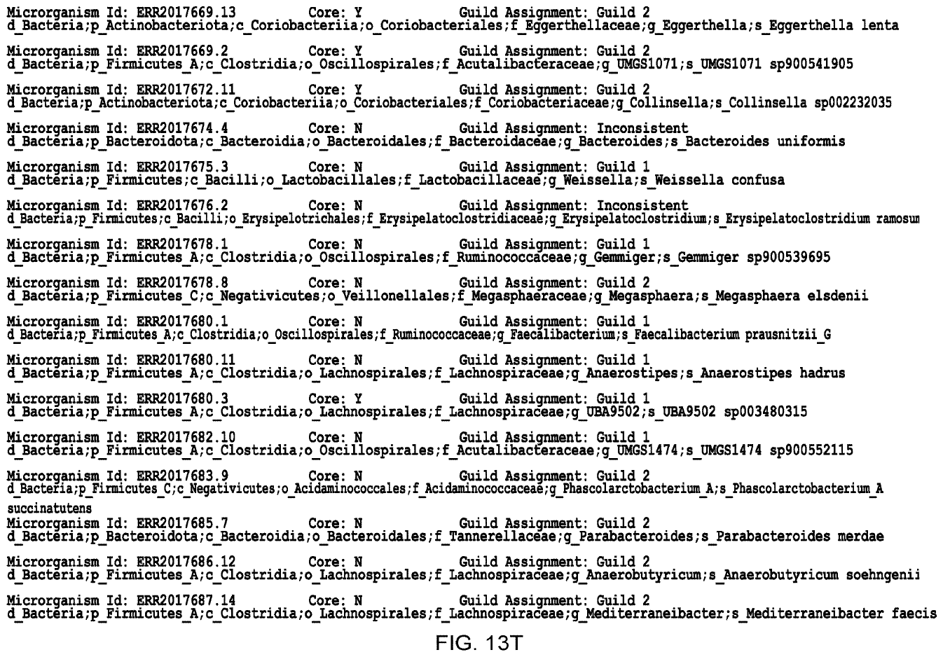

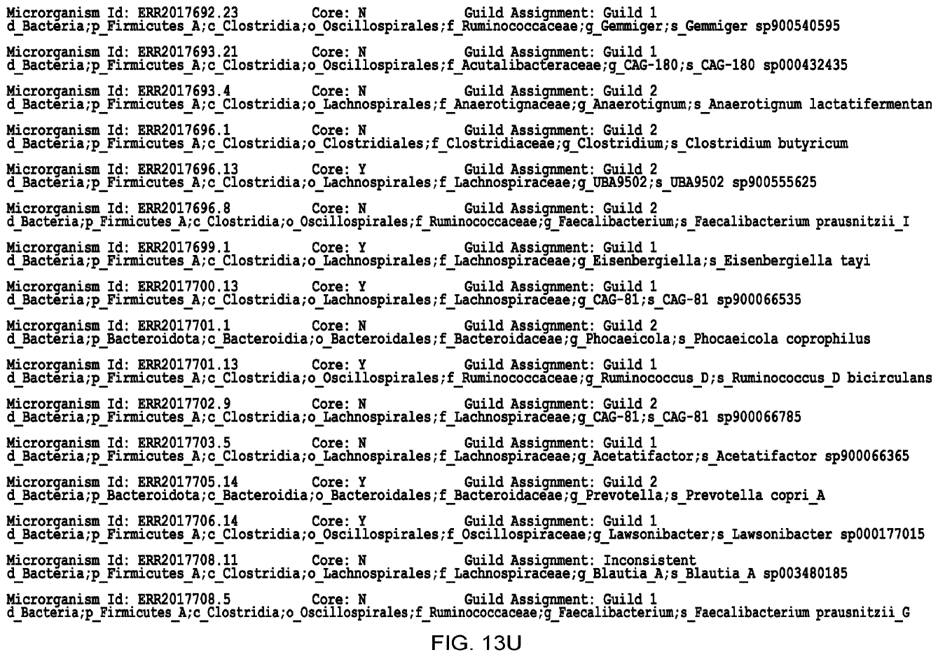

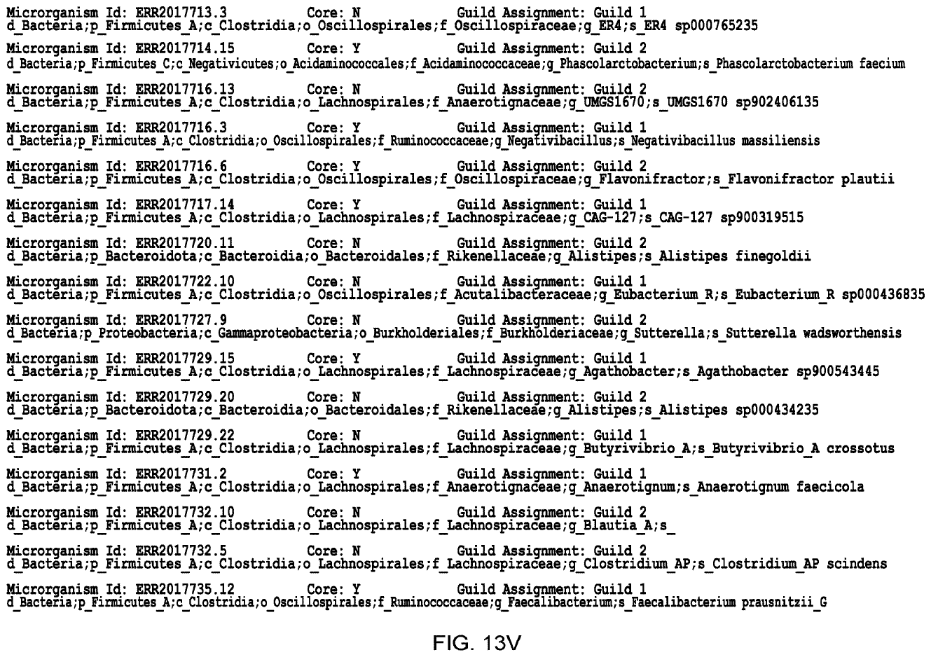

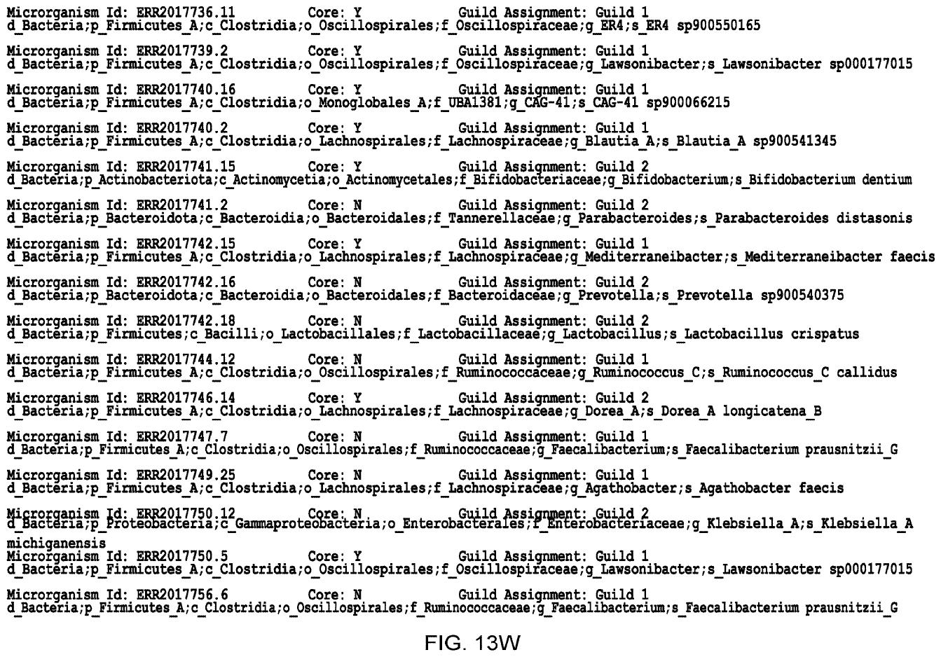

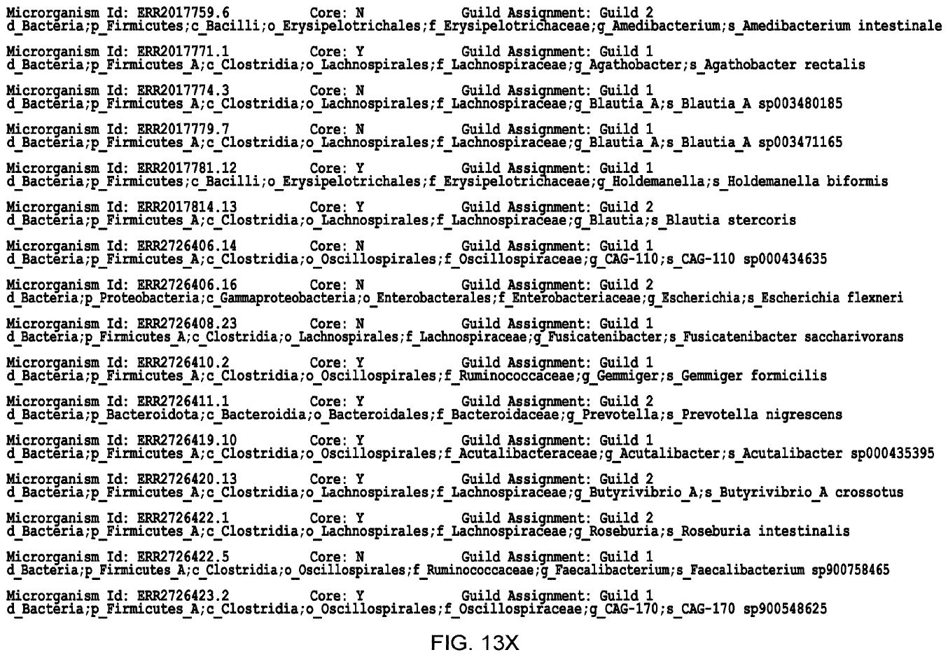

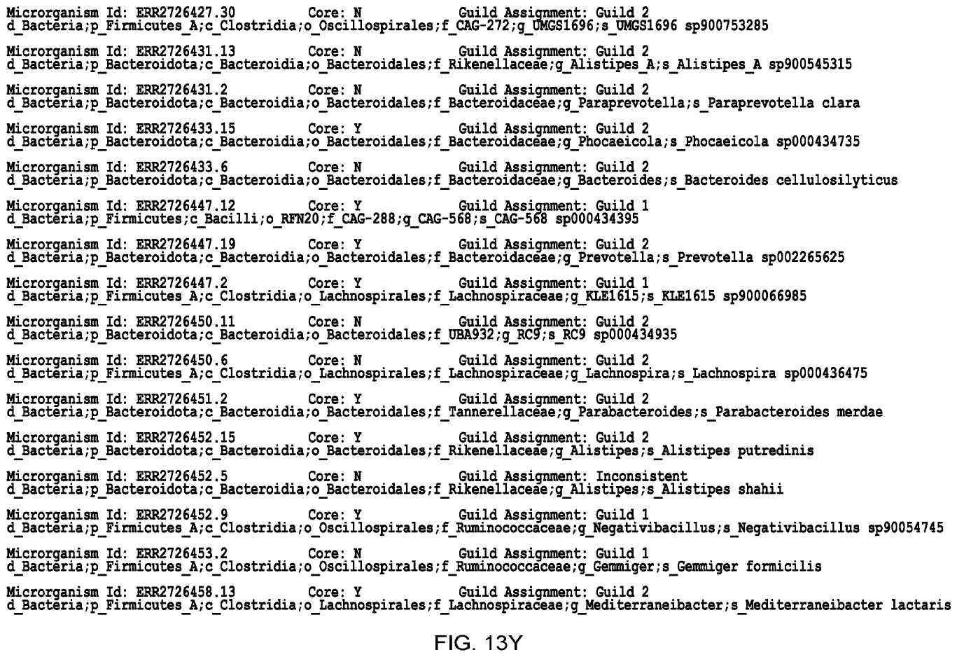

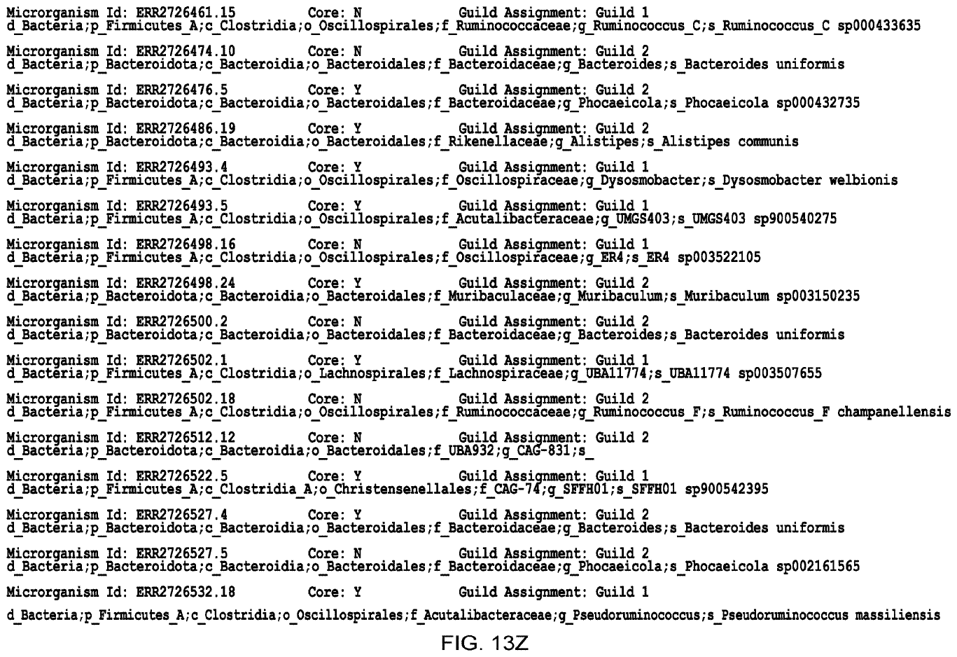

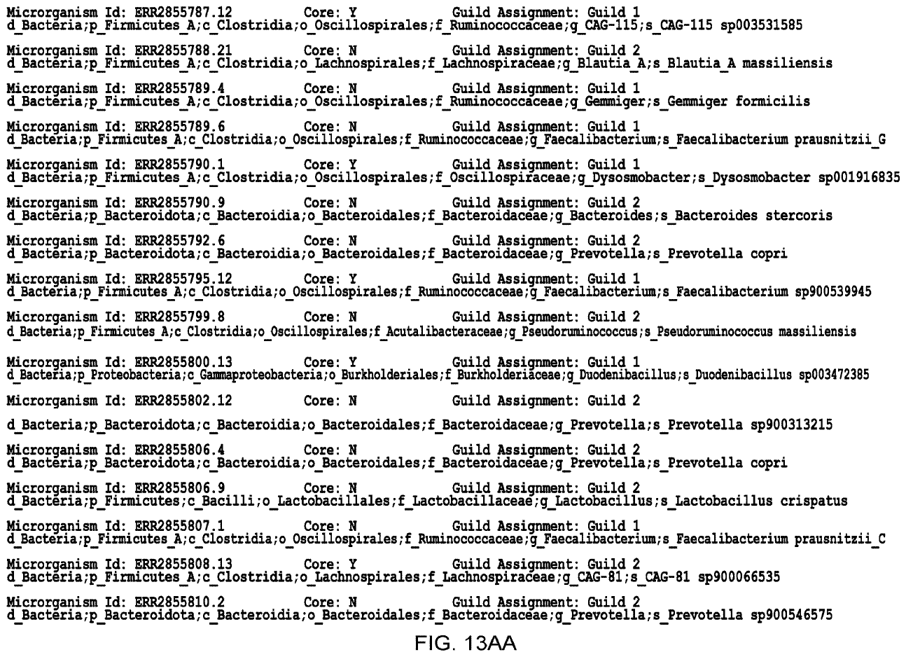

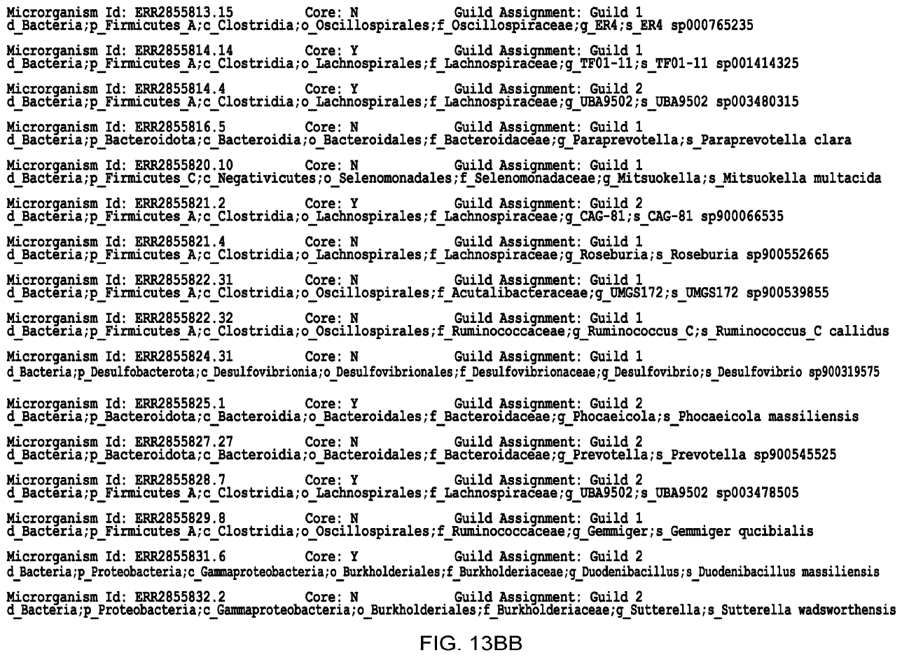

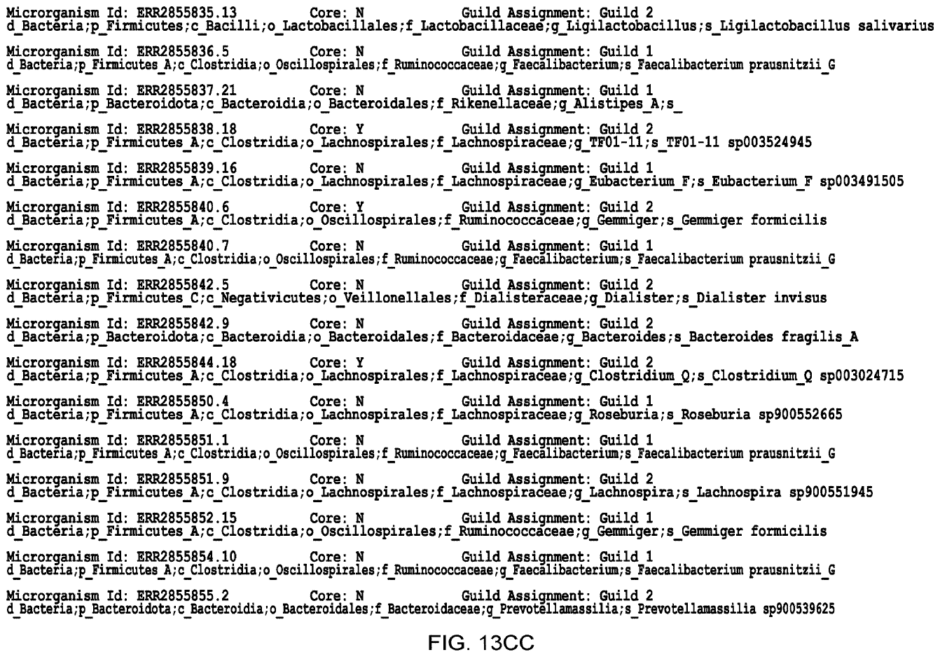

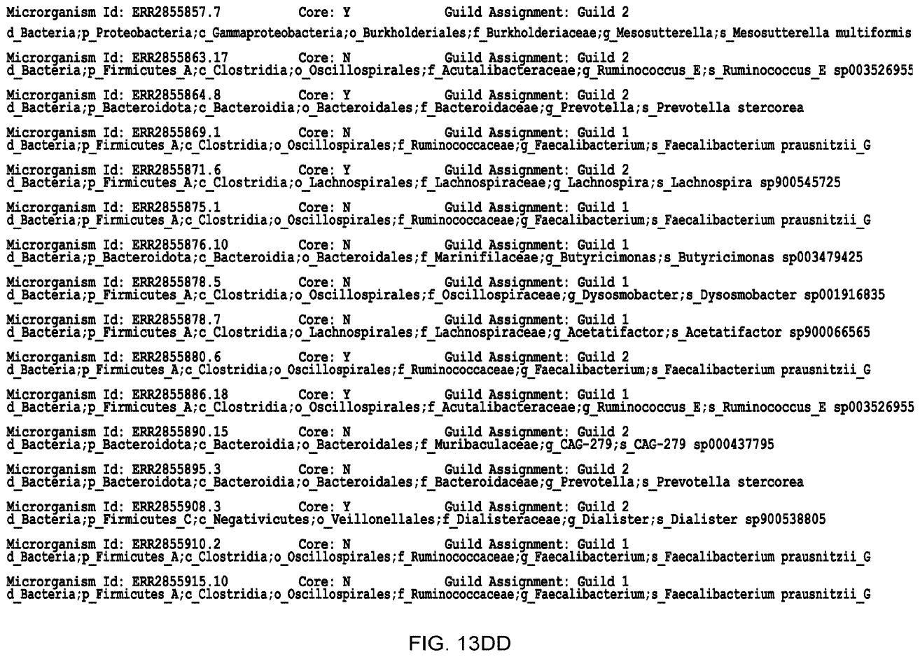

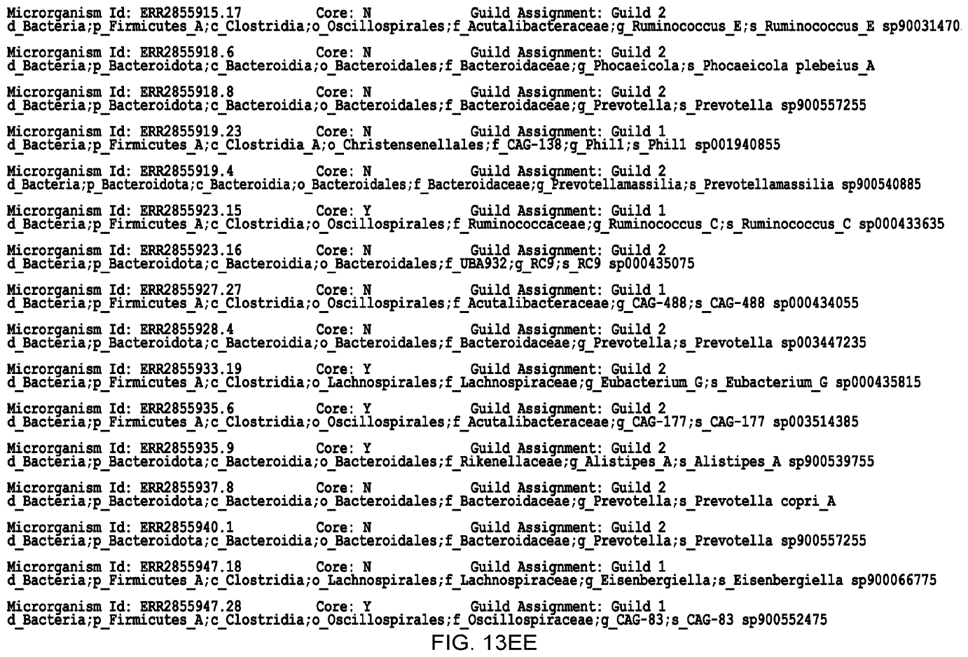

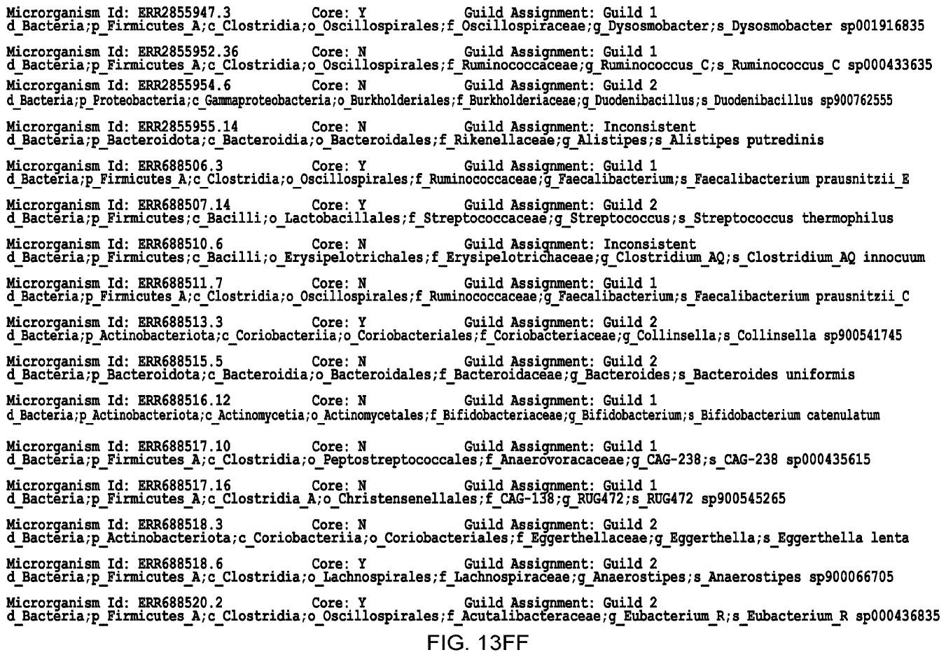

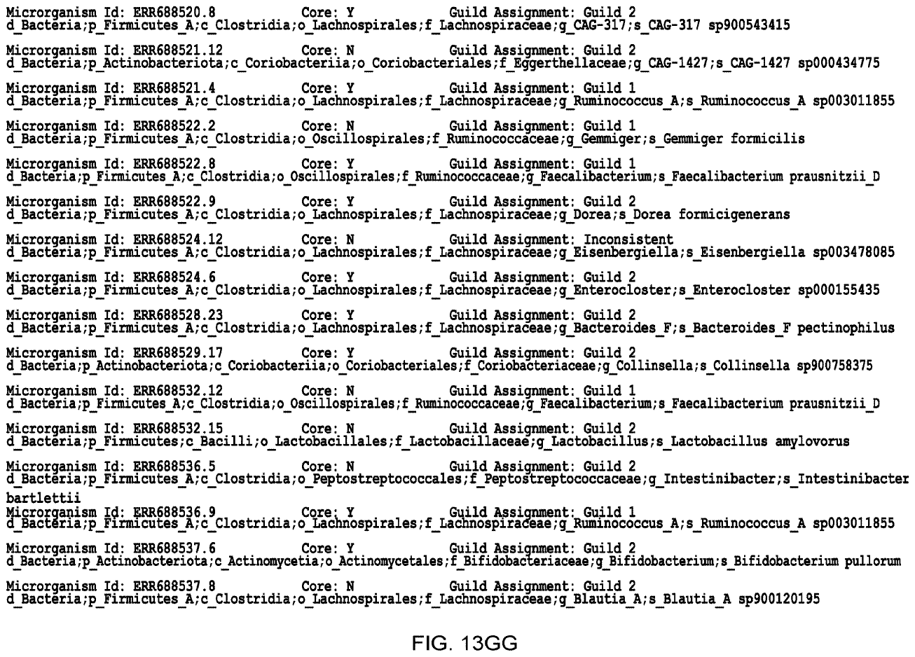

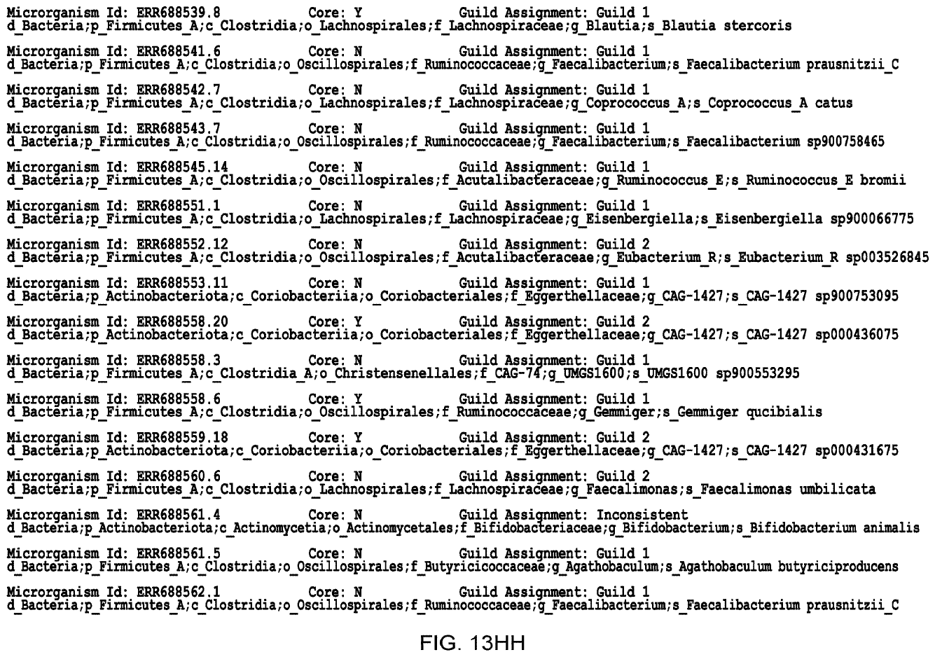

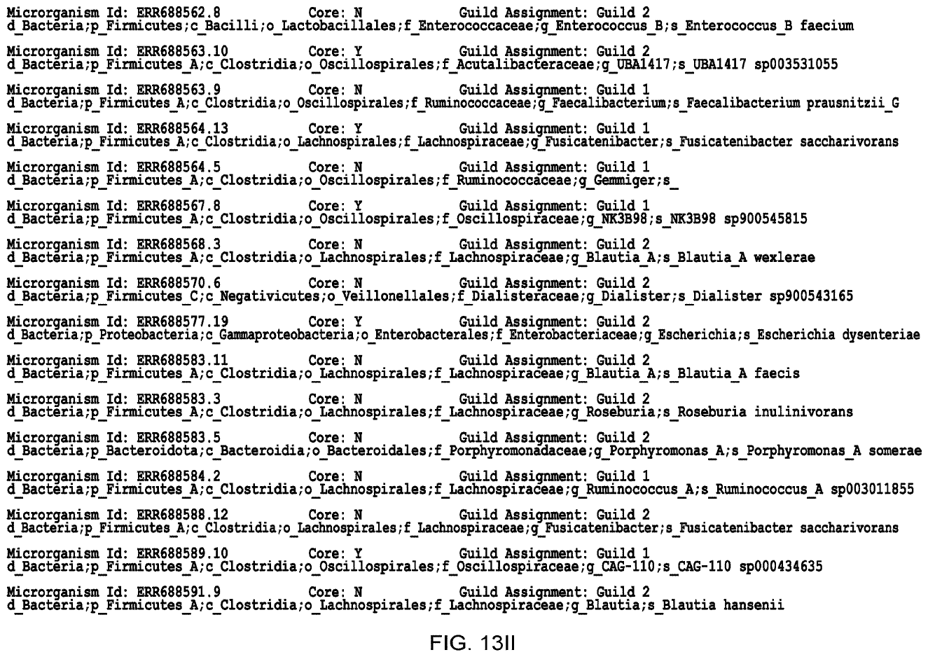

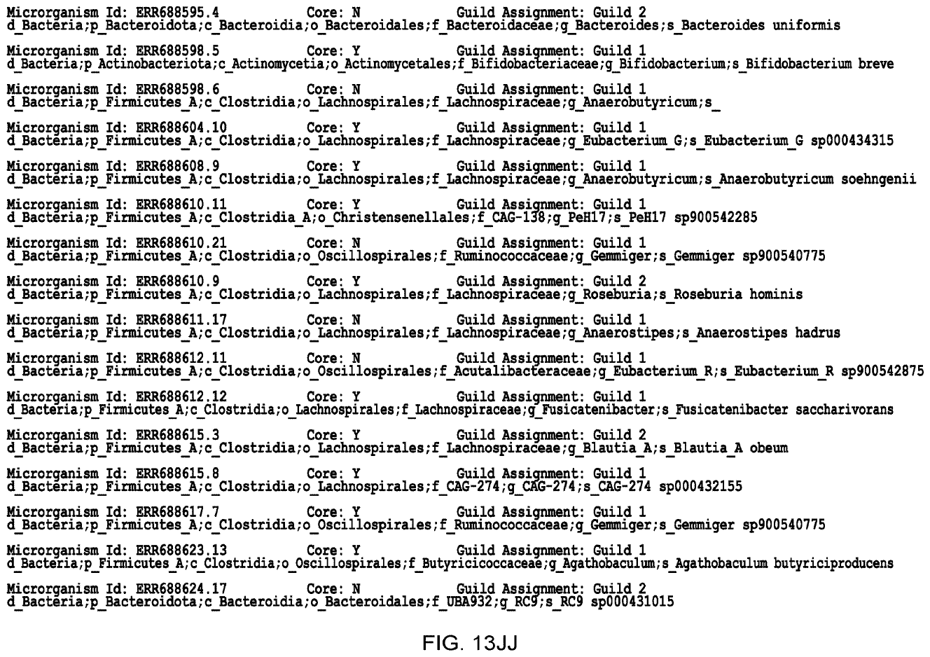

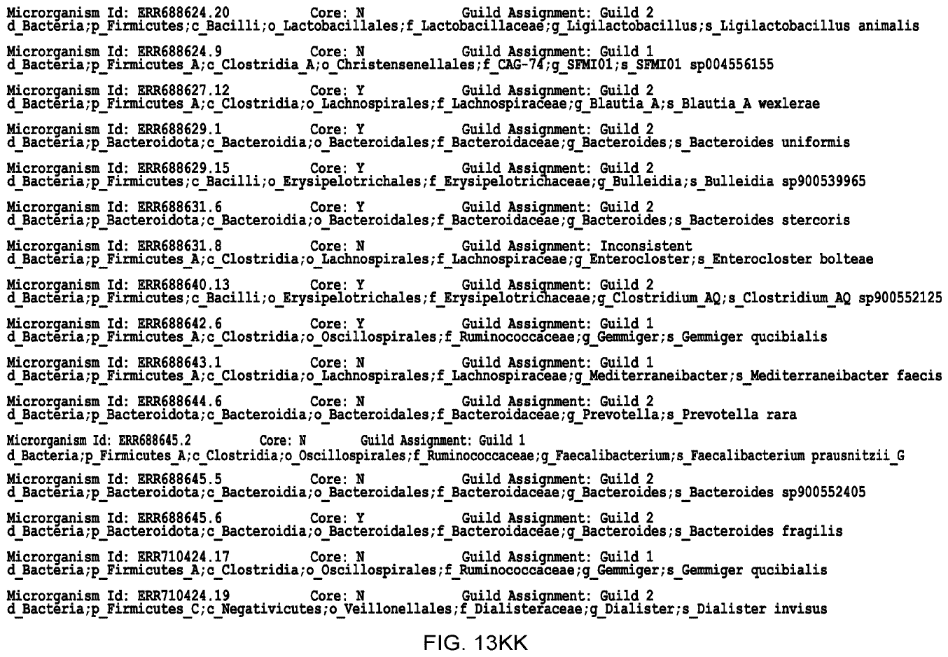

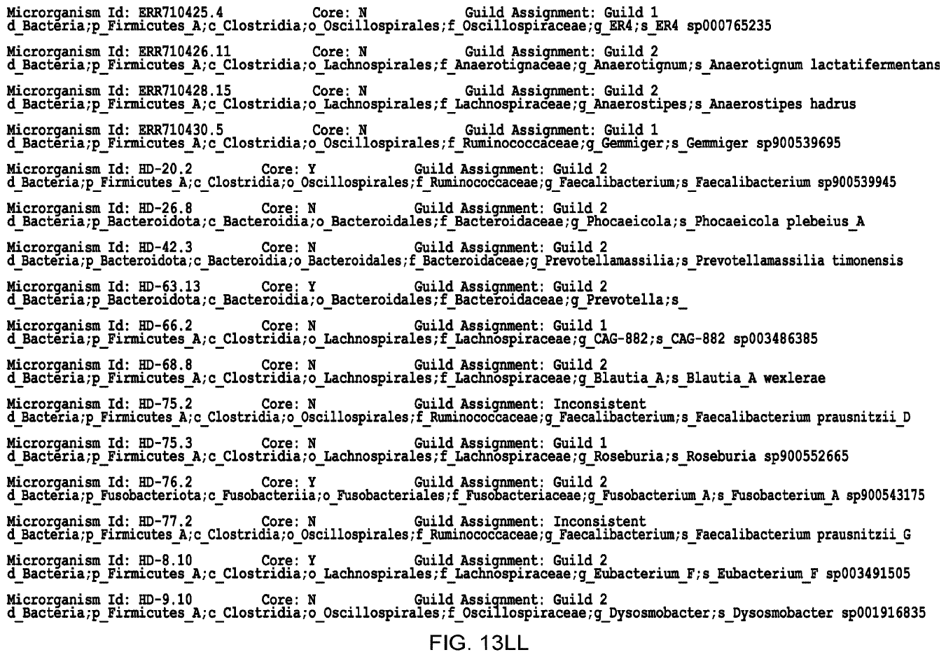

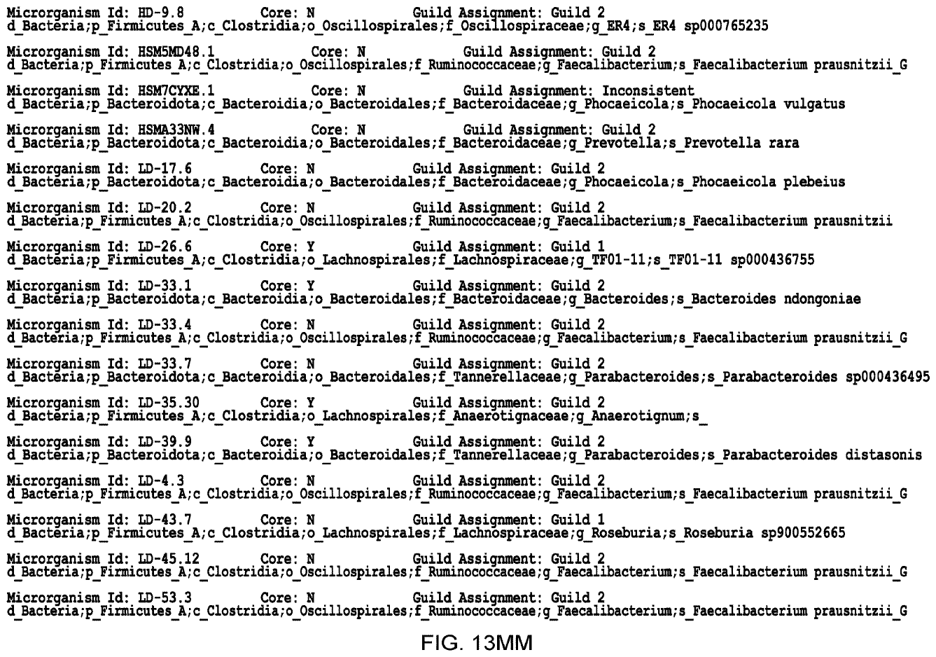

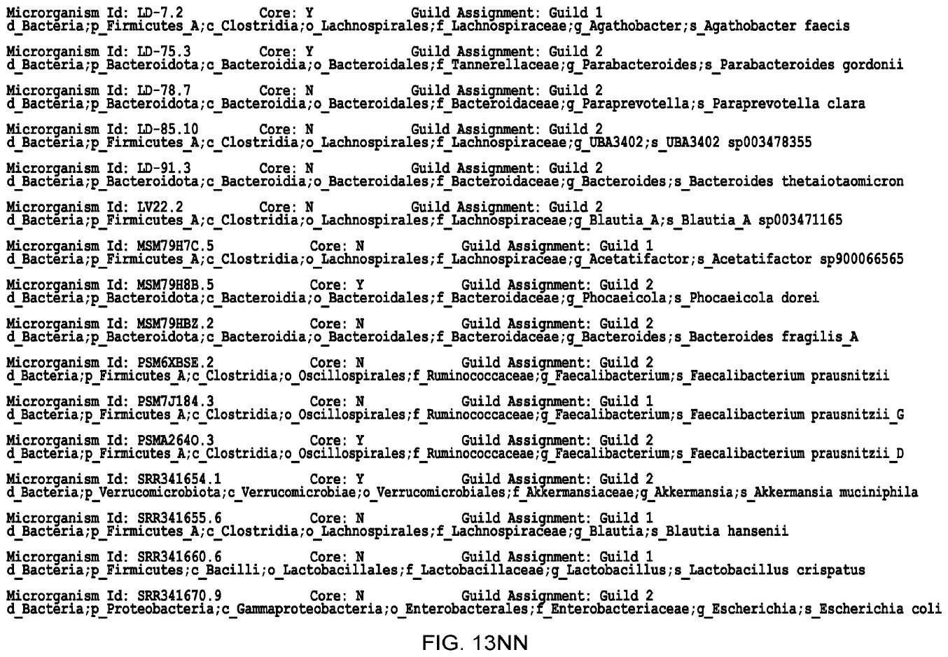

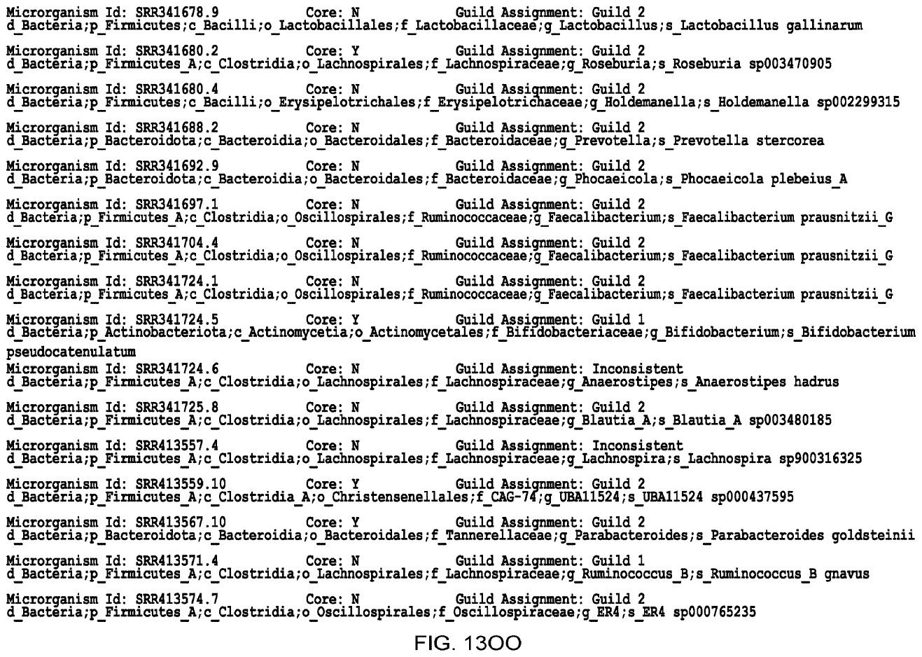

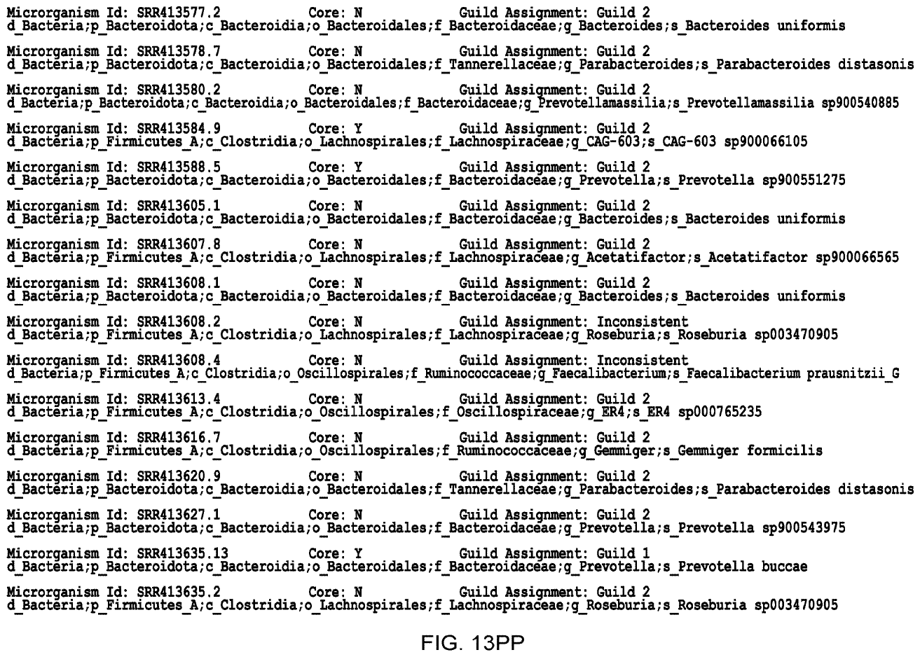

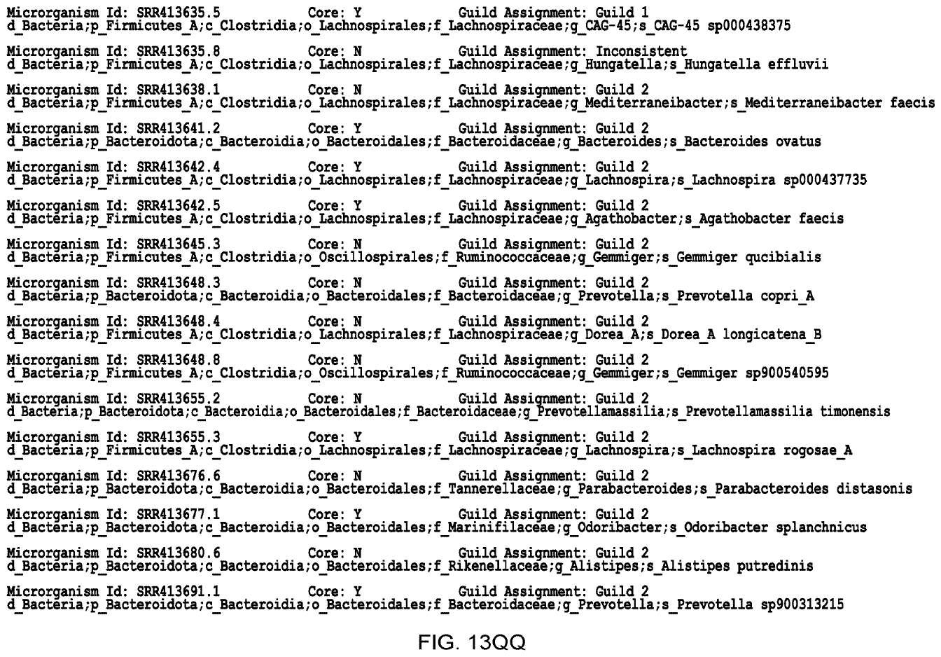

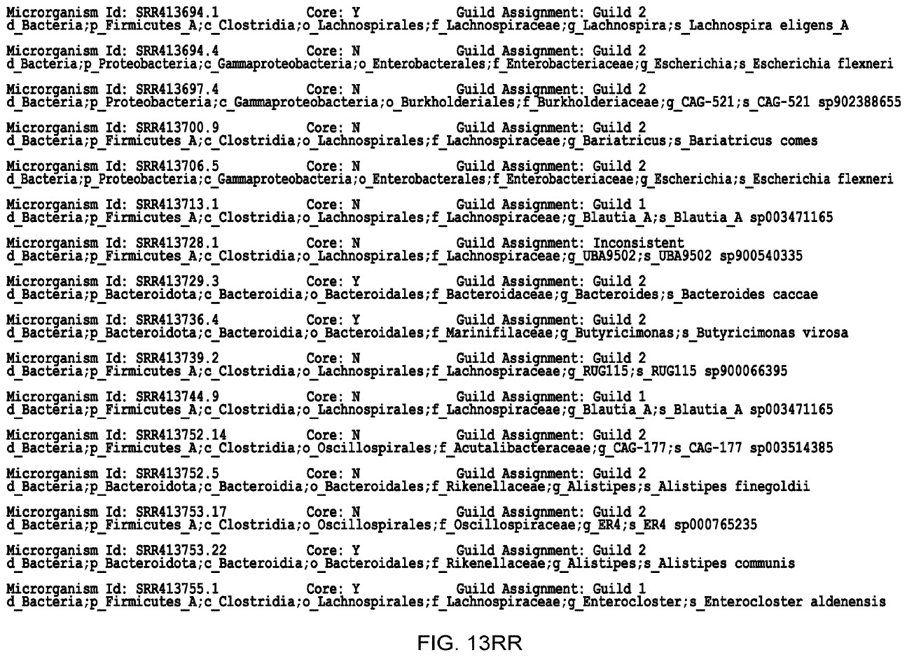

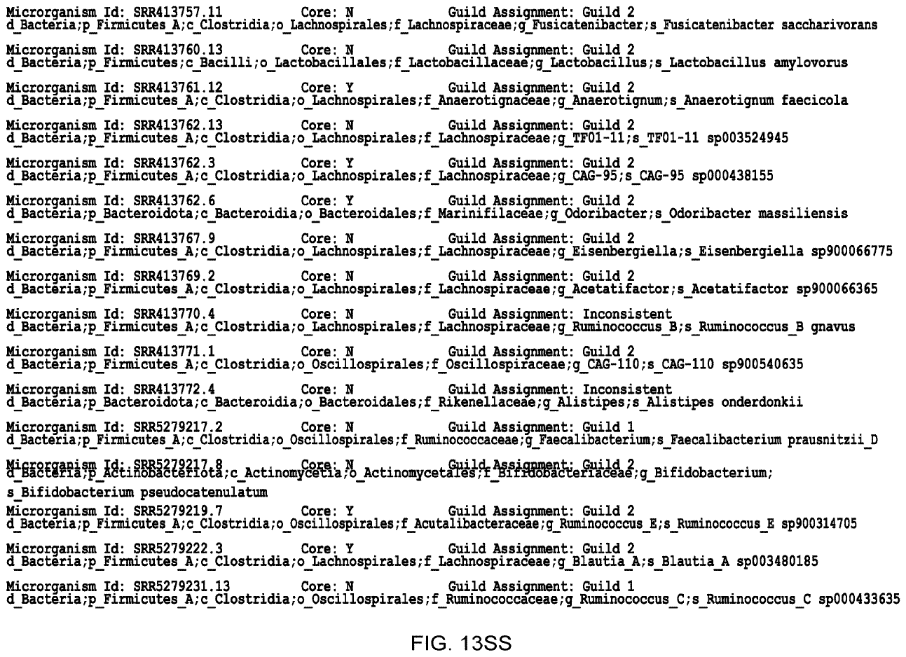

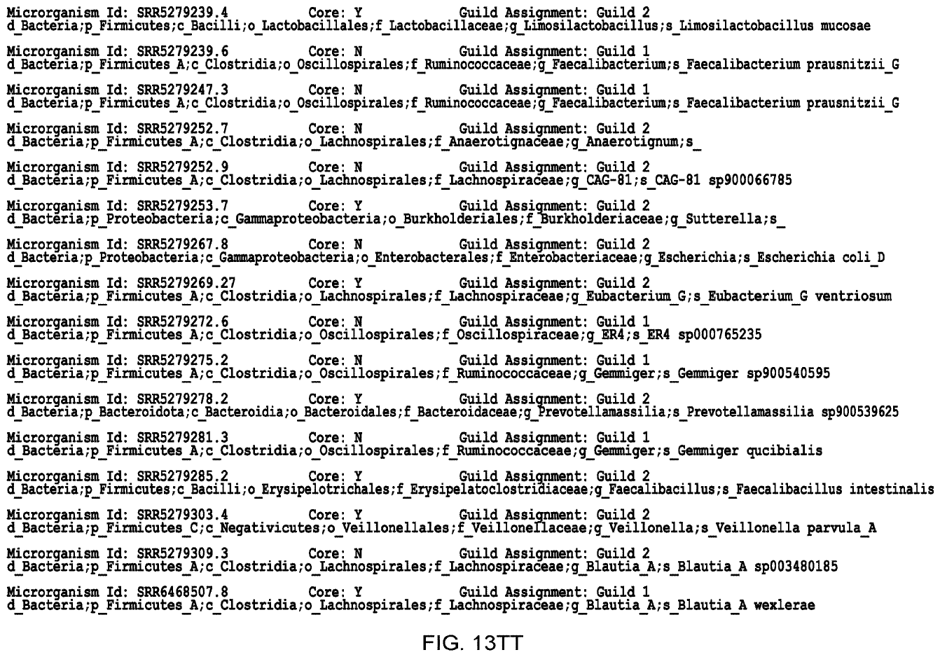

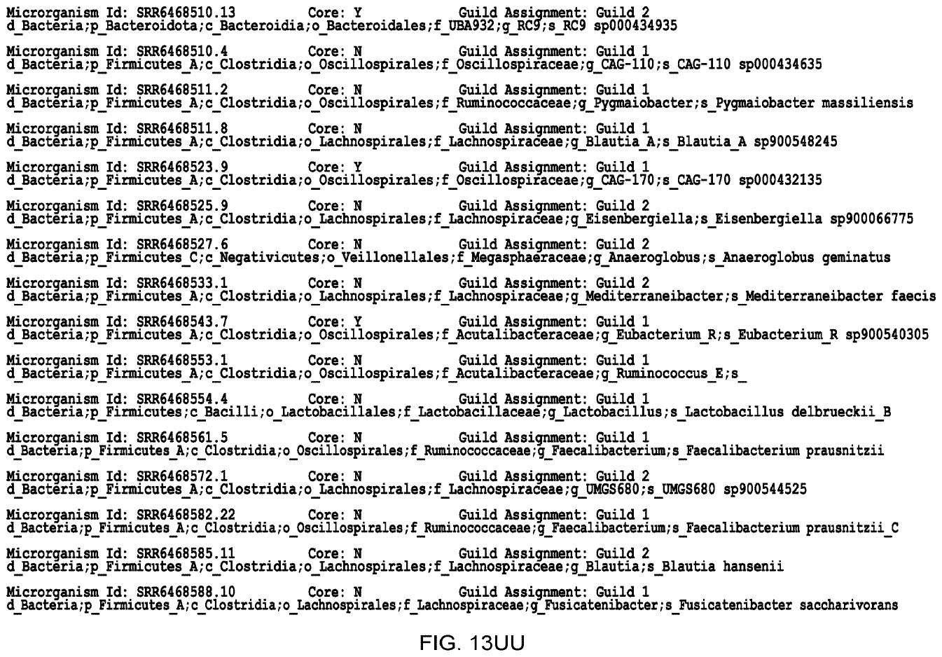

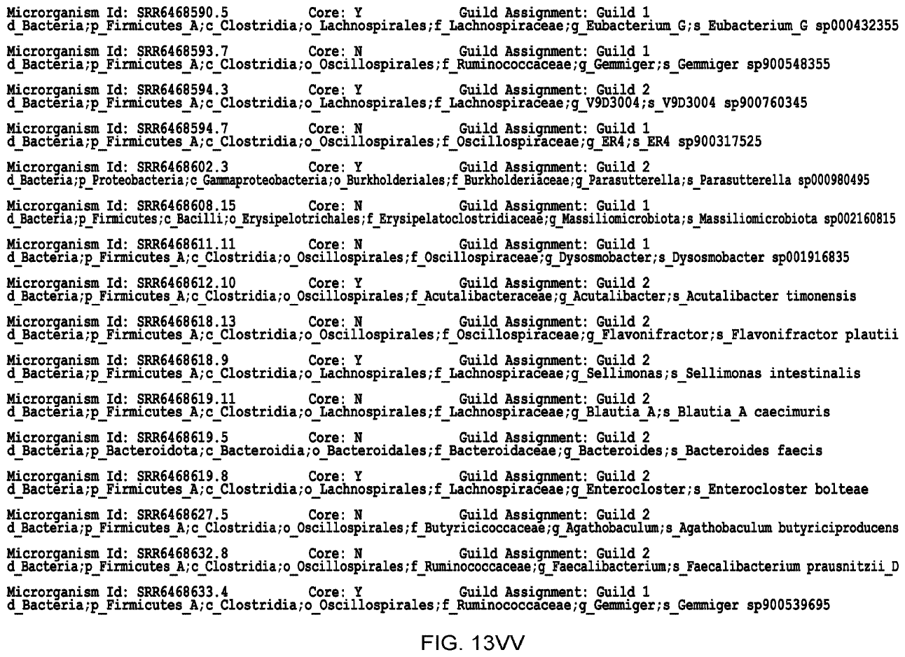

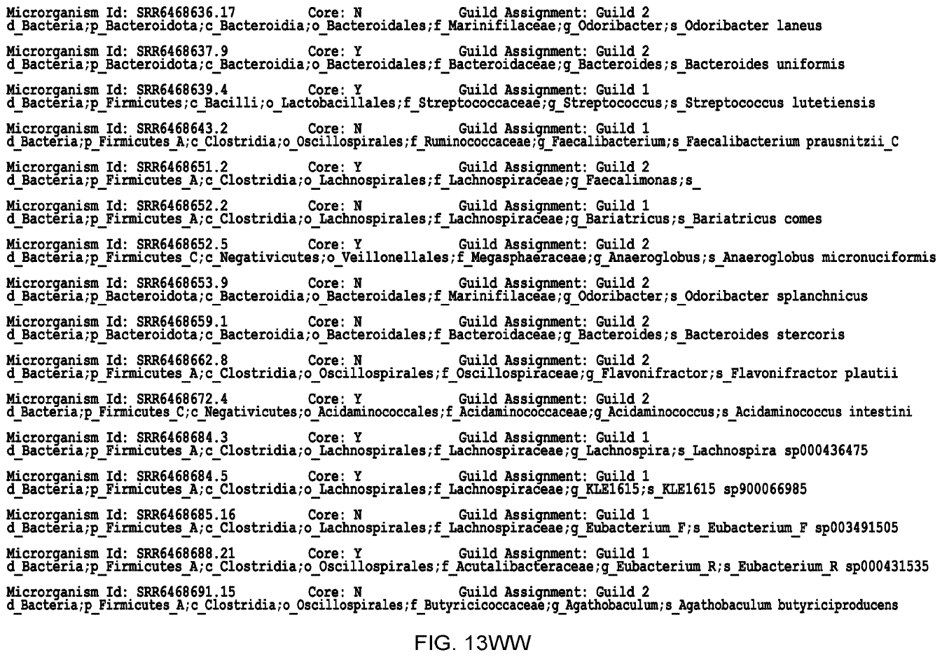

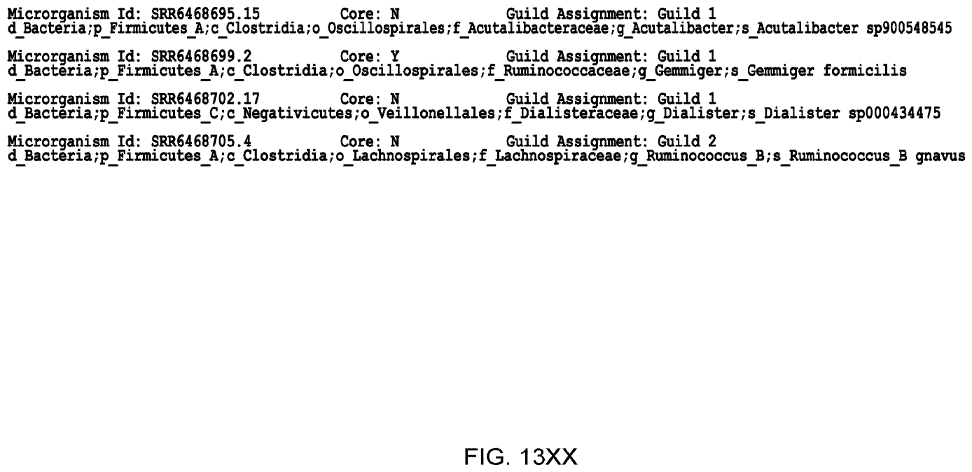

In some embodiments, the corresponding plurality of nucleic acid sequences are obtained through targeted panel sequencing. An example of targeted panel sequencing is described in U.S. Patent Application Publication No. 2019/0316209. In some embodiments, the targeted panel sequencing comprises hybridizing genomic DNA isolated from a biological sample from the gut of a subject with a panel of probes that include one or more probes that hybridize to a unique sequence in the genome of each microorganism being quantified, prior to sequencing recovered nucleic acids. In some embodiments, the microorganisms include a plurality of the microorganisms listed in Table 1, Table 2, and/or FIGS. 13A-13XX. In some embodiments, a combination of semi-unique sequences (e.g., sequences found in a small number of the microorganism genomes) can be used to deconvolute genomic abundance values using an algorithm, e.g., a system of equations. In some embodiments, the panel of probes includes at least 1 probe that hybridizes to a sequence unique to each microorganism genome being detected. In some embodiments, the panel of probes includes at least 2, at least 3, at least 4, at least 5, at least 10, at least 25, at least 50, or more probes that hybridize to a different sequence unique to each microorganism genome being detected. In some embodiments, the panel of probes includes at least 20, at least 30, at least 40, at least 50, at least 75, at least 100, at least 125, at least 150, at least 200, at least 150, at least 300, at least 400, at least 500, at least 750, at least 1000, at least 1250, at least 1500, at least 2000, at least 2500, at least 3000, at least 4000, at least 5000, at least 7500, at least 10,000 or more unique probes.

In some embodiments, the sequencing genomic DNA from the corresponding biological sample comprises a partial or complete sequencing platform adapter sequence at their termini useful for sequencing using a sequencing platform of interest. Sequencing platforms of interest include, but are not limited to, the HiSeq™, MiSeq™ and Genome Analyzer™ sequencing systems from Illumina®; the Ion PGM™ and Ion Proton™ sequencing systems from Ion Torrent™; the PACBIO RS II Sequel system from Pacific Biosciences, the SOLID sequencing systems from Life Technologies™, the 454 GS FLX+ and GS Junior sequencing systems from Roche, the MinION™ system from Oxford Nanopore, or any other sequencing platform of interest.

Referring to block 204, in some embodiments, the methods include obtaining, for each respective training subject in the plurality of training subjects, in electronic form, a corresponding plurality of nucleic acid sequences for genomic DNA from the corresponding biological sample from the gut of the respective training subject.

Referring to block 206, in some embodiments, the methods include determining, for each respective gut microorganism in the plurality of gut microorganisms, the corresponding value for the abundance of the genome of the respective gut microorganism from the corresponding plurality of nucleic acid sequences.

In some embodiments, the genomic abundance values determined for each respective subject in the plurality of training subjects comprise at least 20, at least 25, at least 50, at least 75, at least 100, at least 125, at least 150, at least 175, at least 200, at least 250, at least 300, at least 350, at least 400, at least 500, at least 600, at least 700, at least 700, at least 800, at least 900, at least 1000, at least 1500, at least 2000, at least 25000, at least 5,000 or at least 10,000 genome abundance values, where each genome abundance value corresponds to different gut microorganism. In some embodiments, the genomic abundance values determined for each respective subject in the plurality of training subjects comprise no more than 250,000, no more than 100,000, no more than 50,000, no more than 25,000, no more than 10,000, no more than 5000, no more than 2500, no more than 1000, no more than 750, no more than 500, or fewer genome abundance values. In some embodiments, the genomic abundance values determined for each respective subject in the plurality of training subjects consist of from 10 to 40, from 20 to 50, from 30 to 80, from 40 to 100, from 50 to 150, from 60 to 200, from 80 to 300, from 90 to 500, from 100 to 1000, from 500 to 2,000, or from 1,000 to 5,000 genome abundance values. In some embodiments, the genomic abundance values determined for each respective subject in the plurality of training subjects fall within another range starting no lower than 10 genome abundance values and ending no higher than 250,000 genome abundance values.

Referring to block 208, in some embodiments, for each respective training subject in the plurality of training subjects, the methods include assembling, in electronic form, a corresponding plurality of gut microorganism genomes by metagenomic de novo sequence assembly from the corresponding plurality of nucleic acid sequences, and calculating, for each respective gut microorganism in the plurality of gut microorganisms, the corresponding value for the abundance of the genome of the respective gut microorganism based on the prevalence of respective nucleic acid sequences, in the plurality of nucleic acid sequences, used to assemble a respective gut microorganism genome in the plurality of gut microorganism genomes corresponding to the respective gut microorganism. In some embodiments, metagenomic de novo sequence assembly further comprise generating contigs based on the sequencing reads generated by a shotgun sequencing technique. Such a technique is described, for example, in U.S. Pat. No. 10,529,443, the content of which is incorporated herein by reference in its entirety. In some embodiments, the first plurality of nucleic acid sequences is assembled into full genomes of the plurality of gut microorganisms. In some embodiments, the plurality of nucleic acid sequences is assembled into partial genomes of the plurality of gut microorganisms.

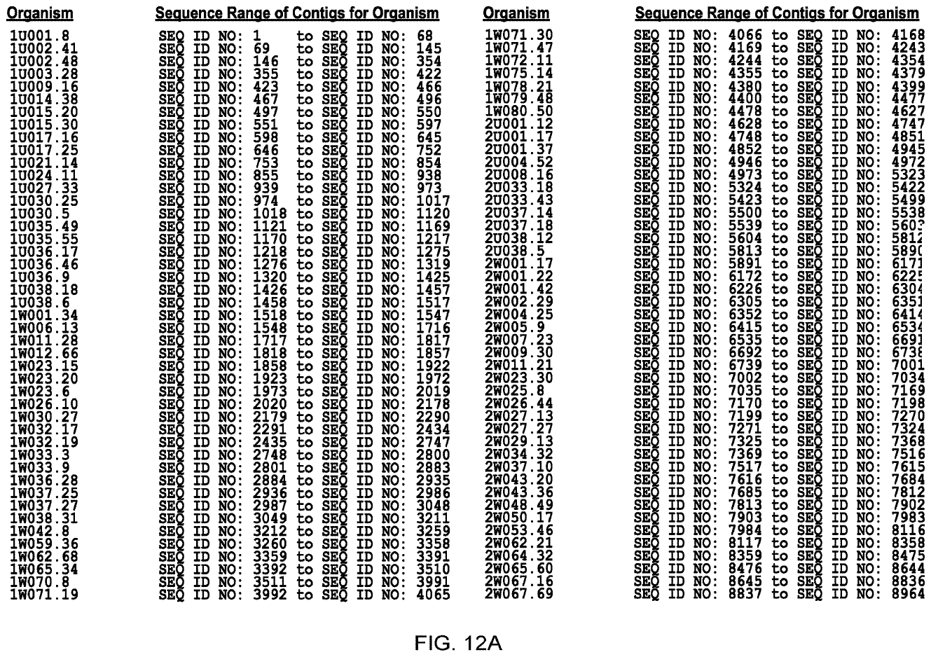

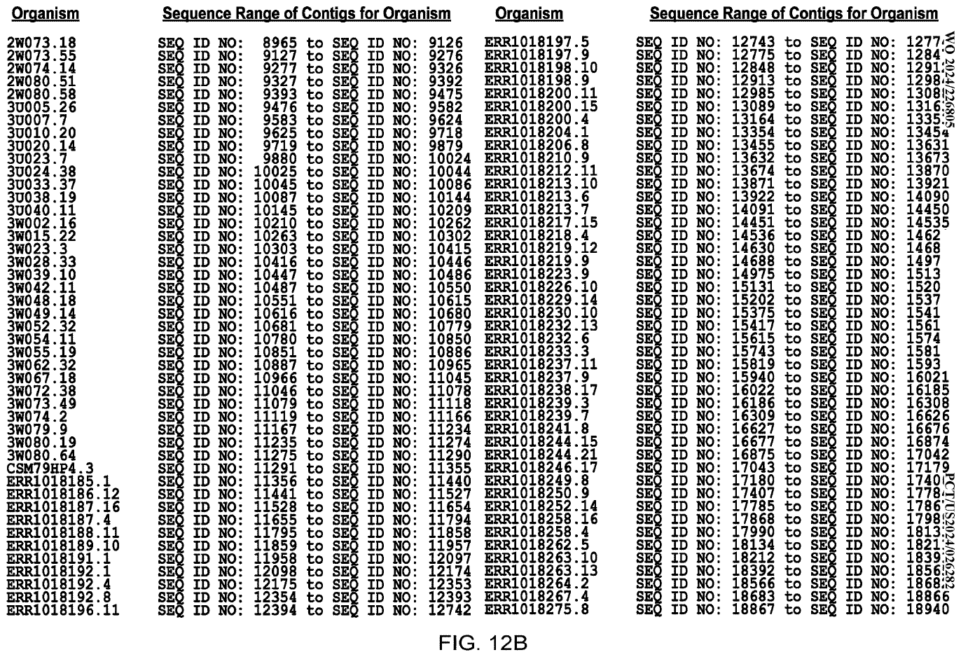

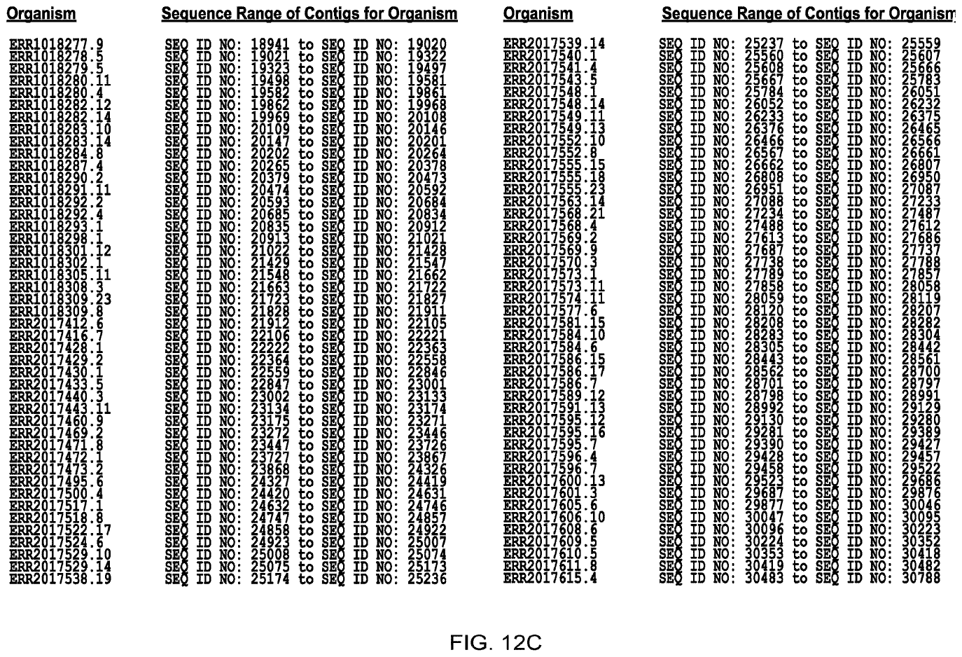

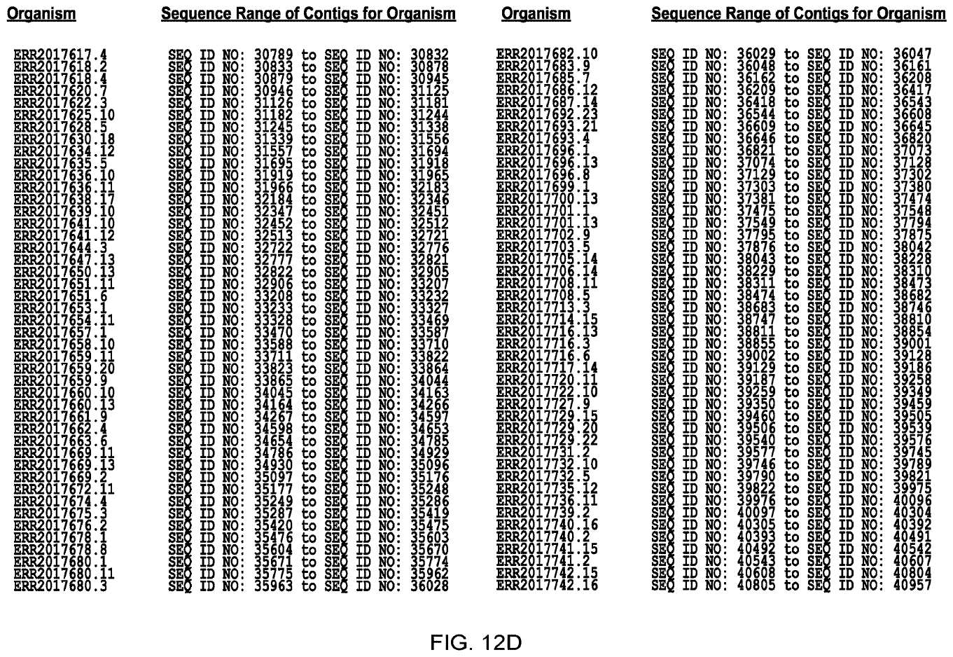

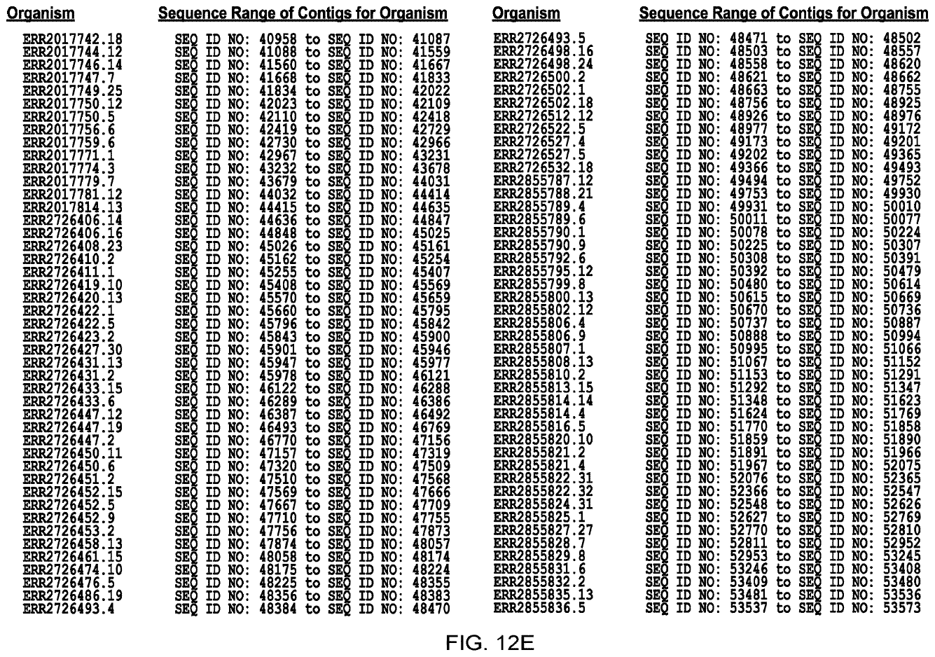

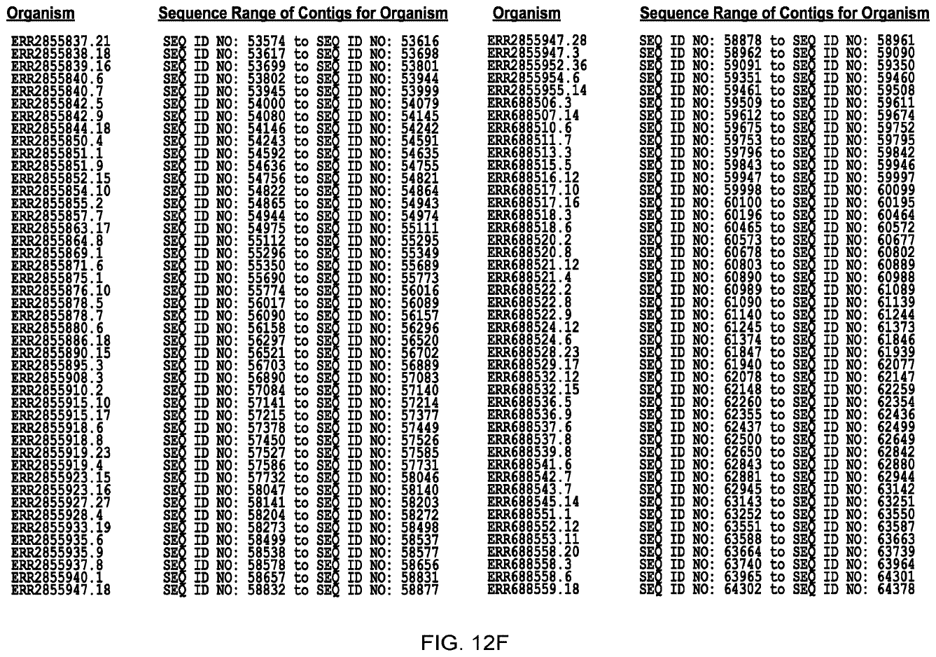

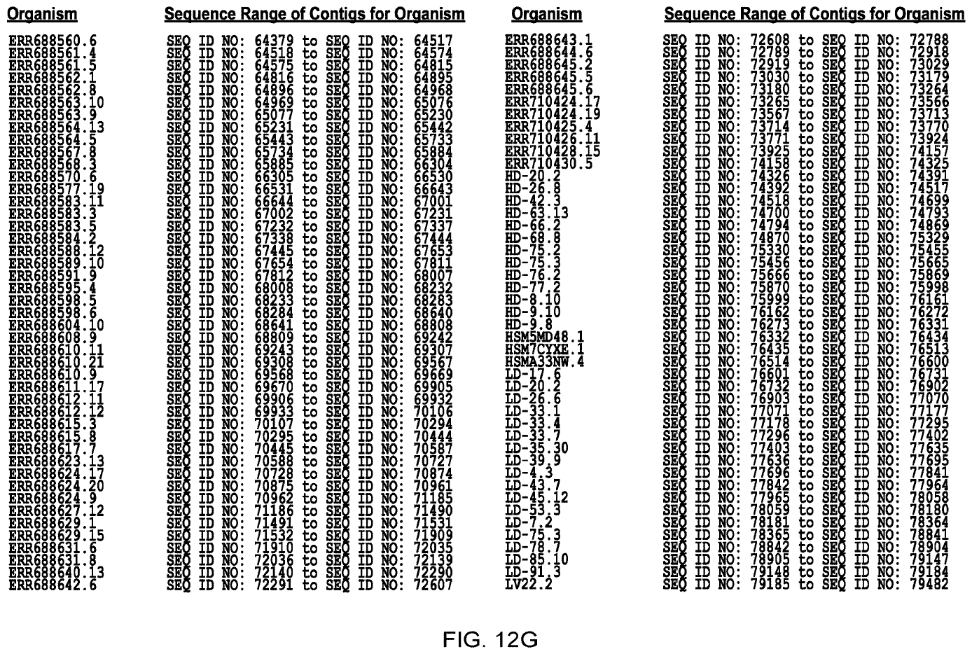

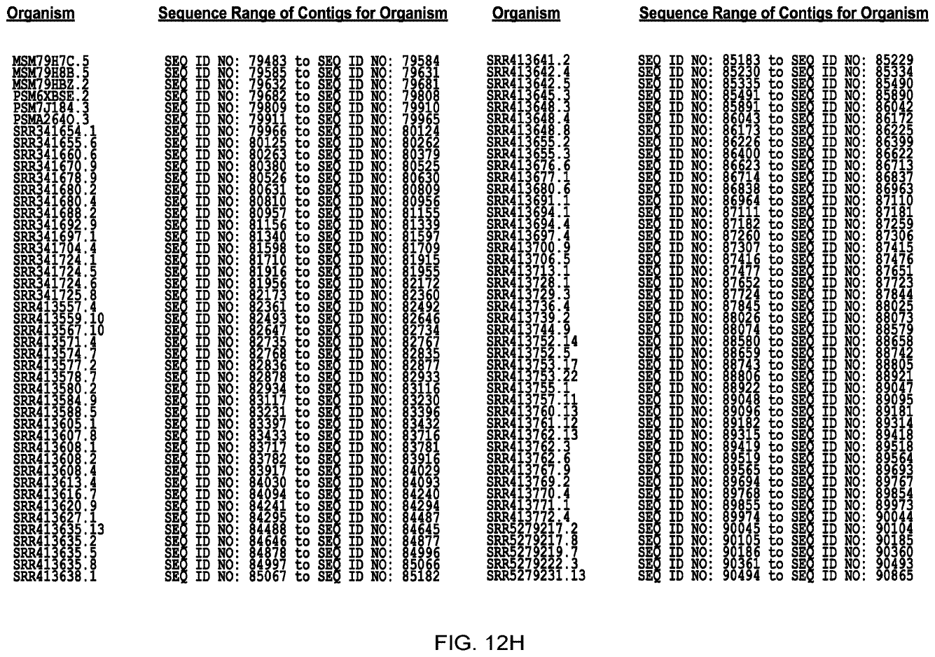

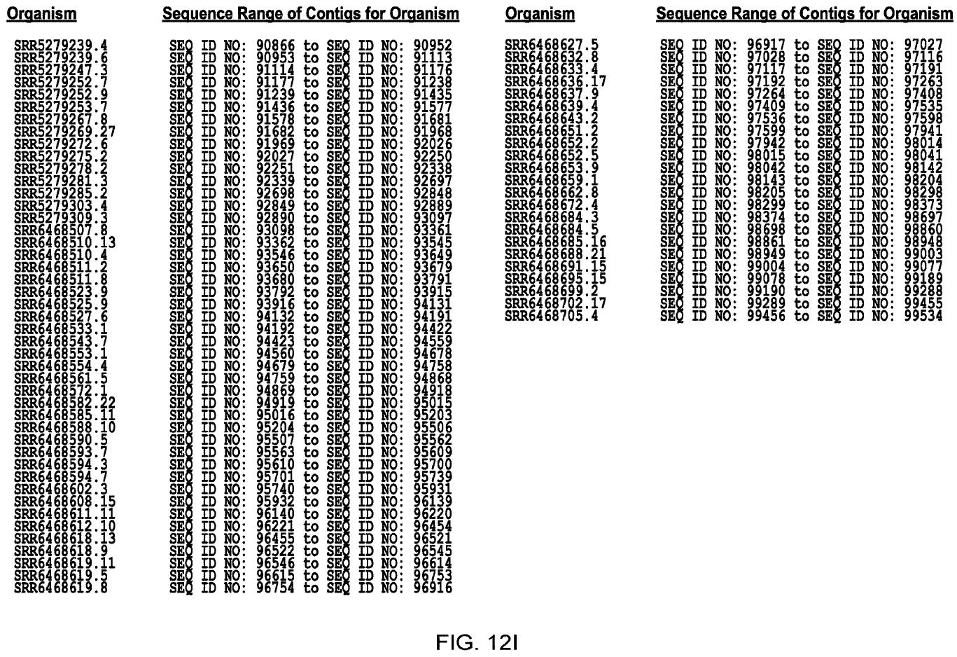

Referring to block 210, in some embodiments, for each respective subject in the plurality of training subjects, the methods including assigning each respective nucleic acid sequence in the corresponding plurality of nucleic acid sequences to a respective gut microorganism in the plurality of gut microorganisms, thereby generating, for each respective gut microorganism in the plurality of gut microorganism, a corresponding count of respective nucleic acid sequences in the corresponding plurality of nucleic acid sequences assigned to the respective gut microorganism, and determining, for each respective gut microorganism in the plurality of gut microorganisms, the corresponding genomic abundance value for the respective gut microorganism based on the corresponding count of respective nucleic acid sequences assigned to the respective gut microorganism. In some embodiments, the assigning each respective nucleic acids to a respective gut microorganism includes mapping the nucleic acid to a reference nucleic acid (e.g., a contig listed in FIG. 12) In some embodiments, the assigning each respective nucleic acids a respective gut microorganism includes annotating genome information based on existing databases. In some embodiments, nucleic acid sequences are analyzed, and annotations are to define taxonomic assignments using sequence similarity and phylogenetic placement methods or a combination of the two strategies.

Sequence similarity-based methods for assigning each nucleic acid sequence to a respective gut microorganism include those familiar to individuals skilled in the art including, but not limited to BLAST, BLASTx, tBLASTn, tBLASTx, RDP-classifier, DNAclust, and various implementations of these algorithms such as Qiime or Mothur. These methods rely on mapping a sequence read to a reference database and selecting the match with the best score and e-value. In some embodiments, phylogenetic methods are used in combination with sequence similarity methods to improve the calling accuracy of an annotation or taxonomic assignment. Common databases include, but not limited to, GT-DBTK, National Center for Biotechnology Information (NCBI) Genbank, European Bioinformatics Institute-European Nucleotide Archive (European Bioinformatics Institute-European Nucleotide Archive; EBI-ENA), National Institute of Genetics, U.S. Department of ENERGY (USDOE) Integrated Microbial Genomes (Integrated Microbial Genomes) &Microbiomes; IMG/M) and other available databases in the art.

In some embodiments, the plurality of genomic abundance values is determined using a microarray comprising a probe sequence capable of detecting a unique genomic sequence of each respective genome for the plurality of gut microorganisms. In some embodiments, the panel of probes on a microarray includes at least 1 probe that hybridizes to a sequence unique to each microorganism genome being detected. In some embodiments, the panel of probes includes at least 2, at least 3, at least 4, at least 5, at least 10, at least 25, at least 50, or more probes that hybridize to a different sequence unique to each microorganism genome being detected. In some embodiments, the panel of probes includes at least 20, at least 30, at least 40, at least 50, at least 75, at least 100, at least 125, at least 150, at least 200, at least 150, at least 300, at least 400, at least 500, at least 750, at least 1000, at least 1250, at least 1500, at least 2000, at least 2500, at least 3000, at least 4000, at least 5000, at least 7500, at least 10,000 or more unique probes.

Referring to block 212, in some embodiments, the plurality of gut microorganisms comprises at least 20 gut microorganisms selected from Table 1, Table 2, or FIG. 13A-13XX. In some embodiments, gut microorganisms of at least about 20, 25, 30, 35, 40, 45, 50, 55, 60, 65, 70, 75, 80, 85, 90, 95, 100 or greater are selected from Table 1, Table 2 or FIG. 13A-13XX. In some embodiments, the plurality of gut microorganisms comprises at least 25 gut microorganisms selected from Table 1, Table 2, or FIGS. 13A-13XX. In some embodiments, the plurality of gut microorganisms comprises at least 30 gut microorganisms selected from Table 1, Table 2, or FIGS. 13A-13XX. In some embodiments, the plurality of gut microorganisms comprises at least 40 gut microorganisms selected from Table 1, Table 2, or FIGS. 13A-13XX. In some embodiments, the plurality of gut microorganisms comprises at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, at least 600, at least 700, at least 800, or all of the gut microorganisms selected from Table 1, Table 2 or FIGS. 13A-13XX. In some embodiments, the plurality of gut microorganisms are at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, at least 600, at least 700, at least 800, or all of the gut microorganisms selected from Table 1, Table 2 or FIGS. 13A-13XX. In some embodiments, the plurality of gut microorganisms are all of the gut microorganisms listed in Table 1. In some embodiments, the plurality of gut microorganisms are all of the gut microorganisms listed in Table 2. In some embodiments, the plurality of gut microorganisms are all of the gut microorganisms listed in FIGS. 13A-13XX.

TABLE 1
Taxonomy Assignment of 141 non-redundant genomes identified in two competing guilds

| | Taxonomy Assignment (d = domain; p = phylum; c = class; o = order; | |
|---|---|---|
| ID | f = family; g = genus; s = species) | Guilds |
| 1U030.25 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_G | |
| 1U035.49 | d_{——}Bacteria; p_{——}Firmicutes_{——}A; c_{——}Clostridia; o_{——}Oscillospirales; | Guild 1 |
| | f_{——}Oscillospiraceae; g_{——}CAG-103; s | |
| 1U036.46 | d_{——}Bacteria; p_{——}Firmicutes_{——}A; c_{——}Clostridia; o_{——}Oscillospirales; | Guild 1 |
| | f_{——}Oscillospiraceae; g_{——}CAG-103; s | |
| 1W012.66 | d_{——}Bacteria; p_{——}Firmicutes_{——}A; c_{——}Clostridia; o_{——}Oscillospirales; | Guild 1 |
| | f_{——}Oscillospiraceae; g_{——}ER4; s_{——}ER4 sp900317525 | |
| 1W030.27 | d_{——}Bacteria; p_{——}Firmicutes_{——}A; c_{——}Clostridia; o_{——}Oscillospirales; | Guild 1 |
| | f_{——}Oscillospiraceae; g_{——}Oscillibacter; s_{——}Oscillibacter sp001916835 | |
| 1W037.25 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_K | |
| 1W059.36 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_K | |
| 1W065.34 | d_{——}Bacteria; p_{——}Firmicutes_{——}A; c_{——}Clostridia; o_{——}Oscillospirales; | Guild 1 |
| | f_{——}Oscillospiraceae; g_{——}Oscillibacter; s_{——}Oscillibacter sp001916835 | |
| 1W071.30 | d_{——}Bacteria; p_{——}Firmicutes_{——}A; c_{——}Clostridia; o_{——}Oscillospirales; | Guild 1 |
| | f_{——}Ruminococcaceae; g_{——}Gemmiger; s | |
| 1W071.47 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_G | |
| 2U004.52 | d_{——}Bacteria; p_{——}Firmicutes_{——}A; c_{——}Clostridia; o_{——}Oscillospirales; | Guild 1 |
| | f_{——}Oscillospiraceae; g_{——}CAG-103; s | |
| 2U017.37 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_G | |
| 2U033.43 | d_{——}Bacteria; p_{——}Firmicutes_{——}A; c_{——}Clostridia; o_{——}Oscillospirales; | Guild 1 |
| | f_{——}Ruminococcaceae; g_{——}Ruminococcus_C; s | |
| 2W001.22 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_K | |
| 2W007.23 | d_{——}Bacteria; p_{——}Firmicutes_{——}A; c_{——}Clostridia; o_{——}Oscillospirales; | Guild 1 |
| | f_{——}Oscillospiraceae; g_{——}CAG-110; s_{——}CAG-110 sp000434635 | |
| 2W009.30 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_G | |
| 2W023.30 | d_{——}Bacteria; p_{——}Firmicutes_{——}A; c_{——}Clostridia; o_{——}Oscillospirales; | Guild 1 |
| | f_{——}Acutalibacteraceae; g_{——}UBA1417; s | |
| 2W027.13 | d_{——}Bacteria; p_{——}Firmicutes_{——}A; c_{——}Clostridia; o_{——}Lachnospirales; | Guild 1 |
| | f_{——}Lachnospiraceae; g_{——}Eisenbergiella; s_{——}Eisenbergiella sp900066775 | |
| 2W027.27 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_D | |
| 2W029.13 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_C | |
| 2W034.32 | d_{——}Bacteria; p_{——}Firmicutes_{——}A; c_{——}Clostridia; o_{——}Oscillospirales; | Guild 1 |
| | f_{——}Oscillospiraceae; g_{——}CAG-110; s | |
| 2W062.21 | d_{——}Bacteria; p_{——}Firmicutes_{——}A; c_{——}Clostridia; o_{——}Oscillospirales; | Guild 1 |
| | f_{——}Oscillospiraceae; g_{——}Oscillibacter; s_{——}Oscillibacter sp001916835 | |
| 2W064.32 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_D | |
| 2W067.69 | d_{——}Bacteria; p_{——}Firmicutes_{——}A; c_{——}Clostridia; o_{——}Oscillospirales; | Guild 1 |
| | f_{——}Oscillospiraceae; g_{——}ER4; s_{——}ER4 sp000765235 | |
| 2W073.18 | d_{——}Bacteria; p_{——}Firmicutes_{——}A; c_{——}Clostridia; o_{——}Christensenellales; | Guild 1 |
| | f_{——}UBA1750; g_{——}UBA7102; s | |
| 2W073.55 | d_{——}Bacteria; p_{——}Firmicutes_{——}A; c_{——}Clostridia; o_{——}Oscillospirales; | Guild 1 |
| | f_{——}Butyricicoccaceae; g_{——}Butyricicoccus_A; s_{——}Butyricicoccus_A sp002395695 | |
| 2W074.14 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_K | |
| 2W080.51 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_I | |
| 3U005.26 | d_{——}Bacteria; p_{——}Firmicutes_{——}A; c_{——}Clostridia; o_{——}Oscillospirales; | Guild 1 |
| | f_{——}Ruminococcaceae; g_{——}Gemmiger; s_{——}Gemmiger formicilis | |
| 3U010.20 | d_{——}Bacteria; p_{——}Firmicutes_{——}A; c_{——}Clostridia; o_{——}Oscillospirales; | Guild 1 |
| | f_{——}Oscillospiraceae; g_{——}CAG-110; s | |
| 3U024.38 | d_{——}Bacteria; p_{——}Firmicutes_{——}A; c_{——}Clostridia; o_{——}Oscillospirales; | Guild 1 |
| | f_{——}Acutalibacteraceae; g_{——}CAG-180; s_{——}CAG-180 sp000432435 | |
| 3U033.37 | d_{——}Bacteria; p_{——}Firmicutes_{——}A; c_{——}Clostridia; o_{——}Oscillospirales; | Guild 1 |
| | f_{——}Oscillospiraceae; g_{——}CAG-110; s | |
| 3W015.22 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_J | |
| 3W048.18 | d_{——}Bacteria; p_{——}Firmicutes_{——}A; c_{——}Clostridia; o_{——}Oscillospirales; | Guild 1 |
| | f_{——}Ruminococcaceae; g_{——}Gemmiger; s | |
| 3W052.32 | d_{——}Bacteria; p_{——}Firmicutes_{——}A; c_{——}Clostridia; o_{——}Oscillospirales; | Guild 1 |
| | f_{——}Ruminococcaceae; g_{——}Gemmiger; s | |
| 3W062.32 | d_{——}Bacteria; p_{——}Firmicutes_{——}A; c_{——}Clostridia; o_{——}Oscillospirales; | Guild 1 |
| | f_{——}Ruminococcaceae; g_{——}CAG-353; s_{——}CAG-353 sp900066885 | |
| 3W072.38 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_G | |
| 3W073.49 | d_{——}Bacteria; p_{——}Firmicutes_{——}A; c_{——}Clostridia; o_{——}Christensenellales; | Guild 1 |
| | f_{——}CAG-138; g_{——}Phil1; s_{——}Phil1 sp001940855 | |
| 1U009.16 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_G | |
| 1W011.28 | d_{——}Bacteria; p_{——}Firmicutes_{——}A; c_{——}Clostridia; o_{——}Oscillospirales; | Guild 1 |
| | f_{——}Oscillospiraceae; g_{——}Oscillibacter; s_{——}Oscillibacter sp001916835 | |
| 1W001.34 | d_{——}Bacteria; p_{——}Firmicutes_{——}A; c_{——}Clostridia; o_{——}Oscillospirales; | Guild 1 |
| | f_{——}Acutalibacteraceae; g_{——}CAG-177; s_{——}CAG-177 sp003514385 | |
| 1U014.38 | d_{——}Bacteria; p_{——}Firmicutes_{——}A; c_{——}Clostridia; o_{——}Oscillospirales; | Guild 1 |
| | f_{——}Ruminococcaceae; g_{——}Faecalibacterium; s_{——}Faecalibacterium prausnitzii_K | |
| 1W037.27 | d_{——}Bacteria; p_{——}Firmicutes_{——}A; c_{——}Clostridia; o_{——}Oscillospirales; | Guild 1 |
| | f_{——}Ruminococcaceae; g_{——}Gemmiger; s_{——}Gemmiger formicilis | |
| 1U002.48 | d_{——}Bacteria; p_{——}Firmicutes_{——}A; c_{——}Clostridia; o_{——}Oscillospirales; | Guild 1 |
| | f_{——}Ruminococcaceae; g_{——}Ruminococcus_C; s_{——}Ruminococcus_C callidus | |
| 2W066.63 | d_{——}Bacteria; p_{——}Firmicutes_{——}A; c_{——}Clostridia; o_{——}Oscillospirales; | Guild 1 |
| | f_{——}Oscillospiraceae; g_{——}ER4; s_{——}ER4 sp900317525 | |
| 2W043.36 | d_{——}Bacteria; p_{——}Firmicutes_{——}A; c_{——}Clostridia; o_{——}Oscillospirales; | Guild 1 |
| | f_{——}Ruminococcaceae; g_{——}Gemmiger; s_{——}Gemmiger formicilis | |
| 2W043.20 | d_{——}Bacteria; p_{——}Firmicutes_{——}A; c_{——}Clostridia; o_{——}Lachnospirales; | Guild 1 |
| | f_{——}Lachnospiraceae; g_{——}Eisenbergiella; s_{——}Eisenbergiella sp900066775 | |
| 2W001.17 | d_{——}Bacteria; p_{——}Firmicutes_{——}A; c_{——}Clostridia; o_{——}Oscillospirales; | Guild 1 |
| | f_{——}Ruminococcaceae; g_{——}Gemmiger sp003476825 | |
| 1U002.41 | d_{——}Bacteria; p_{——}Firmicutes_{——}A; c_{——}Clostridia; o_{——}Lachnospirales; | Guild 1 |
| | f_{——}Lachnospiraceae; g_{——}Dorea; s_{——}Dorea longicatena_B | |
| 1W071.19 | d_{——}Bacteria; p_{——}Firmicutes_{——}A; c_{——}Clostridia; o_{——}Lachnospirales; | Guild 1 |
| | f_{——}Lachnospiraceae; g_{——}Coprococcus_A; s_{——}Coprococcus_A catus | |
| 1U017.16 | d_{——}Bacteria; p_{——}Firmicutes_{——}A; c_{——}Clostridia; o_{——}Lachnospirales; | Guild 2 |
| | f_{——}Lachnospiraceae; g_{——}Blautia; s | |
| 1U017.25 | d_{——}Bacteria; p_{——}Firmicutes_{——}A; c_{——}Clostridia; o_{——}Lachnospirales; | Guild 2 |
| | f_{——}Lachnospiraceae; g_{——}Tyzzerella; s_{——}Tyzzerellasp000209385 | |
| 1U028.21 | d_{——}Bacteria; p_{——}Firmicutes_{——}A; c_{——}Clostridia; o_{——}Lachnospirales; | Guild 2 |
| | f_{——}Lachnospiraceae; g_{——}Dorea; s_{——}Dorea scindens | |
| 1U030.5 | d_{——}Bacteria; p_{——}Proteobacteria; c_{——}Gammaproteobacteria; o_{——}Enterobacterales; | Guild 2 |
| | f_{——}Enterobacteriaceae; g_{——}Escherichia; s_{——}Escherichia coli_D | |
| 1U035.55 | d_{——}Bacteria; p_{——}Firmicutes; c_{——}Bacilli; o_{——}Lactobacillies; | Guild 2 |
| | f_{——}Streptococcaceae; g_{——}Streptococcus; s_{——}Streptococcus macedonicus | |
| 1U038.18 | d_{——}Bacteria; p_{——}Firmicutes_{——}A; c_{——}Clostridia; o_{——}Lachnospirales; | Guild 2 |
| | f_{——}Lachnospiraceae; g_{——}UBA7096; s | |
| 1U038.6 | d_{——}Bacteria; p_{——}Proteobacteria; c_{——}Gammaproteobacteria; o_{——}Enterobacterales; | Guild 2 |
| | f_{——}Enterobacteriaceae; g_{——}Escherichia; s_{——}Escherichia coli_D | |
| 1W006.13 | d_{——}Bacteria; p_{——}Firmicutes_{——}A; c_{——}Clostridia; o_{——}Lachnospirales; | Guild 2 |
| | f_{——}Lachnospiraceae; g_{——}Faecalicatena; s_{——}Faecalicatena faecis | |
| 1W032.17 | d_{——}Bacteria; p_{——}Bacteroidota; c_{——}Bacteroidia; o_{——}Bacteroidales; | Guild 2 |
| | f_{——}Bacteroidaceae; g_{——}Paraprevotella; s | |
| 1W032.19 | d_{——}Bacteria; p_{——}Firmicutes_{——}A; c_{——}Clostridia; o_{——}Lachnospirales; | Guild 2 |
| | f_{——}Lachnospiraceae; g_{——}Eubacterium_G; s_{——}Eubacterium_G sp000434315 | |
| 1W033.3 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s | |
| 1W033.9 | d_{——}Bacteria; p_{——}Firmicutes_{——}A; c_{——}Clostridia; o_{——}Oscillospirales; | Guild 2 |
| | f_{——}Oscillospiraceae; g_{——}Flavonifractor; s_{——}Flavonifractor plautii | |
| 1W036.28 | d_{——}Bacteria; p_{——}Firmicutes_{——}A; c_{——}Clostridia; o_{——}Lachnospirales; | Guild 2 |
| | f_{——}Lachnospiraceae; g_{——}Tyzzerella; s_{——}Tyzzerella nexilis | |
| 1W038.31 | d_{——}Bacteria; p_{——}Fusobacteriota; c_{——}Fusobacteriia; o_{——}Fusobacteriales; | Guild 2 |
| | f_{——}Fusobacteriaceae; g_{——}Fusobacterium_A; s | |
| 1W042.8 | d_{——}Bacteria; p_{——}Firmicutes_{——}A; c_{——}Clostridia; o_{——}Lachnospirales; | Guild 2 |
| | f_{——}Lachnospiraceae; g_{——}Clostridium_M; s_{——}Clostridium_M sp001517625 | |
| 1047.42 | d_{——}Bacteria; p_{——}Firmicutes_{——}C; c_{——}Negativicutes; o_{——}Acidaminococcales; | Guild 2 |
| | f_{——}Acidaminococcaceae; g_{——}Acidaminococcus; s_{——}Acidaminococcus intestini | |
| 1W062.68 | d_{——}Bacteria; p_{——}Firmicutes; c_{——}Bacilli; o_{——}Lactobacillies; | Guild 2 |
| | f_{——}Streptococcaceae; g_{——}Streptococcus; s_{——}Streptococcus sp002300045 | |
| 1W070.8 | d_{——}Bacteria; p_{——}Firmicutes_{——}A; c_{——}Clostridia; o_{——}Peptostreptococcales; | Guild 2 |
| | f_{——}Peptostreptococcaceae; g_{——}Intestinibacter; s_{——}Intestinibacter bartlettii | |
| 1W072.11 | d_{——}Bacteria; p_{——}Firmicutes; c_{——}Bacilli; o_{——}Erysipelotrichales; | Guild 2 |
| | f_{——}Erysipelotrichaceae; g_{——}Absiella; s | |
| 1W075.10 | d_{——}Bacteria; p_{——}Firmicutes_{——}A; c_{——}Clostridia; o_{——}Lachnospirales; | Guild 2 |
| | f_{——}Lachnospiraceae; g_{——}Blautia_A; s_{——}Blautia_A wexlerae | |
| 1W075.14 | d_{——}Bacteria; p_{——}Bacteroidota; c_{——}Bacteroidia; o_{——}Bacteroidales; | Guild 2 |
| | f_{——}Marinifilaceae; g_{——}Odoribacter; s | |
| 1W078.21 | d_{——}Bacteria; p_{——}Firmicutes_{——}C; c_{——}Negativicutes; o_{——}Veillonellales; | Guild 2 |
| | f_{——}Veillonellaceae; g_{——}Veillonella; s_{——}Veillonella parvula_A | |
| 1W080.50 | d_{——}Bacteria; p_{——}Proteobacteria; c_{——}Alphaproteobacteria; o_{——}RF32; | Guild 2 |
| | f_{——}CAG-239; g_{——}51-20; s_{——}51-20 sp001917175 | |
| 2U001.12 | d_{——}Bacteria; p_{——}Firmicutes_{——}A; c_{——}Clostridia; o_{——}Lachnospirales; | Guild 2 |
| | f_{——}Lachnospiraceae; g_{——}Blautia_A; s_{——}Blautia_A hydrogenotrophica | |
| 2U001.17 | d_{——}Bacteria; p_{——}Firmicutes_{——}A; c_{——}Clostridia; o_{——}Lachnospirales; | Guild 2 |
| | f_{——}Lachnospiraceae; g_{——}Clostridium_M; s_{——}Clostridium_M sp001517625 | |
| 2U001.37 | d_{——}Bacteria; p_{——}Firmicutes; c_{——}Bacilli; o_{——}Lactobacillales; | Guild 2 |
| | f_{——}Lactobacillaceae; g_{——}Lactobacillus_H; s_{——}Lactobacillus_H oris | |
| 2U008.16 | d_{——}Bacteria; p_{——}Fusobacteriota; c_{——}Fusobacteriia; o_{——}Fusobacteriales; | Guild 2 |
| | f_{——}Fusobacteriaceae; g_{——}Fusobacterium_A; s_{——}Fusobacterium_A ulcerans | |
| 2U017.6 | d_{——}Bacteria; p_{——}Proteobacteria; c_{——}Gammaproteobacteria; o_{——}Enterobacterales; | Guild 2 |
| | f_{——}Enterobacteriaceae; g_{——}Klebsiella; s_{——}Klebsiella variicola | |
| 2U033.18 | d_{——}Bacteria; p_{——}Bacteroidota; c_{——}Bacteroidia; o_{——}Bacteroidales; | Guild 2 |
| | f_{——}Bacteroidaceae; g_{——}Bacteroides_A; s_{——}Bacteroides_A sp000434735 | |
| 2U037.14 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides clarus | |
| 2U037.18 | d_{——}Bacteria; p_{——}Bacteroidota; c_{——}Bacteroidia; o_{——}Bacteroidales; | Guild 2 |
| | f_{——}Bacteroidaceae; g_{——}Bacteroides_A; s_{——}Bacteroides_A plebeius | |
| 2U037.6 | d_{——}Bacteria; p_{——}Proteobacteria; c_{——}Gammaproteobacteria; o_{——}Enterobacterales; | Guild 2 |
| | f_{——}Enterobacteriaceae; g_{——}Escherichia; s_{——}Escherichia flexneri | |
| 2U038.12 | d_{——}Bacteria; p_{——}Firmicutes_{——}A; c_{——}Clostridia; o_{——}Lachnospirales; | Guild 2 |
| | f_{——}Lachnospiraceae; g_{——}Clostridium_Q; s_{——}Clostridium_Q symbiosum | |
| 2U038.5 | d_{——}Bacteria; p_{——}Firmicutes_{——}A; c_{——}Clostridia; o_{——}Lachnospirales; | Guild 2 |
| | f_{——}Lachnospiraceae; g_{——}Blautia; s_{——}Blautia sp001304935 | |
| 2W001.42 | d_{——}Bacteria; p_{——}Proteobacteria; c_{——}Alphaproteobacteria; o_{——}RF32; | Guild 2 |
| | f_{——}CAG-239; g_{——}CAG-495; s_{——}CAG-495 sp000432275 | |
| 2W002.29 | d_{——}Bacteria; p_{——}Actinobacteriota; c_{——}Actinobacteria; o_{——}Actinomycetales; | Guild 2 |
| | f_{——}Bifidobacteriaceae; g_{——}Bifidobacterium; s_{——}Bifidobacterium longum | |
| 2W003.7 | d_{——}Bacteria; p_{——}Bacteroidota; c_{——}Bacteroidia; o_{——}Bacteroidales; | Guild 2 |
| | f_{——}Bacteroidaceae; g_{——}Bacteroides B; s_{——}Bacteroides B vulgatus | |
| 2W004.25 | d_{——}Bacteria; p_{——}Firmicutes C; c_{——}Negativicutes; o_{——}Veillonellales; | Guild 2 |
| | f_{——}Megasphaeraceae; g_{——}Caecibacter; s | |
| 2W005.9 | d_{——}Bacteria; p_{——}Bacteroidota; c_{——}Bacteroidia; o_{——}Bacteroidales; | Guild 2 |
| | f_{——}Tannerellaceae; g_{——}Parabacteroides; s_{——}Parabacteroides johnsonii | |
| 2W011.21 | d_{——}Bacteria; p_{——}Firmicutes; c_{——}Bacilli; o_{——}Lactobacillales; | Guild 2 |
| | f_{——}Enterococcaceae; g_{——}Enterococcus_B; s_{——}Enterococcus_B hirae | |
| 2W012.75 | d_{——}Bacteria; p_{——}Firmicutes; c_{——}Bacilli; o_{——}Lactobacillus; | Guild 2 |
| | f_{——}Streptococcaceae; g_{——}Streptococcus; s_{——}Streptococcus thermophilus | |
| 2W025.8 | d_{——}Bacteria; p_{——}Proteobacteria; c_{——}Gammaproteobacteria; o_{——}Enterobacterales; | Guild 2 |
| | f_{——}Enterobacteriaceae; g_{——}Enterobacter; s_{——}Enterobacter kobei | |
| 2W026.44 | d_{——}Bacteria; p_{——}Firmicutes; c_{——}Bacilli; o_{——}Lactobacillus; | Guild 2 |
| | f_{——}Streptococcaceae; g_{——}Streptococcus; s_{——}Streptococcus infantarius | |
| 2W037.10 | d_{——}Bacteria; p_{——}Bacteroidota; c_{——}Bacteroidia; o_{——}Bacteroidales; | Guild 2 |
| | f_{——}Tannerellaceae; g_{——}Parabacteroides; s_{——}Parabacteroides sp000436495 | |
| 2W048.49 | d_{——}Bacteria; p_{——}Firmicutes_{——}A; c_{——}Clostridia; o_{——}Peptostreptococcales; | Guild 2 |
| | f_{——}Anaerovoracaceae; g_{——}UBA1191; s | |
| 2W050.17 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides stercoris | |
| 2W053.46 | d_{——}Bacteria; p_{——}Proteobacteria; c_{——}Gammaproteobacteria; o_{——}Burkholderiales; | Guild 2 |
| | f_{——}Burkholderiaceae; g_{——}Oxalobacter; s_{——}Oxalobacter formigenes_A | |
| 2W054.20 | d_{——}Bacteria; p_{——}Firmicutes_{——}A; c_{——}Clostridia; o_{——}Lachnospirales; | Guild 2 |
| | f_{——}Lachnospiraceae; g_{——}Blautia_A; s_{——}Blautia_A sp900066165 | |
| 2W065.60 | d_{——}Bacteria; p_{——}Firmicutes; c_{——}Bacilli; o_{——}Lactobacillies; | Guild 2 |
| | f_{——}Streptococcaceae; g_{——}Streptococcus; s_{——}Streptococcus salivarius | |
| 2W067.20 | d_{——}Bacteria; p_{——}Bacteroidota; c_{——}Bacteroidia; o_{——}Bacteroidales; | Guild 2 |
| | f_{——}Bacteroidaceae; g_{——}Bacteroides_A; s_{——}Bacteroides_A coprocola | |
| 2W075.14 | d_{——}Bacteria; p_{——}Firmicutes_{——}A; c_{——}Clostridia; o_{——}Lachnospirales; | Guild 2 |
| | f_{——}Lachnospiraceae; g_{——}Faecalicatena; s_{——}Faecalicatena gnavus | |
| 2W080.58 | d_{——}Bacteria; p_{——}Proteobacteria; c_{——}Alphaproteobacteria; o_{——}RF32; | Guild 2 |
| | f_{——}CAG-239; g_{——}CAG-495; s | |
| 3U007.7 | d_{——}Bacteria; p_{——}Firmicutes_{——}A; c_{——}Clostridia; o_{——}Lachnospirales; | Guild 2 |
| | f_{——}Lachnospiraceae; g_{——}Clostridium_M; s_{——}Clostridium_M bolteae | |
| 3U020.14 | 1_Bacteria; p_{——}Firmicutes_{——}A; c_{——}Clostridia; o_{——}Lachnospirales; | Guild 2 |
| | f_{——}Lachnospiraceae; g_{——}Eubacterium_I; s_{——}Eubacterium_I ramulus | |
| 3U023.7 | d_{——}Bacteria; p_{——}Firmicutes_{——}A; c_{——}Clostridia; o_{——}Lachnospirales; | Guild 2 |
| | f_{——}Lachnospiraceae; g_{——}Clostridium_M; s | |
| 3U038.19 | d_{——}Bacteria; p_{——}Firmicutes_{——}A; c_{——}Clostridia; o_{——}Lachnospirales; | Guild 2 |
| | f_{——}Lachnospiraceae; g_{——}Blautia_A; s_{——}Blautia_A sp000433815 | |
| 3U040.11 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides stercoris | |
| 3W002.16 | d_{——}Bacteria; p_{——}Firmicutes_{——}A; c_{——}Clostridia; o_{——}Lachnospirales; | Guild 2 |
| | f_{——}Lachnospiraceae; g_{——}Clostridium_Q; s_{——}Clostridium_Q saccharolyticum | |
| 3W023.3 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides cellulosilyticus | |
| 3W028.33 | d_{——}Bacteria; p_{——}Firmicutes; c_{——}Bacilli; o_{——}Lactobacillales; | Guild 2 |
| | f_{——}Streptococcaceae; g_{——}Streptococcus; s_{——}Streptococcus pasteurianus | |
| 3W036.5 | d_{——}Bacteria; p_{——}Bacteroidota; c_{——}Bacteroidia; o_{——}Bacteroidales; | Guild 2 |
| | f_{——}Bacteroidaceae; g_{——}Bacteroides_B; s_{——}Bacteroides_B dorei | |
| 3W036.6 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides fragilis | |
| 3W039.10 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides eggerthii | |
| 3W042.11 | d_{——}Bacteria; p_{——}Bacteroidota; c_{——}Bacteroidia; o_{——}Bacteroidales; | Guild 2 |
| | f_{——}Bacteroidaceae; g_{——}Bacteroides_A; s_{——}Bacteroides_A plebeius_A | |
| 3W042.9 | d_{——}Bacteria; p_{——}Firmicutes; c_{——}Bacilli; o_{——}Erysipelotrichales; | Guild 2 |
| | f_{——}Erysipelatoclostridiaceae; g_{——}Erysipelatoclostridium; | |
| | s_{——}Erysipelatoclostridium ramosum | |
| 3W049.14 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides stercoris | |
| 3W054.11 | d_{——}Bacteria; p_{——}Proteobacteria; c_{——}Gammaproteobacteria; o_{——}Enterobacterales; | Guild 2 |
| | f_{——}Enterobacteriaceae; g_{——}Escherichia; s_{——}Escherichia flexneri | |
| 3W055.19 | d_{——}Bacteria; p_{——}Bacteroidota; c_{——}Bacteroidia; o_{——}Bacteroidales; | Guild 2 |
| | f_{——}Barnesiellaceae; g_{——}Barnesiella; s_{——}Barnesiella intestinihominis | |
| 3W067.18 | d_{——}Bacteria; p_{——}Bacteroidota; c_{——}Bacteroidia; o_{——}Bacteroidales; | Guild 2 |
| | f_{——}Muribaculaceae; g_{——}CAG-279; s | |
| 3W074.2 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides thetaiotaomicron | |
| 3W079.9 | d_{——}Bacteria; p_{——}Bacteroidota; c_{——}Bacteroidia; o_{——}Bacteroidales; | Guild 2 |
| | f_{——}Bacteroidaceae; g_{——}Bacteroides_A; s_{——}Bacteroides_A coprophilus | |
| 3W080.19 | d_{——}Bacteria; p_{——}Firmicutes; c_{——}Bacilli; o_{——}Lactobacillus; | Guild 2 |
| | f_{——}Enterococcaceae; g_{——}Enterococcus_D; s_{——}Enterococcus_D sp002850555 | |
| 3W080.64 | d_{——}Bacteria; p_{——}Firmicutes; c_{——}Bacilli; o_{——}RFN20; | Guild 2 |
| | f_{——}CAG-288; g_{——}UBA7642; s | |
| 1U003.28 | d_{——}Bacteria; p_{——}Actinobacteriota; c_{——}Actinobacteria; o_{——}Actinomycetales; | Guild 2 |
| | f_{——}Micrococcaceae; g_{——}Rothia; s_{——}Rothia mucilaginosa B | |
| 1U017.15 | d_{——}Bacteria; p_{——}Firmicutes_{——}A; c_{——}Clostridia; o_{——}Lachnospirales; | Guild 2 |
| | f_{——}Lachnospiraceae; g_{——}Sellimonas; s_{——}Sellimonas intestinalis | |
| 1U001.8 | d_{——}Bacteria; p_{——}Proteobacteria; c_{——}Gammaproteobacteria; o_{——}Enterobacterales; | Guild 2 |
| | f_{——}Enterobacteriaceae; g_{——}Escherichia; s_{——}Escherichia coli | |
| 1U017.10 | d_{——}Bacteria; p_{——}Firmicutes_{——}A; c_{——}Clostridia; o_{——}Lachnospirales; | Guild 2 |
| | f_{——}Lachnospiraceae; g_{——}Dorea; s_{——}Dorea sp000509125 | |
| 1U027.33 | d_{——}Bacteria; p_{——}Firmicutes_{——}A; c_{——}Clostridia; o_{——}Oscillospirales; | Guild 2 |
| | f_{——}Acutalibacteraceae; g_{——}Eubacterium_R; s_{——}Eubacterium_R sp000431535 | |
| 1U015.30 | d_{——}Bacteria; p_{——}Firmicutes; c_{——}Bacilli; o_{——}Erysipelotrichales; | Guild 2 |
| | f_{——}Erysipelotrichaceae; g_{——}Absiella; s_{——}Absiella dolichum | |
| 1U036.9 | d_{——}Bacteria; p_{——}Bacteroidota; c_{——}Bacteroidia; o_{——}Bacteroidales; | Guild 2 |
| | f_{——}Bacteroidaceae; g_{——}Paraprevotella; s_{——}Paraprevotella clara | |
| 1U015.20 | d_{——}Bacteria; p_{——}Firmicutes_{——}A; c_{——}Clostridia; o_{——}Lachnospirales; | Guild 2 |
| | f_{——}Lachnospiraceae; g_{——}Faecalicatena; s_{——}Faecalicatena glycyrrhizinilyticum | |
| 1W023.6 | d_{——}Bacteria; p_{——}Proteobacteria; c_{——}Gammaproteobacteria; o_{——}Enterobacterales; | Guild 2 |
| | f_{——}Enterobacteriaceae; g_{——}Klebsiella; s_{——}Klebsiella pneumoniae | |
| 1W023.15 | d_{——}Bacteria; p_{——}Bacteroidota; c_{——}Bacteroidia; o_{——}Bacteroidales; | Guild 2 |
| | f_{——}Bacteroidaceae; g_{——}Bacteroides_A; s_{——}Bacteroides_A plebeius | |
| 1U024.11 | d_{——}Bacteria; p_{——}Actinobacteriota; c_{——}Coriobacteriia; o_{——}Coriobacteriales; | Guild 2 |
| | f_{——}Eggerthellaceae; g_{——}Eggerthella; s_{——}Eggerthella lenta | |
| 1W023.20 | d_{——}Bacteria; p_{——}Firmicutes_{——}A; c_{——}Clostridia; o_{——}Oscillospirales; | Guild 2 |
| | f_{——}Oscillospiraceae; g_{——}Lawsonibacter; s | |
| 1U036.17 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides stercoris | |
| 1U021.14 | d_{——}Bacteria; p_{——}Bacteroidota; c_{——}Bacteroidia; o_{——}Bacteroidales; | Guild 2 |
| | f_{——}Bacteroidaceae; g_{——}Bacteroides_A; s_{——}Bacteroides_A coprocola | |
| 1W026.10 | d_{——}Bacteria; p_{——}Firmicutes_{——}A; c_{——}Clostridia; o_{——}Lachnospirales; | Guild 2 |
| | f_{——}Lachnospiraceae; g_{——}Blautia_A; s | |
| 1U014.18 | d_{——}Bacteria; p_{——}Firmicutes_{——}A; c_{——}Clostridia; o_{——}Lachnospirales; | Guild 2 |
| | f_{——}Lachnospiraceae; g_{——}Clostridium_M; s_{——}Clostridium_M sp000431375 | |
| 2W067.16 | d_{——}Bacteria; p_{——}Bacteroidota; c_{——}Bacteroidia; o_{——}Bacteroidales; | Guild 2 |
| | f_{——}Coprobacteraceae; g_{——}Coprobacter; s_{——}Coprobacter fastidiosus | |
| 1W079.48 | d_{——}Bacteria; p_{——}Bacteroidota; c_{——}Bacteroidia; o_{——}Bacteroidales; | Guild 2 |
| | f_{——}UBA932; g_{——}RC9; s_{——}RC9 sp000431015 | |

TABLE 2
Taxonomy Assignment of 284 core microbiome

| | Taxonomy Assignment (d = domain; p = phylum; c = class; o = order; | |
|---|---|---|
| ID | f = family; g = genus; s = species) | Assignment |
| 1U003.28 | d_{——}Bacteria; p_{——}Actinobacteriota; c_{——}Actinobacteria; o_{——}Actinomycetales; | Guild 2 |
| | f_{——}Micrococcaceae; g_{——}Rothia; s_{——}Rothia mucilaginosa_B | |
| 1U015.20 | d_{——}Bacteria; p_{——}Firmicutes_A; c_{——}Clostridia; o_{——}Lachnospirales; | Guild 2 |
| | f_{——}Lachnospiraceae; g_{——}Faecalicatena; s_{——}Faecalicatena glycyrrhizinilyticum | |
| 1U024.11 | d_{——}Bacteria; p_{——}Actinobacteriota; c_{——}Coriobacteriia; o_{——}Coriobacteriales; | Guild 2 |
| | f_{——}Eggerthellaceae; g_{——}Eggerthella; s_{——}Eggerthella lenta | |
| 1U035.49 | d_{——}Bacteria; p_{——}Firmicutes_A; c_{——}Clostridia; o_{——}Oscillospirales; | Guild 1 |
| | f_{——}Oscillospiraceae; g_{——}CAG-103; s | |
| 1U035.55 | d_{——}Bacteria; p_{——}Firmicutes; c_{——}Bacilli; o_{——}Lactobacillales; | Guild 2 |
| | f_{——}Streptococcaceae; g_{——}Streptococcus; s_{——}Streptococcus macedonicus | |
| 1U038.18 | d_{——}Bacteria; p_{——}Firmicutes_A; c_{——}Clostridia; o_{——}Lachnospirales; | Guild 2 |
| | f_{——}Lachnospiraceae; g_{——}UBA7096; s | |
| 1W011.28 | d_{——}Bacteria; p_{——}Firmicutes_A; c_{——}Clostridia; o_{——}Oscillospirales; | Guild 1 |
| | f_{——}Oscillospiraceae; g_{——}Oscillibacter; s Oscillibacter sp001916835 | |
| 1W023.20 | d_{——}Bacteria; p_{——}Firmicutes_A; c_{——}Clostridia; o_{——}Oscillospirales; | Guild 2 |
| | f_{——}Oscillospiraceae; g_{——}Lawsonibacter; s | |
| 1W030.27 | d_{——}Bacteria; p_{——}Firmicutes_A; c_{——}Clostridia; o_{——}Oscillospirales; | Guild 1 |
| | f_{——}Oscillospiraceae; g_{——}Oscillibacter; s_{——}Oscillibacter sp001916835 | |
| 1W032.19 | d_{——}Bacteria; p_{——}Firmicutes_A; c_{——}Clostridia; o_{——}Lachnospirales; | Guild 2 |
| | f_{——}Lachnospiraceae; g_{——}Eubacterium_G; s_{——}Eubacterium_G sp000434315 | |
| 1W033.9 | d_{——}Bacteria; p_{——}Firmicutes_A; c_{——}Clostridia; o_{——}Oscillospirales; | Guild 2 |
| | f_{——}Oscillospiraceae; g_{——}Flavonifractor; s_{——}Flavonifractor plautii | |
| 1W042.8 | d_{——}Bacteria; p_{——}Firmicutes_A; c_{——}Clostridia; o_{——}Lachnospirales; | Guild 2 |
| | f_{——}Lachnospiraceae; g_{——}Clostridium_M; s_{——}Clostridium_M sp001517625 | |
| 1W065.34 | d_{——}Bacteria; p_{——}Firmicutes_A; c_{——}Clostridia; o_{——}Oscillospirales; | Guild 1 |
| | f_{——}Oscillospiraceae; g_{——}Oscillibacter; s_{——}Oscillibacter sp001916835 | |
| 1W072.11 | d_{——}Bacteria; p_{——}Firmicutes; c_{——}Bacilli; o_{——}Erysipelotrichales; | Guild 2 |
| | f_{——}Erysipelotrichaceae; g_{——}Absiella; s | |
| 1W075.14 | d_{——}Bacteria; p_{——}Bacteroidota; c_{——}Bacteroidia; o_{——}Bacteroidales; | Guild 2 |
| | f_{——}Marinifilaceae; g_{——}Odoribacter; s | |
| 1W078.21 | d_{——}Bacteria; p_{——}Firmicutes_C; c_{——}Negativicutes; o_{——}Veillonellales; | Guild 2 |
| | f_{——}Veillonellaceae; g_{——}Veillonella; s_{——}Veillonella parvula_A | |
| 1W080.50 | d_{——}Bacteria; p_{——}Proteobacteria; c_{——}Alphaproteobacteria; o_{——}RF32; | Guild 2 |
| | f_{——}CAG-239; g_{——}51-20; s_{——}51-20 sp001917175 | |
| 2U001.17 | d_{——}Bacteria; p_{——}Firmicutes_A; c_{——}Clostridia; o_{——}Lachnospirales; | Guild 2 |
| | f_{——}Lachnospiraceae; g_{——}Clostridium_M; s_{——}Clostridium_M sp001517625 | |
| 2U008.16 | d_{——}Bacteria; p_{——}Fusobacteriota; c_{——}Fusobacteriia; o_{——}Fusobacteriales; | Guild 2 |
| | f_{——}Fusobacteriaceae; g_{——}Fusobacterium_A; s_{——}Fusobacterium Aulcerans | |
| 2U033.18 | d_{——}Bacteria; p_{——}Bacteroidota; c_{——}Bacteroidia; o_{——}Bacteroidales; | Guild 2 |
| | f_{——}Bacteroidaceae; g_{——}Bacteroides_A; s_{——}Bacteroides_A sp000434735 | |
| 2U037.14 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides clarus | |
| 2W001.42 | d_{——}Bacteria; p_{——}Proteobacteria; c_{——}Alphaproteobacteria; o_{——}RF32; | Guild 2 |
| | f_{——}CAG-239; g_{——}CAG-495; s_{——}CAG-495 sp000432275 | |
| 2W011.21 | d_{——}Bacteria; p_{——}Firmicutes; c_{——}Bacilli; o_{——}Lactobacillies; | Guild 2 |
| | f_{——}Enterococcaceae; g_{——}Enterococcus_B; s_{——}Enterococcus_B hirae | |
| 2W023.30 | d_{——}Bacteria; p_{——}Firmicutes_A; c_{——}Clostridia; o_{——}Oscillospirales; | Guild 1 |
| | f_{——}Acutalibacteraceae; g_{——}UBA1417; s | |
| 2W027.27 | d_{——}Bacteria; p_{——}Firmicutes_A; c_{——}Clostridia; o_{——}Oscillospirales; | Guild 1 |
| | f_{——}Ruminococcaceae; g_{——}Faecalibacterium; s_{——}Faecalibacterium prausnitzii_D | |
| 2W048.49 | d_{——}Bacteria; p_{——}Firmicutes_A; c_{——}Clostridia; o_{——}Peptostreptococcales; | Guild 2 |
| | f_{——}Anaerovoracaceae; g_{——}UBA1191; s | |
| 2W053.46 | d_{——}Bacteria; p_{——}Proteobacteria; c_{——}Gammaproteobacteria; o_{——}Burkholderiales; | Guild 2 |
| | f_{——}Burkholderiaceae; g_{——}Oxalobacter; s_{——}Oxalobacter formigenes_A | |
| 2W065.60 | d_{——}Bacteria; p_{——}Firmicutes; c_{——}Bacilli; o_{——}Lactobacillies; | Guild 2 |
| | f_{——}Streptococcaceae; g_{——}Streptococcus; s_{——}Streptococcus salivarius | |
| 2W073.55 | d_{——}Bacteria; p_{——}Firmicutes_A; c_{——}Clostridia; o_{——}Oscillospirales; | Guild 1 |
| | f_{——}Butyricicoccaceae; g_{——}Butyricicoccus_A; s_{——}Butyricicoccus_A sp002395695 | |
| 3U020.14 | d_{——}Bacteria; p_{——}Firmicutes_A; c_{——}Clostridia; o_{——}Lachnospirales; | Guild 2 |
| | f_{——}Lachnospiraceae; g_{——}Eubacterium_I; s_{——}Eubacterium_I ramulus | |
| 3U023.7 | d_{——}Bacteria; p_{——}Firmicutes_A; c_{——}Clostridia; o_{——}Lachnospirales; | Guild 2 |
| | f_{——}Lachnospiraceae; g_{——}Clostridium_M; s | |
| 3W002.16 | d_{——}Bacteria; p_{——}Firmicutes_A; c_{——}Clostridia; o_{——}Lachnospirales; | Guild 2 |
| | f_{——}Lachnospiraceae; g_{——}Clostridium_Q; s_{——}Clostridium_Q saccharolyticum | |
| 3W015.22 | d_{——}Bacteria; p_{——}Firmicutes_A; c_{——}Clostridia; o_{——}Oscillospirales; | Guild 1 |
| | f_{——}Ruminococcaceae; g_{——}Faecalibacterium; s_{——}Faecalibacterium prausnitzii_J | |
| 3W039.10 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides eggerthii | |
| 3W055.19 | d_{——}Bacteria; p_{——}Bacteroidota; c_{——}Bacteroidia; o_{——}Bacteroidales; | Guild 2 |
| | f_{——}Barnesiellaceae; g_{——}Barnesiella; s_{——}Barnesiella intestinihominis | |
| 3W062.32 | d_{——}Bacteria; p_{——}Firmicutes_A; c_{——}Clostridia; o_{——}Oscillospirales; | Guild 1 |
| | f_{——}Ruminococcaceae; g_{——}CAG-353; s_{——}CAG-353 sp900066885 | |
| 3W067.18 | d_{——}Bacteria; p_{——}Bacteroidota; c_{——}Bacteroidia; o_{——}Bacteroidales; | Guild 2 |
| | f_{——}Muribaculaceae; g_{——}CAG-279; s | |
| 3W074.2 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides thetaiotaomicron | |
| ERR1018189.10 | d_{——}Bacteria; p_{——}Firmicutes_A; c_{——}Clostridia; o_{——}Oscillospirales; | Guild 2 |
| | f_{——}Oscillospiraceae; g_{——}CAG-110; s_{——}CAG-110 sp900546075 | |
| ERR1018192.4 | d_{——}Bacteria; p_{——}Firmicutes_A; c_{——}Clostridia; o_{——}Lachnospirales; | Guild 2 |
| | f_{——}Lachnospiraceae; g_{——}Blautia_A; s_{——}Blautia_A sp900066145 | |
| ERR1018196.11 | d_{——}Bacteria; p_{——}Firmicutes_C; c_{——}Negativicutes; o_{——}Veillonellales; | Guild 2 |
| | f_{——}Dialisteraceae; g_{——}Dialister; s_{——}Dialister succinatiphilus | |
| ERR1018197.5 | d_{——}Bacteria; p_{——}Firmicutes_A; c_{——}Clostridia; o_{——}Oscillospirales; | Guild 1 |
| | f_{——}Acutalibacteraceae; g_{——}UMGS263; s_{——}UMGS263 sp900540535 | |
| ERR1018197.9 | d_{——}Bacteria; p_{——}Firmicutes_A; c_{——}Clostridia; o_{——}Oscillospirales; | Guild 1 |
| | f_{——}Acutalibacteraceae; g_{——}Clostridium_A; s_{——}Clostridium_A leptum | |
| ERR1018200.15 | d_{——}Bacteria; p_{——}Firmicutes_A; c_{——}Clostridia; o_{——}Oscillospirales; | Guild 2 |
| | f_{——}Acutalibacteraceae; g_{——}UMGS1071; s_{——}UMGS1071 sp900542375 | |
| ERR1018200.4 | d_{——}Bacteria; p_{——}Firmicutes_A; c_{——}Clostridia; o_{——}Lachnospirales; | Guild 2 |
| | f_{——}Lachnospiraceae; g_{——}Eisenbergiella; s_{——}Eisenbergiella tayi | |
| ERR1018204.1 | d_{——}Bacteria; p_{——}Firmicutes_A; c_{——}Clostridia; o_{——}Lachnospirales; | Guild 2 |
| | f_{——}Lachnospiraceae; g_{——}Enterocloster; s_{——}Enterocloster clostridioformisa | |
| ERR1018210.9 | d_{——}Bacteria; p_{——}Actinobacteriota; c_{——}Actinomycetia; o_{——}Actinomycetales; | Guild 2 |
| | f_{——}Bifidobacteriaceae; g_{——}Bifidobacterium; s_{——}Bifidobacterium pseudocatenulatum | |
| ERR1018213.10 | d_{——}Bacteria; p_{——}Firmicutes; c_{——}Bacilli; o_{——}Lactobacillies; | Guild 2 |
| | f_{——}Streptococcaceae; g_{——}Streptococcus; s_{——}Streptococcus salivarius | |
| ERR1018218.4 | d_{——}Bacteria; p_{——}Bacteroidota; c_{——}Bacteroidia; o_{——}Bacteroidales; | Guild 2 |
| | f_{——}Bacteroidaceae; g_{——}Prevotella; s_{——}Prevotella hominis | |
| ERR1018219.12 | d_{——}Bacteria; p_{——}Firmicutes_A; c_{——}Clostridia; o_{——}Oscillospirales; | Guild 2 |
| | f_{——}Acutalibacteraceae; g_{——}Eubacterium_R; s_{——}Eubacterium_R sp000434995 | |
| ERR1018219.9 | d_{——}Bacteria; p_{——}Desulfobacterota; c_{——}Desulfovibrionia; o_{——}Desulfovibrionales; | Guild 2 |
| | f_{——}Desulfovibrionaceae; g_{——}Bilophila; s_{——}Bilophila wadsworthia | |
| ERR1018229.14 | d_{——}Bacteria; p_{——}Firmicutes_C; c_{——}Negativicutes; o_{——}Veillonellales; | Guild 2 |
| | f_{——}Megasphaeraceae; g_{——}Caecibacter; s_{——}Caecibacter hominis | |
| ERR1018232.13 | d_{——}Bacteria; p_{——}Bacteroidota; c_{——}Bacteroidia; o_{——}Bacteroidales; | Guild 2 |
| | f_{——}Bacteroidaceae; g_{——}Prevotella; s_{——}Prevotella copri | |
| ERR1018237.11 | d_{——}Bacteria; p_{——}Firmicutes_A; c_{——}Clostridia; o_{——}Lachnospirales; | Guild 2 |
| | f_{——}Lachnospiraceae; g_{——}Eubacterium_G; s_{——}Eubacterium_G ventriosum | |
| ERR1018238.17 | d_{——}Bacteria; p_{——}Firmicutes_A; c_{——}Clostridia; o_{——}Lachnospirales; | Guild 2 |
| | f_{——}Lachnospiraceae; g_{——}Anaerostipes; s_{——}Anaerostipes hadrus | |
| ERR1018239.7 | d_{——}Bacteria; p_{——}Firmicutes_A; c_{——}Clostridia; o_{——}Lachnospirales; | Guild 2 |
| | f_{——}Lachnospiraceae; g_{——}Blautia_A; s_{——}Blautia_A sp003480185 | |
| ERR1018250.9 | d_{——}Bacteria; p_{——}Firmicutes_A; c_{——}Clostridia; o_{——}Lachnospirales; | Guild 2 |
| | f_{——}Lachnospiraceae; g_{——}Enterocloster; s_{——}Enterocloster sp900541315 | |
| ERR1018258.4 | d_{——}Bacteria; p_{——}Bacteroidota; c_{——}Bacteroidia; o_{——}Bacteroidales; | Guild 2 |
| | f_{——}Tannerellaceae; g_{——}Parabacteroides; s_{——}Parabacteroides distasonis | |
| ERR1018262.5 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides uniformis | |
| ERR1018267.4 | d_{——}Bacteria; p_{——}Bacteroidota; c Bacteroidia; o_{——}Bacteroidales; | Guild 2 |
| | f_{——}Coprobacteraceae; g_{——}Coprobacter; s_{——}Coprobacter fastidiosus | |
| ERR1018278.5 | d_{——}Bacteria; p_{——}Firmicutes_C; c_{——}Negativicutes; o_{——}Veillonellales; | Guild 2 |
| | f_{——}Dialisteraceae; g_{——}Dialister; s_{——}Dialister sp900541605 | |
| ERR1018291.11 | d_{——}Bacteria; p_{——}Firmicutes_A; c_{——}Clostridia; o_{——}Oscillospirales; | Guild 1 |
| | f_{——}Oscillospiraceae; g_{——}ER4; s_{——}ER4 sp900317525 | |
| ERR1018292.2 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides intestinalis | |
| ERR1018298.1 | d_{——}Bacteria; p_{——}Firmicutes; c_{——}Bacilli; o_{——}Erysipelotrichales; | Guild 2 |
| | f_{——}Erysipelotrichaceae; g_{——}Faecalitalea; s_{——}Faecalitalea cylindroides | |
| ERR1018301.12 | d_{——}Bacteria; p_{——}Bacteroidota; c_{——}Bacteroidia; o_{——}Bacteroidales; | Guild 2 |
| | f_{——}Marinifilaceae; g_{——}Butyricimonas; s_{——}Butyricimonas virosa | |
| ERR1018308.3 | d_{——}Bacteria; p_{——}Firmicutes_A; c_{——}Clostridia; o_{——}Lachnospirales; | Guild 2 |
| | f_{——}Lachnospiraceae; g_{——}Agathobacter; s_{——}Agathobacter sp900548765 | |
| ERR1018309.23 | d_{——}Bacteria; p_{——}Firmicutes_A; c_{——}Clostridia; o_{——}Monoglobales_A; | Guild 2 |
| | f_{——}UBA1381; g_{——}CAG-41; s_{——}CAG-41 sp900066215 | |
| ERR2017433.5 | d_{——}Bacteria; p_{——}Firmicutes_A; c_{——}Clostridia; o_{——}Lachnospirales; | Guild 1 |
| | f_{——}Lachnospiraceae; g_{——}Lachnospira; s_{——}Lachnospira sp000437735 | |
| ERR2017443.11 | d_{——}Bacteria; p_{——}Firmicutes_C; c_{——}Negativicutes; o_{——}Acidaminococcales; | Guild 2 |
| | f_{——}Acidaminococcaceae; g_{——}Phascolarctobacterium; s_{——}Phascolarctobacterium faecium | |
| ERR2017473.2 | d_{——}Bacteria; p_{——}Fusobacteriota; c_{——}Fusobacteriia; o_{——}Fusobacteriales; | Guild 2 |
| | f_{——}Fusobacteriaceae; g_{——}Fusobacterium_A; s_{——}Fusobacterium_A mortiferum | |
| ERR2017500.4 | d_{——}Bacteria; p_{——}Bacteroidota; c_{——}Bacteroidia; o_{——}Bacteroidales; | Guild 2 |
| | f_{——}Bacteroidaceae; g_{——}Phocaeicola; s_{——}Phocaeicola massiliensis | |
| ERR2017517.1 | d_{——}Bacteria; p_{——}Firmicutes_A; c_{——}Clostridia; o_{——}Lachnospirales; | Guild 1 |
| | f_{——}Lachnospiraceae; g_{——}RUG115; s_{——}RUG115 sp900066395 | |
| ERR2017522.17 | d_{——}Bacteria; p_{——}Firmicutes; c_{——}Bacilli; o_{——}Erysipelotrichales; | Guild 2 |
| | f_{——}Erysipelotrichaceae; g_{——}Longicatena; s_{——}Longicatena caecimuris | |
| ERR2017539.14 | d_{——}Bacteria; p_{——}Firmicutes_A; c_{——}Clostridia; o_{——}Oscillospirales; | Guild 1 |
| | f_{——}Ruminococcaceae; g_{——}Ruminococcus_C; s_{——}Ruminococcus_C sp000433635 | |
| ERR2017540.1 | d_{——}Bacteria; p_{——}Bacteroidota; c_{——}Bacteroidia; o_{——}Bacteroidales; | Guild 2 |
| | f_{——}Rikenellaceae; g_{——}Alistipes_A; s_{——}Alistipes_A indistinctus | |
| ERR2017541.4 | d_{——}Bacteria; p_{——}Firmicutes_A; c_{——}Clostridia; o_{——}Lachnospirales; | Guild 1 |
| | f_{——}Lachnospiraceae; g_{——}CAG-95; s_{——}CAG-95 sp900066375 | |
| ERR2017552.8 | d_{——}Bacteria; p_{——}Firmicutes_A; c_{——}Clostridia; o_{——}Oscillospirales; | Guild 1 |
| | f_{——}Ruminococcaceae; g_{——}Gemmiger; s_{——}Gemmiger formicilis | |
| ERR2017555.15 | d_{——}Bacteria; p_{——}Firmicutes_A; c_{——}Clostridia; o_{——}Oscillospirales; | Guild 1 |
| | f_{——}Ruminococcaceae; g_{——}Faecalibacterium; s_{——}Faecalibacterium sp900540455 | |
| ERR2017555.23 | d_{——}Bacteria; p_{——}Firmicutes_A; c_{——}Clostridia; o_{——}Oscillospirales; | Guild 2 |
| | f_{——}Ruminococcaceae; g_{——}Ruthenibacterium; s_{——}Ruthenibacterium lactatiformans | |
| ERR2017563.14 | d_{——}Bacteria; p_{——}Firmicutes_A; c_{——}Clostridia; o_{——}Oscillospirales; | Guild 1 |
| | f_{——}Ruminococcaceae; g_{——}Ruminococcus_D; s_{——}Ruminococcus_D bicirculans | |
| ERR2017569.9 | d_{——}Bacteria; p_{——}Firmicutes_C; c_{——}Negativicutes; o_{——}Acidaminococcales; | Guild 2 |
| | f_{——}Acidaminococcaceae; g_{——}Phascolarctobacterium; | |
| | s_{——}Phascolarctobacterium sp900545535 | |
| ERR2017573.11 | d_{——}Bacteria; p_{——}Bacteroidota; c_{——}Bacteroidia; o_{——}Bacteroidales; | Guild 1 |
| | f_{——}Bacteroidaceae; g_{——}Prevotella; s_{——}Prevotella pectinovora | |
| ERR2017577.6 | d_{——}Bacteria; p_{——}Firmicutes; c_{——}Bacilli; o_{——}Erysipelotrichales; | Guild 2 |
| | f_{——}Erysipelatoclostridiaceae; g_{——}Coprobacillus; s_{——}Coprobacillus cateniformis | |
| ERR2017581.15 | d_{——}Bacteria; p_{——}Firmicutes_A; c_{——}Clostridia; o_{——}Oscillospirales; | Guild 1 |
| | f_{——}Oscillospiraceae; g_{——}ER4; s_{——}ER4 sp000765235 | |
| ERR2017584.6 | d_{——}Bacteria; p_{——}Firmicutes_A; c_{——}Clostridia; o_{——}Lachnospirales; | Guild 1 |
| | f_{——}Lachnospiraceae; g_{——}Anaerobutyricum; s_{——}Anaerobutyricum hallii | |
| ERR2017586.7 | d_{——}Bacteria; p_{——}Firmicutes; c_{——}Bacilli; o_{——}Erysipelotrichales; | Guild 2 |
| | f_{——}Erysipelatoclostridiaceae; g_{——}Longibaculum; s_{——}Longibaculum muris | |
| ERR2017591.13 | d_{——}Bacteria; p_{——}Bacteroidota; c_{——}Bacteroidia; o_{——}Bacteroidales; | Guild 2 |
| | f_{——}Bacteroidaceae; g_{——}Phocaeicola; s_{——}Phocaeicola sp900542985 | |
| ERR2017596.4 | d_{——}Bacteria; p_{——}Actinobacteriota; c_{——}Actinomycetia; o_{——}Actinomycetales; | Guild 2 |
| | f_{——}Bifidobacteriaceae; g_{——}Bifidobacterium; s_{——}Bifidobacterium longum | |
| ERR2017601.3 | d_{——}Bacteria; p_{——}Firmicutes; c_{——}Bacilli; o_{——}Lactobacillaes; | Guild 2 |
| | f_{——}actobacillaceae; g_{——}Limosilactobacillus; s_{——}Limosilactobacillus fermentum | |
| ERR2017608.6 | d_{——}Bacteria; p_{——}Firmicutes_A; c_{——}Clostridia; o_{——}Oscillospirales; | Guild 1 |
| | f_{——}Oscillospiraceae; g_{——}UBA5446; s_{——}UBA5446 sp900546485 | |
| ERR2017610.5 | d_{——}Bacteria; p_{——}Proteobacteria; c_{——}Gammaproteobacteria; o_{——}Enterobacterales; | Guild 2 |
| | f_{——}Enterobacteriaceae; g_{——}Klebsiella; s_{——}Klebsiella aerogenes | |
| ERR2017617.4 | d_{——}Bacteria; p_{——}Firmicutes_C; c_{——}Negativicutes; o_{——}Veillonellales; | Guild 2 |
| | f_{——}Veillonellaceae; g_{——}Veillonella; s_{——}Veillonella parvula_A | |
| ERR2017618.4 | d_{——}Bacteria; p_{——}Firmicutes_A; c_{——}Clostridia; o_{——}Oscillospirales; | Guild 1 |
| | f_{——}Ruminococcaceae; g_{——}Faecalibacterium; s_{——}Faecalibacterium prausnitzii_D | |
| ERR2017622.3 | d_{——}Bacteria; p_{——}Firmicutes_A; c_{——}Clostridia; o_{——}Lachnospirales; | Guild 1 |
| | f_{——}Lachnospiraceae; g_{——}Coprococcus; s_{——}Coprococcus sp900066115 | |
| ERR2017636.11 | d_{——}Bacteria; p_{——}Firmicutes_A; c_{——}Clostridia; o_{——}Lachnospirales; | Guild 2 |
| | f_{——}Lachnospiraceae; g_{——}Blautia_A; s_{——}Blautia_A sp003471165 | |
| ERR2017638.17 | d_{——}Bacteria; p_{——}Firmicutes_A; c_{——}Clostridia; o_{——}Oscillospirales; | Guild 1 |
| | f_{——}Acutalibacteraceae; g_{——}Ruminococcus_E; s_{——}Ruminococcus_E bromii_B | |
| ERR2017641.10 | d_{——}Bacteria; p_{——}Firmicutes_A; c_{——}Clostridia; o_{——}Lachnospirales; | Guild 1 |
| | f_{——}Lachnospiraceae; g_{——}COE1; s_{——}COE1 sp001916965 | |
| ERR2017647.13 | d_{——}Bacteria; p_{——}Firmicutes_A; c_{——}Clostridia; o_{——}Oscillospirales; | Guild 1 |
| | f_{——}Acutalibacteraceae; g_{——}CAG-217; s_{——}CAG-217 sp000436335 | |
| ERR2017650.13 | d_{——}Bacteria; p_{——}Firmicutes_A; c_{——}Clostridia; o_{——}Lachnospirales; | Guild 2 |
| | f_{——}Lachnospiraceae; g_{——}CAG-81; s_{——}CAG-81 sp000435795 | |
| ERR2017651.6 | d_{——}Bacteria; p_{——}Firmicutes_A; c_{——}Clostridia; o_{——}Lachnospirales; | Guild 2 |
| | f_{——}Lachnospiraceae; g_{——}Clostridium_AP; s_{——}Clostridium_AP sp000509125 | |
| ERR2017654.11 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides uniformis | |
| ERR2017659.20 | d_{——}Bacteria; p_{——}Firmicutes_A; c_{——}Clostridia; o_{——}Oscillospirales; | Guild 1 |
| | f_{——}Acutalibacteraceae; g_{——}CAG-180; s_{——}CAG-180 sp004556705 | |
| ERR2017660.10 | d_{——}Bacteria; p_{——}Firmicutes_A; c_{——}Clostridia; o_{——}Lachnospirales; | Guild 1 |
| | f_{——}Lachnospiraceae; g_{——}Eubacterium_G; s_{——}Eubacterium_G sp000434315 | |
| ERR2017669.11 | d_{——}Bacteria; p_{——}Firmicutes; c_{——}Bacilli; o_{——}Erysipelotrichales; | Guild 1 |
| | f_{——}Erysipelotrichaceae; g_{——}Clostridium_AQ; s_{——}Clostridium_AQ sp003481775 | |
| ERR2017669.13 | d_{——}Bacteria; p_{——}Actinobacteriota; c_{——}Coriobacteriia; o_{——}Coriobacteriales; | Guild 2 |
| | f_{——}Eggerthellaceae; g_{——}Eggerthella; s_{——}Eggerthella lenta | |
| ERR2017669.2 | d_{——}Bacteria; p_{——}Firmicutes_A; c_{——}Clostridia; o_{——}Oscillospirales; | Guild 2 |
| | f_{——}Acutalibacteraceae; g_{——}UMGS1071; s_{——}UMGS1071 sp900541905 | |
| ERR2017672.11 | d_{——}Bacteria; p_{——}Actinobacteriota; c_{——}Coriobacteriia; o_{——}Coriobacteriales; | Guild 2 |
| | f_{——}Coriobacteriaceae; g_{——}Collinsella; s_{——}Collinsella sp002232035 | |
| ERR2017680.3 | d_{——}Bacteria; p_{——}Firmicutes_A; c_{——}Clostridia; o_{——}Lachnospirales; | Guild 1 |
| | f_{——}Lachnospiraceae; g_{——}UBA9502; s_{——}UBA9502 sp003480315 | |
| ERR2017696.13 | d_{——}Bacteria; p_{——}Firmicutes_A; c_{——}Clostridia; o_{——}Lachnospirales; | Guild 2 |
| | f_{——}Lachnospiraceae; g_{——}UBA9502; s_{——}UBA9502 sp900555625 | |
| ERR2017699.1 | d_{——}Bacteria; p_{——}Firmicutes_A; c_{——}Clostridia; o_{——}Lachnospirales; | Guild 1 |
| | f_{——}Lachnospiraceae; g_{——}Eisenbergiella; s_{——}Eisenbergiella tayi | |
| ERR2017700.13 | d_{——}Bacteria; p_{——}Firmicutes_A; c_{——}Clostridia; o_{——}Lachnospirales; | Guild 1 |
| | f_{——}Lachnospiraceae; g_{——}CAG-81; s_{——}CAG-81 sp900066535 | |
| ERR2017701.13 | d_{——}Bacteria; p_{——}Firmicutes_A; c_{——}Clostridia; o_{——}Oscillospirales; | Guild 1 |
| | f_{——}Ruminococcaceae; g_{——}Ruminococcus_D; s_{——}Ruminococcus_D bicirculans | |
| ERR2017705.14 | d_{——}Bacteria; p_{——}Bacteroidota; c_{——}Bacteroidia; o_{——}Bacteroidales; | Guild 2 |
| | f_{——}Bacteroidaceae; g_{——}Prevotella; s_{——}Prevotella copri_A | |
| ERR2017706.14 | d_{——}Bacteria; p_{——}Firmicutes_A; c_{——}Clostridia; o_{——}Oscillospirales; | Guild 1 |
| | f_{——}Oscillospiraceae; g_{——}Lawsonibacter; s_{——}Lawsonibacter sp000177015 | |
| ERR2017714.15 | d_{——}Bacteria; p_{——}Firmicutes_C; c_{——}Negativicutes; o_{——}Acidaminococcales; | Guild 2 |
| | f_{——}Acidaminococcaceae; g_{——}Phascolarctobacterium; | |
| | s_{——}Phascolarctobacterium faecium | |
| ERR2017716.3 | d_{——}Bacteria; p_{——}Firmicutes_A; c_{——}Clostridia; o_{——}Oscillospirales; | Guild 1 |
| | f_{——}Ruminococcaceae; g_{——}Negativibacillus; s_{——}Negativibacillus massiliensis | |
| ERR2017716.6 | d_{——}Bacteria; p_{——}Firmicutes_A; c_{——}Clostridia; o_{——}Oscillospirales; | Guild 2 |
| | f_{——}Oscillospiraceae; g_{——}Flavonifractor; s_{——}Flavonifractor plautii | |
| ERR2017717.14 | d_{——}Bacteria; p_{——}Firmicutes_A; c_{——}Clostridia; o_{——}Lachnospirales; | Guild 1 |
| | f_{——}Lachnospiraceae; g_{——}CAG-127; s_{——}CAG-127 sp900319515 | |
| ERR2017729.15 | d_{——}Bacteria; p_{——}Firmicutes_A; c_{——}Clostridia; o_{——}Lachnospirales; | Guild 1 |
| | f_{——}Lachnospiraceae; g_{——}Agathobacter; s_{——}Agathobacter sp900543445 | |
| ERR2017731.2 | d_{——}Bacteria; p_{——}_{_{—}}Firmicutes_A; c_{——}Clostridia; o_{——}Lachnospirales; | Guild 1 |
| | f_{——}Anaerotignaceae; g_{——}Anaerotignum; s_{——}Anaerotignum faecicola | |
| ERR2017735.12 | d_{——}Bacteria; p_{——}Firmicutes_A; c_{——}Clostridia; o_{——}Oscillospirales; | Guild 1 |
| | f_{——}Ruminococcaceae; g_{——}Faecalibacterium; s_{——}Faecalibacterium prausnitzii_G | |
| ERR2017736.11 | d_{——}Bacteria; p_{——}Firmicutes_A; c_{——}Clostridia; o_{——}Oscillospirales; | Guild 1 |
| | f_{——}Oscillospiraceae; g_{——}ER4; s_{——}ER4 sp900550165 | |
| ERR2017739.2 | d_{——}Bacteria; p_{——}Firmicutes_A; c_{——}Clostridia; o_{——}Oscillospirales; | Guild 1 |
| | f_{——}Oscillospiraceae; g_{——}Lawsonibacter; s_{——}Lawsonibacter sp000177015 | |
| ERR2017740.16 | d_{——}Bacteria; p_{——}Firmicutes_A; c_{——}Clostridia; o_{——}Monoglobales_A; | Guild 1 |
| | f_{——}UBA1381; g_{——}CAG-41; s_{——}CAG-41 sp900066215 | |
| ERR2017740.2 | d_{——}Bacteria; p_{——}Firmicutes_A; c_{——}Clostridia; o_{——}Lachnospirales; | Guild 1 |
| | f_{——}Lachnospiraceae; g_{——}Blautia_A; s_{——}Blautia_A sp900541345 | |
| ERR2017741.15 | d_{——}Bacteria; p_{——}Actinobacteriota; c_{——}Actinomycetia; o_{——}Actinomycetales; | Guild 2 |
| | f_{——}Bifidobacteriaceae; g_{——}Bifidobacterium; s_{——}Bifidobacterium dentium | |
| ERR2017742.15 | d_{——}Bacteria; p_{——}Firmicutes_A; c_{——}Clostridia; o_{——}Lachnospirales; | Guild 1 |
| | f_{——}Lachnospiraceae; g_{——}Mediterraneibacter; s_{——}Mediterraneibacter faecis | |
| ERR2017746.14 | d_{——}Bacteria; p_{——}Firmicutes_A; c_{——}Clostridia; o_{——}Lachnospirales; | Guild 2 |
| | f_{——}Lachnospiraceae; g_{——}Dorea_A; s_{——}Dorea_A longicatena_B | |
| ERR2017750.5 | d_{——}Bacteria; p_{——}Firmicutes_A; c_{——}Clostridia; o_{——}Oscillospirales; | Guild 1 |
| | f_{——}Oscillospiraceae; g_{——}Lawsonibacter; s_{——}Lawsonibacter sp000177015 | |
| ERR2017771.1 | d_{——}Bacteria; p_{——}Firmicutes_A; c_{——}Clostridia; o_{——}Lachnospirales; | Guild 1 |
| | f_{——}Lachnospiraceae; g_{——}Agathobacter; s_{——}Agathobacter rectalis | |
| ERR2017781.12 | d_{——}Bacteria; p_{——}Firmicutes; c_{——}Bacilli; o_{——}Erysipelotrichales; | Guild 1 |
| | f_{——}Erysipelotrichaceae; g_{——}Holdemanella; s_{——}Holdemanella biformis | |
| ERR2017814.13 | d_{——}Bacteria; p_{——}Firmicutes_A; c_{——}Clostridia; o_{——}Lachnospirales; | Guild 2 |
| | f_{——}Lachnospiraceae; g_{——}Blautia; s_{——}Blautia stercoris | |
| ERR2726410.2 | d_{——}Bacteria; p_{——}Firmicutes_A; c_{——}Clostridia; o_{——}Oscillospirales; | Guild 1 |
| | f_{——}Ruminococcaceae; g_{——}Gemmiger; s_{——}Gemmiger formicilis | |
| ERR2726411.1 | d_{——}Bacteria; p_{——}Bacteroidota; c_{——}Bacteroidia; o_{——}Bacteroidales; | Guild 2 |
| | f_{——}Bacteroidaceae; g_{——}Prevotella; s_{——}Prevotella nigrescens | |
| ERR2726419.10 | d_{——}Bacteria; p_{——}Firmicutes_A; c_{——}Clostridia; o_{——}Oscillospirales; | Guild 1 |
| | f_{——}Acutalibacteraceae; g_{——}Acutalibacter; s_{——}Acutalibacter sp000435395 | |
| ERR2726420.13 | d_{——}Bacteria; p_{——}Firmicutes_A; c_{——}Clostridia; o_{——}Lachnospirales; | Guild 2 |
| | f_{——}Lachnospiraceae; g_{——}Butyrivibrio_A; s_{——}Butyrivibrio_A crossotus | |
| ERR2726422.1 | d_{——}Bacteria; p_{——}Firmicutes_A; c_{——}Clostridia; o_{——}Lachnospirales; | Guild 2 |
| | f_{——}Lachnospiraceae; g_{——}Roseburia; s_{——}Roseburia intestinalis | |
| ERR2726423.2 | d_{——}Bacteria; p_{——}Firmicutes_A; c_{——}Clostridia; o_{——}Oscillospirales; | Guild 1 |
| | f_{——}Oscillospiraceae; g_{——}CAG-170; s_{——}CAG-170 sp900548625 | |
| ERR2726433.15 | d_{——}Bacteria; p_{——}Bacteroidota; c_{——}Bacteroidia; o_{——}Bacteroidales; | Guild 2 |
| | f_{——}Bacteroidaceae; g_{——}Phocaeicola; s_{——}Phocaeicola sp000434735 | |
| ERR2726447.12 | d_{——}Bacteria; p_{——}Firmicutes; c_{——}Bacilli; o_{——}RFN20; | Guild 1 |
| | f_{——}CAG-288; g_{——}CAG-568; s_{——}CAG-568 sp000434395 | |
| ERR2726447.19 | d_{——}Bacteria; p_{——}Bacteroidota; c_{——}Bacteroidia; o_{——}Bacteroidales; | Guild 2 |
| | f_{——}Bacteroidaceae; g_{——}Prevotella; s_{——}Prevotella sp002265625 | |
| ERR2726447.2 | d_{——}Bacteria; p_{——}Firmicutes_A; c_{——}Clostridia; o_{——}Lachnospirales; | Guild 1 |
| | f_{——}Lachnospiraceae; g_{——}KLE1615; s_{——}KLE1615 sp900066985 | |
| ERR2726451.2 | d_{——}Bacteria; p_{——}Bacteroidota; c_{——}Bacteroidia; o_{——}Bacteroidales; | Guild 2 |
| | f_{——}Tannerellaceae; g_{——}Parabacteroides; s_{——}Parabacteroides merdae | |
| ERR2726452.15 | d_{——}Bacteria; p_{——}Bacteroidota; c_{——}Bacteroidia; o_{——}Bacteroidales; | Guild 2 |
| | f_{——}Rikenellaceae; g_{——}Alistipes; s_{——}Alistipes putredinis | |
| ERR2726452.9 | d_{——}Bacteria; p_{——}Firmicutes_A; c_{——}Clostridia; o_{——}Oscillospirales; | Guild 1 |
| | f_{——}Ruminococcaceae; g_{——}Negativibacillus; s_{——}Negativibacillus sp900547455 | |
| ERR2726458.13 | d_{——}Bacteria; p_{——}Firmicutes_A; c_{——}Clostridia; o_{——}Lachnospirales; | Guild 2 |
| | f_{——}Lachnospiraceae; g_{——}Mediterraneibacter; s_{——}Mediterraneibacter lactaris | |
| ERR2726476.5 | d_{——}Bacteria; p_{——}Bacteroidota; c_{——}Bacteroidia; o_{——}Bacteroidales; | Guild 2 |
| | f_{——}Bacteroidaceae; g_{——}Phocaeicola; s_{——}Phocaeicola sp000432735 | |
| ERR2726486.19 | d_{——}Bacteria; p_{——}Bacteroidota; c_{——}Bacteroidia; o_{——}Bacteroidales; | Guild 2 |
| | f_{——}Rikenellaceae; g_{——}Alistipes; s_{——}Alistipes communis | |
| ERR2726493.4 | d_{——}Bacteria; p_{——}Firmicutes_A; c_{——}Clostridia; o_{——}Oscillospirales; | Guild 1 |
| | f_{——}Oscillospiraceae; g_{——}Dysosmobacter; s_{——}Dysosmobacter welbionis | |
| ERR2726493.5 | d_{——}Bacteria; p_{——}Firmicutes_A; c_{——}Clostridia; o_{——}Oscillospirales; | Guild 1 |
| | f_{——}Acutalibacteraceae; g_{——}UMGS403; s_{——}UMGS403 sp900540275 | |
| ERR2726498.24 | d_{——}Bacteria; p_{——}Bacteroidota; c_{——}Bacteroidia; o_{——}Bacteroidales; | Guild 2 |
| | f_{——}Muribaculaceae; g_{——}Muribaculum; s_{——}Muribaculum sp003150235 | |
| ERR2726502.1 | d_{——}Bacteria; p_{——}Firmicutes_A; c_{——}Clostridia; o_{——}Lachnospirales; | Guild 1 |
| | f_{——}Lachnospiraceae; g_{——}UBA11774; s_{——}UBA11774 sp003507655 | |
| ERR2726522.5 | d_{——}Bacteria; p_{——}Firmicutes_A; c_{——}Clostridia_A; o_{——}Christensenellales; | Guild 1 |
| | f_{——}CAG-74; g_{——}SFFH01; s_{——}SFFH01 sp900542395 | |
| ERR2726527.4 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides uniformis | |
| ERR2726532.18 | d_{——}Bacteria; p_{——}Firmicutes_A; c_{——}Clostridia; o_{——}Oscillospirales; | Guild 1 |
| | f_{——}Acutalibacteraceae; g_{——}Pseudoruminococcus; s_{——}Pseudoruminococcus massiliensis | |
| ERR2855787.12 | d_{——}Bacteria; p_{——}Firmicutes_A; c_{——}Clostridia; o_{——}Oscillospirales; | Guild 1 |
| | f_{——}Ruminococcaceae; g_{——}CAG-115; s_{——}CAG-115 sp003531585 | |
| ERR2855790.1 | d_{——}Bacteria; p_{——}Firmicutes_A; c_{——}Clostridia; o_{——}Oscillospirales; | Guild 1 |
| | f_{——}Oscillospiraceae; g_{——}Dysosmobacter; s_{——}Dysosmobacter sp001916835 | |
| ERR2855795.12 | d_{——}Bacteria; p_{——}Firmicutes_A; c_{——}Clostridia; o_{——}Oscillospirales; | Guild 1 |
| | f_{——}Ruminococcaceae; g_{——}Faecalibacterium; s_{——}Faecalibacterium sp900539945 | |
| ERR2855800.13 | d_{——}Bacteria; p_{——}Proteobacteria; c_{——}Gammaproteobacteria; o_{——}Burkholderiales; | Guild 1 |
| | f_{——}Burkholderiaceae; g_{——}Duodenibacillus; s_{——}Duodenibacillus sp003472385 | |
| ERR2855808.13 | d_{——}Bacteria; p_{——}Firmicutes_A; c_{——}Clostridia; o_{——}Lachnospirales; | Guild 2 |
| | f_{——}Lachnospiraceae; g_{——}CAG-81; s_{——}CAG-81 sp900066535 | |
| ERR2855814.14 | d_{——}Bacteria; p_{——}Firmicutes_A; c_{——}Clostridia; o_{——}Lachnospirales; | Guild 1 |
| | f_{——}Lachnospiraceae; g_{——}TF01-11; s_{——}TF01-11 sp001414325 | |
| ERR2855814.4 | d_{——}Bacteria; p_{——}Firmicutes_A; c_{——}Clostridia; o_{——}Lachnospirales; | Guild 2 |
| | f_{——}Lachnospiraceae; g_{——}UBA9502; s_{——}UBA9502 sp003480315 | |
| ERR2855821.2 | d_{——}Bacteria; p_{——}Firmicutes_A; c_{——}Clostridia; o_{——}Lachnospirales; | Guild 2 |
| | f_{——}Lachnospiraceae; g_{——}CAG-81; s_{——}CAG-81 sp900066535 | |
| ERR2855825.1 | d_{——}Bacteria; p_{——}Bacteroidota; c_{——}Bacteroidia; o_{——}Bacteroidales; | Guild 2 |
| | f_{——}Bacteroidaceae; g_{——}Phocaeicola; s_{——}Phocaeicola massiliensis | |
| ERR2855828.7 | d_{——}Bacteria; p_{——}Firmicutes_A; c_{——}Clostridia; o_{——}Lachnospirales; | Guild 2 |
| | f_{——}Lachnospiraceae; g_{——}UBA9502; s_{——}UBA9502 sp003478505 | |
| ERR2855831.6 | d_{——}Bacteria; p_{——}Proteobacteria; c_{——}Gammaproteobacteria; o_{——}Burkholderiales; | Guild 2 |
| | f_{——}Burkholderiaceae; g_{——}Duodenibacillus; s_{——}Duodenibacillus massiliensis | |
| ERR2855838.18 | d_{——}Bacteria; p_{——}Firmicutes_A; c_{——}Clostridia; o_{——}Lachnospirales; | Guild 2 |
| | f_{——}Lachnospiraceae; g_{——}TF01-11; s_{——}TF01-11 sp003524945 | |
| ERR2855840.6 | d_{——}Bacteria; p_{——}Firmicutes_A; c_{——}Clostridia; o_{——}Oscillospirales; | Guild 2 |
| | f_{——}Ruminococcaceae; g_{——}Gemmiger; s_{——}Gemmiger formicilis | |
| ERR2855844.18 | d_{——}Bacteria; p_{——}Firmicutes_A; c_{——}Clostridia; o_{——}Lachnospirales; | Guild 2 |
| | f_{——}Lachnospiraceae; g_{——}Clostridium_Q; s_{——}Clostridium_Q sp003024715 | |
| ERR2855857.7 | d_{——}Bacteria; p_{——}Proteobacteria; c_{——}Gammaproteobacteria; o_{——}Burkholderiales; | Guild 2 |
| | f_{——}Burkholderiaceae; g_{——}Mesosutterella; s_{——}Mesosutterella multiformis | |
| ERR2855864.8 | d_{——}Bacteria; p_{——}Bacteroidota; c_{——}Bacteroidia; o_{——}Bacteroidales; | Guild 2 |
| | f_{——}Bacteroidaceae; g_{——}Prevotella; s_{——}Prevotella stercorea | |
| ERR2855871.6 | d_{——}Bacteria; p_{——}Firmicutes_A; c_{——}Clostridia; o_{——}Lachnospirales; | Guild 2 |
| | f_{——}Lachnospiraceae; g_{——}Lachnospira; s_{——}Lachnospira sp900545725 | |
| ERR2855880.6 | d_{——}Bacteria; p_{——}Firmicutes_A; c_{——}Clostridia; o_{——}Oscillospirales; | Guild 2 |
| | f_{——}Ruminococcaceae; g_{——}Faecalibacterium; s_{——}Faecalibacterium prausnitzii_G | |
| ERR2855886.18 | d_{——}Bacteria; p_{——}Firmicutes_A; c_{——}Clostridia; o_{——}Oscillospirales; | Guild 1 |
| | f_{——}Acutalibacteraceae; g_{——}Ruminococcus_E; s_{——}Ruminococcus_E sp003526955 | |
| ERR2855908.3 | d_{——}Bacteria; p_{——}Firmicutes_C; c_{——}Negativicutes; o_{——}Veillonellales; | Guild 2 |
| | f_{——}Dialisteraceae; g_{——}Dialister; s_{——}Dialister sp900538805 | |
| ERR2855923.15 | d_{——}Bacteria; p_{——}Firmicutes_A; c_{——}Clostridia; o_{——}Oscillospirales; | Guild 1 |
| | f_{——}Ruminococcaceae; g_{——}Ruminococcus_C; s_{——}Ruminococcus_C sp000433635 | |
| ERR2855933.19 | d_{——}Bacteria; p_{——}Firmicutes_A; c_{——}Clostridia; o_{——}Lachnospirales; | Guild 2 |
| | f_{——}Lachnospiraceae; g_{——}Eubacterium_G; s_{——}Eubacterium_G sp000435815 | |
| ERR2855935.6 | d_{——}Bacteria; p_{——}Firmicutes_A; c_{——}Clostridia; o_{——}Oscillospirales; | Guild 2 |
| | f_{——}Acutalibacteraceae; g_{——}CAG-177; s_{——}CAG-177 sp003514385 | |
| ERR2855935.9 | d_{——}Bacteria; p_{——}Bacteroidota; c_{——}Bacteroidia; o_{——}Bacteroidales; | Guild 2 |
| | f_{——}Rikenellaceae; g_{——}Alistipes_A; s_{——}Alistipes_A sp900539755 | |
| ERR2855947.28 | d_{——}Bacteria; p_{——}Firmicutes_A; c_{——}Clostridia; o_{——}Oscillospirales; | Guild 1 |
| | f_{——}Oscillospiraceae; g_{——}CAG-83; s_{——}CAG-83 sp900552475 | |
| ERR2855947.3 | d_{——}Bacteria; p_{——}Firmicutes_A; c_{——}Clostridia; o_{——}Oscillospirales; | Guild 1 |
| | f_{——}Oscillospiraceae; g_{——}Dysosmobacter; s_{——}Dysosmobacter sp001916835 | |
| ERR688506.3 | d_{——}Bacteria; p_{——}Firmicutes_A; c_{——}Clostridia; o_{——}Oscillospirales; | Guild 1 |
| | f_{——}Ruminococcaceae; g_{——}Faecalibacterium; s_{——}Faecalibacterium prausnitzii_E | |
| ERR688507.14 | d_{——}Bacteria; p_{——}Firmicutes; c_{——}Bacilli; o_{——}Lactobacilli; | Guild 2 |
| | f_{——}Streptococcaceae; g_{——}Streptococcus; s_{——}Streptococcus thermophilus | |
| ERR688513.3 | d_{——}Bacteria; p_{——}Actinobacteriota; c_{——}Coriobacteriia; o_{——}Coriobacteriales; | Guild 2 |
| | f_{——}Coriobacteriaceae; g_{——}Collinsella; s_{——}Collinsella sp900541745 | |
| ERR688518.6 | d_{——}Bacteria; p_{——}Firmicutes_A; c_{——}Clostridia; o_{——}Lachnospirales; | Guild 2 |
| | f_{——}Lachnospiraceae; g_{——}Anaerostipes; s_{——}Anaerostipes sp900066705 | |
| ERR688520.2 | d_{——}Bacteria; p_{——}Firmicutes_A; c_{——}Clostridia; o_{——}Oscillospirales; | Guild 2 |
| | f_{——}Acutalibacteraceae; g_{——}Eubacterium_R; s_{——}Eubacterium_R sp000436835 | |
| ERR688520.8 | d_{——}Bacteria; p_{——}Firmicutes_A; c_{——}Clostridia; o_{——}Lachnospirales; | Guild 2 |
| | f_{——}Lachnospiraceae; g_{——}CAG-317; s_{——}CAG-317 sp900543415 | |
| ERR688521.4 | d_{——}Bacteria; p_{——}Firmicutes_A; c_{——}Clostridia; o_{——}Lachnospirales; | Guild 1 |
| | f_{——}Lachnospiraceae; g_{——}Ruminococcus_A; s_{——}Ruminococcus_A sp003011855 | |
| ERR688522.8 | d_{——}Bacteria; p_{——}Firmicutes_A; c_{——}Clostridia; o_{——}Oscillospirales; | Guild 1 |
| | f_{——}Ruminococcaceae; g_{——}Faecalibacterium; s_{——}Faecalibacterium prausnitzii_D | |
| ERR688522.9 | d_{——}Bacteria; p_{——}Firmicutes_A; c_{——}Clostridia; o_{——}Lachnospirales; | Guild 2 |
| | f_{——}Lachnospiraceae; g_{——}Dorea; s_{——}Dorea formicigenerans | |
| ERR688524.6 | d_{——}Bacteria; p_{——}Firmicutes_A; c_{——}Clostridia; o_{——}Lachnospirales; | Guild 2 |
| | f_{——}Lachnospiraceae; g_{——}Enterocloster; s_{——}Enterocloster sp000155435 | |
| ERR688528.23 | d_{——}Bacteria; p_{——}Firmicutes_A; c_{——}Clostridia; o_{——}Lachnospirales; | Guild 2 |
| | f_{——}Lachnospiraceae; g_{——}Bacteroides_F; s_{——}Bacteroides_F pectinophilus | |
| ERR688529.17 | d_{——}Bacteria; p_{——}Actinobacteriota; c_{——}Coriobacteriia; o_{——}Coriobacteriales; | Guild 2 |
| | f_{——}Coriobacteriaceae; g_{——}Collinsella; s_{——}Collinsella sp900758375 | |
| ERR688536.9 | d_{——}Bacteria; p_{——}Firmicutes_A; c_{——}Clostridia; o_{——}Lachnospirales; | Guild 1 |
| | f_{——}Lachnospiraceae; g_{——}Ruminococcus_A; s_{——}Ruminococcus_A sp003011855 | |
| ERR688537.6 | d_{——}Bacteria; p_{——}Actinobacteriota; c_{——}Actinomycetia; o_{——}Actinomycetales; | Guild 2 |
| | f_{——}Bifidobacteriaceae; g_{——}Bifidobacterium; s_{——}Bifidobacterium pullorum | |
| ERR688539.8 | d_{——}Bacteria; p_{——}Firmicutes_A; c_{——}Clostridia; o_{——}Lachnospirales; | Guild 1 |
| | f_{——}Lachnospiraceae; g_{——}Blautia; s_{——}Blautia stercoris | |
| ERR688558.20 | d_{——}Bacteria; p_{——}Actinobacteriota; c_{——}Coriobacteriia; o_{——}Coriobacteriales; | Guild 2 |
| | f_{——}Eggerthellaceae; g_{——}CAG-1427; s_{——}CAG-1427 sp000436075 | |
| ERR688558.6 | d_{——}Bacteria; p_{——}Firmicutes_A; c_{——}Clostridia; o_{——}Oscillospirales; | Guild 1 |
| | f_{——}Ruminococcaceae; g_{——}Gemmiger; s_{——}Gemmiger qucibialis | |
| ERR688559.18 | d_{——}Bacteria; p_{——}Actinobacteriota; c_{——}Coriobacteriia; o_{——}Coriobacteriales; | Guild 2 |
| | f_{——}Eggerthellaceae; g_{——}CAG-1427; s_{——}CAG-1427 sp000431675 | |
| ERR688563.10 | d_{——}Bacteria; p_{——}Firmicutes_A; c_{——}Clostridia; o_{——}Oscillospirales; | Guild 2 |
| | f_{——}Acutalibacteraceae; g_{——}UBA1417; s_{——}UBA1417 sp003531055 | |
| ERR688564.13 | d_{——}Bacteria; p_{——}Firmicutes_A; c_{——}Clostridia; o_{——}Lachnospirales; | Guild 1 |
| | f_{——}Lachnospiraceae; g_{——}Fusicatenibacter; s_{——}Fusicatenibacter saccharivorans | |
| ERR688567.8 | d_{——}Bacteria; p_{——}Firmicutes_A; c_{——}Clostridia; o_{——}Oscillospirales; | Guild 1 |
| | f_{——}Oscillospiraceae; g_{——}NK3B98; s_{——}NK3B98 sp900545815 | |
| ERR688577.19 | d_{——}Bacteria; p_{——}Proteobacteria; c_{——}Gammaproteobacteria; o_{——}Enterobacterales; | Guild 2 |
| | f_{——}Enterobacteriaceae; g_{——}Escherichia; s_{——}Escherichia dysenteriae | |
| ERR688589.10 | d_{——}Bacteria; p_{——}Firmicutes_A; c_{——}Clostridia; o_{——}Oscillospirales; | Guild 1 |
| | f_{——}Oscillospiraceae; g_{——}CAG-110; s_{——}CAG-110 sp000434635 | |
| ERR688598.5 | d_{——}Bacteria; p_{——}Actinobacteriota; c_{——}Actinomycetia; o_{——}Actinomycetales; | Guild 1 |
| | f_{——}Bifidobacteriaceae; g_{——}Bifidobacterium; s_{——}Bifidobacterium breve | |
| ERR688604.10 | d_{——}Bacteria; p_{——}Firmicutes_A; c_{——}Clostridia; o_{——}Lachnospirales; | Guild 1 |
| | f_{——}Lachnospiraceae; g_{——}Eubacterium_G; s_{——}Eubacterium_G sp000434315 | |
| ERR688608.9 | d_{——}Bacteria; p_{——}Firmicutes_A; c_{——}Clostridia; o_{——}Lachnospirales; | Guild 1 |
| | f_{——}Lachnospiraceae; g_{——}Anaerobutyricum; s_{——}Anaerobutyricum soehngenii | |
| ERR688610.11 | d_{——}Bacteria; p_{——}Firmicutes_A; c_{——}Clostridia_A; o_{——}Christensenellales; | Guild 1 |
| | f_{——}CAG-138; g_{——}PeH17; s_{——}PeH17 sp900542285 | |
| ERR688610.9 | d_{——}Bacteria; p_{——}Firmicutes_A; c_{——}Clostridia; o_{——}Lachnospirales; | Guild 2 |
| | f_{——}Lachnospiraceae; g_{——}Roseburia; s_{——}Roseburia hominis | |
| ERR688612.12 | d_{——}Bacteria; p_{——}Firmicutes_A; c_{——}Clostridia; o_{——}Lachnospirales; | Guild 1 |
| | f_{——}Lachnospiraceae; g_{——}Fusicatenibacter; s_{——}Fusicatenibacter saccharivorans | |
| ERR688615.3 | d_{——}Bacteria; p_{——}Firmicutes_A; c_{——}Clostridia; o_{——}Lachnospirales; | Guild 2 |
| | f_{——}Lachnospiraceae; g_{——}Blautia_A; s_{——}Blautia_A obeum | |
| ERR688615.8 | d_{——}Bacteria; p_{——}Firmicutes_A; c_{——}Clostridia; o_{——}Lachnospirales; | Guild 1 |
| | f_{——}CAG-274; g_{——}CAG-274; s_{——}CAG-274 sp000432155 | |
| ERR688617.7 | d_{——}Bacteria; p_{——}Firmicutes_A; c_{——}Clostridia; o_{——}Oscillospirales; | Guild 1 |
| | f_{——}Ruminococcaceae; g_{——}Gemmiger; s_{——}Gemmiger sp900540775 | |
| ERR688623.13 | d_{——}Bacteria; p_{——}Firmicutes_A; c_{——}Clostridia; o_Oscillospirales; | Guild 1 |
| | f_{——}Butyricicoccaceae; g_{——}Agathobaculum; s_{——}Agathobaculum butyriciproducens | |
| ERR688627.12 | d_{——}Bacteria; p_{——}Firmicutes_A; c_{——}Clostridia; o_{——}Lachnospirales; | Guild 2 |
| | f_{——}Lachnospiraceae; g_{——}Blautia_A; s_{——}Blautia_A wexlerae | |
| ERR688629.1 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides uniformis | |
| ERR688629.15 | d_{——}Bacteria; p_{——}Firmicutes; c_{——}Bacilli; o_{——}Erysipelotrichales; | Guild 2 |
| | f_{——}Erysipelotrichaceae; g_{——}Bulleidia; s_{——}Bulleidia sp900539965 | |
| ERR688631.6 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides stercoris | |
| ERR688640.13 | d_{——}Bacteria; p_{——}Firmicutes; c_{——}Bacilli; o_{——}Erysipelotrichales; | Guild 2 |
| | f_{——}Erysipelotrichaceae; g_{——}Clostridium_AQ; s_{——}Clostridium_AQ sp900552125 | |
| ERR688642.6 | d_{——}Bacteria; p_{——}Firmicutes_A; c_{——}Clostridia; o_{——}Oscillospirales; | Guild 1 |
| | f_{——}Ruminococcaceae; g_{——}Gemmiger; s_{——}Gemmiger qucibialis | |
| ERR688645.6 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides fragilis | |
| HD-20.2 | d_{——}Bacteria; p_{——}Firmicutes_A; c_{——}Clostridia; o_{——}Oscillospirales; | Guild 2 |
| | f_{——}Ruminococcaceae; g_{——}Faecalibacterium; s_{——}Faecalibacterium sp900539945 | |
| HD-63.13 | d_{——}Bacteria; p_{——}Bacteroidota; c_{——}Bacteroidia; o_{——}Bacteroidales; | Guild 2 |
| | f_{——}Bacteroidaceae; g_{——}Prevotella; s | |
| HD-76.2 | d_{——}Bacteria; p_{——}Fusobacteriota; c_{——}Fusobacteriia; o_{——}Fusobacteriales; | Guild 2 |
| | f_{——}Fusobacteriaceae; g_{——}Fusobacterium_A; s_{——}Fusobacterium_A sp900543175 | |
| HD-8.10 | d_{——}Bacteria; p_{——}Firmicutes_A; c_{——}Clostridia; o_{——}Lachnospirales; | Guild 2 |
| | f_{——}Lachnospiraceae; g_{——}Eubacterium_F; s_{——}Eubacterium_F sp003491505 | |
| LD-26.6 | d_{——}Bacteria; p_{——}Firmicutes_A; c_{——}Clostridia; o_{——}Lachnospirales; | Guild 1 |
| | f_{——}Lachnospiraceae; g_{——}TF01-11; s_{——}TF01-11 sp000436755 | |
| LD-33.1 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides ndongoniae | |
| LD-35.30 | d_{——}Bacteria; p_{——}Firmicutes_A; c_{——}Clostridia; o_{——}Lachnospirales; | Guild 2 |
| | f_{——}Anaerotignaceae; g_{——}Anaerotignum; s | |
| LD-39.9 | d_{——}Bacteria; p_{——}Bacteroidota; c_{——}Bacteroidia; o_{——}Bacteroidales; | Guild 2 |
| | f_{——}Tannerellaceae; g_{——}Parabacteroides; s_{——}Parabacteroides distasonis | |
| LD-7.2 | d_{——}Bacteria; p_{——}Firmicutes_A; c_{——}Clostridia; o_{——}Lachnospirales; | Guild 1 |
| | f_{——}Lachnospiraceae; g_{——}Agathobacter; s_{——}Agathobacter faecis | |
| LD-75.3 | d_{——}Bacteria; p_{——}Bacteroidota; c_{——}Bacteroidia; o_{——}Bacteroidales; | Guild 2 |
| | f_{——}Tannerellaceae; g_{——}Parabacteroides; s_{——}Parabacteroides gordonii | |
| MSM79H8B.5 | d_{——}Bacteria; p_{——}Bacteroidota; c_{——}Bacteroidia; o_{——}Bacteroidales; | Guild 2 |
| | f_{——}Bacteroidaceae; g_{——}Phocaeicola; s_{——}Phocaeicola dorei | |
| PSMA2640.3 | d_{——}Bacteria; p_{——}Firmicutes_A; c_{——}Clostridia; o_{——}Oscillospirales; | Guild 2 |
| | f_{——}Ruminococcaceae; g_{——}Faecalibacterium; s_{——}Faecalibacterium prausnitzii_D | |
| SRR341654.1 | d_{——}Bacteria; p_{——}Verrucomicrobiota; c_{——}Verrucomicrobiae; o_{——}Verrucomicrobiales; | Guild 2 |
| | f_{——}Akkermansiaceae; g_{——}Akkermansia; s_{——}Akkermansia muciniphila | |
| SRR341680.2 | d_{——}Bacteria; p_{——}Firmicutes_A; c_{——}Clostridia; o_{——}Lachnospirales; | Guild 2 |
| | f_{——}Lachnospiraceae; g_{——}Roseburia; s_{——}Roseburia sp003470905 | |
| SRR341724.5 | d_{——}Bacteria; p_{——}Actinobacteriota; c_{——}Actinomycetia; o_{——}Actinomycetales; | Guild 1 |
| | f_{——}Bifidobacteriaceae; g_{——}Bifidobacterium; s_{——}Bifidobacterium pseudocatenulatum | |
| SRR413559.10 | d_{——}Bacteria; p_{——}Firmicutes_A; c_{——}Clostridia_A; o_{——}Christensenellales; | Guild 2 |
| | f_{——}CAG-74; g_{——}UBA11524; s_{——}UBA11524 sp000437595 | |
| SRR413567.10 | d_{——}Bacteria; p_{——}Bacteroidota; c_{——}Bacteroidia; o_{——}Bacteroidales; | Guild 2 |
| | f_{——}Tannerellaceae; g_{——}Parabacteroides; s_{——}Parabacteroides goldsteinii | |
| SRR413584.9 | d_{——}Bacteria; p_{——}Firmicutes_A; c_{——}Clostridia; o_{——}Lachnospirales; | Guild 2 |
| | f_{——}Lachnospiraceae; g_{——}CAG-603; s_{——}CAG-603 sp900066105 | |
| SRR413588.5 | d_{——}Bacteria; p_{——}Bacteroidota; c_{——}Bacteroidia; o_{——}Bacteroidales; | Guild 2 |
| | f_{——}Bacteroidaceae; g_{——}Prevotella; s_{——}Prevotella sp900551275 | |
| SRR413635.13 | d_{——}Bacteria; p_{——}Bacteroidota; c_{——}Bacteroidia; o_{——}Bacteroidales; | Guild 1 |
| | f_{——}Bacteroidaceae; g_{——}Prevotella; s_{——}Prevotella buccae | |
| SRR413635.5 | d_{——}Bacteria; p_{——}Firmicutes_A; c_{——}Clostridia; o_{——}Lachnospirales; | Guild 1 |
| | f_{——}Lachnospiraceae; g_{——}CAG-45; s_{——}CAG-45 sp000438375 | |
| SRR413641.2 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides ovatus | |
| SRR413642.4 | d_{——}Bacteria; p_{——}Firmicutes_A; c_{——}Clostridia; o_{——}Lachnospirales; | Guild 2 |
| | f_{——}Lachnospiraceae; g_{——}Lachnospira; s_{——}Lachnospira sp000437735 | |
| SRR413642.5 | d_{——}Bacteria; p_{——}Firmicutes_A; c_{——}Clostridia; o_{——}Lachnospirales; | Guild 2 |
| | f_{——}Lachnospiraceae; g_{——}Agathobacter; s_{——}Agathobacter faecis | |
| SRR413655.3 | d_{——}Bacteria; p_{——}Firmicutes_A; c_{——}Clostridia; o_{——}Lachnospirales; | Guild 2 |
| | f_{——}Lachnospiraceae; g_{——}Lachnospira; s_{——}Lachnospira rogosae_A | |
| SRR413677.1 | d_{——}Bacteria; p_{——}Bacteroidota; c_{——}Bacteroidia; o_{——}Bacteroidales; | Guild 2 |
| | f_{——}Marinifilaceae; g_{——}Odoribacter; s_{——}Odoribacter splanchnicus | |
| SRR413691.1 | d_{——}Bacteria; p_{——}Bacteroidota; c_{——}Bacteroidia; o_{——}Bacteroidales; | Guild 2 |
| | f_{——}Bacteroidaceae; g_{——}Prevotella; s_{——}Prevotella sp900313215 | |
| SRR413694.1 | d_{——}Bacteria; p_{——}Firmicutes_A; c_{——}Clostridia; o_{——}Lachnospirales; | Guild 2 |
| | f_{——}Lachnospiraceae; g_{——}Lachnospira; s_{——}Lachnospira eligens_A | |
| SRR413729.3 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides caccae | |
| SRR413736.4 | d_{——}Bacteria; p_{——}Bacteroidota; c_{——}Bacteroidia; o_{——}Bacteroidales; | Guild 2 |
| | f_{——}Marinifilaceae; g Butyricimonas; s_{——}Butyricimonas virosa | |
| SRR413753.22 | d_{——}Bacteria; p_{——}Bacteroidota; c_{——}Bacteroidia; o_{——}Bacteroidales; | Guild 2 |
| | f_{——}Rikenellaceae; g_{——}Alistipes; s_{——}Alistipes communis | |
| SRR413755.1 | d_{——}Bacteria; p_{——}Firmicutes_A; c_{——}Clostridia; o_{——}Lachnospirales; | Guild 1 |
| | f_{——}Lachnospiraceae; g_{——}Enterocloster; s_{——}Enterocloster aldenensis | |
| SRR413761.12 | d_{——}Bacteria; p_{——}Firmicutes_A; c_{——}Clostridia; o_{——}Lachnospirales; | Guild 2 |
| | f_{——}Anaerotignaceae; g_{——}Anaerotignum; s_{——}Anaerotignum faecicola | |
| SRR413762.3 | d_{——}Bacteria; p_{——}Firmicutes_A; c_{——}Clostridia; o_{——}Lachnospirales; | Guild 2 |
| | f_{——}Lachnospiraceae; g_{——}CAG-95; s_{——}CAG-95 sp000438155 | |
| SRR413762.6 | d_{——}Bacteria; p_{——}Bacteroidota; c_{——}Bacteroidia; o_{——}Bacteroidales; | Guild 2 |
| | f_{——}Marinifilaceae; g_{——}Odoribacter; s_{——}Odoribacter massiliensis | |
| SRR5279219.7 | d_{——}Bacteria; p_{——}Firmicutes_A; c_{——}Clostridia; o_{——}Oscillospirales; | Guild 2 |
| | f_{——}Acutalibacteraceae; g_{——}Ruminococcus_E; s_{——}Ruminococcus_E sp900314705 | |
| SRR5279222.3 | d_{——}Bacteria; p_{——}Firmicutes_A; c_{——}Clostridia; o_{——}Lachnospirales; | Guild 2 |
| | f_{——}Lachnospiraceae; g_{——}Blautia_A; s_{——}Blautia_A sp003480185 | |
| SRR5279239.4 | d_{——}Bacteria; p_{——}Firmicutes; c_{——}Bacilli; o_{——}Lactobacilli; | Guild 2 |
| | f_{——}Lactobacillaceae; g_{——}Limosilactobacillus; s_{——}Limosilactobacillus mucosae | |
| SRR5279253.7 | d_{——}Bacteria; p_{——}Proteobacteria; c_{——}Gammaproteobacteria; o_{——}Burkholderiales; | Guild 2 |
| | f_{——}Burkholderiaceae; g_{——}Sutterella; s | |
| SRR5279269.27 | d_{——}Bacteria; p_{——}Firmicutes_A; c_{——}Clostridia; o_{——}Lachnospirales; | Guild 2 |
| | f_{——}Lachnospiraceae; g_{——}Eubacterium_G; s_{——}Eubacterium_G ventriosum | |
| SRR5279278.2 | d_{——}Bacteria; p_{——}Bacteroidota; c_{——}Bacteroidia; o_{——}Bacteroidales; | Guild 2 |
| | f_{——}Bacteroidaceae; g_{——}Prevotellamassilia; s_{——}Prevotellamassilia sp900539625 | |
| SRR5279285.2 | d_{——}Bacteria; p_{——}Firmicutes; c_{——}Bacilli; o_{——}Erysipelotrichales; | Guild 2 |
| | f_{——}Erysipelatoclostridiaceae; g_{——}Faecalibacillus; s_{——}Faecalibacillus intestinalis | |
| SRR5279303.4 | d_{——}Bacteria; p_{——}Firmicutes_C; c_{——}Negativicutes; o_{——}Veillonellales; | Guild 2 |
| | f_{——}Veillonellaceae; g_{——}Veillonella; s_{——}Veillonella parvula_A | |
| SRR6468507.8 | d_{——}Bacteria; p_{——}Firmicutes_A; c_{——}Clostridia; o_{——}Lachnospirales; | Guild 1 |
| | f_{——}Lachnospiraceae; g_{——}Blautia_A; s_{——}Blautia_A wexlerae | |
| SRR6468510.13 | d_{——}Bacteria; p_{——}Bacteroidota; c_{——}Bacteroidia; o_{——}Bacteroidales; | Guild 2 |
| | f_{——}UBA932; g_{——}RC9; s_{——}RC9 sp000434935 | |
| SRR6468523.9 | d_{——}Bacteria; p_{——}Firmicutes_A; c_{——}Clostridia; o_{——}Oscillospirales; | Guild 1 |
| | f_{——}Oscillospiraceae; g_{——}CAG-170; s_{——}CAG-170 sp000432135 | |
| SRR6468543.7 | d_{——}Bacteria; p_{——}Firmicutes_A; c_{——}Clostridia; o_{——}Oscillospirales; | Guild 1 |
| | f_{——}Acutalibacteraceae; g_{——}Eubacterium_R; s_{——}Eubacterium_R sp900540305 | |
| SRR6468590.5 | d_{——}Bacteria; p_{——}Firmicutes_A; c_{——}Clostridia; o_{——}Lachnospirales; | Guild 1 |
| | f_{——}Lachnospiraceae; g_{——}Eubacterium_G; s_{——}Eubacterium_G sp000432355 | |
| SRR6468594.3 | d_{——}Bacteria; p_{——}Firmicutes_A; c_{——}Clostridia; o_{——}Lachnospirales; | Guild 2 |
| | f_{——}Lachnospiraceae; g_{——}V9D3004; s_{——}V9D3004 sp900760345 | |
| SRR6468602.3 | d_{——}Bacteria; p_{——}Proteobacteria; c_{——}Gammaproteobacteria; o_{——}Burkholderiales; | Guild 2 |
| | f_{——}Burkholderiaceae; g_{——}Parasutterella; s_{——}Parasutterella sp000980495 | |
| SRR6468612.10 | d_{——}Bacteria; p_{——}Firmicutes_A; c_{——}Clostridia; o_{——}Oscillospirales; | Guild 2 |
| | f_{——}Acutalibacteraceae; g_{——}Acutalibacter; s_{——}Acutalibacter timonensis | |
| SRR6468618.9 | d_{——}Bacteria; p_{——}Firmicutes_A; c_{——}Clostridia; o_{——}Lachnospirales; | Guild 2 |
| | f_{——}Lachnospiraceae; g_{——}Sellimonas; s_{——}Sellimonas intestinalis | |
| SRR6468619.8 | d_{——}Bacteria; p_{——}Firmicutes_A; c_{——}Clostridia; o_{——}Lachnospirales; | Guild 2 |
| | f_{——}Lachnospiraceae; g_{——}Enterocloster; s_{——}Enterocloster bolteae | |
| SRR6468633.4 | d_{——}Bacteria; p_{——}Firmicutes_A; c_{——}Clostridia; o_{——}Oscillospirales; | Guild 1 |
| | f_{——}Ruminococcaceae; g_{——}Gemmiger; s_{——}Gemmiger sp900539695 | |
| SRR6468637.9 | d_{——}Bacteria; p_{——}Bacteroidota; c_{——}Bacteroidia; o_{——}Bacteroidales; | Guild 2 |
| | f_{——}Bacteroidaceae; g_{——}Bacteroides; s_{——}Bacteroides uniformis | |
| SRR6468639.4 | d_{——}Bacteria; p_{——}Firmicutes; c_{——}Bacilli; o_{——}Lactobacillales; | Guild 1 |
| | f_{——}Streptococcaceae; g_{——}Streptococcus; s_{——}Streptococcus lutetiensis | |
| SRR6468651.2 | d_{——}Bacteria; p_{——}Firmicutes_A; c_{——}Clostridia; o_{——}Lachnospirales; | Guild 2 |
| | f_{——}Lachnospiraceae; g_{——}Faecalimonas; s | |
| SRR6468652.5 | d_{——}Bacteria; p_{——}Firmicutes_C; c_{——}Negativicutes; o_{——}Veillonellales; | Guild 2 |
| | f_{——}Megasphaeraceae; g_{——}Anaeroglobus; s_{——}Anaeroglobus micronuciformis | |
| SRR6468672.4 | d_{——}Bacteria; p_{——}Firmicutes_C; c_{——}Negativicutes; o_{——}Acidaminococcales; | Guild 2 |
| | f_{——}Acidaminococcaceae; g_{——}Acidaminococcus; s_{——}Acidaminococcus intestini | |
| SRR6468684.3 | d_{——}Bacteria; p_{——}Firmicutes_A; c_{——}Clostridia; o_{——}Lachnospirales; | Guild 1 |
| | f_{——}Lachnospiraceae; g_{——}Lachnospira; s_{——}Lachnospira sp000436475 | |
| SRR6468684.5 | d_{——}Bacteria; p_{——}Firmicutes_A; c_{——}Clostridia; o_{——}Lachnospirales; | Guild 1 |
| | f_{——}Lachnospiraceae; g_{——}KLE1615; s_{——}KLE1615 sp900066985 | |
| SRR6468688.21 | d_{——}Bacteria; p_{——}Firmicutes_A; c_{——}Clostridia; o_{——}Oscillospirales; | Guild 1 |
| | f_{——}Acutalibacteraceae; g_{——}Eubacterium_R; s_{——}Eubacterium_R sp000431535 | |
| SRR6468699.2 | d_{——}Bacteria; p_{——}Firmicutes_A; c_{——}Clostridia; o_{——}Oscillospirales; | Guild 1 |
| | f_{——}Ruminococcaceae; g_{——}Gemmiger; s_{——}Gemmiger formicilis | |

The bacterial species listed in Table 1, Table 2, and FIGS. 13A-13XX were identified by metagenomic sequencing of genomic DNA isolated from human fecal samples and determined to be part of two competing microbiota guilds relative to at least one biological characteristic, as described in the Examples. Briefly, genomic DNA was isolated from each fecal sample was sequenced by next generation sequencing and contigs for microorganism genome sequences were constructed de novo. Generally, the contigs identified for each microorganism are predicted to represent greater than 95% of the entire genome for the microorganism. Genomic constructs having less than 1% sequence divergence from each other were combined and defined to be from the same microorganism. Genomic contigs for each microorganism listed in Table 1, Table 2, and FIGS. 13A-13XX are provided in the sequence listing filed with the application. The taxonomic assignment of each microorganism is given in Table 1, Table 2, or FIGS. 13A-13XX. Correspondence between the sequence identifier assigned to each contig and the microorganism to which it belongs is provided in FIG. 12. For example, the contigs provided as SEQ ID NOS: 1-68 correspond to the genomic sequence of microorganism 1U001.8 (as indicated in FIG. 12A), which is a microorganism classified as domain Bacteria, phylum Proteobacteria, class Gammaproteobacteria, order Enterobacterales, family Enterobacteria, genus Escherichia, and species Escherichia coli and is in Guild 2 of the 141 core microorganisms identified in Table 1.

Accordingly, in some embodiments of the methods described herein, a genome identified in a metagenomic analysis is classified as corresponding to a microorganism listed in Table 1, Table 2, and/or FIGS. 13A-13XX if the identified genomic constructs have at least 97% sequence identity when compared to the contigs for the microorganism provided in the sequence listing, as denoted in FIG. 12. In some embodiments, a genome identified in a metagenomic analysis is classified as corresponding to a microorganism listed in Table 1, Table 2, and/or FIGS. 13A-13XX if the identified genomic constructs have at least 98% sequence identity when compared to the contigs for the microorganism provided in the sequence listing, as denoted in FIG. 12. In some embodiments, a genome identified in a metagenomic analysis is classified as corresponding to a microorganism listed in Table 1, Table 2, and/or FIGS. 13A-13XX if the identified genomic constructs have at least 99% sequence identity when compared to the contigs for the microorganism provided in the sequence listing, as denoted in FIG. 12. In some embodiments, a genome identified in a metagenomic analysis is classified as corresponding to a microorganism listed in Table 1, Table 2, and/or FIGS. 13A-13XX if the identified genomic constructs have at least 99.5% sequence identity when compared to the contigs for the microorganism provided in the sequence listing, as denoted in FIG. 12. In some embodiments, a genome identified in a metagenomic analysis is classified as corresponding to a microorganism listed in Table 1, Table 2, and/or FIGS. 13A-13XX if the identified genomic constructs have at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8%, at least 99.9%, or more sequence identity when compared to the contigs for the microorganism provided in the sequence listing, as denoted in FIG. 12.

Referring to block 214, in some embodiments, the plurality of gut microorganisms comprises at least 20 microorganisms selected from those microorganisms in Table 1, Table 2, or FIG. 13A-13XX having a connectivity of at least 2. In some embodiments, the set of identified gut microorganisms are selected from those microorganisms having a connectivity of at least 2, at least 3, at least 4, at least 5, at least 10, at least 15, at least 20, at least 25, or more.

Referring to block 216, in some embodiments, the biological sample from the gut of the respective subject is a fecal sample from the respective training subject. In some embodiments, said biological sample is a sample obtained from the small or large intestine, preferably colon or rectum, more preferably obtained in the form of a fecal sample or rectal swab or in the form of a biopsy specimen of gastrointestinal mucosa.

Referring to block 218, in some embodiments, the therapy is a biological therapy, an immunotherapy, a chemotherapy, a radiation therapy, a gene therapy, a hormone therapy, a photodynamic therapy, a targeted therapy, small molecules, antibodies, polynucleotide, natural compound, immune modulator, bone marrow therapy, stem cell therapy, surgery therapy, induction therapy, maintenance therapy, or a combination thereof.

Referring to block 220, in some embodiments, the disorder is selected from the group consisting of type-2 diabetes, hypertension, schizophrenia, atherosclerotic cardiovascular disease (ACVD), liver cirrhosis (LC), inflammatory bowel diseases (IBD), colorectal cancer (CRC), ankylosing spondylitis (AS), and Parkinson's disease (PD), inflammatory bowel disease (IBD, rheumatoid arthritis (RA), or advanced melanoma and B cell lymphoma. In some embodiments, the disorder is, e.g., hypertension (HT), schizophrenia (SCZ), Multiple Sclerosis (MS), Gaucher disease type II (GDII), COVID-19 (COV), Behcet's disease (BD), autism spectrum disorder (ASD), or pancreatic cancer (PC). In some embodiments, the disorder is cancer, Alzheimer diseases, a cardiovascular disease, an autoimmune disease, a mental health disease, an infectious disease, or a genetic disorder.

In some embodiments, the disorder is categorized by any indicator of a biological state, function, structure, process, response, or condition in a patient. Such indicators include any of the numerous variables (parameters) that are commonly measured in medicine to evaluate a patient for purposes such as diagnosis, prognosis, and/or treatment. Typically, indicators of interest herein are those whose values (which may be quantitative or qualitative) reflect, characterize, or are related to the function or structure of organs and organ systems and/or whose values reflect, characterize, or are related to the presence or severity of conditions. In some embodiments, the disease is categorized by the progression or prognosis of a disease or disorder, e.g., different stages of cancer, type, frequency, or degree of severity of the conditions that can be objectively measured or experienced by a subject. In certain embodiments, the disorder may be acquired by a medical device, which may be used to analyze the status of a body part, wound, or lesion, or of a physical substrate such as a test strip, dipstick, filter, or other substrate that provides means for detecting or measuring the presence of a substance in a biological sample obtained from a subject. In some embodiments, it is contemplated to detect antibodies against pathogens (e.g., viruses, bacteria, fungi), abnormal tissues (e.g., tumor site), or biomarkers in a biological sample and/or to detect the presence in a biological sample from a patient for purposes such as diagnosing the presence of a disorder or a disease.

Referring to block 222, in some embodiments, the disorder is cancer.

Referring to block 224, in some embodiments, the methods include inputting, for each respective training subject in the plurality of training subjects, information about the respective training subject into a model comprising a plurality of parameters. The model applies the plurality of parameters to the information, e.g., through at least 10,000 computations, to obtain a corresponding output for the respective training subject from the model. The corresponding output comprises a prediction of the respective training subject's response to the therapy, and the information about the respective training subject comprises the corresponding genomic abundance value for each respective gut microorganism in the plurality of gut microorganisms, and the plurality of gut microorganisms are selected from Table 1, Table 2, or FIG. 13A-13XX.

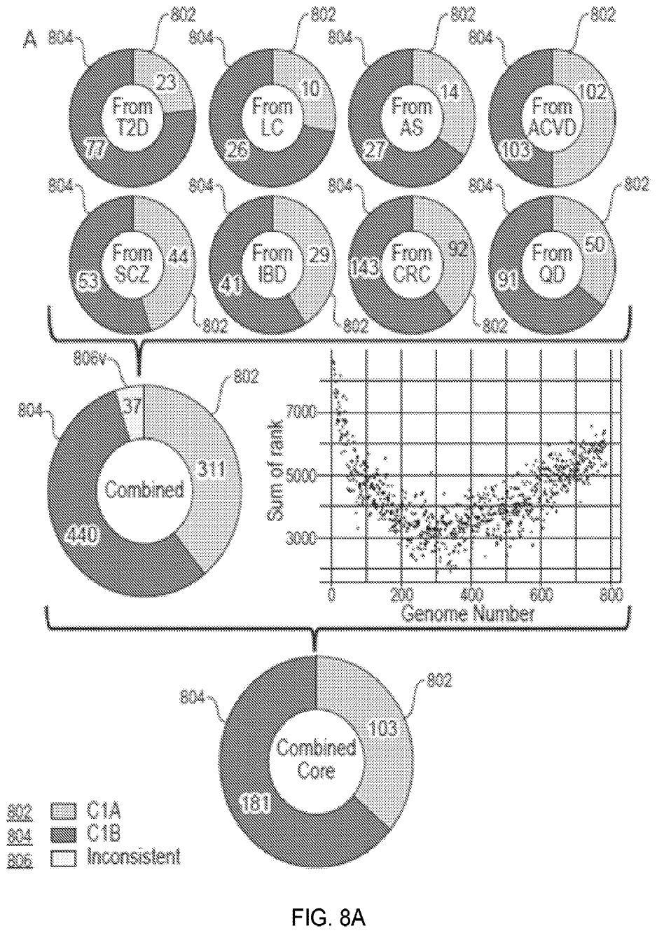

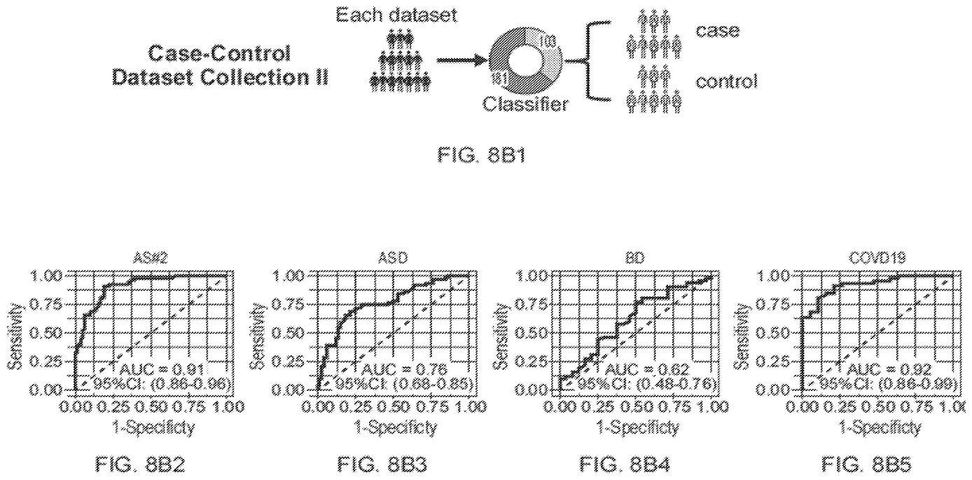

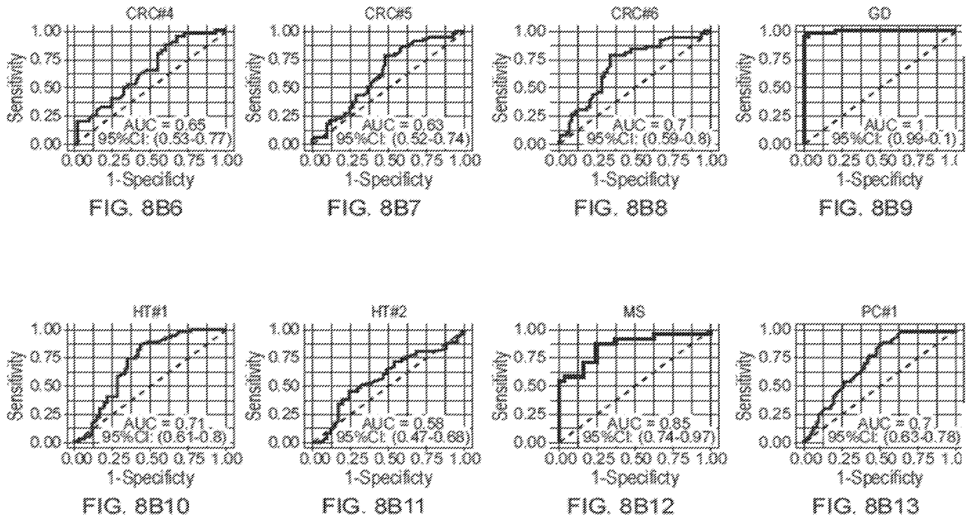

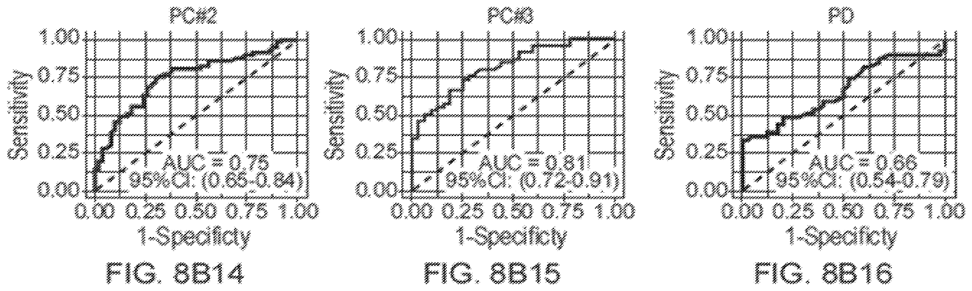

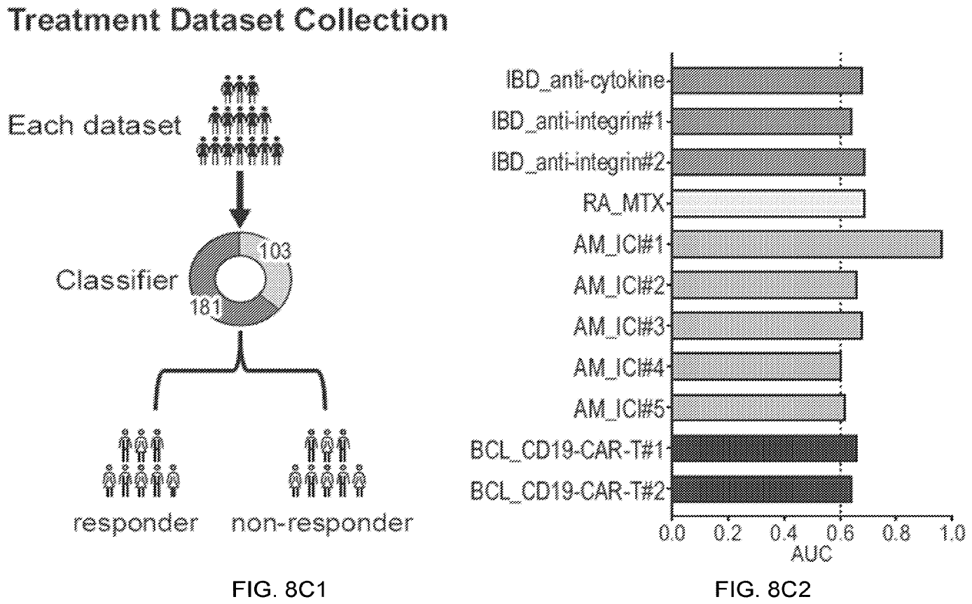

In some embodiments, the model is trained against datasets collected across a plurality of therapies to disorders and the model is trained to distinguish between a responsive state and a non-responsive state for the therapy. In some embodiments, the model comprises a learning statistical classifier system. In some embodiments, the learning statistical classifier system is random forest, classification and regression tree, boosted tree, or neural network. For example, as described in Example 3, a random forest classifier was trained against datasets from 11 different studies collectively looking at microbiomes in 4 different disorders. As shown in FIG. 8C, the resulting model was powered to predict responder or non-responder to anti-cytokine or anti-integrin therapy, methotrexate treatment in new-onset Rheumatoid Arthritis, immune checkpoint inhibitor (ICI) treatment on advanced melanoma, and CD19-CAR-T immunotherapy on B cell lymphoma.

Referring to block 226, in some embodiments, the prediction of the respective training subject's response is a class output of a respective response, in a plurality of possible responses, of the respective training subject. The method allows the setting of a single “cut-off” value permitting discrimination between responder or non-responder to a treatment. In some embodiments, the prediction of the respective training subject's response includes a prediction of an objective response rate of the human subject to the treatment or therapy, and wherein the prediction of the objective response rate includes an indication or classification of a complete response or an amount of a partial response to the treatment.

Referring to block 228, in some embodiments, the prediction of the respective training subject's response is a probability output for the respective training subject's response. As it is disclosed above, the method allows the setting of a single “cut-off” value permitting discrimination between responder or non-responder to a treatment. In some embodiments, the methods comprise utilizing the model to calculate a probability value for a subject; compare the probability value to a threshold value derived from a cohort of responders/non-responders to determine whether or not the probability value is above/below the threshold value; classify the subject as responder/non-responder if the probability value is above/below the threshold. In embodiments, the threshold value may be about a probability value of at least 50%, 55%, 50%, 65%, 70%, 75% or about 80% or more. In other embodiments, the probability value is a positive predictive value as measured by area under the curve (AUC) of receiver operating characteristic (ROC) curves. In certain embodiments, the probability value is calculated using a multivariate logistic regression model, a neural network model, a random forest model or a decision tree model.

Referring to block 230, in some embodiments, the model is a neural network algorithm, a support vector machine algorithm, a Naïve Bayes algorithm, a nearest neighbor algorithm, a boosted trees algorithm, a Random Forest algorithm, a convolutional neural network algorithm, a decision tree algorithm, a regression algorithm, or a clustering algorithm.

Referring to block 232, in some embodiments, the plurality of parameters is at least 1000, at least 10,000, at least 15,000, at least 50,000, at least 100,000, at least 250,000, at least 500,000, or at least 1,000,000 parameters, at least 2,500,000 parameters, at least 5,000,000 parameters, at least 10,000,000 parameters, or more.

Referring to block 234, in some embodiments, the model applies the plurality of parameters to the information through at least 1000 computation, at least 5000 computations, at least 10,000 computations, at least 25,000 computations, at least 50,000 computations, at least 100,000 computations, at least 250,000 computations, at least 500,000 computations, at least 1,000,000 computations, at least 2,500,000 computations, at least 5,000,000 computations, at least 10,000,000 computations, or more to obtain a corresponding output for the respective training subject from the model.

Referring to block 236, in some embodiments, the methods include adjusting the plurality of parameters based on, for each respective training subject in the plurality of training subjects, one or more differences between (i) the corresponding output from the model and (ii) the corresponding indication of the respective training subject's response to the therapy.

In some embodiments where deep learning techniques utilize a neural network as described above, the training of the neural network to improve the accuracy of its prediction involves modifying one or more parameters, including, but not limited to, weights in the filters in convolutional layers as well as biases in network layers. In some embodiments, the weights and biases are further constrained with various forms of regularization such as L1, L2, weight decay, and dropout.

For instance, in some embodiments, the neural network or any of the models disclosed herein optionally, where training data is labeled (e.g., with an indication of the state of the biological characteristic), have their parameters (e.g., weights) tuned (adjusted to potentially minimize the error between the system's predicted indications and the training data's measured indications). Various methods used to minimize error function, such as gradient descent methods, include, but are not limited to, log-loss, sum of squares error, hinge-loss methods. In some embodiments, these methods further include second-order methods or approximations such as momentum, Hessian-free estimation, Nesterov's accelerated gradient, adagrad, etc. In some embodiments, the methods also combine unlabeled generative pretraining and labeled discriminative training.

Accordingly, in some embodiments, the training of the neural network comprises adjusting one or more parameters in the plurality of parameters by back-propagation through a loss function. In some embodiments, the loss function is a regression task and/or a classification task. Non-limiting examples of loss functions suitable for the regression task include, but are not limited to, a mean squared error loss function, a mean absolute error loss function, a Huber loss function, a Log-Cosh loss function, or a quantile loss function. See, Wang et al., 2020, “A Comprehensive Survey of Loss Functions in Machine Learning,” Annals of Data Science, doi.org/10.1007/s40745-020-00253-5, last accessed Sep. 15, 2021, which is hereby incorporated by reference in its entirety. Non-limiting examples of loss functions suitable for the classification task include, but are not limited to, a binary cross entropy loss function, a hinge loss function, or a squared hinged loss function. In some embodiments, the loss function is any suitable regression task loss function or classification task loss function.

Other suitable methods for training the neural network that are contemplated for use in the present disclosure are further described herein (see, e.g., Definitions: Untrained model, above).

In some embodiments, the parameters of the neural network are randomly initialized prior to training.

In some embodiments, the neural network comprises a dropout regularization parameter. For example, in some embodiments, a regularization is performed by adding a penalty to the loss function, where the penalty is proportional to the values of the parameters in the trained or untrained model. Generally, regularization reduces the complexity of the model by adding a penalty to one or more parameters to decrease the importance of the respective hidden neurons associated with those parameters. Such practice can result in a more generalized model and reduce overfitting of the data. In some embodiments, the regularization includes an L1 or L2 penalty.

In some embodiments, the training the neural network comprises an optimizer. In some embodiments, the optimizer may employ the loss function to update the parameters of the neural network or other model via back-propagation. In some embodiments, the training the neural network comprises a learning rate.

In some embodiments, the learning rate is at least 0.0001, at least 0.0005, at least 0.001, at least 0.005, at least 0.01, at least 0.05, at least 0.1, at least 0.2, at least 0.3, at least 0.4, at least 0.5, at least 0.6, at least 0.7, at least 0.8, at least 0.9, or at least 1. In some embodiments, the learning rate is no more than 1, no more than 0.9, no more than 0.8, no more than 0.7, no more than 0.6, no more than 0.5, no more than 0.4, no more than 0.3, no more than 0.2, no more than 0.1 no more than 0.05, no more than 0.01, or less. In some embodiments, the learning rate is from 0.0001 to 0.01, from 0.001 to 0.5, from 0.001 to 0.01, from 0.005 to 0.8, or from 0.005 to 1. In some embodiments, the learning rate falls within another range starting no lower than 0.0001 and ending no higher than 1.

In some embodiments, the learning rate further comprises a learning rate decay (e.g., a reduction in the learning rate over one or more epochs). For example, a learning decay rate can be a reduction in the learning rate of 0.5 or 0.1. In some embodiments, the learning rate is a differential learning rate. In some embodiments, the training the neural network further uses a scheduler that conditionally applies the learning rate decay based on an evaluation of a performance metric over a threshold number of training epochs (e.g., the learning rate decay is applied when the performance metric fails to satisfy a threshold performance value for at least a threshold number of training epochs).

In some embodiments, the performance of the neural network is measured at one or more time points using a performance metric, including, but not limited to, a training loss metric, a validation loss metric, and/or a mean absolute error. In some embodiments, the performance metric is an area under receiving operating characteristic (AUROC) and/or an area under precision-recall curve (AUPRC).

For instance, in some embodiments, the performance of the neural network is measured by validating the model using a validation (e.g., development) dataset. In some such embodiments, the training the neural network forms a trained neural network when the neural network satisfies a minimum performance requirement based on a validation.

In some embodiments, any suitable method for validation can be used, including but not limited to K-fold cross-validation, advanced cross-validation, random cross-validation, grouped cross-validation (e.g., K-fold grouped cross-validation), bootstrap bias corrected cross-validation, random search, and/or Bayesian hyperparameter optimization.

In some embodiments, a method is provided for training a model comprising a plurality of parameters by a procedure comprising (i) inputting corresponding genomic abundance value for each respective gut microorganism in a plurality of gut microorganisms for each respective training subject in a plurality of training subjects, thereby obtaining as output from the model, for each respective training subject in the plurality of training subjects, a corresponding prediction of a training subject's response to a therapy, and (ii) refining the plurality of model parameters based on a differential between the corresponding actual response to a therapy of the training subject and the corresponding predicted response to a therapy of the training subject.

### 2. Methods of Applying a Model for Predicting a Subject's Response to a Therapy for a Disorder

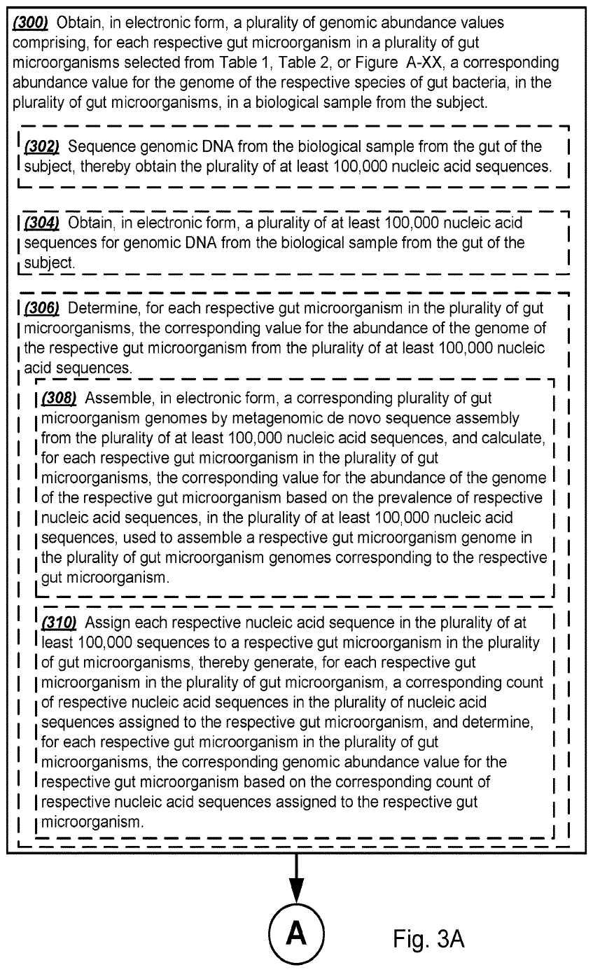

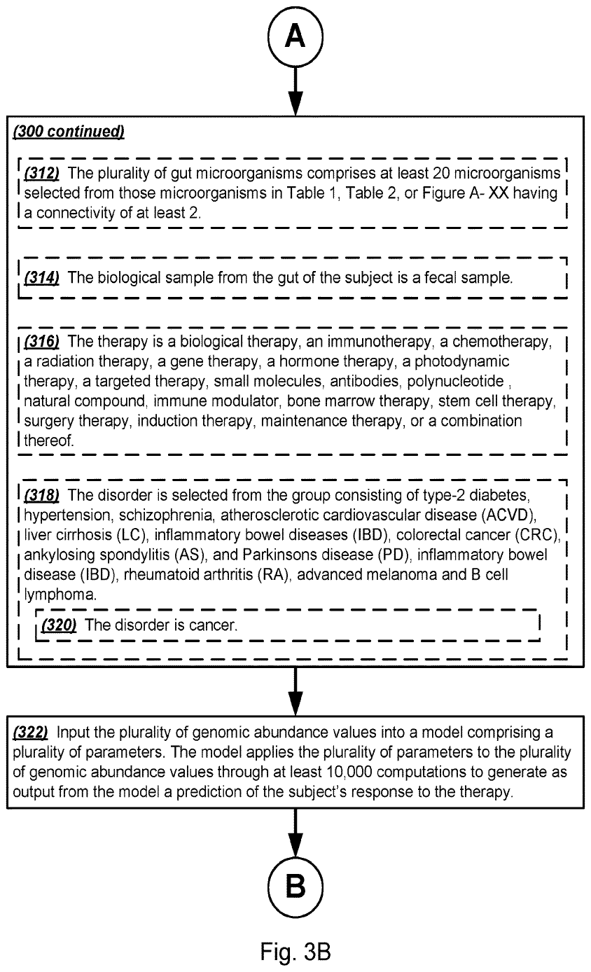

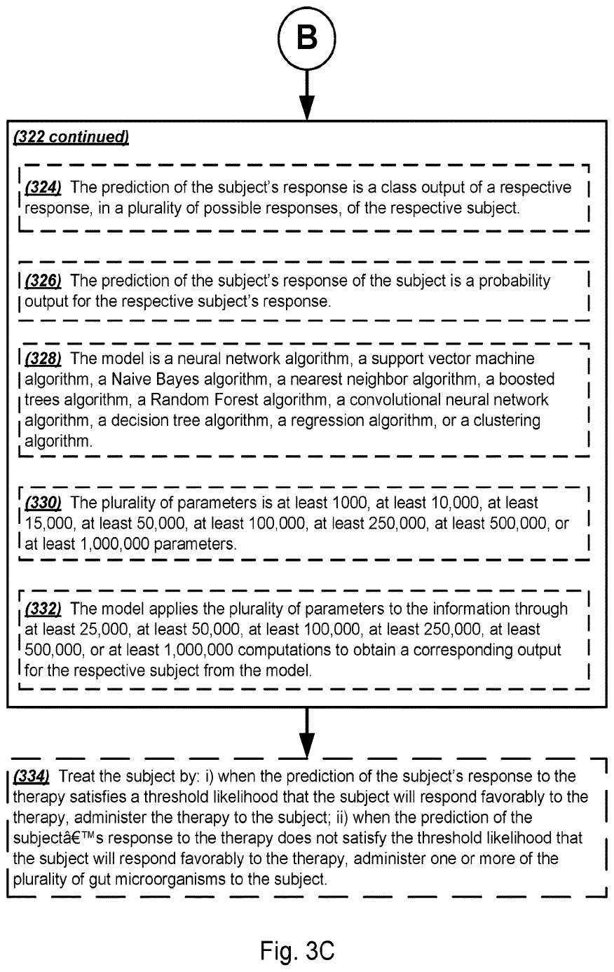

FIG. 3 is a schematic diagram of a method for applying a model for predicting a subject's response to a therapy for a disorder as discussed below. The method 300 may be implemented using a computer system (e.g., the computer system 100 shown and described above in reference to FIG. 1).

Referring to block 300, in some embodiments, the methods include obtaining, in electronic form, a plurality of genomic abundance values comprising, for each respective gut microorganism in a plurality of gut microorganisms selected from Table 1, Table 2, or FIG. 13A-13XX, a corresponding abundance value for the genome of the respective species of gut bacteria, in the plurality of gut microorganisms, in a biological sample from the subject.

In some embodiment, a corresponding biological sample from the gut of the respective subject was taken prior to a treatment or a therapy. In some embodiments, the biological sample is taken no more than 15 minutes, 30 minutes, 1 hour, 2 hours, 3 hours, 4 hours, 5 hours, 6 hours, 12 hours, or 24 hours prior to a treatment or a therapy. In some embodiments, the biological sample is taken 1 day, 2, days, 3 days, 4 days, 5 days, 6 days, 1 week, 2 weeks, 3 weeks, 4 weeks, or more prior to a treatment or a therapy. In some embodiments, the biological sample is taken about any of 1 month, 2 months, 3 months, 4 months, 5 months, 6 months, or more prior to a treatment or a therapy.

In some embodiments, sample data (including plasma, stool specimens) and corresponding clinical information (including gender/age/body fat count/underlying disease/histopathological characteristics, etc.) were collected for each subject prior to receiving a therapy. Individual biological samples were subjected to full microbiome analysis. In some embodiments, the sample is a tissue biopsy, an intestinal, or mucosal sample. See, for example, Tang Q, Jet al., Current Sampling Methods for Gut Microbiota: A Call for More Precise Devices, Front Cell Infect Microbiol., 10:151 (2020), the content of which is incorporated herein by reference in its entirety. In some embodiment, the biological sample from the gut of the respective subject is a fecal sample from the respective subject.

In some embodiments, the plurality of gut microorganisms comprises at least 25 gut microorganisms selected from Table 1, Table 2, or FIGS. 13A-13XX. In some embodiments, the plurality of gut microorganisms comprises at least 30 gut microorganisms selected from Table 1, Table 2, or FIGS. 13A-13XX. In some embodiments, the plurality of gut microorganisms comprises at least 40 gut microorganisms selected from Table 1, Table 2, or FIGS. 13A-13XX. In some embodiments, the plurality of gut microorganisms comprises at least 25 gut microorganisms selected from Table 1, Table 2, or FIGS. 13A-13XX. In some embodiments, the plurality of gut microorganisms comprises at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, at least 600, at least 700, at least 800, or all of the gut microorganisms selected from Table 1, Table 2 or FIGS. 13A-13XX. In some embodiments, the plurality of gut microorganisms are at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, at least 600, at least 700, at least 800, or all of the gut microorganisms selected from Table 1, Table 2 or FIGS. 13A-13XX. In some embodiments, the plurality of gut microorganisms are all of the gut microorganisms listed in Table 1. In some embodiments, the plurality of gut microorganisms are all of the gut microorganisms listed in Table 2. In some embodiments, the plurality of gut microorganisms are all of the gut microorganisms listed in FIGS. 13A-13XX.

In some of the embodiments, the corresponding value for the abundance of the genome is a value representative of the absolute abundance of a microorganism genome. In some of the embodiments, the corresponding value for the abundance of the genome is a value representative of a normalized abundance value, or a relative abundance value (e.g., an abundance of one microorganism normalized against the abundance of total microbiome of interest). In some of the embodiments, corresponding value for the abundance of the genome is a value representative of an averaged abundance value (e.g., average of abundances obtained at different time points or from different biological samples from the patients, or average of abundances obtained using different probes, etc.), or a combination of any of above. The corresponding value for the abundance of the genome is measured by any technique known in the art. In some embodiments, the genomic abundance value for the genome is measured by quantitative PCR (qPCR), such as bacterial 16S rRNA qPCR, RT-PCR, or qRT-PCR, for quantifying the abundance of region of interests in the genome, e.g., as described in U.S. Pat. No. 11,427,865, the disclosure of which is hereby incorporated by reference in its entirety. In some embodiments, the genomic abundance value is measured by targeted sequencing (e.g., 16S rRNA sequencing, or any other suitable biomarker), partial genome sequencing or whole genome sequencing, thereby quantifying the number of reads of the targeted regions in a microorganism genome to determine the abundance of the genome, e.g., as disclosed in U.S. Patent Application Publication No. 2021/0403986 or U.S. Pat. No. 11,332,783, the disclosures of which are hereby incorporated by reference in their entireties. In some embodiments, deep sequencing is employed to determine the abundance of targeted sequences, e.g., as disclosed in U.S. Patent Application Publication No. 2018/0237863, the disclosure of which is incorporated herein by reference in its entirety. In some embodiments, the sequencing depth is at least 2×, at least 3×, at least 4×, at least 5×, at least 6×, at least 7×, at least 8×, at least 9×, at least 10×, at least 11×, at least 12×, at least 13×, at least 14×, at least 15×, at least 16×, at least 17×, at least 18×, at least 19×, at least 20×, at least 21×, at least 22×, at least 23×, at least 24×, at least 25×, at least 26×, at least 27×, at least 28×, at least 29×, at least 30×, at least 31×, at least 32×, at least 33×, at least 34×, at least 35×, at least 36×, at least 37×, at least 38×, at least 39×, at least 40×, at least 41×, at least 42×, at least 43×, at least 44×, at least 45×, at least 46×, at least 47×, at least 48×, at least 49×, at least 50×, at least 51×, at least 52×, at least 53×, at least 54×, at least 55×, at least 56×, at least 57×, at least 58×, at least 59×, at least 60×, at least 70×, at least 80×, at least 90×, at least 100×, at least 110×, at least 120×, at least 130×, at least 150×, at least 200×, at least 300×, at least 400×, at least 500×, at least 750×, at least 1000×, or more. In some embodiments, shotgun metagenomic sequencing is employed to provide sequence reads for genomes in a sample, e.g., as described in U.S. Pat. No. 11,028,449, the content of which is incorporated herein by reference in its entirety.

In some embodiments of the methods described herein, a genome identified in a metagenomic analysis is classified as corresponding to a microorganism listed in Table 1, Table 2, and/or FIGS. 13A-13XX if the identified genomic constructs have at least 97% sequence identity when compared to the contigs for the microorganism provided in the sequence listing, as denoted in FIG. 12. In some embodiments, a genome identified in a metagenomic analysis is classified as corresponding to a microorganism listed in Table 1, Table 2, and/or FIGS. 13A-13XX if the identified genomic constructs have at least 98% sequence identity when compared to the contigs for the microorganism provided in the sequence listing, as denoted in FIG. 12. In some embodiments, a genome identified in a metagenomic analysis is classified as corresponding to a microorganism listed in Table 1, Table 2, and/or FIGS. 13A-13XX if the identified genomic constructs have at least 99% sequence identity when compared to the contigs for the microorganism provided in the sequence listing, as denoted in FIG. 12. In some embodiments, a genome identified in a metagenomic analysis is classified as corresponding to a microorganism listed in Table 1, Table 2, and/or Figures AXX if the identified genomic constructs have at least 99.5% sequence identity when compared to the contigs for the microorganism provided in the sequence listing, as denoted in FIG. 12. In some embodiments, a genome identified in a metagenomic analysis is classified as corresponding to a microorganism listed in Table 1, Table 2, and/or FIGS. 13A-13XX if the identified genomic constructs have at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8%, at least 99.9%, or more sequence identity when compared to the contigs for the microorganism provided in the sequence listing, as denoted in FIG. 12.

Referring to block 302, in some embodiments, the methods include sequencing genomic DNA from the biological sample from the gut of the subject, thereby obtaining the plurality of nucleic acid sequences. In some embodiments, the plurality of nucleic acid sequences comprises at least 10,000, at least 100,000, at least 250,000, at least 500,000, at least 1,000,000, at least 2,500,000, at least 5,000,000, at least 10,000,000 or at least 50,000,000 nucleic acid sequences. In some embodiments, the plurality of nucleic acid sequences comprises no more than 250,000,000, no more than 100,000,000, no more than 50,000,000, no more than 25,000,000, no more than 10,000,000, no more than 5,000,000, no more than 1,000,000, no more than 100,000 nucleic acid sequences. In some embodiments, the plurality of nucleic acid sequences consists of from 100,000 to 1,000,000, from 200,000 to 5,000,000, from 500,000 from 10,000,000, from 1,000,000 to 20,000,000, from 5,000,000 to 50,000,000, from 10,000,000 to 100,000,000, or from 50,000,000 to 250,000,000 nucleic acid sequences. In some embodiments, the plurality of nucleic acid sequences falls within another range starting no lower than 1000 nucleic acid sequences and ending no higher than 250,000,000 nucleic acid sequences.

In some embodiments, the corresponding plurality of nucleic acid sequences are obtained through metagenomic sequencing, e.g., as disclosed in U.S. Patent Application Publication No. 2016/0239602 or U.S. Pat. No. 11,495,326, the contents of which are incorporated herein by reference in their entireties. In some embodiments, metagenomes sequencing further comprise generating the plurality of metagenomic fragment reads. In some embodiments, metagenomic sequencing further comprise fragmenting microbial genomes into random fragments of targeted sizes. The resulting fragments can vary in size. In one embodiment, fragments of approximately 500 nucleotides can be obtained. In some embodiments, fragments of from 100-2000 nucleotides, e.g., 200-800, 100-900, 100-1000, 300-800, 400-900 nucleotides can be obtained. In some embodiments, the method may further comprise extracting the metagenomic fragments from the corresponding biological sample. In some embodiments, metagenomes sequencing further comprise sequencing the fragments using high throughput sequencing methods to generate a plurality of sequencing reads.

In some embodiments, the corresponding plurality of nucleic acid sequences are obtained through targeted panel sequencing. An example of targeted panel sequencing is described in U.S. Patent Application Publication No. 2019/0316209. In some embodiments, the targeted panel sequencing comprises hybridizing genomic DNA isolated from a biological sample from the gut of a subject with a panel of probes that include one or more probes that hybridize to a unique sequence in the genome of each microorganism being quantified, prior to sequencing recovered nucleic acids. In some embodiments, the microorganisms include a plurality of the microorganisms listed in Table 1, Table 2, and/or FIGS. 13A-13XX. In some embodiments, a combination of semi-unique sequences (e.g., sequences found in a small number of the microorganism genomes) can be used to deconvolute genomic abundance values using an algorithm, e.g., a system of equations. In some embodiments, the panel of probes includes at least 1 probe that hybridizes to a sequence unique to each microorganism genome being detected. In some embodiments, the panel of probes includes at least 2, at least 3, at least 4, at least 5, at least 10, at least 25, at least 50, or more probes that hybridize to a different sequence unique to each microorganism genome being detected. In some embodiments, the panel of probes includes at least 20, at least 30, at least 40, at least 50, at least 75, at least 100, at least 125, at least 150, at least 200, at least 150, at least 300, at least 400, at least 500, at least 750, at least 1000, at least 1250, at least 1500, at least 2000, at least 2500, at least 3000, at least 4000, at least 5000, at least 7500, at least 10,000 or more unique probes.

In some embodiments, the sequencing genomic DNA from the corresponding biological sample comprise a partial or complete sequencing platform adapter sequence at their termini useful for sequencing using a sequencing platform of interest. Sequencing platforms of interest include, but are not limited to, the HiSeq™, MiSeq™ and Genome Analyzer™ sequencing systems from Illumina®; the Ion PGM™ and Ion Proton™ sequencing systems from Ion Torrent™; the PACBIO RS II Sequel system from Pacific Biosciences, the SOLID sequencing systems from Life Technologies™, the 454 GS FLX+ and GS Junior sequencing systems from Roche, the MinION™ system from Oxford Nanopore, or any other sequencing platform of interest.

Referring to block 304, in some embodiments, the methods include obtaining, in electronic form, a plurality of nucleic acid sequences for genomic DNA from the biological sample from the gut of the subject.

Referring to block 306, in some embodiments, the methods include determining, for each respective gut microorganism in the plurality of gut microorganisms, the corresponding value for the abundance of the genome of the respective gut microorganism from the plurality of nucleic acid sequences. In some embodiments, the genomic abundance values determined for the subject comprise at least 20, at least 25, at least 50, at least 75, at least 100, at least 125, at least 150, at least 175, at least 200, at least 250, at least 300, at least 350, at least 400, at least 500, at least 600, at least 700, at least 700, at least 800, at least 900, at least 1000, at least 1500, at least 2000, at least 25000, at least 5,000 or at least 10,000 genome abundance values, where each genome abundance value corresponds to different gut microorganism. In some embodiments, the genomic abundance values comprise no more than 250,000, no more than 100,000, no more than 50,000, no more than 25,000, no more than 10,000, no more than 5000, no more than 2500, no more than 1000, no more than 750, no more than 500, or fewer genome abundance values. In some embodiments, the genomic abundance values consist of from 10 to 40, from 20 to 50, from 30 to 80, from 40 to 100, from 50 to 150, from 60 to 200, from 80 to 300, from 90 to 500, from 100 to 1000, from 500 to 2,000, or from 1,000 to 5,000 genome abundance values. In some embodiments, the number of genomic abundance values fall within another range starting no lower than 10 genome abundance values and ending no higher than 250,000 genome abundance values.

Referring to block 308, in some embodiments, the methods include assembling, in electronic form, a corresponding plurality of gut microorganism genomes by metagenomic de novo sequence assembly from the plurality of nucleic acid sequences, and calculating, for each respective gut microorganism in the plurality of gut microorganisms, the corresponding value for the abundance of the genome of the respective gut microorganism based on the prevalence of respective nucleic acid sequences, in the plurality of nucleic acid sequences, used to assemble a respective gut microorganism genome in the plurality of gut microorganism genomes corresponding to the respective gut microorganism. In some embodiments, metagenomic de novo sequence assembly further comprise generating contigs based on the sequencing reads generated by a shotgun sequencing technique. Such a technique is described, for example, in U.S. Pat. No. 10,529,443, the content of which is incorporated herein by reference in its entirety. In some embodiments, the plurality of nucleic acid sequences can be assembled into full genomes of the plurality of gut microorganisms. In some embodiments, the plurality of nucleic acid sequences can be assembled into partial genomes of the plurality of gut microorganisms.

Referring to block 310, in some embodiments, the methods include assigning each respective nucleic acid sequence in the plurality of nucleic acid sequences to a respective gut microorganism in the plurality of gut microorganisms, thereby generating, for each respective gut microorganism in the plurality of gut microorganism, a corresponding count of respective nucleic acid sequences in the plurality of nucleic acid sequences assigned to the respective gut microorganism, and determining, for each respective gut microorganism in the plurality of gut microorganisms, the corresponding genomic abundance value for the respective gut microorganism based on the corresponding count of respective nucleic acid sequences assigned to the respective gut microorganism. In some embodiments, the assigning each respective nucleic acids to a respective gut microorganism includes mapping the nucleic acid to a reference nucleic acid. In some embodiments, the assigning each respective nucleic acids a respective gut microorganism includes annotating genome information based on existing databases. In some embodiments, nucleic acid sequences are analyzed, and annotations are to define taxonomic assignments using sequence similarity and phylogenetic placement methods or a combination of the two strategies.

Sequence similarity based methods for assigning each respective nucleic acid sequence in a respective gut microorganism include those familiar to individuals skilled in the art including, but not limited to BLAST, BLASTx, tBLASTn, tBLASTx, RDP-classifier, DNAclust, and various implementations of these algorithms such as Qiime or Mothur. These methods rely on mapping a sequence read to a reference database and selecting the match with the best score and e-value. In some embodiments, phylogenetic methods are used in combination with sequence similarity methods to improve the calling accuracy of an annotation or taxonomic assignment. Common databases include, but not limited to, GT-DBTK, National Center for Biotechnology Information (NCBI) Genbank, European Bioinformatics Institute-European Nucleotide Archive (European Bioinformatics Institute-European Nucleotide Archive; EBI-ENA), National Institute of Genetics, U.S. Department of ENERGY (USDOE) Integrated Microbial Genomes (Integrated Microbial Genomes) &Microbiomes; IMG/M) and other available databases in the art.

Referring to block 312, in some embodiments, the plurality of gut microorganisms comprises at least 20 microorganisms selected from those microorganisms in Table 1, Table 2, or FIG. 13A-13XX having a connectivity of at least 2, at least 3, at least 4, at least 5, at least 10, at least 15, at least 20, at least 25, or more. In some embodiments, the plurality of gut microorganisms comprises at least 20 microorganisms selected from those microorganisms listed in Table 1, Table 2, or FIG. 13A-13XX as having a connectivity of at least 2. In some embodiments, the plurality of gut microorganisms comprises at least 20 microorganisms selected from those microorganisms listed in Table 1, Table 2, or FIG. 13A-13XX as having a connectivity of at least 3, at least 4, at least 5, at least 10, at least 15, at least 20, at least 25, or more.

Referring to block 314, in some embodiments, the biological sample from the gut of the subject is a fecal sample. In some embodiments, the sample is a tissue biopsy, an intestinal, or mucosal sample. In some embodiments, said biological sample is a sample obtained from the small or large intestine, preferably colon or rectum, more preferably obtained in the form of a fecal sample or rectal swab or in the form of a biopsy specimen of gastrointestinal mucosa.

Referring to block 316, in some embodiments, the therapy is a biological therapy, an immunotherapy, a chemotherapy, a radiation therapy, a gene therapy, a hormone therapy, a photodynamic therapy, a targeted therapy, small molecules, antibodies, polynucleotide, natural compound, immune modulator, bone marrow therapy, stem cell therapy, surgery therapy, induction therapy, maintenance therapy, or a combination thereof.

Referring to block 318, in some embodiments, the disorder is selected from the group consisting of type-2 diabetes, hypertension, schizophrenia, atherosclerotic cardiovascular disease (ACVD), liver cirrhosis (LC), inflammatory bowel diseases (IBD), colorectal cancer (CRC), ankylosing spondylitis (AS), and Parkinson's disease (PD), inflammatory bowel disease (IBD), rheumatoid arthritis (RA), advanced melanoma and B cell lymphoma. In some embodiments, the disorder is, e.g., hypertension (HT), schizophrenia (SCZ), Multiple Sclerosis (MS), Gaucher disease type II (GDII), COVID-19 (COV), Behcet's disease (BD), autism spectrum disorder (ASD), or pancreatic cancer (PC). In some embodiments, the disorder is cancer, Alzheimer diseases, a cardiovascular disease, an autoimmune disease, a mental health disease, an infectious disease, or a genetic disorder.

In some embodiments, the disorder is categorized by any indicator of a biological state, function, structure, process, response, or condition in a patient. Such indicators include any of the numerous variables (parameters) that are commonly measured in medicine to evaluate a patient for purposes such as diagnosis, prognosis, and/or treatment. Typically, indicators of interest herein are those whose values (which may be quantitative or qualitative) reflect, characterize, or are related to the function or structure of organs and organ systems and/or whose values reflect, characterize, or are related to the presence or severity of conditions. In some embodiments, the disease is categorized by the progression or prognosis of a disease or disorder, e.g., different stages of cancer, type, frequency, or degree of severity of the conditions that can be objectively measured or experienced by a subject. In certain embodiments, the disorder may be acquired by a medical device, which may be used to analyze the status of a body part, wound, or lesion, or of a physical substrate such as a test strip, dipstick, filter, or other substrate that provides means for detecting or measuring the presence of a substance in a biological sample obtained from a subject. In some embodiments, it is contemplated to detect antibodies against pathogens (e.g., viruses, bacteria, fungi), abnormal tissues (e.g., tumor site), or biomarkers in a biological sample and/or to detect the presence in a biological sample from a patient for purposes such as diagnosing the presence of a disorder or a disease.

Referring to block 320, in some embodiments, the disorder is cancer.

Referring to block 322, in some embodiments, the methods include inputting the plurality of genomic abundance values into a model comprising a plurality of parameters. The model applies the plurality of parameters to the plurality of genomic abundance values through, e.g., at least 10,000 computations, to generate as output from the model a prediction of the subject's response to the therapy.

In some embodiments, the model is trained against datasets collected across a plurality of therapies to disorders and the model is trained to distinguish between a responsive state and a non-responsive state for the therapy. In some embodiments, the model comprises a learning statistical classifier system. In some embodiments, the learning statistical classifier system is random forest classification and regression tree, boosted tree, neural network. For example, as described in Example 3, a random forest classifier was trained against datasets from 11 different studies collectively looking at microbiomes in 4 different disorders. As shown in FIG. 8C, the resulting model was powered to predict responder or non-responder to anti-cytokine or anti-integrin therapy, methotrexate treatment in new-onset Rheumatoid Arthritis, immune checkpoint inhibitor (ICI) treatment on advanced melanoma, and CD19-CAR-T immunotherapy on B cell lymphoma.

In some embodiments, the indication of subject's response is characterized by clinical outcome measures include, but are not limited to, complete remission, partial remission, non-remission, survival, development of adverse events, or any combination thereof. In some embodiments, one responder has complete remission in response to the treatment, and the non-responders has non-remission or partial remission in response to the treatment. In some embodiment, patients were subjected to routine clinical examinations, laboratory analyses, and computed tomography. Tumor responses were evaluated using RECIST criteria. In some embodiment, complete response was defined as complete radiographic disappearance of measurable or evaluable disease or stable, minimal radiographic findings; partial response was defined as reduction of the longest dimension of measurable disease by at least 50%; stable disease was defined as reduction of the longest dimension by less than 25%; Progressive disease was defined as growth of the tumor by more than 25% in the longest dimension or development of new lesions. In some embodiment, overall response rate was defined as the sum of the complete and partial response rates and the tumor control rate was defined as the sum of overall response rates with stable disease rates.

In some embodiments, the indication of subject's response is characterized by the actual treatment efficacy of an therapy, including progression-free survival (PFS), the duration of the progression free survival under treatment, total Survival (OS), response to therapy (RT), overall response rate (ORR), sustained clinical effect (DCB), Disease Activity Score, or any combination thereof, or any other methods for evaluating the progression or prognosis of a disease or disorder known in the art.

In some embodiments, “progression free survival” (PFS) has its art-understood meaning relating to the length of time during and after the treatment of a disease, such as cancer, that a patient lives with the disease but it does not get worse. In some embodiments, measuring the progression-free survival is utilized as an assessment of how well a new treatment works. In some embodiments, PFS is determined in a randomized clinical trial; in some such embodiments, PFS refers to time from randomization until objective tumor progression and/or death.

In some embodiments, ORR may be defined as the proportion of patients in whom partial (PR) or complete (CR) responses are identified as a best overall response (BOR) according to some metric, such as Response Evaluation Criteria in Solid Tumors (RECIST 1.1). Stable disease (SD) was categorized as non-response together with progressive disease (PD). In some embodiments, ORR has its art-understood meaning referring to the proportion of patients with tumor size reduction of a predefined amount and for a minimum time period. In some embodiments, response duration usually measured from the time of initial response until documented tumor progression. In some embodiments, ORR involves the sum of partial responses plus complete responses.

In some embodiments, “clinical effect” refers to a clinical benefit. In some embodiments, such a clinical benefit is or comprises reduction in tumor size, increase in progression free survival, increase in overall survival, decrease in overall tumor burden, decrease in the symptoms caused by tumor growth such as pain, organ failure, bleeding, damage to the skeletal system, and other related sequelae of metastatic cancer and combinations thereof. In some embodiments, the clinical effect is a “sustained clinical effect” (DCB) that is maintained for a relevant period of time. In some embodiments, the relevant period of time is at least 1 month, 2 months, 3 months, 4 months, 5 months, 6 months, 7 months, 8 months, 9 months, 10 months, 11 months, 1 year, 2 years, 3 years, 4 years, 5 years, or longer.

In some embodiments, the subject's response is measured by Disease Activity Score (DAS) (see, e.g., Van der Heijde D. M. et al., J Rheumatol, 1993, 20(3):579-81; Prevoo M. L. et al, Arthritis Rheum, 1995, 38:44-8). The DAS system represents both current state of disease activity and change. The DAS scoring system uses a weighted mathematical formula, derived from clinical trials in RA. For example, the DAS 28 is 0.56 (T28)+0.28 (SW28)+0.70 (Ln ESR)+0.014 GH wherein T represents tender joint number, SW is swollen joint number, ESR is erythrocyte sedimentation rate, and GH is global health. Various values of the DAS represent high or low disease activity as well as remission, and the change and endpoint score result in a categorization of the patient by degree of response (none, moderate, good).

In some embodiments, the indication of the subject's response is measured by the level of the immune response or immune parameters of a cancer-bearing patient resulting from an immunotherapy. In some embodiments, the immune response or immune parameters are characterized by expression level of various biological markers of the host immune response in conjunction with the occurrence of a cancer at a given stage of cancer development (i.e. treatment efficacy). In some embodiments, the expression level of a biological marker is compared with a reference value for the same biological marker, and when required with reference values. The reference value for the same biological marker is thus predetermined and is already known to be indicative of a reference value that is pertinent for discriminating between a low level and a high level of the immune response of a patient with cancer, for said biological marker. Said predetermined reference value for said biological marker is correlated with a responder to treatment in a cancer patient, or conversely is correlated with non-responder to treatment in a cancer patient.

In some embodiments, a change of a combination of biological markers are quantified. In some embodiments, a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more distinct biological markers are quantified.

In certain embodiments, biological markers are quantified with immunohistochemical techniques. Example biological markers include 18s, ACE, ACTB, AGTR1, AGTR2, APC, APOA1, ARF1, AX1N1, BAX, BCL2, BCL2L1, CXCR5, BMP2, BRCA1, BTLA, C3, CASP3, CASp9, CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCL5, CCL7, CCL8, CCNB1, CCND1, CCNE1, CCR1, CCR10, CCR2, CCR3, CCR4, CCR5, CCR6, CCR7, CCR8, CCR9, CCRL2, CD154, CD19, CD1a, CD2, CD226, CD244, PDCD1LG1, CD28, CD34, CD36, CD38, CD3E, CD3G, CD3Z, CD4, CD40LG, CD5, CD54, CD6, CD68, CD69, CLIP, CD80, CD83, SLAMF5, CD86, CD8A, CDH1, CDH7, CDK2, CDK4, CDKN1A, CDKN1B, CDKN2A, CDKN2B, CEACAM1, COL4A5, CREBBP, CRLF2, CSF1, CSF2, CSF3, CTLA4, CTNN81, CTSC, CX3CL1, CX3CR1, CXCL1, CXCL10, CXCL11, CXCL12, CXCL13, CXCL14, CXCL16, CXCL2, CXCL3, CXCL5, CXCL6, CXCL9, CXCR3, CXCR4, CXCR6, CYP1A2, CYP7A1, DCC, DCN, DEFA6, DICER1, DKK1, Dok-1, Dok-2, DOK6, DVL1, E2F4, EBI3, ECE1, ECGF1, EDN1, EGF, EGFR, EIF4E, CD105, ENPEP, ERBB2, EREG, FCGR3A, CGR3B, FN1, FOXP3, FYN, FZD1, GAPD, GLI2, GNLY, GOLPH4, GRB2, GSK3B, GSTP1, GUSB, GZMA, GZMH, GZMK, HLA-B, HLA-C, HLA-, MA, HLA-DMB, HLA-DOA, HLA-DOB, HLA-DPA1, HLA-DQA2, HLA-DRA, HLX1, HMOX1, HRAS, HSPB3, HUWE1, ICAM1, ICAM-2, ICOS, ID1, ifna1, ifna17, ifna2, ifna5, ifna6, ifna8, IFNAR1, IFNAR2, IFNG, IFNGR1, IFNGR2, IGF1, IHH, IKBKB, IL10, IL12A, IL12B, IL12RB1, IL12RB2, IL13, IL13RA2, IL15, IL15RA, IL17, IL17R, IL17RB, IL18, IL1A, IL1B, IL1R1, IL2, IL21, IL21R, IL23A, IL23R, IL24, IL27, IL2RA, IL2RB, IL2RG, IL3, IL31RA, IL4, IL4RA, IL5, IL6, IL7, IL7RA, IL8, CXCR1, CXCR2, IL9, IL9R, IRF1, ISGF3G, ITGA4, ITGA7, integrin, alpha E (antigen CD103, human mucosal lymphocyte, antigen 1; alpha polypeptide), Gene hCG33203, ITGB3, JAK2, JAK3, KLRB1, KLRC4, KLRF1, KLRG1, KRAS, LAG3, LAIR2, LEF1, LGALS9, LILRB3, LRP2, LTA, SLAMF3, MADCAM1, MADH3, MADH7, MAF, MAP2K1, MDM2, MICA, MICB, MKI67, MMP12, MMP9, MTA1, MTSS1, MYC, MYD88, MYH6, NCAM1, NFATC1, NKG7, NLK, NOS2A, P2X7, PDCD1, PECAM-, CXCL4, PGK1, PIAS1, PIAS2, PIAS3, PIAS4, PLAT, PML, PP1A, CXCL7, PPP2CA, PRF1, PROM1, PSMB5, PTCH, PTGS2, PTP4A3, PTPN6, PTPRC, RAB23, RAC/RHO, RAC2, RAF, RB1, RBL1, REN, Drosha, SELE, SELL, SELP, SERPINE1, SFRP1, SIRP beta 1, SKI, SLAMF1, SLAMF6, SLAMF7, SLAMF8, SMAD2, SMAD4, SMO, SMOH, SMURF1, SOCS1, SOCS2, SOCS3, SOCS4, SOCS5, SOCS6, SOCS7, SOD1, SOD2, SOD3, SOS1, SOX17, CD43, ST14, STAM, STAT1, STAT2, STAT3, STAT4, STAT5A, STAT5B, STAT6, STK36, TAP1, TAP2, TBX21, TCF7, TERT, TFRC, TGFA, TGFB1, TGFBR1, TGFBR2, TIMP3, TLR1, TLRO1, TLR2, TLR3, TLR4, TLR5, TLR6, TLR7, TLR8, TLR9, TNF, TNFRSF10A, TNFRSF11A, TNFRSF18, TNFRSF1A, TNFRSF1B, OX-40, TNFRSF5, TNFRSF6, TNFRSF7, TNFRSF8, TNFRSF9, TNFSF10, TNFSF6, TOB1, TP53, TSLP, VCAM1, VEGF, WIF1, WNT1, WNT4, XCL1, XCR1, ZAP70 and ZIC2.

Referring to block 324, in some embodiments, the prediction of the subject's response is a class output of a respective response, in a plurality of possible responses, of the respective subject. The method allows the setting of a single “cut-off” value permitting discrimination between responder or non-responder to a treatment. In some embodiments, the prediction of the respective subject's response includes a prediction of an objective response rate of the human subject to the treatment or therapy, and wherein the prediction of the objective response rate includes an indication or classification of a complete response or an amount of a partial response to the treatment.

Referring to block 326, in some embodiments, the prediction of the subject's response of the subject is a probability output for the respective subject's response. As it is disclosed above, the method allows the setting of a single “cut-off” value permitting discrimination between responder or non-responder to a treatment. In some embodiments, the methods comprise utilizing the model to calculate a probability value for a subject; compare the probability value to a threshold value derived from a cohort of responders/non-responders to determine whether or not the probability value is above or below the threshold value; classify the subject as responder/non-responder if the probability value is above or below the threshold. In embodiments, the threshold value may be about a probability value of at least 50%, 55%, 50%, 65%, 70%, 75% or about 80% or more. In other embodiments, the probability value is a positive predictive value as measured by area under the curve (AUC) of receiver operating characteristic (ROC) curves. In certain embodiments, the probability value is calculated using a multivariate logistic regression model, a neural network model, a random forest model or a decision tree model.

Referring to block 328, in some embodiments, the model is a neural network algorithm, a support vector machine algorithm, a Naïve Bayes algorithm, a nearest neighbor algorithm, a boosted trees algorithm, a Random Forest algorithm, a convolutional neural network algorithm, a decision tree algorithm, a regression algorithm, or a clustering algorithm.

Referring to block 330, in some embodiments, the plurality of parameters is at least 1000, at least 10,000, at least 15,000, at least 50,000, at least 100,000, at least 250,000, at least 500,000, at least 1,000,000 parameters, at least 2,500,000 parameters, at least 5,000,000 parameters, at least 10,000,000 parameters, or more.

Referring to block 332, in some embodiments, the model applies the plurality of parameters to the information through at least 1000 computation, at least 5000 computations, at least 10,000 computations, at least 25,000 computations, at least 50,000 computations, at least 100,000 computations, at least 250,000 computations, at least 500,000 computations, at least 1,000,000 computations, at least 2,500,000 computations, at least 5,000,000 computations, at least 10,000,000 computations, or more to obtain a corresponding output for the respective subject from the model.

Referring to block 334, in some embodiments, the method further comprises treating the subject by: i) when the prediction of the subject's response to the therapy satisfies a threshold likelihood that the subject will respond favorably to the therapy, administering the therapy to the subject; ii) when the prediction of the subject's response to the therapy does not satisfy the threshold likelihood that the subject will respond favorably to the therapy, administering one or more of the plurality of gut microorganisms to the subject.

In some embodiments, the administering comprises identifying one or more of the plurality of gut microorganisms that is underrepresented in the subject, e.g., as determined based on the corresponding genomic abundance value for the microorganism, and administering the identified one or more gut microorganism to the subject. In some embodiments, the identifying includes determining whether the abundance of a gut microorganism, e.g., as determined based on the corresponding genomic abundance value for the microorganism, satisfies a corresponding threshold amount. When the abundance of the microorganism does not satisfy the corresponding threshold amount, identifying that microorganism for administration. In some embodiments, the corresponding threshold amount is a relative abundance. In some embodiments, the corresponding threshold amount is an amount relative to the abundance of one or more different gut microorganisms in the subject. In some embodiments, the corresponding threshold amount is an amount relative to the total abundance of the plurality of gut microorganisms in the subject.

In some embodiments, the administering comprises administering a pre-defined set of microorganisms. In some embodiments, the predefined set of microorganisms includes at least 5 gut microorganisms selected from Table 1, Table 2, or FIGS. 13A-13XX. In some embodiments, the predefined set of microorganisms includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 175, 180, 190, 200, 225, 250, 275, 300, 350, 400, 450, 500, 600, 700, or more gut microorganisms selected from Table 1, Table 2, or FIGS. 13A-13XX.

In some embodiments, the predefined set of microorganisms only includes gut microorganisms selected from Table 1, Table 2, or FIGS. 13A-13XX. that are assigned to Guild 1. That is, the predefined set of microoganisms does not include microorganisms selected from Table 1, Table 2, or FIGS. 13A-13XX. that are assigned to Guild 2. In some embodiments, the predefined set of microorganisms includes at least 5 gut microorganisms selected from Table 1, Table 2, or FIGS. 13A-13XX that are assigned to Guild 1. In some embodiments, the predefined set of microorganisms includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 175, 180, 190, 200, 225, 250, 275, 300, 350, 400, or more gut microorganisms selected from Table 1, Table 2, or FIGS. 13A-13XX that are assigned to Guild 1.

In some embodiments, the predefined set of microorganisms only includes gut microorganisms selected from Table 1, Table 2, or FIGS. 13A-13XX. that are assigned to Guild 2. That is, the predefined set of microoganisms does not include microorganisms selected from Table 1, Table 2, or FIGS. 13A-13XX. that are assigned to Guild 1. In some embodiments, the predefined set of microorganisms includes at least 5 gut microorganisms selected from Table 1, Table 2, or FIGS. 13A-13XX that are assigned to Guild 2. In some embodiments, the predefined set of microorganisms includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 175, 180, 190, 200, 225, 250, 275, 300, 350, 400, or more gut microorganisms selected from Table 1, Table 2, or FIGS. 13A-13XX that are assigned to Guild 2.

In some embodiments, when the prediction of the subject's response to the therapy does not satisfy the threshold likelihood that the subject will respond favorably to the therapy, the method further comprises administering the therapy to the subject. In some embodiments, the therapy is administered to the subject around the same time as the one or more of the plurality of gut microorganisms are administered. In some embodiments, the therapy is administered to the subject after the one or more of the plurality of gut microorganisms are administered. In some embodiments, the therapy is administered to the subject at least 1 day, at least 2 days, at least 3 days, at least 4 days, at least 5 days, at least 6 days, at least 7 days, at least 1 week, at least 2 weeks, at least 3 weeks, at least 4 weeks, at least 5 weeks, at least 6 weeks, at least 7 weeks, at least 8 weeks, or more after the one or more of the plurality of gut microorganisms are administered. In some embodiments, the therapy is administered to the subject no more than 3 months, no more than 2 months, no more than one month, no more than 4 weeks, no more than 3 weeks, no more than 2 weeks, no more than 1 week, no more than 6 days, no more than 5 days, no more than 4 days, no more than 3 days, or no more than 2 days after the one or more of the plurality of gut microorganisms are administered. In some embodiments, the therapy is administered to the subject from 1 day to 2 months, from 1 day to 1 month, from 1 day to 3 weeks, from 1 day to 2 weeks, from 1 day to 1 week, from 1 day to 3 days, from 2 days to 2 months, from 2 days to 1 month, from 2 days to 3 weeks, from 2 days to 2 weeks, from 2 days to 1 week, from 2 days to 3 days, from 3 days to 2 months, from 3 days to 1 month, from 3 days to 3 weeks, from 3 days to 2 weeks, from 3 days to 1 week, from 1 week to 2 months, from 1 week to 1 month, from 1 week to 3 weeks, or from 1 week to 2 weeks after the one or more of the plurality of gut microorganisms are administered.

In some embodiments, if a subject is classified ahead of treatment as a predicted non-responder, then a clinician may treat that subject differently to a subject classified as a predicted responder. Classifying the subject as a predicted non-responder or as a predicted responder may allow the adoption of a particular, or an alternative, treatment regime more suited to the patient.

In some embodiments, a treatment regime or a therapy can be administered via any common route so long as the target tissue or cell is available via that route. In some embodiment, a treatment regime or a therapy can be administered via any common route so long as the target tissue or cell is available via that route. This includes, but is not limited to, intravenous, catheterization, orthotopic, intradermal, subcutaneous, intramuscular, intraperitoneal intertumoral, oral, nasal, buccal, rectal, vaginal, or topical administration. Selection of therapeutic agents and dosage regimes may depend on various factors, such as the drug combination employed, the particular disease being treated, and the condition and prior history of the patient.

In some embodiments, a non-responder is administered with one or more of the pluralities of gut microorganisms via, but is not limited to, oral administration or by colonoscopy. A gut microorganism therapeutic composition for use as described herein can be prepared and administered using methods known in the art. In general, compositions are formulated for oral, colonoscopic, or nasogastric delivery although any appropriate method can be used.

In some embodiments, a non-responder receives fecal microbiota transplantation from a responder population through methods as disclosed in e.g., US20230109343, US20200147151, or US2021036172. In some embodiments, a non-responder receives an effective amount of pre-selected isolated population of gut microorganisms from fecal matters of a responder. In some embodiments, a non-responder receives an effective amount of pre-selected isolated population of gut microorganisms from Table 1, Table 2 or FIG. 13A-13XX. In some embodiments, the one or more of the pluralities of gut microorganisms administered to a non-responder comprise a therapeutically effective or sufficient amount of at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, at least 600, at least 700, at least 800, or all of the gut microorganisms isolated or purified populations of gut microorganisms selected from Table 1, Table 2 or FIGS. 13A-13XX. In some embodiments, the one or more of the pluralities of gut microorganisms administered to a non-responder comprise at least about 1×10³ viable colony forming units (CFU) of bacteria or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ viable CFU (or any derivable range therein). In some embodiments, a single dose will contain an amount of gut microorganisms (such as a specific bacteria or species, genus, or family described herein) of at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ viable CFU (or any derivable range therein) of a specified bacteria. In some embodiments, a single dose will contain at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ viable CFU (or any derivable range therein) of total gut microorganisms.

In some embodiments, the pluralities of gut microorganisms are administered concomitantly or sequentially with one or more therapies to a disease or a disorder. In some embodiments, some, most, or substantially all of the subject's colon, gut or intestinal microbiota are removed prior to the administering of the composition.

In some embodiments, the pluralities of gut microorganisms are administered more than once. In certain aspects, the composition is administered daily, weekly, or monthly. In some embodiments, the pluralities of gut microorganisms are administered for two, three, or four months to induce and/or maintain an appropriate microbiome in the non-responder's GI tract.

### Compositions

In one aspect, the disclosure provides a pharmaceutical composition comprising a first gut microorganism selected from those microorganisms listed in FIG. 13A-13XX. In FIG. 13, each entry identifies a species of gut microorganism identified in the examples below, whether that species is present in the core set of 284 microbiota (core 284 genomes), which of the two guilds described in the examples the microorganism belongs, and provides a taxonomic classification of the microorganism, where d=domain, p=phylum, c=class, o=order, f=family, g=genus, and s=species. For example, the first entry in FIG. 13A is reproduced below:
Microrganism Id: 1U001.8 Core: N Guild Assignment: Guild 2
d_Bacteria;p_Proteobacteria;c_Gammaproteobacteria;o_Enterobacterales;f_Enter obacteriaceae;g_Escherichia;s Escherichia coli

This defines organism 1U001.8, which is not part of the core set of microorganisms, is part of guild 2, and has the taxonomic classification of domain=Bacteria, phylum=Proteobacteria, class=Gammaproteobacteria, order=Enterobacterales, family=Enterobacteriaceae, genus=Escherichia, and species Escherichia coli.

Genomic sequences for each organism listed in FIG. 13 can be found in the sequence listing filed herewith, as mapped according to the associated entry in FIG. 12. For example, as shown in FIG. 12A, organism 1U001.8 has genomic sequences corresponding to those in SEQ ID NOS: 1-68. As described in the examples, species were defined as those organisms having at least a threshold percentage of similarity in their genomic sequences. For example, in some embodiments, a microorganism is defined as organism 1U001.8 when their genome shares at least 99% identity with the sequences of SEQ ID NOS: 1-68. In some embodiments, a microorganism is defined as a microorganism listed in FIG. 13A when its genome has at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8%, or at least 99.9% sequence identity with the genomic sequences corresponding to that organism in the sequence listing, as mapped in FIG. 12.

In some embodiments, the pharmaceutical composition includes more than one microorganism listed in FIG. 13. In some embodiments, the pharmaceutical composition includes at least 2, at least 3, at least 4, at least 5, at least 10, at least 15, at least 20, at least 25, at least 30, at least 40, at least 50, at least 75, at least 100, at least 125, at least 150, at least 175, at least 200, at least 225, at least 250, at least 300, at least 350, at least 400, at least 450, at least 500, at least 550, at least 600, at least 650, at least 700, at least 750, or at least 800 of the microorganisms listed in FIG. 13.

In some embodiments, the majority of microorganisms in the pharmaceutical composition are those listed in FIG. 13. In some embodiments, at least 80% of the microorganisms in the pharmaceutical composition are microorganisms listed in FIG. 13. In some embodiments, at least 85% of the microorganisms in the pharmaceutical composition are microorganisms listed in FIG. 13. In some embodiments, at least 90% of the microorganisms in the pharmaceutical composition are microorganisms listed in FIG. 13. In some embodiments, at least 95% of the microorganisms in the pharmaceutical composition are microorganisms listed in FIG. 13. In some embodiments, at least 98% of the microorganisms in the pharmaceutical composition are microorganisms listed in FIG. 13. In some embodiments, at least 99% of the microorganisms in the pharmaceutical composition are microorganisms listed in FIG. 13. In some embodiments, at least 99.5% of the microorganisms in the pharmaceutical composition are microorganisms listed in FIG. 13. In some embodiments, at least 99.8% of the microorganisms in the pharmaceutical composition are microorganisms listed in FIG. 13. In some embodiments, at least 99.9% of the microorganisms in the pharmaceutical composition are microorganisms listed in FIG. 13. In some embodiments, at least 99.99% of the microorganisms in the pharmaceutical composition are microorganisms listed in FIG. 13. In some embodiments, the pharmaceutical composition only includes microorganisms listed in FIG. 13.

In some embodiments, the majority of microorganisms in the pharmaceutical composition are those listed as core microorganisms in FIG. 13. In some embodiments, at least 80% of the microorganisms in the pharmaceutical composition are microorganisms listed as core microorganisms in FIG. 13. In some embodiments, at least 85% of the microorganisms in the pharmaceutical composition are microorganisms listed as core microorganisms in FIG. 13. In some embodiments, at least 90% of the microorganisms in the pharmaceutical composition are microorganisms listed as core microorganisms in FIG. 13. In some embodiments, at least 95% of the microorganisms in the pharmaceutical composition are microorganisms listed as core microorganisms in FIG. 13. In some embodiments, at least 98% of the microorganisms in the pharmaceutical composition are microorganisms listed as core microorganisms in FIG. 13. In some embodiments, at least 99% of the microorganisms in the pharmaceutical composition are microorganisms listed as core microorganisms in FIG. 13. In some embodiments, at least 99.5% of the microorganisms in the pharmaceutical composition are microorganisms listed as core microorganisms in FIG. 13. In some embodiments, at least 99.8% of the microorganisms in the pharmaceutical composition are microorganisms listed as core microorganisms in FIG. 13. In some embodiments, at least 99.9% of the microorganisms in the pharmaceutical composition are microorganisms listed as core microorganisms in FIG. 13. In some embodiments, at least 99.99% of the microorganisms in the pharmaceutical composition are microorganisms listed as core microorganisms in FIG. 13. In some embodiments, the pharmaceutical composition only includes microorganisms listed as core microorganisms in FIG. 13.

In some embodiments, the pharmaceutical composition includes at least 2, at least 3, at least 4, at least 5, at least 10, at least 15, at least 20, at least 25, at least 30, at least 40, at least 50, at least 75, at least 100, at least 125, at least 150, at least 175, at least 200, at least 225, at least 250, at least 275, or all of the microorganisms listed as core microorganisms in FIG. 13.

In some embodiments, the majority of microorganisms in the pharmaceutical composition are those listed as guild 1 microorganisms in FIG. 13. In some embodiments, at least 80% of the microorganisms in the pharmaceutical composition are microorganisms listed as guild 1 microorganisms in FIG. 13. In some embodiments, at least 85% of the microorganisms in the pharmaceutical composition are microorganisms listed as guild 1 microorganisms in FIG. 13. In some embodiments, at least 90% of the microorganisms in the pharmaceutical composition are microorganisms listed as guild 1 microorganisms in FIG. 13. In some embodiments, at least 95% of the microorganisms in the pharmaceutical composition are microorganisms listed as guild 1 microorganisms in FIG. 13. In some embodiments, at least 98% of the microorganisms in the pharmaceutical composition are microorganisms listed as guild 1 microorganisms in FIG. 13. In some embodiments, at least 99% of the microorganisms in the pharmaceutical composition are microorganisms listed as guild 1 microorganisms in FIG. 13. In some embodiments, at least 99.5% of the microorganisms in the pharmaceutical composition are microorganisms listed as guild 1 microorganisms in FIG. 13. In some embodiments, at least 99.8% of the microorganisms in the pharmaceutical composition are microorganisms listed as guild 1 microorganisms in FIG. 13. In some embodiments, at least 99.9% of the microorganisms in the pharmaceutical composition are microorganisms listed as guild 1 microorganisms in FIG. 13. In some embodiments, at least 99.99% of the microorganisms in the pharmaceutical composition are microorganisms listed as guild 1 microorganisms in FIG. 13. In some embodiments, the pharmaceutical composition only includes microorganisms listed as guild 1 microorganisms in FIG. 13.

In some embodiments, the pharmaceutical composition includes at least 2, at least 3, at least 4, at least 5, at least 10, at least 15, at least 20, at least 25, at least 30, at least 40, at least 50, at least 75, at least 100, at least 125, at least 150, at least 175, at least 200, at least 225, at least 250, at least 275, at least 300, at least 400, or all of the microorganisms listed as guild 1 microorganisms in FIG. 13.

In some embodiments, the majority of microorganisms in the pharmaceutical composition are those listed as guild 1 and core microorganisms in FIG. 13. In some embodiments, at least 80% of the microorganisms in the pharmaceutical composition are microorganisms listed as guild 1 and core microorganisms in FIG. 13. In some embodiments, at least 85% of the microorganisms in the pharmaceutical composition are microorganisms listed as guild 1 and core microorganisms in FIG. 13. In some embodiments, at least 90% of the microorganisms in the pharmaceutical composition are microorganisms listed as guild 1 and core microorganisms in FIG. 13. In some embodiments, at least 95% of the microorganisms in the pharmaceutical composition are microorganisms listed as guild 1 and core microorganisms in FIG. 13. In some embodiments, at least 98% of the microorganisms in the pharmaceutical composition are microorganisms listed as guild 1 and core microorganisms in FIG. 13. In some embodiments, at least 99% of the microorganisms in the pharmaceutical composition are microorganisms listed as guild 1 and core microorganisms in FIG. 13. In some embodiments, at least 99.5% of the microorganisms in the pharmaceutical composition are microorganisms listed as guild 1 and core microorganisms in FIG. 13. In some embodiments, at least 99.8% of the microorganisms in the pharmaceutical composition are microorganisms listed as guild 1 and core microorganisms in FIG. 13. In some embodiments, at least 99.9% of the microorganisms in the pharmaceutical composition are microorganisms listed as guild 1 and core microorganisms in FIG. 13. In some embodiments, at least 99.99% of the microorganisms in the pharmaceutical composition are microorganisms listed as guild 1 and core microorganisms in FIG. 13. In some embodiments, the pharmaceutical composition only includes microorganisms listed as guild 1 and core microorganisms in FIG. 13.

In some embodiments, the pharmaceutical composition includes at least 2, at least 3, at least 4, at least 5, at least 10, at least 15, at least 20, at least 25, at least 30, at least 40, at least 50, at least 75, at least 100, at least 125, at least 150, at least 175, or all of the microorganisms listed as guild 1 and core microorganisms in FIG. 13.

In some embodiments, the majority of microorganisms in the pharmaceutical composition are those listed as guild 2 microorganisms in FIG. 13. In some embodiments, at least 80% of the microorganisms in the pharmaceutical composition are microorganisms listed as guild 2 microorganisms in FIG. 13. In some embodiments, at least 85% of the microorganisms in the pharmaceutical composition are microorganisms listed as guild 2 microorganisms in FIG. 13. In some embodiments, at least 90% of the microorganisms in the pharmaceutical composition are microorganisms listed as guild 2 microorganisms in FIG. 13. In some embodiments, at least 95% of the microorganisms in the pharmaceutical composition are microorganisms listed as guild 2 microorganisms in FIG. 13. In some embodiments, at least 98% of the microorganisms in the pharmaceutical composition are microorganisms listed as guild 2 microorganisms in FIG. 13. In some embodiments, at least 99% of the microorganisms in the pharmaceutical composition are microorganisms listed as guild 2 microorganisms in FIG. 13. In some embodiments, at least 99.5% of the microorganisms in the pharmaceutical composition are microorganisms listed as guild 2 microorganisms in FIG. 13. In some embodiments, at least 99.8% of the microorganisms in the pharmaceutical composition are microorganisms listed as guild 2 microorganisms in FIG. 13. In some embodiments, at least 99.9% of the microorganisms in the pharmaceutical composition are microorganisms listed as guild 2 microorganisms in FIG. 13. In some embodiments, at least 99.99% of the microorganisms in the pharmaceutical composition are microorganisms listed as guild 2 microorganisms in FIG. 13. In some embodiments, the pharmaceutical composition only includes microorganisms listed as guild 2 microorganisms in FIG. 13.

In some embodiments, the pharmaceutical composition includes at least 2, at least 3, at least 4, at least 5, at least 10, at least 15, at least 20, at least 25, at least 30, at least 40, at least 50, at least 75, at least 100, at least 125, at least 150, at least 175, at least 200, at least 225, at least 250, at least 275, at least 300, at least 400, or all of the microorganisms listed as guild 2 microorganisms in FIG. 13.

In some embodiments, the majority of microorganisms in the pharmaceutical composition are those listed as guild 2 and core microorganisms in FIG. 13. In some embodiments, at least 80% of the microorganisms in the pharmaceutical composition are microorganisms listed as guild 2 and core microorganisms in FIG. 13. In some embodiments, at least 85% of the microorganisms in the pharmaceutical composition are microorganisms listed as guild 2 and core microorganisms in FIG. 13. In some embodiments, at least 90% of the microorganisms in the pharmaceutical composition are microorganisms listed as guild 2 and core microorganisms in FIG. 13. In some embodiments, at least 95% of the microorganisms in the pharmaceutical composition are microorganisms listed as guild 2 and core microorganisms in FIG. 13. In some embodiments, at least 98% of the microorganisms in the pharmaceutical composition are microorganisms listed as guild 2 and core microorganisms in FIG. 13. In some embodiments, at least 99% of the microorganisms in the pharmaceutical composition are microorganisms listed as guild 2 and core microorganisms in FIG. 13. In some embodiments, at least 99.5% of the microorganisms in the pharmaceutical composition are microorganisms listed as guild 2 and core microorganisms in FIG. 13. In some embodiments, at least 99.8% of the microorganisms in the pharmaceutical composition are microorganisms listed as guild 2 and core microorganisms in FIG. 13. In some embodiments, at least 99.9% of the microorganisms in the pharmaceutical composition are microorganisms listed as guild 2 and core microorganisms in FIG. 13. In some embodiments, at least 99.99% of the microorganisms in the pharmaceutical composition are microorganisms listed as guild 2 and core microorganisms in FIG. 13. In some embodiments, the pharmaceutical composition only includes microorganisms listed as guild 2 and core microorganisms in FIG. 13.

In some embodiments, the pharmaceutical composition includes at least 2, at least 3, at least 4, at least 5, at least 10, at least 15, at least 20, at least 25, at least 30, at least 40, at least 50, at least 75, at least 100, at least 125, at least 150, at least 175, or all of the microorganisms listed as guild 2 and core microorganisms in FIG. 13.

In some embodiments, the pharmaceutical compositions are prepared from cultures of the microorganism or microorganisms. For example, in an embodiment where the pharmaceutical composition contains a single microorganisms, the microorganism is cultured alone and the culture is used to prepare the composition, e.g., for fecal microbiota transplant (FMT). In some embodiments, where the pharmaceutical composition contains multiple microorganisms, each microorganism is cultured separately and then combined to generate the pharmaceutical composition. In some embodiments, where the pharmaceutical composition contains multiple microorganisms, two or more microorganisms are cultured together and, optionally, mixed with other microorganisms cultured separately. In some embodiments, where the pharmaceutical composition contains multiple microorganisms, all of the microorganisms are cultured together.

In some embodiments, the pharmaceutical composition is for fecal microbiota transplant. A review of the use of FMT is provided by Al-Ali D, Ahmed A, Shafiq A, Mc Veigh C, Chaari A, Zakaria D, and Bendriss G, “Fecal microbiota transplants: A review of emerging clinical data on applications, efficacy, and risks (2015-2020),” Qatar Med J., 2021(1):5 (2021), the disclosure of which is incorporated herein by reference.

In some embodiments, a pharmaceutical composition for FMT is a fecal sample that is supplemented with one or more of the microorganisms disclosed in FIG. 13. In some embodiments, at least half of the microorganisms in the supplemented fecal sample are from the supplementing. In some embodiments, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at east 90%, at least 95%, at least 98%, at least 99%, at least 99.5%, at least 99.8%, or at least 99.9% of the microorganisms in the supplemented fecal sample are from the supplementing. In some embodiments, the fecal sample is sterilized prior to supplementing with one or more microorganisms listed in Table 13, to kill the majority (e.g., at least 50%, at least 75%, at least 90%, at least 95%, at least 98%, at least 99%, at least 99.5, at least 99.8%, at least 99.9%, or all) of the microorganisms from the fecal sample prior to supplementation.

In some embodiments, the pharmaceutical composition is a synthetic fecal sample (e.g., a synthetic stool). An example description of the use of synthetic stool is provided in Gweon T G, Na S Y, “Next Generation Fecal Microbiota Transplantation,” Clin Endosc., 54(2):152-156 (2021), the disclosure of which is incorporated herein by reference.

In some embodiments, the composition further includes a pharmaceutically acceptable excipient.

In some embodiments, the first gut microorganism belongs to Guild 1, as identified in FIGS. 13A-13XX. In some embodiments, the first gut microorganism belongs to Guild 2, as identified in FIGS. 13A-13XX.

In some embodiments, the first gut microorganism has a genome having at least 99% sequence identity to a set of contigs for a microorganism listed in FIGS. 12A-12I.

In some embodiments, the first gut microorganism comprises at least 50% of the total amount of gut microorganisms in the composition. In some embodiments, wherein the first gut microorganism comprises at least 75% of the total amount of gut microorganisms in the composition. In some embodiments, the first gut microorganism comprises at least 90% of the total amount of gut microorganisms in the composition. In some embodiments, the first gut microorganism comprises at least 95% of the total amount of gut microorganisms in the composition. In some embodiments, the first gut microorganism comprises at least 99% of the total amount of gut microorganisms in the composition. In some embodiments, the first gut microorganism comprises at least 99.5% of the total amount of gut microorganisms in the composition. In some embodiments, the first gut microorganism comprises at least 99.9% of the total amount of gut microorganisms in the composition.

In some embodiments, the composition further includes a second gut microorganism selected from those microorganisms listed in FIG. 13A-13XX. In some embodiments, the second gut microorganism belongs to the same Guild as the first gut microorganism, as identified in FIGS. 13A-13XX.

In one aspect, the disclosure provides a composition comprising a first gut microorganism selected from those microorganisms listed in FIG. 13A-13XX. In some embodiments, the composition includes more than one microorganism listed in FIG. 13. In some embodiments, the composition includes at least 2, at least 3, at least 4, at least 5, at least 10, at least 15, at least 20, at least 25, at least 30, at least 40, at least 50, at least 75, at least 100, at least 125, at least 150, at least 175, at least 200, at least 225, at least 250, at least 300, at least 350, at least 400, at least 450, at least 500, at least 550, at least 600, at least 650, at least 700, at least 750, or at least 800 of the microorganisms listed in FIG. 13.

In some embodiments, the majority of microorganisms in the composition are those listed in FIG. 13. In some embodiments, at least 80% of the microorganisms in the composition are microorganisms listed in FIG. 13. In some embodiments, at least 85% of the microorganisms in the composition are microorganisms listed in FIG. 13. In some embodiments, at least 90% of the microorganisms in the composition are microorganisms listed in FIG. 13. In some embodiments, at least 95% of the microorganisms in the composition are microorganisms listed in FIG. 13. In some embodiments, at least 98% of the microorganisms in the composition are microorganisms listed in FIG. 13. In some embodiments, at least 99% of the microorganisms in the composition are microorganisms listed in FIG. 13. In some embodiments, at least 99.5% of the microorganisms in the composition are microorganisms listed in FIG. 13. In some embodiments, at least 99.8% of the microorganisms in the composition are microorganisms listed in FIG. 13. In some embodiments, at least 99.9% of the microorganisms in the composition are microorganisms listed in FIG. 13. In some embodiments, at least 99.99% of the microorganisms in the composition are microorganisms listed in FIG. 13. In some embodiments, the composition only includes microorganisms listed in FIG. 13.

In some embodiments, the majority of microorganisms in the composition are those listed as core microorganisms in FIG. 13. In some embodiments, at least 80% of the microorganisms in the composition are microorganisms listed as core microorganisms in FIG. 13. In some embodiments, at least 85% of the microorganisms in the composition are microorganisms listed as core microorganisms in FIG. 13. In some embodiments, at least 90% of the microorganisms in the composition are microorganisms listed as core microorganisms in FIG. 13. In some embodiments, at least 95% of the microorganisms in the composition are microorganisms listed as core microorganisms in FIG. 13. In some embodiments, at least 98% of the microorganisms in the composition are microorganisms listed as core microorganisms in FIG. 13. In some embodiments, at least 99% of the microorganisms in the composition are microorganisms listed as core microorganisms in FIG. 13. In some embodiments, at least 99.5% of the microorganisms in the composition are microorganisms listed as core microorganisms in FIG. 13. In some embodiments, at least 99.8% of the microorganisms in the composition are microorganisms listed as core microorganisms in FIG. 13. In some embodiments, at least 99.9% of the microorganisms in the composition are microorganisms listed as core microorganisms in FIG. 13. In some embodiments, at least 99.99% of the microorganisms in the composition are microorganisms listed as core microorganisms in FIG. 13. In some embodiments, the composition only includes microorganisms listed as core microorganisms in FIG. 13.

In some embodiments, the composition includes at least 2, at least 3, at least 4, at least 5, at least 10, at least 15, at least 20, at least 25, at least 30, at least 40, at least 50, at least 75, at least 100, at least 125, at least 150, at least 175, at least 200, at least 225, at least 250, at least 275, or all of the microorganisms listed as core microorganisms in FIG. 13.

In some embodiments, the majority of microorganisms in the composition are those listed as guild 1 microorganisms in FIG. 13. In some embodiments, at least 80% of the microorganisms in the composition are microorganisms listed as guild 1 microorganisms in FIG. 13. In some embodiments, at least 85% of the microorganisms in the composition are microorganisms listed as guild 1 microorganisms in FIG. 13. In some embodiments, at least 90% of the microorganisms in the composition are microorganisms listed as guild 1 microorganisms in FIG. 13. In some embodiments, at least 95% of the microorganisms in the composition are microorganisms listed as guild 1 microorganisms in FIG. 13. In some embodiments, at least 98% of the microorganisms in the composition are microorganisms listed as guild 1 microorganisms in FIG. 13. In some embodiments, at least 99% of the microorganisms in the composition are microorganisms listed as guild 1 microorganisms in FIG. 13. In some embodiments, at least 99.5% of the microorganisms in the composition are microorganisms listed as guild 1 microorganisms in FIG. 13. In some embodiments, at least 99.8% of the microorganisms in the composition are microorganisms listed as guild 1 microorganisms in FIG. 13. In some embodiments, at least 99.9% of the microorganisms in the composition are microorganisms listed as guild 1 microorganisms in FIG. 13. In some embodiments, at least 99.99% of the microorganisms in the composition are microorganisms listed as guild 1 microorganisms in FIG. 13. In some embodiments, the composition only includes microorganisms listed as guild 1 microorganisms in FIG. 13.

In some embodiments, the composition includes at least 2, at least 3, at least 4, at least 5, at least 10, at least 15, at least 20, at least 25, at least 30, at least 40, at least 50, at least 75, at least 100, at least 125, at least 150, at least 175, at least 200, at least 225, at least 250, at least 275, at least 300, at least 400, or all of the microorganisms listed as guild 1 microorganisms in FIG. 13.

In some embodiments, the majority of microorganisms in the composition are those listed as guild 1 and core microorganisms in FIG. 13. In some embodiments, at least 80% of the microorganisms in the composition are microorganisms listed as guild 1 and core microorganisms in FIG. 13. In some embodiments, at least 85% of the microorganisms in the composition are microorganisms listed as guild 1 and core microorganisms in FIG. 13. In some embodiments, at least 90% of the microorganisms in the composition are microorganisms listed as guild 1 and core microorganisms in FIG. 13. In some embodiments, at least 95% of the microorganisms in the composition are microorganisms listed as guild 1 and core microorganisms in FIG. 13. In some embodiments, at least 98% of the microorganisms in the composition are microorganisms listed as guild 1 and core microorganisms in FIG. 13. In some embodiments, at least 99% of the microorganisms in the composition are microorganisms listed as guild 1 and core microorganisms in FIG. 13. In some embodiments, at least 99.5% of the microorganisms in the composition are microorganisms listed as guild 1 and core microorganisms in FIG. 13. In some embodiments, at least 99.8% of the microorganisms in the composition are microorganisms listed as guild 1 and core microorganisms in FIG. 13. In some embodiments, at least 99.9% of the microorganisms in the composition are microorganisms listed as guild 1 and core microorganisms in FIG. 13. In some embodiments, at least 99.99% of the microorganisms in the composition are microorganisms listed as guild 1 and core microorganisms in FIG. 13. In some embodiments, the composition only includes microorganisms listed as guild 1 and core microorganisms in FIG. 13.

In some embodiments, the composition includes at least 2, at least 3, at least 4, at least 5, at least 10, at least 15, at least 20, at least 25, at least 30, at least 40, at least 50, at least 75, at least 100, at least 125, at least 150, at least 175, or all of the microorganisms listed as guild 1 and core microorganisms in FIG. 13.

In some embodiments, the majority of microorganisms in the composition are those listed as guild 2 microorganisms in FIG. 13. In some embodiments, at least 80% of the microorganisms in the composition are microorganisms listed as guild 2 microorganisms in FIG. 13. In some embodiments, at least 85% of the microorganisms in the composition are microorganisms listed as guild 2 microorganisms in FIG. 13. In some embodiments, at least 90% of the microorganisms in the composition are microorganisms listed as guild 2 microorganisms in FIG. 13. In some embodiments, at least 95% of the microorganisms in the composition are microorganisms listed as guild 2 microorganisms in FIG. 13. In some embodiments, at least 98% of the microorganisms in the composition are microorganisms listed as guild 2 microorganisms in FIG. 13. In some embodiments, at least 99% of the microorganisms in the composition are microorganisms listed as guild 2 microorganisms in FIG. 13. In some embodiments, at least 99.5% of the microorganisms in the composition are microorganisms listed as guild 2 microorganisms in FIG. 13. In some embodiments, at least 99.8% of the microorganisms in the composition are microorganisms listed as guild 2 microorganisms in FIG. 13. In some embodiments, at least 99.9% of the microorganisms in the composition are microorganisms listed as guild 2 microorganisms in FIG. 13. In some embodiments, at least 99.99% of the microorganisms in the composition are microorganisms listed as guild 2 microorganisms in FIG. 13. In some embodiments, the composition only includes microorganisms listed as guild 2 microorganisms in FIG. 13.

In some embodiments, the composition includes at least 2, at least 3, at least 4, at least 5, at least 10, at least 15, at least 20, at least 25, at least 30, at least 40, at least 50, at least 75, at least 100, at least 125, at least 150, at least 175, at least 200, at least 225, at least 250, at least 275, at least 300, at least 400, or all of the microorganisms listed as guild 2 microorganisms in FIG. 13.

In some embodiments, the majority of microorganisms in the composition are those listed as guild 2 and core microorganisms in FIG. 13. In some embodiments, at least 80% of the microorganisms in the composition are microorganisms listed as guild 2 and core microorganisms in FIG. 13. In some embodiments, at least 85% of the microorganisms in the composition are microorganisms listed as guild 2 and core microorganisms in FIG. 13. In some embodiments, at least 90% of the microorganisms in the composition are microorganisms listed as guild 2 and core microorganisms in FIG. 13. In some embodiments, at least 95% of the microorganisms in the composition are microorganisms listed as guild 2 and core microorganisms in FIG. 13. In some embodiments, at least 98% of the microorganisms in the composition are microorganisms listed as guild 2 and core microorganisms in FIG. 13. In some embodiments, at least 99% of the microorganisms in the composition are microorganisms listed as guild 2 and core microorganisms in FIG. 13. In some embodiments, at least 99.5% of the microorganisms in the composition are microorganisms listed as guild 2 and core microorganisms in FIG. 13. In some embodiments, at least 99.8% of the microorganisms in the composition are microorganisms listed as guild 2 and core microorganisms in FIG. 13. In some embodiments, at least 99.9% of the microorganisms in the composition are microorganisms listed as guild 2 and core microorganisms in FIG. 13. In some embodiments, at least 99.99% of the microorganisms in the composition are microorganisms listed as guild 2 and core microorganisms in FIG. 13. In some embodiments, the composition only includes microorganisms listed as guild 2 and core microorganisms in FIG. 13.

In some embodiments, the composition includes at least 2, at least 3, at least 4, at least 5, at least 10, at least 15, at least 20, at least 25, at least 30, at least 40, at least 50, at least 75, at least 100, at least 125, at least 150, at least 175, or all of the microorganisms listed as guild 2 and core microorganisms in FIG. 13.

In some embodiments, the compositions are prepared from cultures of the microorganism or microorganisms. For example, in an embodiment where the composition contains a single microorganisms, the microorganism is cultured alone and the culture is used to prepare the composition, e.g., for fecal microbiota transplant (FMT). In some embodiments, where the composition contains multiple microorganisms, each microorganism is cultured separately and then combined to generate the composition. In some embodiments, where the composition contains multiple microorganisms, two or more microorganisms are cultured together and, optionally, mixed with other microorganisms cultured separately. In some embodiments, where the composition contains multiple microorganisms, all of the microorganisms are cultured together.

In some embodiments, the composition is a cell culture.

### Methods of Treatment

In one aspect, the disclosure provides a method for treating a subject in need thereof, the method comprising administering to the subject a therapeutically effective amount of a pharmaceutical composition as described herein. In some embodiments, the administering is by fecal microbiome transplantation. In some embodiments, the administering is by direct transplantation into the gut of the subject. In some embodiments, the administering is by oral ingestion.

In some embodiments, the subject has a condition selected from the group consisting of type-2 diabetes (T2D), hypertension (HT), schizophrenia (SCZ), atherosclerotic cardiovascular disease (ACVD), liver cirrhosis (LC), inflammatory bowel diseases (IBD), colorectal cancer (CRC), ankylosing spondylitis (AS), Parkinson's disease (PD), Multiple Sclerosis (MS), Gaucher disease type II (GDII), COVID-19 (COV), Behcet's disease (BD), autism spectrum disorder (ASD), and pancreatic cancer (PC). In some embodiments, the subject has cancer.

In some embodiments, the method further includes administering a second therapeutic agent to the subject.

In some embodiments, a method is provided for treating a subject in need thereof comprising administering to the subject a pharmaceutical composition comprising a first gut microorganism selected from those microorganisms listed in FIG. 13A-13XX. In some embodiments, the administering comprises fecal microbiota transplant of the pharmaceutical composition.

In some embodiments, the subject has a Clostridium difficile infection. In some embodiments, the subject has a recurrent Clostridium difficile infection. In some embodiments, the subject has inflammatory bowel disease (IBD). In some embodiments, the subject has ulcerative colitis (UC). In some embodiments, the subject has Crohn's disease (CD). In some embodiments, the subject has a functional gastrointestinal disorder (FGID).

In some embodiments, the FGID is an esophageal disorder. In some embodiments, the esophageal disorder is functional chest pain, functional heartburn, reflux hypersensitivity, globus, or functional dysphagia.

In some embodiments, the FGID is a gastroduodenal disorder. In some embodiments, the gastroduodenal disorder is functional dyspepsia, postprandial distress syndrome (PDS), or epigastric pain syndrome (EPS).

In some embodiments, the FGID is a belching disorder. In some embodiments, the belching disorder is excessive supragastric belching or excessive gastric belching.

In some embodiments, the FGID is a nausea and vomiting disorder. In some embodiments, the nausea and vomiting disorder is chronic nausea vomiting syndrome (CNVS), cyclic vomiting syndrome (CVS), cannabinoid hyperemesis syndrome (CHS), or rumination syndrome.

In some embodiments, the FGID is a bowel disorder. In some embodiments, the bowel disorder is irritable bowel syndrome (IBS), IBS with predominant constipation (IBS-C), IBS with predominant diarrhea (IBS-D), IBS with mixed bowel habits (IBS-M), IBS unclassified (IBS-U), functional constipation, functional diarrhea, functional abdominal bloating/distension, unspecified functional bowel disorder, or opioid-induced constipation.

In some embodiments, the FGID is a centrally mediated disorders of gastrointestinal pain. In some embodiments, the centrally mediated disorders of gastrointestinal pain is centrally mediated abdominal pain syndrome (CAPS) or narcotic bowel syndrome (NBS)/Opioid-induced GI hyperalgesia.

In some embodiments, the FGID is a gallbladder and sphincter of Oddi disorder. In some embodiments, the gallbladder and sphincter of Oddi disorder is biliary pain, functional gallbladder disorder, functional biliary sphincter of Oddi disorder, or functional pancreatic sphincter of Oddi disorder.

In some embodiments, the FGID is an anorectal disorder. In some embodiments, the anorectal disorder is fecal incontinence, functional anorectal pain, levator ani syndrome, unspecified functional anorectal pain, proctalgia fugax, a functional defecation disorder, inadequate defecatory propulsion, or dyssynergic defecation.

In some embodiments, the FGID is a childhood functional GI disorder. In some embodiments, the childhood functional GI disorder is infant regurgitation, rumination syndrome, cyclic vomiting syndrome (CVS), infant colic, functional diarrhea, infant dyschezia, or functional constipation.

In some embodiments, the childhood functional GI disorder is a functional nausea and vomiting disorder, cyclic vomiting syndrome (CVS), functional nausea and functional vomiting, functional nausea, functional vomiting, rumination syndrome, aerophagia, a functional abdominal pain disorder, functional dyspepsia, postprandial distress syndrome, epigastric pain syndrome, irritable bowel syndrome (IBS), abdominal migraine, functional abdominal pain-NOS, a functional defecation disorder, functional constipation, or nonretentive fecal incontinence.

In one aspect, the disclosure provides methods for isolating a gut microorganism. In some embodiments, the method includes culturing a single microorganism isolated from a sample, e.g., a gut microbiome sample, sequencing all or a portion of the genome of the microorganism, and determining whether the sequenced portion of the genome has sufficient homology with a genomic sequence for a microorganisms listed in FIG. 13, as provided in the sequence listing mapped to each organism in FIG. 12. In some embodiments, sufficient homology is at least 97% sequence identity, at least 98% sequence identity, at least 99% sequence identity, at least 99.5% sequence identity, at least 99.8% sequence identity, at least 99.9% sequence identity, at least 99.99% sequence identity, or 100% sequence identity.

In some embodiments, the comparison sequence for the microorganism is a sequence identified as unique to that microorganism. In some embodiments, the comparison sequence for the microorganism is at least 500 bp, at least 1 kb, at least 2.5 kb, at least 5 kb, at least 10 kb, at least 25 kb, at least 50 kb, at least 100 kb, at least 250 kb, at least 500 kb, at least 1 M or longer.

Methods for culturing a single microorganism from a sample, e.g., a gut microbiota sample, are known in the art. For example, microorganisms may be plated and diluted until single colonies can be distinguished from one another, each colony being grown up from a single microorganism.

### EXAMPLES

### Example 1—the Two Competing Guilds Identified in the QD Trial (QD-TCG) Distinguish Cases from Controls in 10 Independent Case-Control Metagenomic Datasets of 6 Different Diseases

1.1 Reversible Changes in the Gut Microbiota Associate with Reversible Changes of Host Metabolic Phenotypes

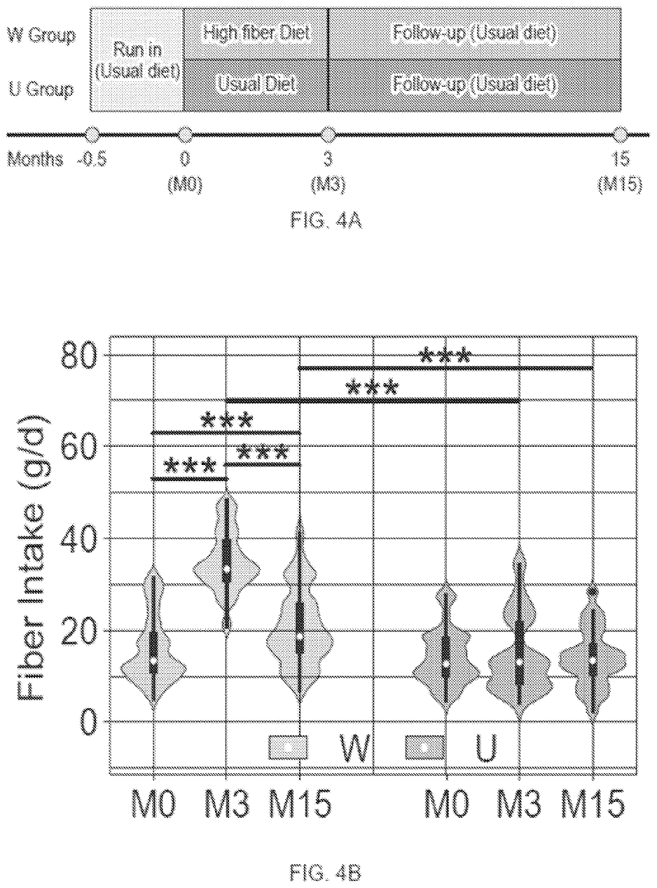

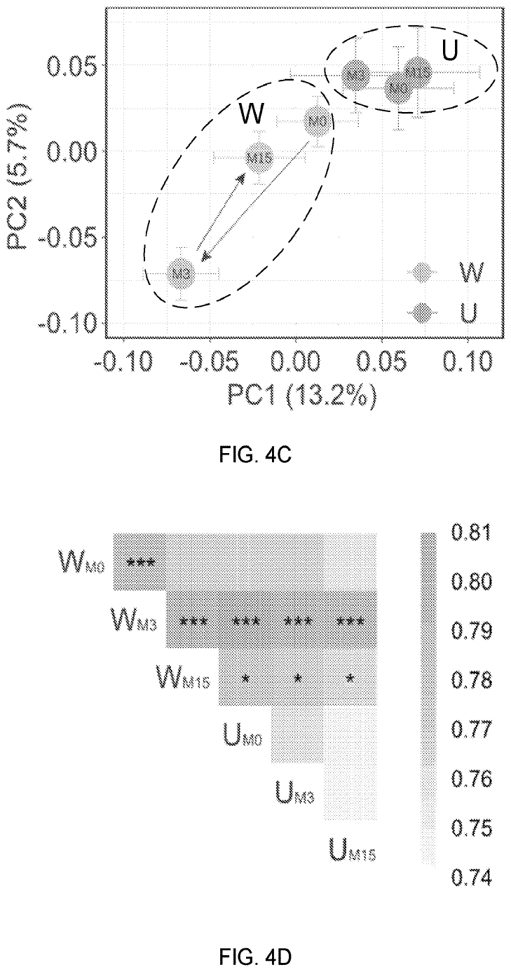

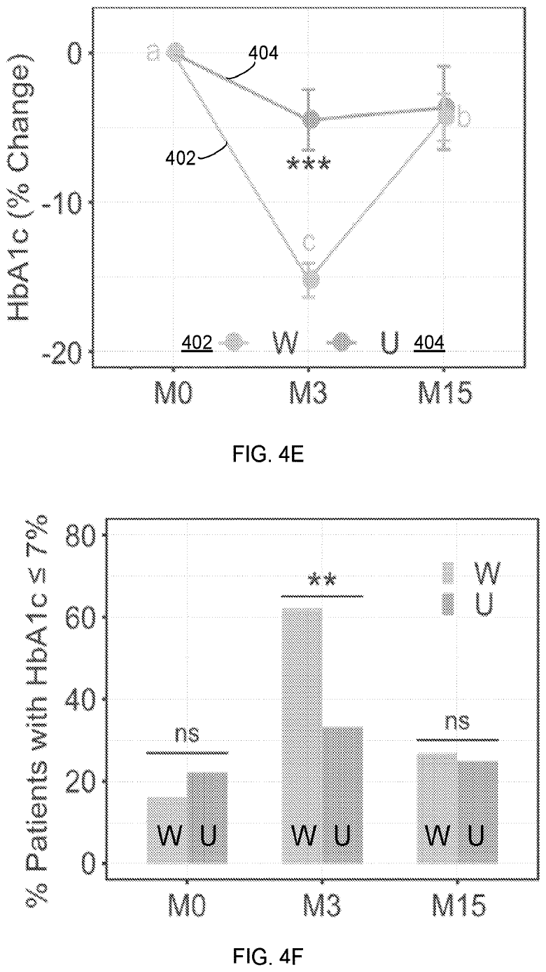

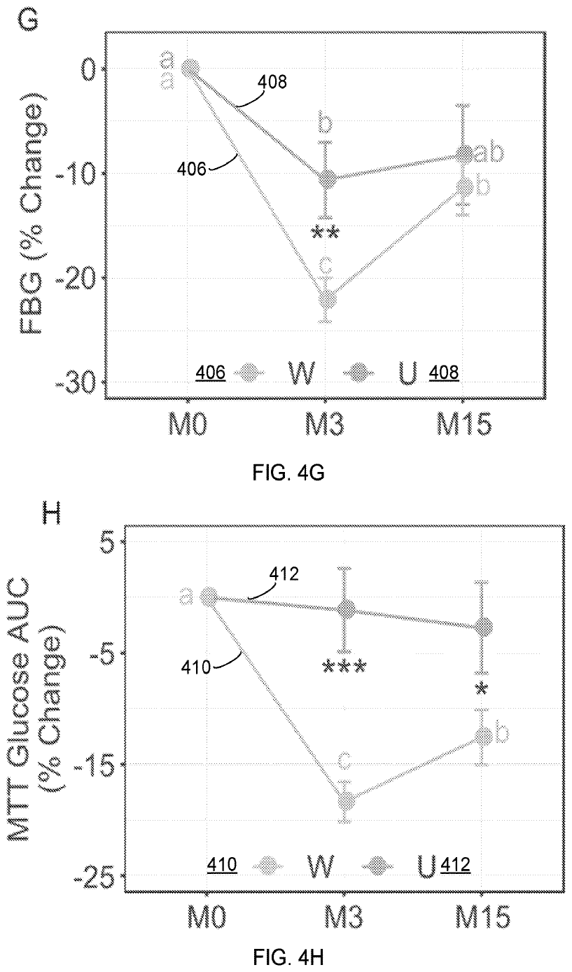

QD is an open label, randomized, and controlled interventional trial, in which T2DM patients were randomized at baseline (M0) to receive either 3 months (M3) of a high fiber intervention (W group; n=74) or standard care (U group; n=36) followed by a one-year follow-up (M15) (FIG. 4A). Dietary fiber intake in the U group remained unchanged throughout the trial, whereas the W group had a significant increase in the intake of dietary fibers from M0 to M3 and a decrease from M3 to M15 (FIG. 4B). Compared with the U group, fiber intake was significantly higher in the W group at both M3 and M15 (FIG. 4B), but energy and protein consumption were similar between the two groups across the study period. Fat intake showed no significant difference between the two groups though a trend of decrease in the W group was observed. Compared with the U group, the W group had similar carbohydrate consumption at M0 and M3, but a higher consumption at M15.

To investigate the gut microbial responses to the introduction and withdrawal of the high fiber intervention, we performed shotgun metagenomic sequencing on 315 fecal samples collected from 110 patients of the W and U group, among whom 95 patients provided samples at all 3 time points and 15 provided samples at M0 and M3 only. To achieve genome-level resolution, we reconstructed 1,845 non-redundant high-quality draft genomes (HQMAGs, two HQMAGs were collapsed into one if the average nucleotide identity, ANI, between them was >99%) from the metagenomic datasets. These HQMAGs accounted for more than 70% of the total reads. In the context of beta-diversity based on Bray-Curtis distance, the overall structure of the gut microbiota in the W group significantly changed from M0 to M3 (PERMANOVA test, P<0.001) and returned to that of M0 at M15; there was no difference in the U group across the 3 timepoints (FIG. 4C-D). Similar changes in abundance-weighted alpha-diversity based on Shannon and Simpson indices were also observed. Regarding to abundance-unweighted indices, richness and Chao1 were increased from M0 to M3 and remained at M15 in both W and U groups. These results showed that the high fiber intervention induced significant structural and abundance changes of the gut microbiota¹¹, however the gut microbiota structure reverted to baseline after the intervention was withdrawn indicating a high resilience in community structure, though there may be lingering in richness increase.

To determine if host metabolic phenotypes would show similar reversible changes as the gut microbiota, we examined 43 bio-clinical parameters of 9 categories across the 3 time points. Hemoglobin A1c (HbA1c) in the U group showed no significant changes throughout the trial. The high fiber intervention significantly reduced the level of HbA1c in the W group from M0 to M3 by 15.22±9.82% (mean±SD). At one-year follow-up of the W group, HbA1c was significantly increased from M3 but remained lower than that at M0 (FIG. 4E). The proportion of patients who achieved adequate glycemic control (HbA1c≤7%) was significantly higher in the W group (61.6% versus 33.3% in the U group) at M3, but showed no difference between the two groups at M15 (FIG. 4F). The level of fasting blood glucose and postprandial glucose in meal tolerance test followed a similar trend as HbA1c (FIGS. 4G, H). Among the rest 40 bio-clinical parameters, 14 also showed an alleviation from M0 to M3 but rebounded at one-year follow-up in the W group. These results indicate that changes of the host metabolic phenotypes were associated with the reversible changes of the gut microbiota in response to the introduction or withdrawal of the high fiber intervention.

1.2 Genome Pairs with Stable Interactions Form a Seesaw-Like Network of Two Competing Guilds.

To facilitate the identification of genome pairs that keep their ecological interactions stable during the trial, particularly in the W group with profound microbiota and host phenotypic changes, we constructed a co-abundance network for each time point based on the abundance matrix of the HQMAGs representing the prevalent microbes. Co-abundance network is a data-driven way to investigate ecological interactions between microbes across habitats. A total of 477 HQMAGs were selected for network construction because they were detectable in more than 75% of the samples at each time point in the W group. These 477 HQMAGs also accounted for ~60% of the total abundance of the 1,845 HQMAGs. In the W group, we calculated pairwise correlations of all 113,526 possible genome pairs among these 477 prevalent HQMAGs based on their abundance across the patients at each time point and constructed 3 co-abundance networks (G_{M0}, G_{M3} and G_{M15}) by using Fastspar, a rapid and scalable correlation estimation tool for compositional data. The three networks were of similar order S, i.e., the total number of nodes (HQMAGs), S_{M0} (442), S_{M3} (421), and S_{M15} (429), but they varied considerably in their size L, i.e., the total number of edges (correlations), L_{M0} (4,231), L_{M3} (2,587) and L_{M15} (4,592). L in G_{M3} decreased to 61.14% of that in G_{M0} and rebounded back in G_{M15} to 108.53% of that in G_{M0}. This pattern was confirmed by changes in connectance, which is defined as the proportion of realized ecological interactions among the potential ones

$$( in undirected network , connectance = \frac{L}{S ( S - 1 ) / 2} , range : [ 0 , 1 ] ) .$$

Connectance decreased from 0.043 in G_{M0} to 0.029 in G_{M3} and rebounded to 0.050 in G_{M15}. Changes in L and connectance showed that the high fiber intervention dramatically reduced the correlations among the prevalent genomes in the network. In addition, we found that the distributions of degree, i.e. the number of edges a node has, fit well with a power-law model (R² values G_{M0}: 0.79, G_{M3}: 0.82, G_{M15}: 0.79), indicating the presence of network hubs²¹. Defining hubs as nodes that connect with more than one-fifth of the total nodes in the network, we found 24 hubs, in which 10 were in G_{M0}. 20 were in G_{M15} but none were in G_{M3}. These results indicate that the overall structure of the gut microbiome undergone profound changes during the trial, particularly, the high fiber intervention resulted in the loss of interactions between genome pairs.

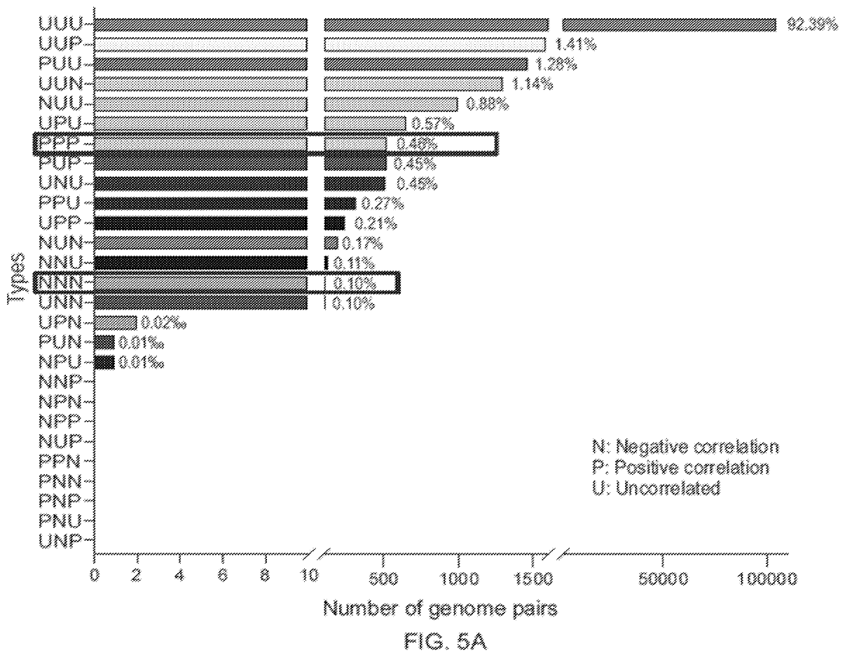

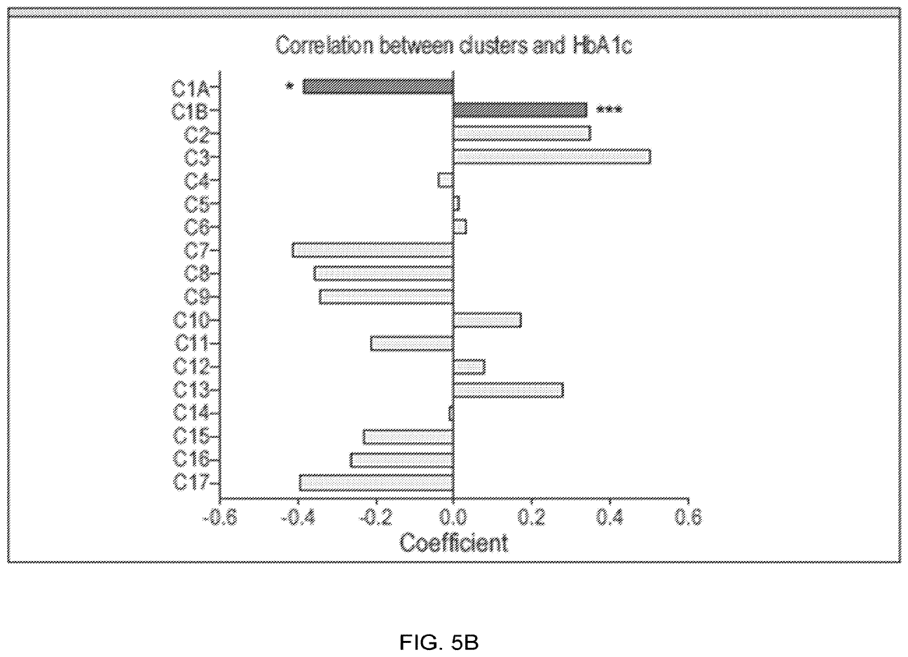

We considered two genomes are connected with robust and stable ecological relationship if they keep the same type of correlation across all the three timepoints. Out of the 113,526 possible genome pairs, 92.39% showed no correlations at any of the three time points, suggesting that it is uncommon for two genomes to establish an ecological relationship even transiently (FIG. 5A. Interestingly, 517 genome pairs showed positive correlations and 118 negative correlations at all the three time points. All the 635 stable correlations involved 184 HQMAGs. These HQMAGs were grouped into 17 unconnected clusters based on the Connected Components Clustering analysis. Cluster C2-C16 had only between 2-9 HQMAGs which had only positive correlations. While cluster C1 was more complex as it had 141 HQMAGs which had both negative and positive correlations. To identify if sub-clusters existed in C1, we further built a clustering tree based on the negative and positive correlations with average linkage method and applied WGCNA analysis. The 141 genomes in C1 were further grouped into 2 subclusters, C1A and C1B. Interestingly, among C1A, C1B and C2-C16, only C1A and C1B were significantly correlated with our primary outcome HbA1c (FIG. 5B. Thus, we focused CIA and C1B for further analysis.

C1A and C1B can be considered as guilds as HQMAGs in each cluster were highly interconnected with only positive correlations no matter which were robust or transient (FIG. 5B). The two guilds were connected by negative edges only, indicating a competitive relationship that structures a seesaw-like network. Such a network feature was termed as two competing guilds (TCG). The members of the TCG had significantly higher degree, betweenness centrality, eigenvector centrality, closeness centrality and stress centrality than the rest of the genomes in the networks. This finding indicates that the two guilds exerted a relatively large amount of control over the interaction of other nodes (reflected by betweenness centrality and eigenvector centrality) and the information flow in the network (reflected by closeness centrality and stress centrality). Removing the two guilds would lead to the collapse of the networks since on average 86.08% of the total edges would have been lost. These suggest that the genomes of the two guilds can be considered as the core nodes of the three large networks (G_{M0}, G_{M3}, and G_{M15}) as they were not only the most stably connected but also the most highly connected nodes in the gut ecosystem network.

The members of the two guilds were also highly prevalent among participants, as 137 of them were in >90%, and 95 were in 100% of the individuals in the W and U groups. In addition, most of these 141 HQMAGs were also predominant members of the gut microbiota as the abundance of 111 of them was higher than the median of the 1,845 HQMAGs and accounted for 20.78% of the total sequencing reads. Based on Bray-Curtis distance, beta-diversity analysis showed significant correlations between the profiles of the two guilds' members and all the 1,845 HQMAGs, as evidenced in the Mantel test (R²=0.62, P=0.001) and Procrustes analysis (P=0.001) (FIG. 4C, D). These indicate that the variations of the two guilds contributed to the major variations of the whole gut microbial community across the 3 time points.

Our data showed that the TCG of the 141 HQMAGs existed in all three ecological networks G_{M0}, G_{M3}, and G_{M15} in the W group. Furthermore, the finding of the TCG in the W group at M0 suggests that such microbial organization exists irrespective of the high fiber intervention in our study. Given the similar overall gut microbiota structure between the W and U groups at M0 and in the U group across 3 timepoints (FIGS. 4C, D), we speculated that the TCG can also be observed in the U group across the trial. Thus, we constructed the co-abundance networks based on the abundance of the 141 HQMAGs across the individuals in the U group at each time point. In the co-abundance networks, 99.8%, 99.51% and 99.74% of the total edges agreed with our TCG, i.e., positive correlations inside the guilds and negative between guilds. This showed that the detection of the TCG was independent of the high fiber intervention, indicating that such a pattern may be an inherent structure of the gut microbiome in our study.

1.3 Dynamics of the Two Competing Guilds Associate with Changes of Host Metabolic Phenotypes.

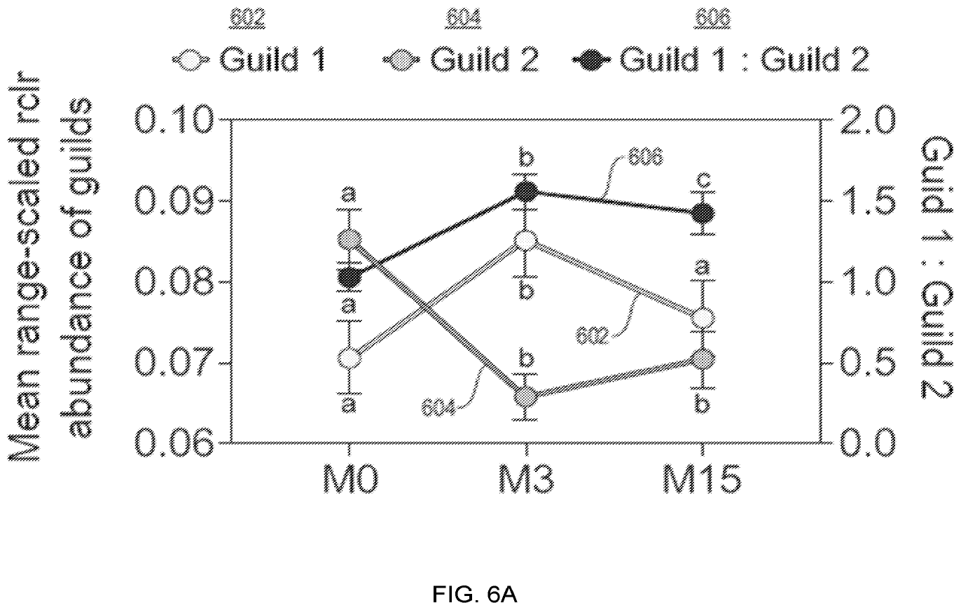

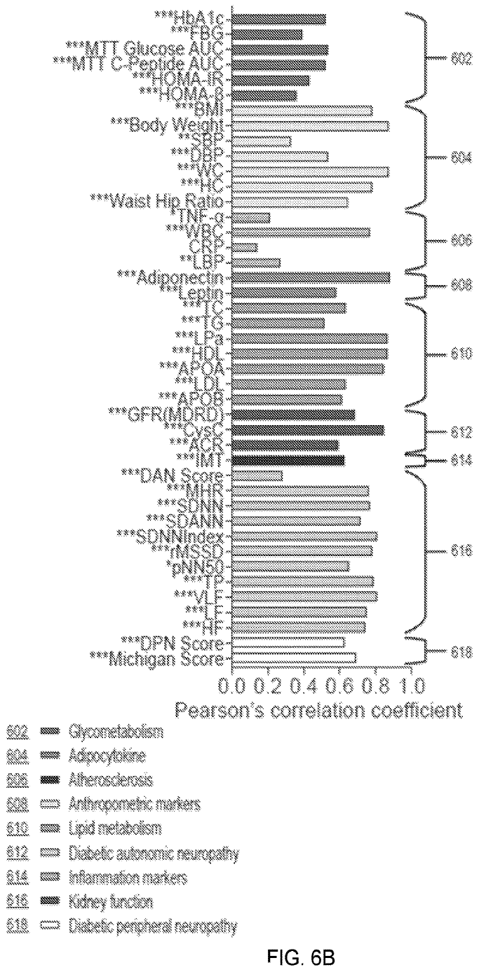

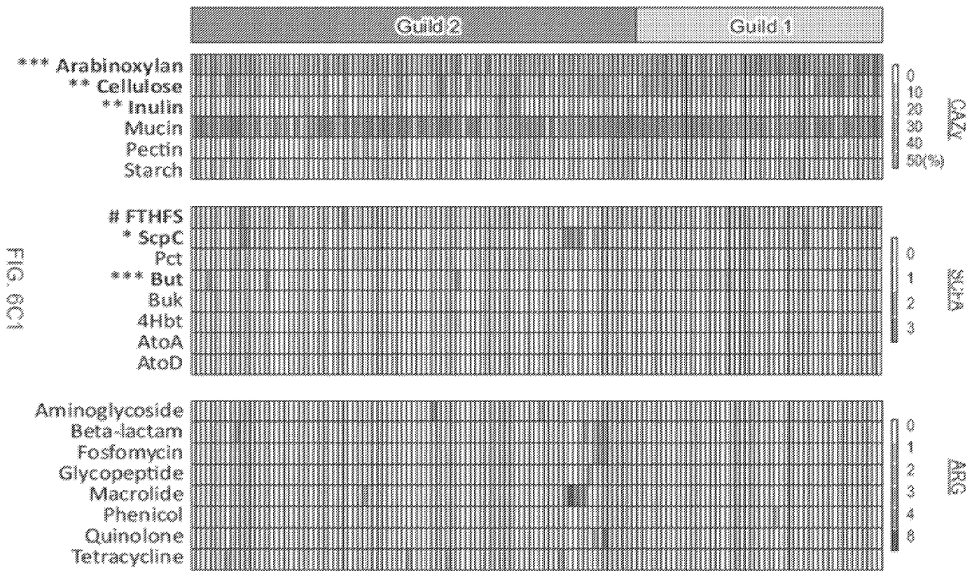

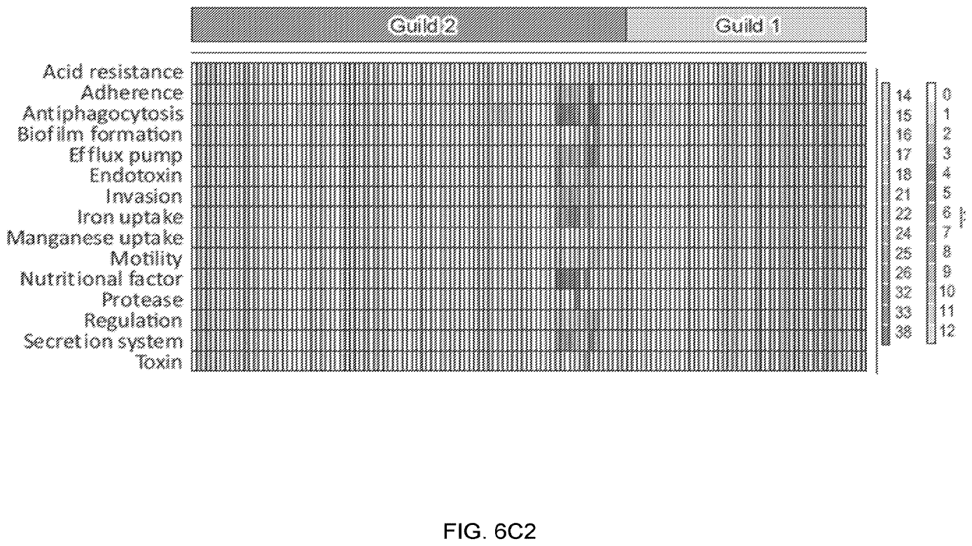

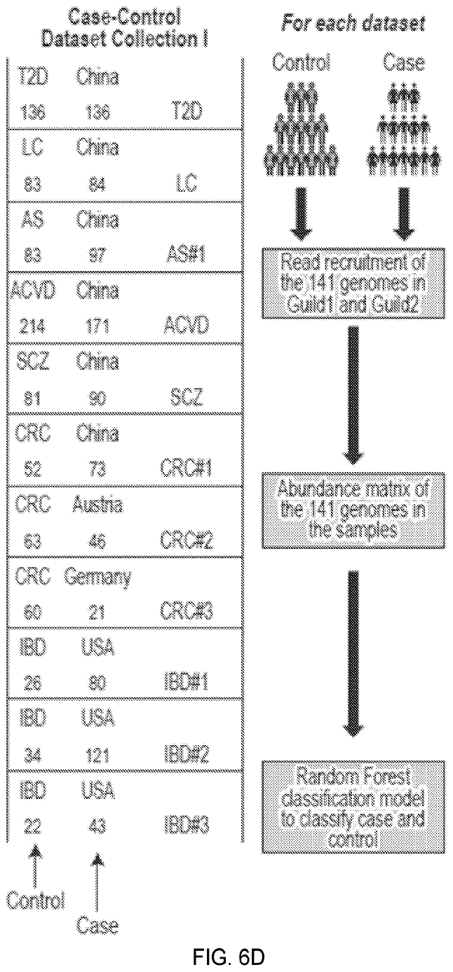

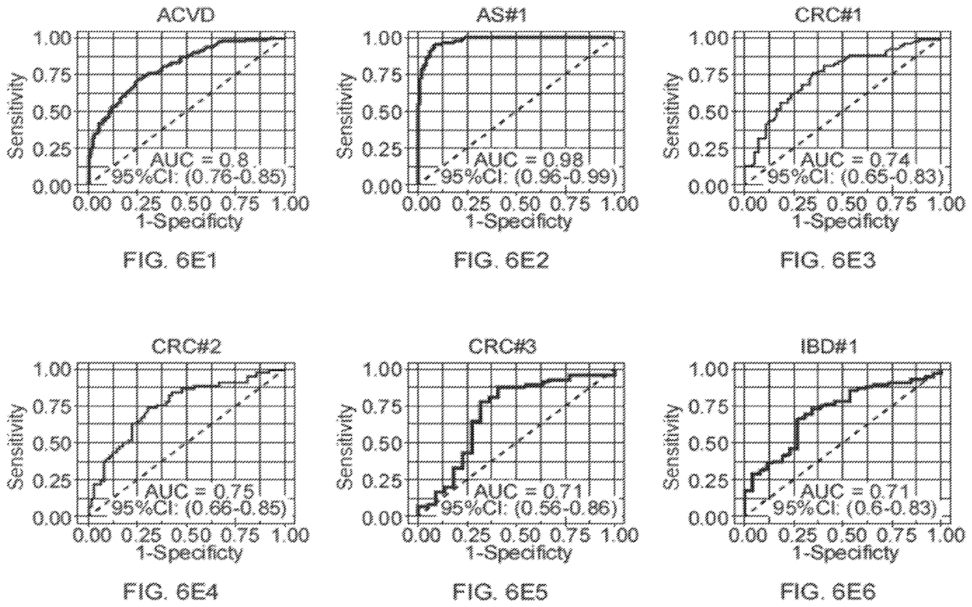

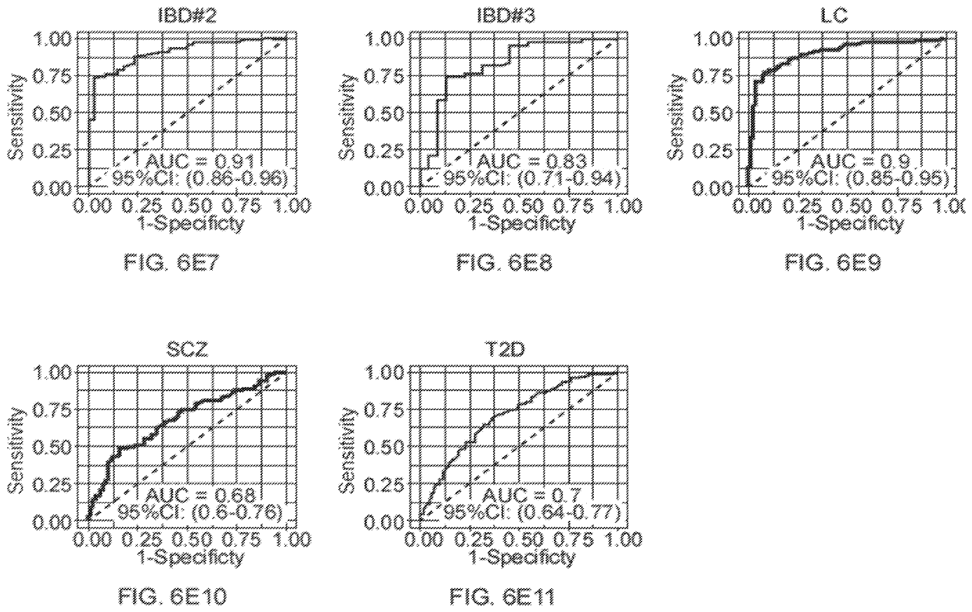

We sought to determine whether the balance of the TCG could be modulated by dietary fiber and describe how the TCG affects the host metabolic phenotypes. In the W group, the abundance of Guild 1 increased and Guild 2 decreased significantly from M0 to M3. Then at M15, Guild 1 decreased to a level similar to that at M0, and Guild 2 increased but were not significantly different from that at M3. Subsequently, from M0 to M3, high fiber intervention significantly increased the Guild 1-to-Guild 2 ratio. At one-year follow-up, the ratio significantly decreased (FIG. 6A). Neither the abundances of the 2 guilds nor their ratio was changed in the U group across the trial. These results showed that the changes of the balance between the two guilds were concomitant with changes in dietary fiber intake, overall gut microbiota and host phenotypes. To further explore the importance of the two guilds to host health, we applied linear mixed effect model to identify the associations between the two guilds and each host bio-clinical parameter. The models were trained using the abundances of the two guilds and bio-clinical parameters at M0 and M3. Subsequently, the models were used to generate predicted values for each bio-clinical parameter based on the guilds' abundance at M15. These predicted values were then correlated with the measured bio-clinical parameters at M15. Forty-two out of the 43 bio-clinical parameters had significant Pearson's correlation coefficient ranged from 0.14 to 0.88 (adjusted P value <0.05) between the predicted and measured values (FIG. 6B). These results showed the TCG constitutes an important microbiome signature for T2DM and the related metabolic phenotypes.

Next, we performed genome-centric analysis of the 141 HQMAGs in the TCG to explore the genetic basis underlying the association between the dynamic changes of the seesaw networked microbiome signature and relationships with the host's metabolic phenotypes. As the balance between the two guilds can be shifted by dietary fibers, we first sought to identify carbohydrate-active enzyme (CAZy)-encoding genes and genes encoding key enzymes in short-chain fatty acid (SCFA) production to compare the genetic capacity for carbohydrate utilization between the two guilds. Compared with genomes in Guild 2 (C1B), those in Guild 1 (C1A) enriched CAZy genes for arabinoxylan (P<0.001), cellulose (P<0.01) and had lower roportion of CAZy genes for inulin utilization (P<0.01) (FIG. 6C). There was no difference in genes for starch, pectin, and mucin utilization between the two guilds. Our previous study showed that gut microbiota benefited patients with T2DM via acetic and butyric acid production from carbohydrate fermentation¹¹. Among the terminal genes for the butyrate biosynthetic pathways from both carbohydrates (i.e., but and buk) and proteins (i.e., atoA/D and 4Hbt), the copy number of but was significantly higher in Guild 1 and there was no difference in the other terminal genes between the two guilds (FIG. 6C). More than one-third of the genomes in Guild 1 harbored the but gene while less then 5% of the genomes in Guild 2 had this gene (Fisher's exact test P<0.001). Compared with Guild 2, Guild 1 also trended higher in its genetic capacity for acetate production (P=0.06) but had a lower genetic capacity for propionate production (P<0.05) (FIG. 6C). These results showed that compared to Guild 2, Guild 1 had significantly higher genetic capacity for utilizing complex plant polysaccharides and producing acetatetrea and butyrate.

From the perspective of pathogenicity, 21 out of all the 1,845 HQMAGs encoded 750 virulence factor (VF) genes. Among the 21 VF-encoding HQMAGs, 3 were in Guild 1 while 18 were in Guild 2. Three out of the 50 HQMAGs in Guild 1 had one VF gene involved in antiphagocytosis. In Guild 2, 18 out of the 91 HQMAGs encoded 747 VF genes across 15 different VF classes i.e., acid resistance, adherence, antiphagocytosis, biofilm formation, efflux pump, endotoxin, invasion, iron uptake, manganese uptake, motility, nutritional factor, protease, regulation, secretion system, and toxin (FIG. 6C). Notably, 98.53% of all the VF genes in Guild 2 were harbored in 8 HQMAGs (1 in Enterobacter kobei, 2 in Escherichia flexneri, 3 in Escherichia coli and 2 in Klebsiella). The highly enriched genes for virulence factors in HQMAGs of Guild 2 (Fisher's Exact test, P<2.2×10⁻¹⁶,) indicates that this guild may play an important role in aggravating the metabolic disease phenotypes.

In terms of antibiotic resistance genes (ARG), in Guild 1, only 1 HQAMG (2.00% of the genomes in this guild) harbored a copy of an ARG related to phenicol (FIG. 6C). In Guild 2, 17 HQMAGs (18.68% of the genomes in this guild) encode 40 ARGs for resistance to 7 different antibiotic classes i.e., aminoglycosides, beta-lactam, fosfomycin, glycopeptide, quinolone, macrolide, and tetracycline. Thus, Guild 2 may serve as a reservoir of ARGs for horizontal transfer to opportunistic pathogens. Taken together, our data showed that the two competing guilds had distinct genetic capacity with Guild 1 being potentially beneficial and Guild 2 detrimental.

1.4 the Two Competing Guilds Identified in the QD Trial (QD-TCG) Distinguish Cases from Controls in 10 Independent Case-Control Metagenomic Datasets of 6 Different Diseases.

As the two competing guilds identified in the QD trial (QD-TCG) were found to be significant microbiome signatures for responding to dietary intervention in T2DM, we investigated whether they could function as biomarkers to distinguish T2DM from controls. We addressed this question in an independent T2DM metagenomic dataset, comprising 136 T2DM cases and 136 controls, using all the 141 HQMAGs from QD-TCG as reference genomes. These were used in a read recruitment analysis, a widely utilized method to estimate the abundance of reference genomes in metagenomes. On average, 35.28% and 32.92% reads were recruited in the cases and controls samples, respectively. Following this, we developed a machine learning classifier based on a Random Forest algorithm, employing the abundance of the 141 HQMAGs to determine if we could distinguish cases from controls. Receiver operating characteristic curve analysis demonstrated a moderate diagnostic capability, with an area under the curve (AUC) of 0.70, ascertained through leave-one-out cross-validation.

Subsequently, we hypothesized that the QD-TCG might represent an intrinsic pattern in the human microbiome, irrespective of disease types. To assess this, we collected 10 independent metagenomic datasets of cases and controls spanning six different diseases: liver cirrhosis (LC), ankylosing spondylitis (AS), atherosclerotic cardiovascular disease (ACVD), schizophrenia (SCZ), colorectal cancer (CRC), and inflammatory bowel disease (IBD). We refer to the collection of these 10 datasets, together with the aforementioned T2DM dataset, as the Case-Control Dataset Collection I (CCDC-I). On average, from these 10 metagenomic datasets, 32.12% and 31.34% reads were recruited to the two guilds in the cases and control samples, respectively (FIG. 6D). Within each dataset, a Random Forest classifier using the 141 HQMAGs demonstrated diagnostic power in distinguishing cases from controls, with the AUC ranging from 0.68 for SCZ to 0.98 for AS #1 (FIG. 6E).

These findings underscore the discriminative power of the QD-TCG in distinguishing cases versus controls across various disease types. This suggests a common microbiome signature that is associated with a broad spectrum of human diseases.

### Materials and Methods

### Clinical Experiment

Study design: QD trial was conducted at the Qidong People's Hospital (Jiangsu, China), examined the effect of a high fiber diet in free-living conditions in a cohort of individuals clinically diagnosed T2DM. The study protocol was approved by Ethics Committee of Shanghai General Hospital (2014KY104), and the study was conducted in accordance with the principles of the Declaration of Helsinki. All participants provided written informed consent. The trial was registered in the Chinese Clinical Trial Registry (ChiCTR-IPC-14005346).

T2DM patients of the Chinese Han ethnicity were recruited for the study (age: 37-70 years; HbA1c: 6.5%-12.0%. More detailed description of inclusion and exclusion criteria were shown in Chinese Clinical Trial registry (http://www.chictr.org.cn).

Patients received either a high-fiber diet (WTP diet) as the treatment group (W group) or the usual care (Usual diet) as the control group (U group) for 3 months. Total caloric and macronutrients prescriptions were based on age-specific Chinese Dietary Reference Intakes (Chinese Nutrition Society, 2013). The WTP diet, based on wholegrains, traditional Chinese medicinal foods and prebiotics, included three ready-to-consume pre-prepared foods¹¹. The usual care included standard dietary and exercise advice that was made according to the Chinese Diabetes Society guidelines for T2DM54. Patients in W group were provided with the WTP diet to perform a self-administered intervention at home for three months, while patients in U group accepted the usual care. W group stopped WTP diet intervention at the end of the third month (at M3). Then W and U continued a one-year follow-up (M15). A meal-based food frequency questionnaire and 24-h dietary recall were used to calculate nutrient intake based on the China Food Composition 200955. Patients in both groups continued with their antidiabetic medications according to their physician prescriptions.

Before a 2-week run-in period, all participants attended a lecture on diabetes intervention and improvements and received diabetes education and metabolic assessments. 119 eligible individuals were enrolled based on the inclusion and exclusion criteria and assigned into two groups in a 2:1 ratio (n=79 in W group, n=40 in U group) determined by SAS software.

Physical examinations were carried out at M0, M3, and M15 in Qidong People's Hospital (Jiangsu, China). Sample collection instructions were provided to the participants at the day before. The participants provided the feces and first early morning urine as requested. After collecting fasting venous blood sample, a 3-h meal tolerance test (Chinese buns containing 75 g of available carbohydrates; MTT test) was conducted and the postprandial venous blood samples at 30, 60, 120, and 180 min were collected. All the blood samples were centrifuged at 3000 rpm for 20 min at 4° C. after standing at room temperature for 30 min to obtain serum. The fasting blood serum were divided into two parts, one used for hospital tests and the other used for lab tests. The feces, urine, and serum samples were stored in dry ice immediately then transported to lab and frozen at −80° C. Subsequently, anthropometric markers and diabetic complication indexes were measured. Ewing test56 and 24-h dynamic electrocardiogram were conducted to estimate diabetic autonomic neuropathy (DAN). B-mode carotid ultrasound was conducted to estimate atherosclerosis. Michigan Neuropathy Screening Instrument⁵⁷ was conducted to estimate diabetic peripheral neuropathy (DPN). In addition, A meal-based food frequency questionnaire and the 24-h dietary review were recorded for nutrient intake calculation.

The fasting venous blood was used to measure HbA1c, fasting blood glucose, fasting insulin, fasting C-Peptide, C-reactive protein (CRP), blood routine examination, blood biochemical examination and five analytes of thyroid. The venous blood samples at 30, 60, 120, and 180 min of MTT were used to measure the postprandial blood glucose, insulin, and C-Peptide. The fasting early morning urine was used to measure the routine urine examination and urinary microalbumin creatinine ratio. The measurements above were completed at Qidong People's Hospital. Fasting venous blood was used to quantify TNF-α (R&D Systems, MN, USA), lipopolysaccharide-binding protein (Hycult Biotech, PA, USA), leptin (P&C, PCDBH0287, China) and adiponectin (P&C, PCDBH0016, China) by enzyme-linked immunosorbent assays (ELISAs) at Shanghai Jiao Tong University.

The homeostatic model assessments of insulin resistance (HOMA-IR) and islet β-cell function (HOMA-β) were calculated based on fasting blood glucose (mmol/L) and fasting C-Peptide (pmol/L)⁵⁸: HOMA-IR=1.5+FBG * Fasting-C-Peptide/2800;

HOMA-β=0.27*Fasting-C-Peptide/(FBG-3.5). Glomerular Filtration Rate was estimated by formula GFR (ml/min per 1.73 m²)=186*Scr^{-1.154}*age−^{0.203}*0.742 (if female)*1.233 (if Chinese)⁵⁹, where Scr (serum creatinine) is in mg/dl and age is in years.

### Gut Microbiome Analysis

Metagenomic sequencing. DNA was extracted from fecal samples using the methods as previously described¹⁰. Metagenomic sequencing was performed using Illumina Hiseq 3000 at GENEWIZ Co. (Beijing, China). Cluster generation, template hybridization, isothermal amplification, linearization, and blocking denaturing and hybridization of the sequencing primers were performed according to the workflow specified by the service provider. Libraries were constructed with an insert size of approximately 500 bp followed by high-throughput sequencing to obtain paired-end reads with 150 bp in the forward and reverse directions.

Data quality control. Prinseq⁶⁰ was used to: 1) trim the reads from the 3′ end until reaching the first nucleotide with a quality threshold of 20; 2) remove read pairs when either read was <60 bp or contained “N” bases; and 3) de-duplicate the reads. Reads that could be aligned to the human genome (H. sapiens, UCSC hg19) were removed (aligned with Bowtie2⁶¹ using--reorder--no-hd--no-contain--dovetail).

De novo assembly, abundance calculation, and taxonomic assignment of genomes. De novo assembly was performed for each sample by using IDBA_UD⁶² (--step 20--mink 20--maxk 100--min_contig 500--pre_correction). The assembled contigs were further binned using MetaBAT⁶³ (--minContig 1500--superspecific-B 20). The quality of the bins was assessed using CheckM⁶⁴. Bins had completeness >95%, contamination <5% and strain heterogeneity <5% were retained as high-quality draft genomes (HQMAGs). The assembled high-quality draft genomes were further dereplicated by using dRep⁶⁵. DiTASiC⁶⁶, which applied kallisto for pseudo-alignment⁶⁷ and a generalized linear model for resolving shared reads among genomes, was used to calculate the abundance of the genomes in each sample, estimated counts with P-value >0.05 were removed, and all samples were downsized to 36 million reads (One sample with read mapping ratio <25%, which could not be well represented by the high quality genomes, were removed in downstream analysis). Taxonomic assignment of the genomes was performed by using GTDB-Tk⁶⁸ with default parameters.

Gut microbiome network construction and analysis. In W group, prevalent genomes shared by more than 75% of the samples at every timepoint were used to construct the co-abundance network at each timepoint. Fastspar⁷⁴, a rapid and scalable correlation estimation tool for microbiome study, was used to calculate the correlations between the genomes with 1,000 permutations at each time point based on the abundances of the genomes across the patients and the correlations with P≤0.001 were retained for further analysis. The networks were visualized with Cytoscape v3.8.1⁷⁵. The layout of the nodes and edges was determined by Edge-weighted Spring Embedded Layout using the correlation coefficient as weights. The links between the nodes are treated as metal springs attached to the pair of nodes. The correlation coefficient was used to determine the repulsion and attraction of the spring⁷⁵. The layout algorithm sets the position of the nodes to minimize the sum of forces in the network. We defined robust stable edges as the unchanged positive/negative correlations between the same two genomes across all the 3 networks at M0, M3, and M15. Nodes in the stable network were clustered by Connected Components Clustering analysis in Cystoscope. To identify if sub-clusters existed in Cluster C1, a clustering tree based on the negative (set as −1) and positive correlations (set as 1) with average linkage method followed by WGCNA analysis.

Case-Control Dataset Collection I (CCDC-I). Eleven independent metagenomic datasets on T2DM, LC, AS, ACVD, SCZ, CRC and IBD were downloaded from SRA or ENA database. The group information, i.e. case or control, was collected from the corresponding papers or form curatedMetagenomicData. Quality control of raw reads was conducted by KneadData (https://bitbucket.org/biobakery/kneaddata). DiTASiC was used to recruit reads and estimate the abundance of the 141 HQMAGs in QD-CTG in each sample, estimated counts with P-value >0.05 were removed and further converted to relative abundance divided by the total number of reads. A random forest classification model to classify case and control was constructed based on the estimated abundances of the HQMAGs in each dataset with leave-one-out cross-validation. High quality reads were de novo assembled for each sample using MEGAHIT ((-min-contig-len 500,-presets meta-large). The assembled contigs were binned using MetaBAT 2 and MaxBin 2. The quality of the bins was assessed using CheckM. Bins had completeness >95%, contamination <5% and strain heterogeneity <5% were retained as HQMAGs, which were further dereplicated by using dRep (Table 3). In the case and control groups, Fastspar with 1,000 permutations was used to calculate the co-abundance correlations based on the HQMAGs shared by more than 75% of the samples in both groups. Correlations with P≤0.001 were retained for further analysis Robust stable edges were defined as the unchanged positive/negative correlations between the same two genomes in both case and control groups. Same clustering analysis performed in the QD trial was conducted on stable network. DiTASiC was used estimate the abundance of HQMAGs in each TCG in each sample. A random forest classification model to classify case and control was constructed with leave-one-out cross-validation to test each TCG.

Case-Control Dataset Collection II (CCDC-II). Fifteen independent metagenomic datasets on AS, ASD, BD, COVID-19, CRC, GD, HT, MS, PC and PD were downloaded from SRA or ENA database (Table 4). The group information, i.e. case or control, was collected from the corresponding papers or form curatedMetagenomicData. Quality control of raw reads was conducted by KneadData. DiTASiC was used to recruit reads and estimate the abundance of HQMAGs in C-TCG and CC-TCG. A random forest classification model to classify case and control was constructed based on the estimated abundances of the HQMAGs in each dataset with leave-one-out cross-validation.

Treatment Dataset Collection (TDC). Eleven independent metagenomic datasets on pre-treatment samples related to four diseases, including IBD, rheumatoid arthritis (RA), advanced melanoma and B cell lymphoma, were download from SRA or ENA database (Table 5). The responder and non-responder categories of each sample were collected from the corresponding paper. Quality control of raw reads was conducted by KneadData. DiTASiC was used to recruit reads and estimate the abundance of HQMAGs in CC-TCG. A random forest classification model to predict responder and non-responder was constructed based on the estimated abundances of the HQMAGs in each dataset with leave-one-out cross-validation.

Gut microbiome functional analysis. Prokka⁶⁹ was used to annotate the HQMAGs. KEGG Orthologue (KO) IDs were assigned to the predicted protein sequences in each HQMAG by HMMSEARCH against KOfam using KofamKOALA with default parameters⁷⁰. KOs were further assigned to KEGG modules. Antibiotic resistance genes were predicted using ResFinder⁷¹ with default parameters. The identification of virulence factors was based on the core set of Virulence Factors of Pathogenic Bacteria Database (VFDB⁷²). The predicted proteins sequences were aligned to the reference sequence in VFDB using BLASTP (best hist with E-value <1e-5, identity >80% and query coverage >70%). Genes encoding carbohydrate-active enzymes (CAZys) were identified using dbCAN (releasee 6.0)⁷³, and the best-hit alignment was retained. Genes encoding formate-tetrahydrofolate ligase, propionyl-CoA: succinate-CoA transferase, propionate CoA-transferase, 4Hbt, AtoA, AtoD, Buk and But were identified as described previously¹¹.

### Statistical Analysis.

Statistical analysis was performed in the R environment (R version3.6.1). Friedman test followed by Nemenyi post-hoc test was used for intra-group comparisons for repeat measurements. Mann-Whitney test (two-sided) was used for comparisons between W and U at the same time point. Pearson Chi-square tests was performed to compare the differences of categorical data between groups or timepoints. PERMANOVA test (9,999 permutations) was used to compare the groups of gut microbiota structure.

Mann-Whitney test (two-sided) and Fisher's exact test (two sided) were used to compare the target functions between Guild 1 and Guild 2. Hierarchical clustering analysis based on Jaccard distance on the KO profiles was conducted to compare HQMAGs in CC-TCG.

Linear mixed effect model with subject id as random effect was applied to explore the associates between the abundance of guilds in QD-TCG and clinical parameters. For each HQMAG belonging to a guild, the robust clr-transformed abundance across each sample was first range-scaled. Subsequently, the guild abundance was then obtained as the mean of the range-scaled abundances of HQMAGs belonging to that guild. The M0 and M3 timepoints was used to train the linear mixed effect model, and M15 was used as testing.

The Random Forest with leave-one-out cross-validation was used to perform classification analysis based on HQMAGs of TCGs in each dataset of CCDC-I, CCDC-II and TDC. The Random Forest with 10-fold cross-validation was used to perform classification analysis based on HQMAGs in CC-TCG for universal model.

### Example 2—The Combined Core Genomes from all the Two Competing Guilds (CC-TCG) Shows Better Performances in Classifying Case Vs Control Across Diseases

2.1 Disease as the Ecosystem Perturbation Leads to Identification of More Sets of Two Competing Guilds with Stable Correlations in Independent Case-Control Datasets.

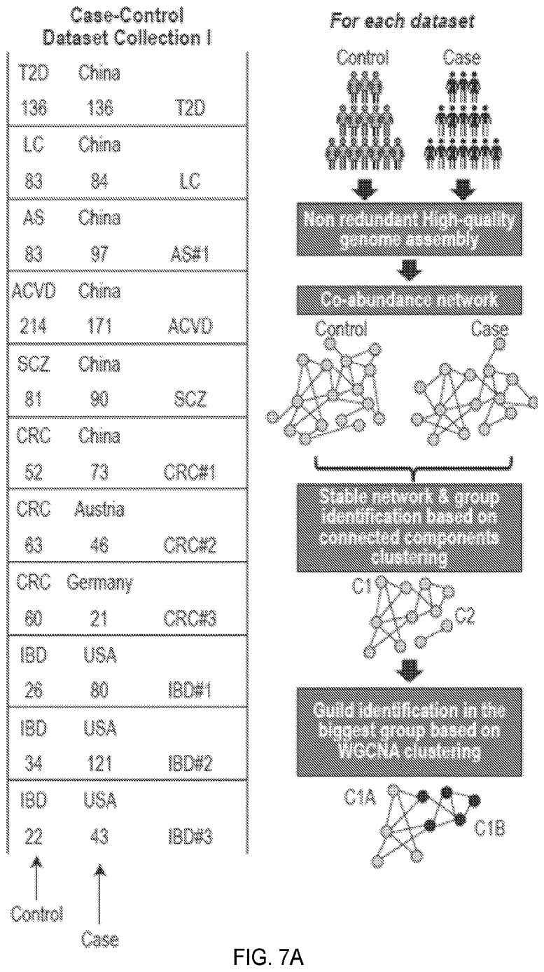

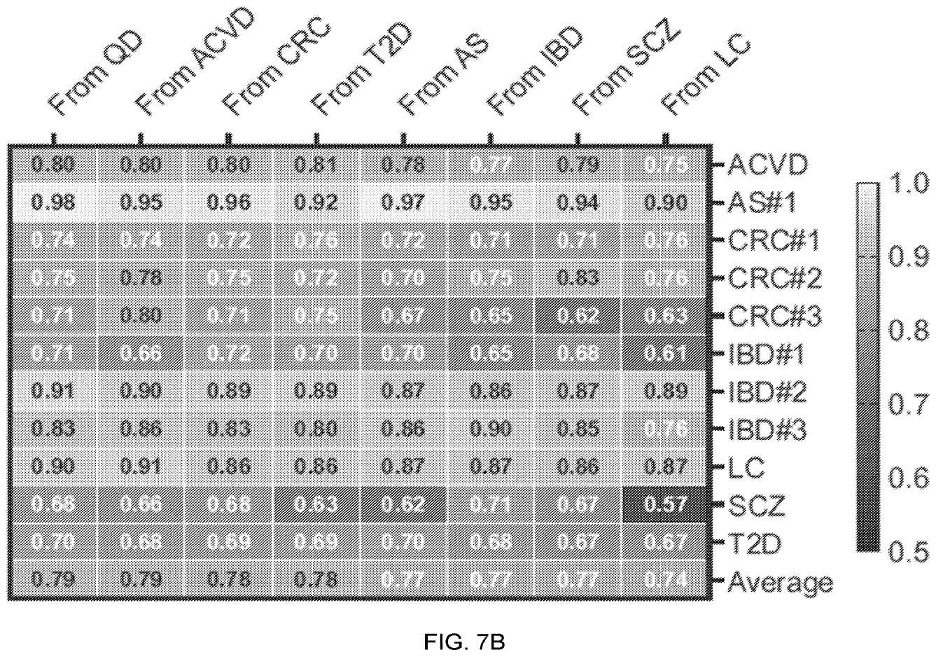

To extend our understanding of the two competing guilds i.e., QD-TCG which were identified based on unveiling stable genome pairs despite dietary interventions, we focused our research on pinpointing stable genome pairs across both cases and controls in cross-sectional datasets, with disease progression acting as a perturbation to the gut ecosystem. For this purpose, we first reconstructed non-redundant HQMAGs from the metagenomic datasets in CCDC-I (FIG. 7A). In total, we obtained 682, 384, 564, 1308, 812, 1644 and 774 HQMAGs in the case and control datasets on T2D, LC, AS, ACVD, SCZ, CRC and IBD. For each disease, we proceeded to construct a co-abundance network for both case and control groups, predicated on the prevalence of the HQMAGs that were shared by over 75% of subjects in both groups. We identified stable HQMAG pairs, which demonstrated positive/negative correlations in both case and control groups, and further grouped them using Components Clustering analysis. Notably, prevalent HQMAGs with stable correlations were classified into 2, 6, 10, 4, 10, 8 and 7 clusters in the studies on T2D, LC, AS, ACVD, SCZ, CRC and IBD.

Within each disease type, we observed one primary cluster which held the majority of genome numbers of the stable network and displayed intricate interactions with both positive and negative correlations. To ascertain if C1s contained sub-clusters, we built a clustering tree using the negative and positive correlations with the average linkage method and applied WGCNA analysis. Each Cluster C1 was subsequently segmented into two sub-clusters, C1A and C1B. Interestingly, every pair of C1A and C1B followed the pattern of two competing guilds that we observed in the QD-TCG. The majority of stable correlations between the HQMAGs belonging to C1A and C1B exhibited positive correlations within each cluster and negative correlations between the two clusters. These correlations accounted for 76.88%, 95.31%, 100%, 96.23%, 96.43%, 96.72%, and 97.10% of the stable correlations within or between the CIA and C1B in the studies on T2D, LC, AS, ACVD, SCZ, CRC and IBD respectively. These results highlight the possibility of detecting TCGs from case-control datasets and demonstrate the existence of TCGs in the human gut microbiome across various diseases, ethnicities and geographical regions.

Next, we investigated whether the TCGs identified from CCDC-I could function as biomarkers to differentiate cases and controls. For this, we used the corresponding HQMAGs as reference genomes to perform read recruitment analysis in the metagenomic datasets of CCDC-I for each TCG. On average, 54.58%, 28.32%, 26.93%, 63.62%, 44.54%, 55.72% and 34.21% of the reads in CCDC-I were recruited to the TCGs discovered in the studies on T2D, LC, AS, ACVD, SCZ, CRC and IBD respectively. For each CCDC-I dataset, we then employed a Random Forest classifier to differentiate cases from controls using the abundance of the HQMAGs in each TCG. Out of the 11 datasets in CCDC-I, the TCGs from the studies on T2D, LC, AS, ACVD, SCZ, CRC and IBD achieved moderate to excellent diagnostic power to classify cases and controls in 8, 9, 9, 9, 8, 7, 7 datasets, with an average AUC of 0.79, 0.78, 0.78, 0.77, 0.77, 0.77 and 0.74 (FIG. 7B). These findings underscore the versatility of the TCGs in classifying cases versus controls across various disease types, suggesting that the TCGs with stable correlations constitute a shared microbiome signature associated with a broad spectrum of human diseases.

2.2 the Combined Core Genomes from all the Two Competing Guilds (CC-TCG) Shows Better Performances in Classifying Case Vs Control Across Diseases.

In total, we amassed 925 HQMAGs pertaining to 8 distinct TCGs from the QD trial and CCDC-I. These were consolidated into a pool of 788 non-redundant HQMAGs after a de-redundancy analysis based on a genomic ANI cutoff >99%. The collective of these 788 non-redundant HQMAGs is hereby referred to as the combined genomes of the two competing guilds (C-TCG), a representation of the confluence of the 8 TCG sets. Within the C-TCG, 701 HQMAGs were unique to one of the 8 sets and 87 shared across multiple sets. Among the unique ones, 301 belonged to C1A and 400 to C1B. Among the shared ones, 10 and 40 consistently belonged to C1A and C1B respectively, while 37 exhibited inconsistent assignments across different TCGs (FIG. 8A). To ascertain if C-TCG would improve case-control classification across diseases, we conducted a read recruitment analysis on the metagenomic datasets of CCDC-I, using corresponding HQMAGs as reference genomes. C-TCG accounted for 84.54% of total abundance on average. Subsequently, a Random Forest classifier was trained on each CCDC-I dataset using the abundance of HQMAGs in C-TCG. Overall, C-TCG demonstrated superior case-control classification capacity in CCDC-I compared to individual TCGs, with significantly higher AUC values than classifiers trained on TCGs from the T2D, AS, IBD, SCZ, and LC studies.

Next, we aimed to identify C-TCG members most relevant to classification performance as the core genomes. We ranked the 788 HQMAGs in C-TCG based on their feature importance in the Random Forest models built for each CCDC-I dataset. Starting from the least significant HQMAGs, we sequentially removed one HQMAG and trained new Random Forest models in each dataset, assessing AUC values for classifying cases and controls. This resulted in 788 different classifiers, each employing a variable number of HQMAGs ranging from 1 to 788 in each CCDC-I dataset. We assigned ranks to HQMAG numbers based on the AUC values of their corresponding models, with lower ranks indicating higher AUC values. Notably, classifiers trained on the top 302 HQMAGs showed the best classification performance in CCDC-I as demonstrated by the smallest cumulative rank (FIG. 8A). From these 302 HQMAGs, 103 were unique to C1A, 181 unique to C1B, and 18 showed inconsistent C1A and C1B assignment across different TCGs. After discarding the 18 inconsistent HQMAGs, we obtained a set of 284 HQMAGs that were not only most relevant to classification performance but also consistently assigned to the two competing guilds. We referred to these HQMAGs as the combined core set of the two competing guilds (CC-TCG). Overall, the Random Forest classifier built on CC-TCG demonstrated superior performance in classifying cases and controls compared to both C-TCG and individual TCGs from the QD trial and CCDC-I, with significantly higher AUC values than classifiers trained on TCGs from the CRC, T2D, AS, IBD, SCZ, and LC studies.

To decipher the genetic underpinnings of the associations between these genomes and host health, we conducted a genome-centric analysis of the HQMAGs belonging to CC-TCG. We first performed targeted functional analysis and compared HQMAGs assigned to CIA and C1B. Similar to QD-TCG findings, C1A had a significantly higher gene copy number for butyrate biosynthesis and lower for propionate production. In relation to carbohydrate-degrading genes, HQMAGs in CIA were rich in CAZy genes for arabinoxylan and cellulose utilization. These findings suggest that compared to C1B, HGMAGs from C1A have a higher genetic capacity for utilizing complex plant polysaccharides and producing butyrate. From the perspective of antibiotic resistance and pathogenicity, CIA had fewer ARGs and VFs than C1B.

Furthermore, we conducted an untargeted functional analysis based on the assignment of KEGG Orthology (KO) to all predicted genes from the 284 core HQMAGs. In total, we found 3,553 and 5,495 KOs in C1A and C1B respectively. Hierarchical clustering analysis based on KO profiles demonstrated significant functional differences between C1A and C1B (PERMANOVA, P=0.001). KOs from C1A and C1B were further mapped to 253 and 291 KEGG modules. There were 250 shared modules between the two groups. CIA had 3 unique modules for acarbose biosynthesis, benzoate degradation and staphyloferrin B biosynthesis. C1B had 41 unique modules including those for multidrug resistance, KDO2-lipid A modification, pathogenicity signature and gamma-aminobutyrate production. In conclusion, these results show that the CC-TCG has distinct genetic capacities, with CIA being potentially beneficial and C1B detrimental.

2.3 the Combined Core Genomes in the Two Competing Guilds (CC-TCG) Differentiate Cases from Controls for Additional Datasets.

In order to further substantiate the capability of CC-TCG in distinguishing cases from controls across a spectrum of diseases, we compiled an additional 15 independent metagenomic datasets. These comprised case and control data from 10 different diseases, including Ankylosing Spondylitis (AS), Autism Spectrum Disorder (ASD), Behcet's Disease (BD), COVID-19, Colorectal Cancer (CRC), Graves' Disease (GD), Hypertension (HT), Multiple Sclerosis (MS), Pancreatic Cancer (PC), and Parkinson's Disease (PD). These 15 datasets are collectively referred to as the Case-Control Dataset Collection II (CCDC-II). Employing the 284 HQMAGs that belong to CC-TCG as reference genomes, we conducted a read recruitment analysis on the metagenomic datasets of the CCDC-II. On average, we found that 34.41% and 34.76% of reads were recruited in the case and control samples respectively. For each dataset in the CCDC-II, we then utilized the abundance of the 284 HQMAGs in CC-TCG to train a Random Forest classifier to discern cases from controls. The CC-TCG showed moderate to excellent diagnostic power in 10 of the 15 datasets, specifically those related to AS, ASD, COVID-19, CRC, GD, HT, MS, and PC, although it only achieved an AUC value of 0.58 for HT #2, and AUC values between 0.6-0.7 for BD, PD, CRC #4 and CRC #5 datasets (FIG. 8B).

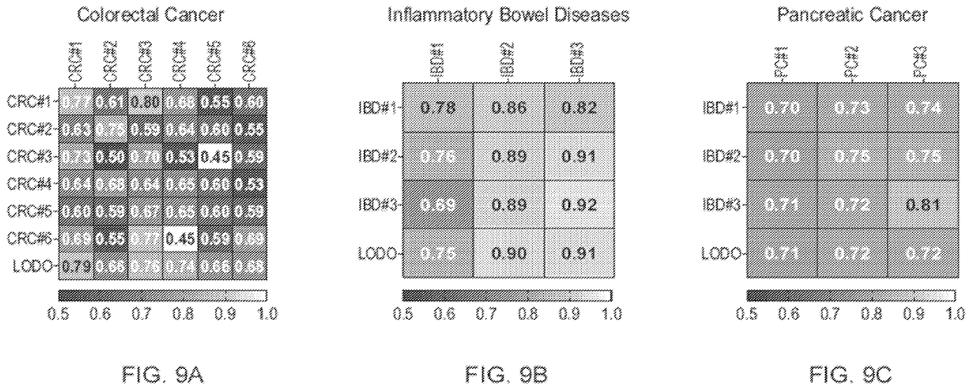

In terms of diseases like CRC, IBD, and PC, which had more than two different datasets, we also employed cross-dataset analysis (with one dataset used for model training and the other for testing) and leave-one-dataset-out (LODO) analysis to assess the universal applicability of the diagnostic power of CC-TCG for these diseases. For CRC, we found the model's transportability from one individual dataset to another to be insufficient, similar to the microbiome signature report in CRC by Thomas et al. However, in the case of IBD and PC, the classification model trained on CC-TCG in one dataset exhibited moderate to outstanding performance when classifying cases and controls in the other datasets. Pooling the training datasets, as done in the LODO analysis, improved the performance of CC-TCG-based models in classifying cases versus controls in CRC, with AUC values ranging from 0.66 to 0.79 (FIG. 9A) LODO analysis produced AUC values ranging from 0.75 to 0.91 for IBD, and 0.71-0.72 for PC (FIG. 9B-C). These results attest to the classification power of CC-TCG in completely independent datasets.

### Materials and Methods

### Gut Microbiome Analysis

Metagenomic sequencing. DNA was extracted from fecal samples using the methods as previously described¹⁰. Metagenomic sequencing was performed using Illumina Hiseq 3000 at GENEWIZ Co. (Beijing, China). Cluster generation, template hybridization, isothermal amplification, linearization, and blocking denaturing and hybridization of the sequencing primers were performed according to the workflow specified by the service provider. Libraries were constructed with an insert size of approximately 500 bp followed by high-throughput sequencing to obtain paired-end reads with 150 bp in the forward and reverse directions.

Data quality control. Prinseq⁶⁰ was used to: 1) trim the reads from the 3′ end until reaching the first nucleotide with a quality threshold of 20; 2) remove read pairs when either read was <60 bp or contained “N” bases; and 3) de-duplicate the reads. Reads that could be aligned to the human genome (H. sapiens, UCSC hg19) were removed (aligned with Bowtie2⁶¹ using--reorder--no-hd--no-contain--dovetail).

De novo assembly, abundance calculation, and taxonomic assignment of genomes. De novo assembly was performed for each sample by using IDBA_UD⁶² (--step 20--mink 20--maxk 100--min_contig 500--pre_correction). The assembled contigs were further binned using MetaBAT⁶³ (--minContig 1500--superspecific-B 20). The quality of the bins was assessed using CheckM⁶⁴. Bins had completeness >95%, contamination <5% and strain heterogeneity <5% were retained as high-quality draft genomes The assembled high-quality draft genomes were further dereplicated by using dRep⁶⁵. DiTASiC⁶⁶, which applied kallisto for pseudo-alignment⁶⁷ and a generalized linear model for resolving shared reads among genomes, was used to calculate the abundance of the genomes in each sample, estimated counts with P-value >0.05 were removed, and all samples were downsized to 36 million reads (One sample with read mapping ratio <25%, which could not be well represented by the high quality genomes, were removed in downstream analysis). Taxonomic assignment of the genomes was performed by using GTDB-Tk⁶⁸ with default parameters.

Gut microbiome network construction and analysis. In W group, prevalent genomes shared by more than 75% of the samples at every timepoint were used to construct the co-abundance network at each timepoint. Fastspar⁷⁴, a rapid and scalable correlation estimation tool for microbiome study, was used to calculate the correlations between the genomes with 1,000 permutations at each time point based on the abundances of the genomes across the patients and the correlations with P≤0.001 were retained for further analysis. The networks were visualized with Cytoscape v3.8.1⁷⁵. The layout of the nodes and edges was determined by Edge-weighted Spring Embedded Layout using the correlation coefficient as weights. The links between the nodes are treated as metal springs attached to the pair of nodes. The correlation coefficient was used to determine the repulsion and attraction of the spring⁷⁵. The layout algorithm sets the position of the nodes to minimize the sum of forces in the network. We defined robust stable edges as the unchanged positive/negative correlations between the same two genomes across all the 3 networks at M0, M3, and M15. Nodes in the stable network were clustered by Connected Components Clustering analysis in Cystoscope. To identify if sub-clusters existed in Cluster C1, a clustering tree based on the negative (set as −1) and positive correlations (set as 1) with average linkage method followed by WGCNA analysis.

Case-Control Dataset Collection I (CCDC-I). Eleven independent metagenomic datasets on T2DM, LC, AS, ACVD, SCZ, CRC and IBD were downloaded from SRA or ENA database. The group information, i.e. case or control, was collected from the corresponding papers or form curatedMetagenomicData. Quality control of raw reads was conducted by KneadData (https://bitbucket.org/biobakery/kneaddata). DiTASiC was used to recruit reads and estimate the abundance of the 141 HQMAGs in QD-CTG in each sample, estimated counts with P-value >0.05 were removed and further converted to relative abundance divided by the total number of reads. A random forest classification model to classify case and control was constructed based on the estimated abundances of the HQMAGs in each dataset with leave-one-out cross-validation. High quality reads were de novo assembled for each sample using MEGAHIT ((-min-contig-len 500,-presets meta-large). The assembled contigs were binned using MetaBAT 2 and MaxBin 2. The quality of the bins was assessed using CheckM. Bins had completeness >95%, contamination <5% and strain heterogeneity <5% were retained as HQMAGs, which were further dereplicated by using dRep (Table 3). In the case and control groups, Fastspar with 1,000 permutations was used to calculate the co-abundance correlations based on the HQMAGs shared by more than 75% of the samples in both groups. Correlations with P≤0.001 were retained for further analysis Robust stable edges were defined as the unchanged positive/negative correlations between the same two genomes in both case and control groups. Same clustering analysis performed in the QD trial was conducted on stable network. DiTASiC was used estimate the abundance of HQMAGs in each TCG in each sample. A random forest classification model to classify case and control was constructed with leave-one-out cross-validation to test each TCG.

Case-Control Dataset Collection II (CCDC-II). Fifteen independent metagenomic datasets on AS, ASD, BD, COVID-19, CRC, GD, HT, MS, PC and PD were downloaded from SRA or ENA database (Table 4). The group information, i.e. case or control, was collected from the corresponding papers or form curatedMetagenomicData. Quality control of raw reads was conducted by KneadData. DiTASiC was used to recruit reads and estimate the abundance of HQMAGs in C-TCG and CC-TCG. A random forest classification model to classify case and control was constructed based on the estimated abundances of the HQMAGs in each dataset with leave-one-out cross-validation.

Treatment Dataset Collection (TDC). Eleven independent metagenomic datasets on pre-treatment samples related to four diseases, including IBD, rheumatoid arthritis (RA), advanced melanoma and B cell lymphoma, were download from SRA or ENA database (Table 5). The responder and non-responder categories of each sample were collected from the corresponding paper. Quality control of raw reads was conducted by KneadData. DiTASiC was used to recruit reads and estimate the abundance of HQMAGs in CC-TCG. A random forest classification model to predict responder and non-responder was constructed based on the estimated abundances of the HQMAGs in each dataset with leave-one-out cross-validation.

Gut microbiome functional analysis. Prokka⁶⁹ was used to annotate the HQMAGs. KEGG Orthologue (KO) IDs were assigned to the predicted protein sequences in each HQMAG by HMMSEARCH against KOfam using KofamKOALA with default parameters⁷⁰. KOs were further assigned to KEGG modules. Antibiotic resistance genes were predicted using ResFinder⁷¹ with default parameters. The identification of virulence factors was based on the core set of Virulence Factors of Pathogenic Bacteria Database (VFDB⁷²). The predicted proteins sequences were aligned to the reference sequence in VFDB using BLASTP (best hist with E-value <1e-5, identity >80% and query coverage >70%). Genes encoding carbohydrate-active enzymes (CAZys) were identified using dbCAN (releasee 6.0)⁷³, and the best-hit alignment was retained. Genes encoding formate-tetrahydrofolate ligase, propionyl-CoA: succinate-CoA transferase, propionate CoA-transferase, 4Hbt, AtoA, AtoD, Buk and But were identified as described previously¹¹.

### Statistical Analysis.

Statistical analysis was performed in the R environment (R version3.6.1). Friedman test followed by Nemenyi post-hoc test was used for intra-group comparisons for repeat measurements. Mann-Whitney test (two-sided) was used for comparisons between W and U at the same time point. Pearson Chi-square tests was performed to compare the differences of categorical data between groups or timepoints. PERMANOVA test (9,999 permutations) was used to compare the groups of gut microbiota structure.

Mann-Whitney test (two-sided) and Fisher's exact test (two sided) were used to compare the target functions between Guild 1 and Guild 2. Hierarchical clustering analysis based on Jaccard distance on the KO profiles was conducted to compare HQMAGs in CC-TCG.

Linear mixed effect model with subject id as random effect was applied to explore the associates between the abundance of guilds in QD-TCG and clinical parameters. For each HQMAG belonging to a guild, the robust clr-transformed abundance across each sample was first range-scaled. Subsequently, the guild abundance was then obtained as the mean of the range-scaled abundances of HQMAGs belonging to that guild. The M0 and M3 timepoints was used to train the linear mixed effect model, and M15 was used as testing.

The Random Forest with leave-one-out cross-validation was used to perform classification analysis based on HQMAGs of TCGs in each dataset of CCDC-I, CCDC-II and TDC. The Random Forest with 10-fold cross-validation was used to perform classification analysis based on HQMAGs in CC-TCG for universal model.

### Example 3—the Combined Core Genomes in the Two Competing Guilds (CC-TCG) Predict Immunotherapy Outcomes Across Various Independent Datasets Spanning a Diverse Range of Diseases

There have been reports linking gut microbiota to the efficacy of biological therapies in many diseases. We hypothesized that pre-treatment variations in CC-TCG might be predictive of clinical success in diseases. To test our hypothesis, we compiled 11 pre-treatment metagenomic datasets related to four diseases, including IBD, rheumatoid arthritis (RA), advanced melanoma and B cell lymphoma, along with their corresponding categories indicating responder and non-responder to therapies. These 11 datasets are referred to as the Treatment Dataset Collection (TDC). We used HQMAGs from the CC-TCG as reference genomes to perform read recruitment analysis on the metagenomic datasets of TDC. On average, we found that 16.22% and 13.06% of reads were recruited in the responder and non-responder samples respectively. For each dataset in the TDC, we built a Random Forest classifier based on the abundance of the HQMAGs in CC-TCG to predict therapeutic responses (FIG. 8C).

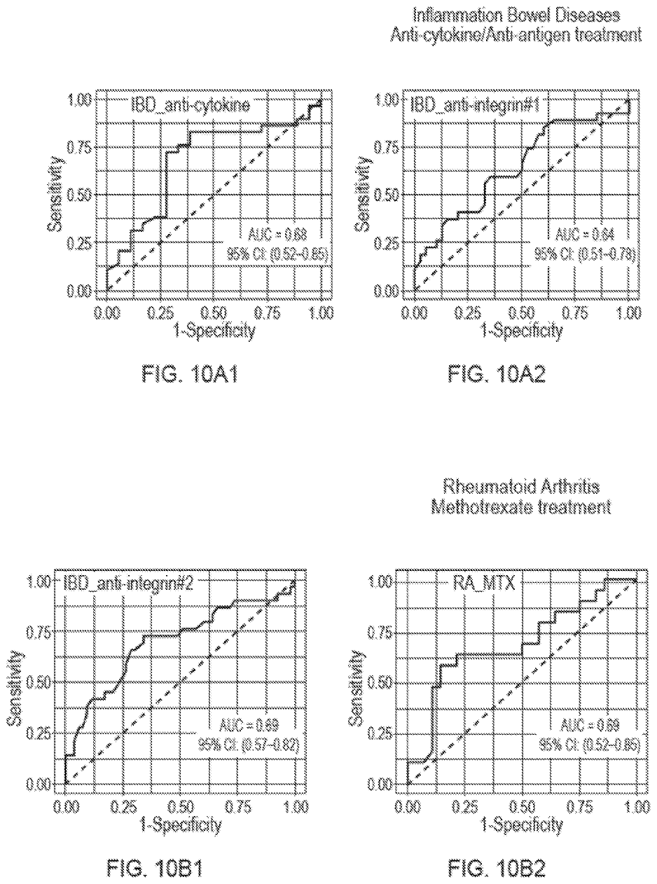

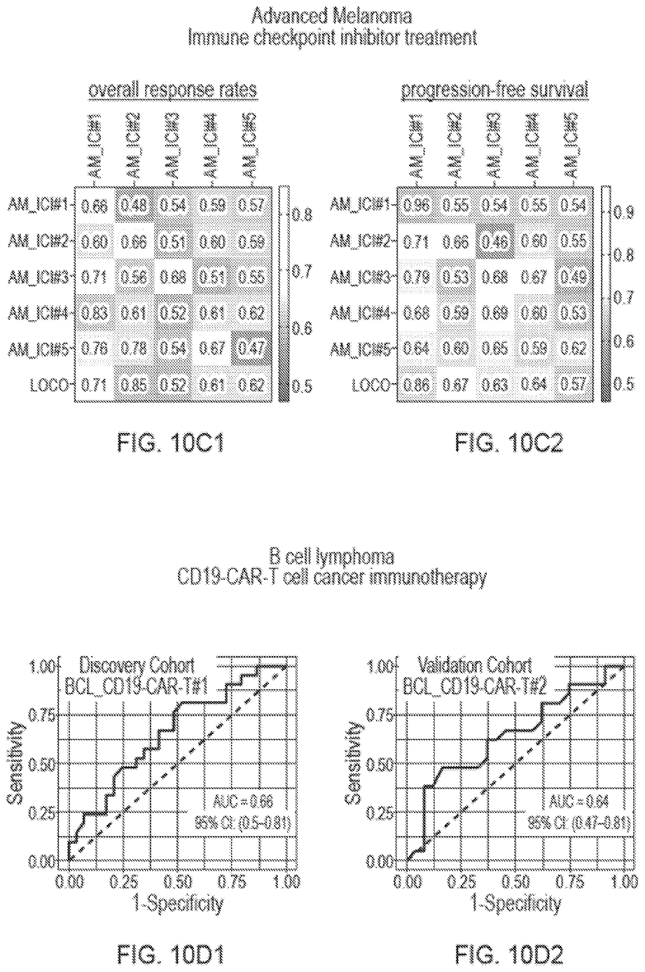

In the 3 datasets of IBD treatment, 14-week remission was used to classify patients as responder or non-responder to anti-cytokine or anti-integrin therapy. CC-TCG predicted remission at week 14 with AUC of 0.68 for IBD_anti-cytokine, 0.64 for IBD_anti-integrin #1 and 0.69 for IBD_anti-integrin #2 (FIG. 8C and FIG. 10A). |In the dataset of methotrexate treatment in new-onset RA, a responder to methotrexate was defined as any patient with new-onset RA with an improvement in the Disease Activity Score in 28 joints. CC-TCG predicted improvement under methotrexate with AUC of 0.69 (FIG. 8C and FIG. 10B). |The cross-cohort datasets of immune checkpoint inhibitor (ICI) treatment on advanced melanoma, conducted by Lee et al, were collected in TDC. The cohorts were from Barcelona (AM_ICI #1), Leeds (AM_ICI #2), Manchester (AM_ICI #3), PRIMM-UK (AM_ICI #4) and PRIMM-NL (AM_ICI #5). Responders and responders were defined based on overall response rate (ORR) or progression-free survival at 12 months (PFS12). Averagely, CC-TCG predicted ORR and PFS12 with AUC of 0.61 and 0.7 within each cohort (FIG. 8C and FIG. 10C). |The transportability of the prediction model from one single cohort to another was found to be insufficient. However, when pooling training datasets, as done in the LODO analysis, the prediction performance improved, reaching average AUC values of 0.66 and 0.67 for ORR and FPS12, respectively.| In the datasets of CD19-CAR-T immunotherapy on B cell lymphoma, responses to therapy were classified as either complete remission or non-complete remission at 180 days after CAR-T cell infusion. We trained a prediction model in the cohort from Germany (BCL_CD19-CAR-T #1) and validated it in the cohort from the United States (BCL_CD19-CAR-T #2). CC-TCG predicted responses to CD19-CAR-T immunotherapy with an AUC of 0.66 in the German cohort, and the model was sufficiently transportable to predict for the US cohort with an AUC of 0.64. These results confirm the associations between pre-treatment gut microbiome and therapeutic effect and highlight the potential of using CC-TCG to predict treatment effects across various diseases.

### Materials and Methods

### Gut Microbiome Analysis

Metagenomic sequencing. DNA was extracted from fecal samples using the methods as previously described¹⁰. Metagenomic sequencing was performed using Illumina Hiseq 3000 at GENEWIZ Co. (Beijing, China). Cluster generation, template hybridization, isothermal amplification, linearization, and blocking denaturing and hybridization of the sequencing primers were performed according to the workflow specified by the service provider. Libraries were constructed with an insert size of approximately 500 bp followed by high-throughput sequencing to obtain paired-end reads with 150 bp in the forward and reverse directions.

Data quality control. Prinseq⁶⁰ was used to: 1) trim the reads from the 3′ end until reaching the first nucleotide with a quality threshold of 20; 2) remove read pairs when either read was <60 bp or contained “N” bases; and 3) de-duplicate the reads. Reads that could be aligned to the human genome (H. sapiens, UCSC hg19) were removed (aligned with Bowtie2⁶¹ using--reorder--no-hd--no-contain--dovetail).

De novo assembly, abundance calculation, and taxonomic assignment of genomes. De novo assembly was performed for each sample by using IDBA_UD⁶² (--step 20--mink 20--maxk 100--min_contig 500--pre_correction). The assembled contigs were further binned using MetaBAT⁶³ (--minContig 1500--superspecific-B 20). The quality of the bins was assessed using CheckM⁶⁴. Bins had completeness >95%, contamination <5% and strain heterogeneity <5% were retained as high-quality draft genomes. The assembled high-quality draft genomes were further dereplicated by using dRep⁶⁵. DiTASiC⁶⁶, which applied kallisto for pseudo-alignment⁶⁷ and a generalized linear model for resolving shared reads among genomes, was used to calculate the abundance of the genomes in each sample, estimated counts with P-value >0.05 were removed, and all samples were downsized to 36 million reads (One sample with read mapping ratio <25%, which could not be well represented by the high quality genomes, were removed in downstream analysis). Taxonomic assignment of the genomes was performed by using GTDB-Tk⁶⁸ with default parameters.

Gut microbiome network construction and analysis. In W group, prevalent genomes shared by more than 75% of the samples at every timepoint were used to construct the co-abundance network at each timepoint. Fastspar74, a rapid and scalable correlation estimation tool for microbiome study, was used to calculate the correlations between the genomes with 1,000 permutations at each time point based on the abundances of the genomes across the patients and the correlations with P≤0.001 were retained for further analysis. The networks were visualized with Cytoscape v3.8.1⁷⁵. The layout of the nodes and edges was determined by Edge-weighted Spring Embedded Layout using the correlation coefficient as weights. The links between the nodes are treated as metal springs attached to the pair of nodes. The correlation coefficient was used to determine the repulsion and attraction of the spring⁷⁵. The layout algorithm sets the position of the nodes to minimize the sum of forces in the network. We defined robust stable edges as the unchanged positive/negative correlations between the same two genomes across all the 3 networks at M0, M3, and M15. Nodes in the stable network were clustered by Connected Components Clustering analysis in Cystoscope. To identify if sub-clusters existed in Cluster C1, a clustering tree based on the negative (set as −1) and positive correlations (set as 1) with average linkage method followed by WGCNA analysis.

Case-Control Dataset Collection I (CCDC-I). Eleven independent metagenomic datasets on T2DM, LC, AS, ACVD, SCZ, CRC and IBD were downloaded from SRA or ENA database. The group information, i.e. case or control, was collected from the corresponding papers or form curatedMetagenomicData. Quality control of raw reads was conducted by KneadData (https://bitbucket.org/biobakery/kneaddata). DiTASiC was used to recruit reads and estimate the abundance of the 141 HQMAGs in QD-CTG in each sample, estimated counts with P-value >0.05 were removed and further converted to relative abundance divided by the total number of reads. A random forest classification model to classify case and control was constructed based on the estimated abundances of the HQMAGs in each dataset with leave-one-out cross-validation. High quality reads were de novo assembled for each sample using MEGAHIT ((-min-contig-len 500,-presets meta-large). The assembled contigs were binned using MetaBAT 2 and MaxBin 2. The quality of the bins was assessed using CheckM. Bins had completeness >95%, contamination <5% and strain heterogeneity <5% were retained as HQMAGs, which were further dereplicated by using dRep (Table 3). In the case and control groups, Fastspar with 1,000 permutations was used to calculate the co-abundance correlations based on the HQMAGs shared by more than 75% of the samples in both groups. Correlations with P≤0.001 were retained for further analysis Robust stable edges were defined as the unchanged positive/negative correlations between the same two genomes in both case and control groups. Same clustering analysis performed in the QD trial was conducted on stable network. DiTASiC was used estimate the abundance of HQMAGs in each TCG in each sample. A random forest classification model to classify case and control was constructed with leave-one-out cross-validation to test each TCG.

Case-Control Dataset Collection II (CCDC-II). Fifteen independent metagenomic datasets on AS, ASD, BD, COVID-19, CRC, GD, HT, MS, PC and PD were downloaded from SRA or ENA database (Table 4). The group information, i.e. case or control, was collected from the corresponding papers or form curatedMetagenomicData. Quality control of raw reads was conducted by KneadData. DiTASiC was used to recruit reads and estimate the abundance of HQMAGs in C-TCG and CC-TCG. A random forest classification model to classify case and control was constructed based on the estimated abundances of the HQMAGs in each dataset with leave-one-out cross-validation.

Treatment Dataset Collection (TDC). Eleven independent metagenomic datasets on pre-treatment samples related to four diseases, including IBD, rheumatoid arthritis (RA), advanced melanoma and B cell lymphoma, were download from SRA or ENA database (Table 5). The responder and non-responder categories of each sample were collected from the corresponding paper. Quality control of raw reads was conducted by KneadData. DiTASiC was used to recruit reads and estimate the abundance of HQMAGs in CC-TCG. A random forest classification model to predict responder and non-responder was constructed based on the estimated abundances of the HQMAGs in each dataset with leave-one-out cross-validation.

Gut microbiome functional analysis. Prokka⁶⁹ was used to annotate the HQMAGs. KEGG Orthologue (KO) IDs were assigned to the predicted protein sequences in each HQMAG by HMMSEARCH against KOfam using KofamKOALA with default parameters⁷⁰. KOs were further assigned to KEGG modules. Antibiotic resistance genes were predicted using ResFinder⁷¹ with default parameters. The identification of virulence factors was based on the core set of Virulence Factors of Pathogenic Bacteria Database (VFDB⁷²). The predicted proteins sequences were aligned to the reference sequence in VFDB using BLASTP (best hist with E-value <1e-5, identity >80% and query coverage >70%). Genes encoding carbohydrate-active enzymes (CAZys) were identified using dbCAN (releasee 6.0)⁷³, and the best-hit alignment was retained. Genes encoding formate-tetrahydrofolate ligase, propionyl-CoA: succinate-CoA transferase, propionate CoA-transferase, 4Hbt, AtoA, AtoD, Buk and But were identified as described previously¹¹.

### Statistical Analysis.

Statistical analysis was performed in the R environment (R version3.6.1). Friedman test followed by Nemenyi post-hoc test was used for intra-group comparisons for repeat measurements. Mann-Whitney test (two-sided) was used for comparisons between W and U at the same time point. Pearson Chi-square tests was performed to compare the differences of categorical data between groups or timepoints. PERMANOVA test (9,999 permutations) was used to compare the groups of gut microbiota structure.

Mann-Whitney test (two-sided) and Fisher's exact test (two sided) were used to compare the target functions between Guild 1 and Guild 2. Hierarchical clustering analysis based on Jaccard distance on the KO profiles was conducted to compare HQMAGs in CC-TCG.

Linear mixed effect model with subject id as random effect was applied to explore the associates between the abundance of guilds in QD-TCG and clinical parameters. For each HQMAG belonging to a guild, the robust clr-transformed abundance across each sample was first range-scaled. Subsequently, the guild abundance was then obtained as the mean of the range-scaled abundances of HQMAGs belonging to that guild. The M0 and M3 timepoints was used to train the linear mixed effect model, and M15 was used as testing.

The Random Forest with leave-one-out cross-validation was used to perform classification analysis based on HQMAGs of TCGs in each dataset of CCDC-I, CCDC-II and TDC. The Random Forest with 10-fold cross-validation was used to perform classification analysis based on HQMAGs in CC-TCG for universal model.

### Example 4—a Universal Model Based on the Combined Core Genomes of the Two Competing Guilds Distinguish Cases from Controls Across Diseases

We demonstrated the efficacy of CC-TCG in distinguishing cases from controls and predicting therapeutic responses. This is substantiated by the moderate to outstanding Random Forest models built for each dataset in CCDC-I, CCDC-II, and TDC. Further validation of the robustness and transportability of CC-TCG trained models was achieved through cross-dataset and LODO analysis for diseases with multiple datasets.

Our findings prompted us to probe whether a universal model based on CC-TCG could discern cases from controls, regardless of the disease types. To this end, we amalgamated all the 26 datasets from CCDC-I and CCDC-II, encompassing 1,780 cases spanning 15 disease types and 1,604 controls. The HQMAGs in CC-TCG were highly prevalent, with 210 of them present in over 80%, and 101 in over 90% of the samples.

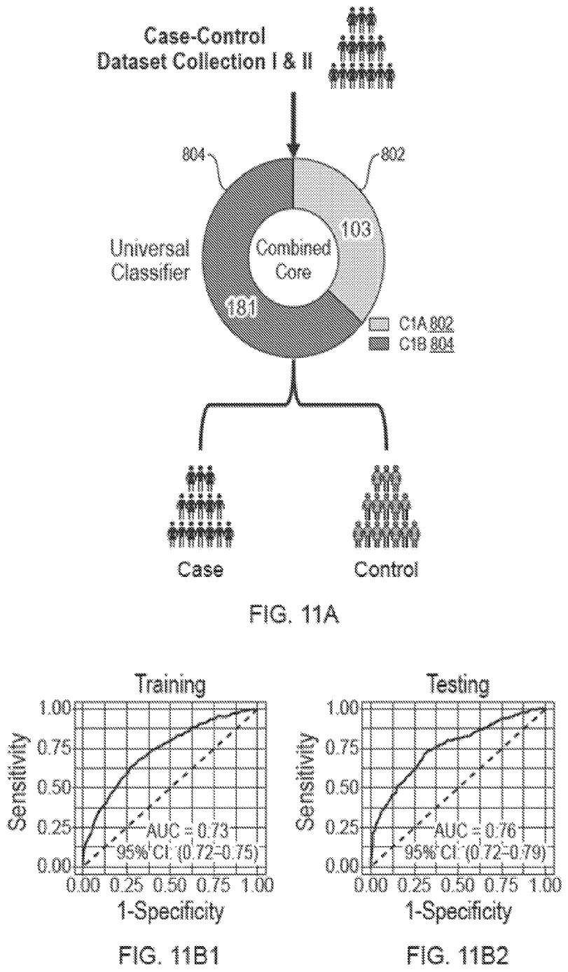

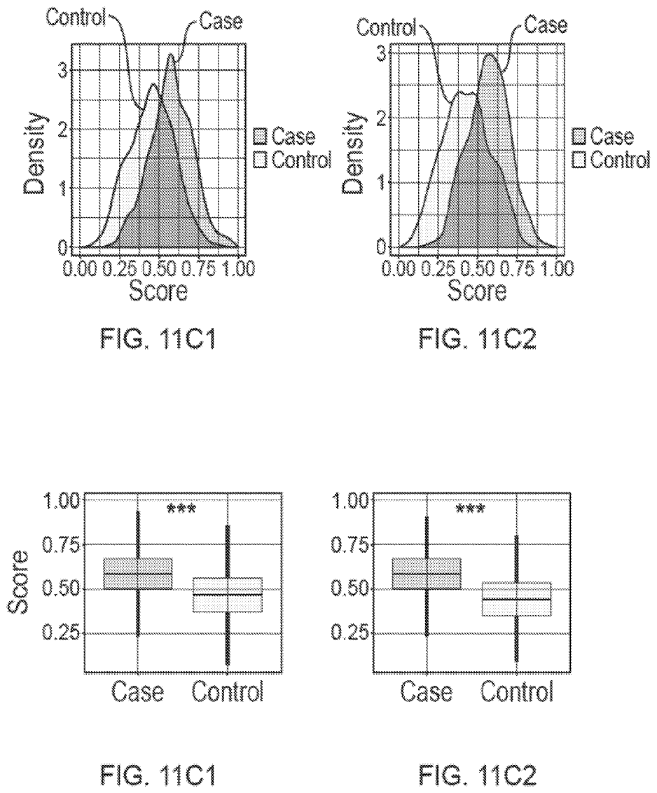

Consistent trends of CC-TCG were observed across 20 of the 26 datasets, where control samples exhibited greater diversity in CC-TCG and a higher C1A-to-C1B ratio compared to case samples. We allocated all the case and control samples randomly, with 80% serving to train a Random Forest classifier based on CC-TCG, and the remaining 20% set aside for testing (FIG. 11A).

By employing 10-fold cross-validation, we found that the universal classifier displayed a moderate ability, with an AUC of 0.73, to distinguish cases from controls (FIG. 11B). The model generated a probability score, indicating the likelihood of a sample being classified as a case, which exhibited a unimodal distribution in both case and control samples, and clear separation between peaks (FIG. 11C). Probability scores were significantly lower in controls compared to cases (FIG. 11D). When applying the universal classifier to the testing data, an AUC of 0.76 was achieved in distinguishing cases from controls (FIG. 11B). Parallel to the training data, similar distributions of probability scores for cases and controls were observed in the testing data (FIG. 11C). Similarly, in the testing data, controls also had significantly lower probability scores than those of cases. For both the training and testing data, a probability score of 0.52 provided a classification specificity of 0.7 and sensitivity of 0.7 for distinguishing cases from controls. These results indicate the feasibility of using CC-TCG to differentiate cases and controls with a universal model, which is agnostic to disease types. In other words, the variation in CC-TCG may serve as a generalized indicator of healthy recovery and maintenance.

### Discussion

Our study unveils the existence of a core gut microbiome structure consisting of two adversarial bacterial guilds, manifested as a seesaw-like network in humans. This discovery was facilitated by a unique methodological combination: genome-centric, reference-free, and interaction-focused approaches. These revealed robust associations between this core microbiome signature and a diverse range of host phenotypes, notably in individuals with Type 2 Diabetes Mellitus (T2DM). Furthermore, our random forest models suggest that these bacterial genomes could help distinguish between cases and controls for multiple diseases and predict immunotherapy outcomes, emphasizing the relevance of this core microbiome signature across different ethnicities, geographical locations, and disease states.

Several lines of evidence support the characterization of these two interconnected guilds as a core gut microbiome signature. Firstly, their presence is consistent across populations, regardless of ethnicity and geography. Secondly, they demonstrate remarkable temporal stability in terms of their members and interaction patterns. Thirdly, despite comprising approximately 10% of the gut microbiome, they exercise substantial influence over the ecological community due to their highly interconnected and stable roles in the gut ecosystem. Lastly, the guilds' structure could be a product of natural selection over a long co-evolutionary history between the microbiomes and their hosts, potentially modulated by dietary fibers, a direct external energy source for the gut ecosystem.

Historical dietary trends in humans, favoring high fiber intake until about 150 years ago, bolster this notion. The high dietary fiber potentially provided an evolutionary advantage to the beneficial bacteria in Guild 1, owing to their superior capacity to degrade plant polysaccharides, thereby granting them an upper hand over the pathobionts in Guild 2. It is important to note that this seesaw-like network isn't solely linked to T2DM or entirely reliant on a high fiber diet. It can be detected in other independent metagenomic datasets, shows correlations with a variety of diseases, and could be a fundamental attribute of the human microbiome.

Guild 1 members, equipped with an exceptional ability to degrade complex plant polysaccharides, produce beneficial metabolites like short-chain fatty acids (SCFAs). These metabolites could potentially restrain the overgrowth of pathobionts in Guild 2, whose unregulated proliferation might harm host health through mechanisms such as inflammation. However, maintaining a certain population of these pathobionts is necessary as they play an essential role in priming our immune system early in life. Guild 1, akin to tall trees acting as foundational species in a dense forest, can be considered the “foundation guild” that structures and stabilizes a gut environment unfavorable to Guild 2, the pathobiont guild. Therefore, maintaining a delicate balance between the Foundation and Pathobiont Guilds is critical to determine whether the gut microbiome is health-promoting or disease-inducing.

The identified network is marked by cooperative and competitive interactions. While cooperation improves overall metabolic efficiency, it can induce dependencies leading to destabilization, which is mitigated by infusing competition into the network. Interestingly, the seesaw-like network maintains stability, but the relative abundances of the foundation guild and the pathobiont guild can be swayed, suggesting the role of external energy inputs, specifically dietary fiber. Understanding how external energy input impacts the balance between order and chaos in a complex adaptive system is vital to comprehend the ecological dynamics within the gut microbiome. With the competing guilds acting as the gut ecosystem's backbone, ‘order’ (stable and predictable interactions) can be achieved with sufficient dietary fiber input, bolstering the foundation guild's dominance over the pathobiont guild. Conversely, ‘chaos’ (instability and potential system disruption) may arise if the energy input to the gut ecosystem shifts from external dietary fibers to host-produced mucin. These dynamics are perpetually counterbalanced by energy inputs, as evidenced by the identified seesaw-like network.

Our analysis is founded on a relationship-centric approach, arguing that stable relationships within the microbiome aid in identifying its core components. This approach involves studying interaction and co-occurrence patterns among various microbial members and interpreting these relationships as ecological role indicators. Using this method, the seesaw-like network of the two competing guilds emerged as a robust signature of the gut microbiome, displaying stability across different ethnicities, geographical locations, and disease states. This finding highlights the significance of relationship-based analyses in microbiome research, showcasing the importance of cooperation and competition dynamics within the gut microbiome. It also depicts how interaction dynamics can be modulated by external energy inputs, like dietary fiber, to maintain a balance between order (stability) and chaos (instability) within the microbiome.

In conclusion, our findings suggest that the seesaw-like network may be a foundational characteristic of the human gut microbiome. We underscore the pivotal role of dietary fibers as the external energy input required to maintain order in the gut microbiota for the benefit of host health. This characteristic, potentially established through natural selection over a lengthy co-evolutionary history between microbiomes and their hosts under a high-fiber dietary context, emphasizes the intricate interplay of these principles within the human gut microbiome.

Enhancing our comprehension of gut microbiome dynamics through our relationship-centric approach could guide the development of interventions targeting this core signature. The ultimate goal is to restore and uphold the dominance of the foundation guild over the pathobiont guild, thereby promoting and preserving human health. Our study underlines the importance of acknowledging stable relationships as key indicators of microbial components and their ecological roles, providing a promising framework for future microbiome research. Further investigation is needed to fully comprehend these complex dynamics and to unlock the enormous potential of microbiome-centered therapeutics.

### Materials and Methods

### Gut Microbiome Analysis

Metagenomic sequencing. DNA was extracted from fecal samples using the methods as previously described¹⁰. Metagenomic sequencing was performed using Illumina Hiseq 3000 at GENEWIZ Co. (Beijing, China). Cluster generation, template hybridization, isothermal amplification, linearization, and blocking denaturing and hybridization of the sequencing primers were performed according to the workflow specified by the service provider. Libraries were constructed with an insert size of approximately 500 bp followed by high-throughput sequencing to obtain paired-end reads with 150 bp in the forward and reverse directions.

Data quality control. Prinseq⁶⁰ was used to: 1) trim the reads from the 3′ end until reaching the first nucleotide with a quality threshold of 20; 2) remove read pairs when either read was <60 bp or contained “N” bases; and 3) de-duplicate the reads. Reads that could be aligned to the human genome (H. sapiens, UCSC hg19) were removed (aligned with Bowtie2⁶¹ using--reorder--no-hd--no-contain--dovetail).

De novo assembly, abundance calculation, and taxonomic assignment of genomes. De novo assembly was performed for each sample by using IDBA_UD⁶² (--step 20--mink 20--maxk 100--min_contig 500--pre_correction). The assembled contigs were further binned using MetaBAT⁶³ (--minContig 1500--superspecific-B 20). The quality of the bins was assessed using CheckM⁶⁴. Bins had completeness >95%, contamination <5% and strain heterogeneity <5% were retained as high-quality draft genomes. The assembled high-quality draft genomes were further dereplicated by using dRep⁶⁵. DiTASiC⁶⁶, which applied kallisto for pseudo-alignment⁶⁷ and a generalized linear model for resolving shared reads among genomes, was used to calculate the abundance of the genomes in each sample, estimated counts with P-value >0.05 were removed, and all samples were downsized to 36 million reads (One sample with read mapping ratio <25%, which could not be well represented by the high quality genomes, were removed in downstream analysis). Taxonomic assignment of the genomes was performed by using GTDB-Tk⁶⁸ with default parameters.

Gut microbiome network construction and analysis. In W group, prevalent genomes shared by more than 75% of the samples at every timepoint were used to construct the co-abundance network at each timepoint. Fastspar⁷⁴, a rapid and scalable correlation estimation tool for microbiome study, was used to calculate the correlations between the genomes with 1,000 permutations at each time point based on the abundances of the genomes across the patients and the correlations with P<0.001 were retained for further analysis. The networks were visualized with Cytoscape v3.8.1⁷⁵. The layout of the nodes and edges was determined by Edge-weighted Spring Embedded Layout using the correlation coefficient as weights. The links between the nodes are treated as metal springs attached to the pair of nodes. The correlation coefficient was used to determine the repulsion and attraction of the spring⁷⁵. The layout algorithm sets the position of the nodes to minimize the sum of forces in the network. We defined robust stable edges as the unchanged positive/negative correlations between the same two genomes across all the 3 networks at M0, M3, and M15. Nodes in the stable network were clustered by Connected Components Clustering analysis in Cystoscope. To identify if sub-clusters existed in Cluster C1, a clustering tree based on the negative (set as −1) and positive correlations (set as 1) with average linkage method followed by WGCNA analysis.

Case-Control Dataset Collection I (CCDC-I). Eleven independent metagenomic datasets on T2DM, LC, AS, ACVD, SCZ, CRC and IBD were downloaded from SRA or ENA database. The group information, i.e. case or control, was collected from the corresponding papers or form curatedMetagenomicData. Quality control of raw reads was conducted by KneadData (https://bitbucket.org/biobakery/kneaddata). DiTASiC was used to recruit reads and estimate the abundance of the 141 HQMAGs in QD-CTG in each sample, estimated counts with P-value >0.05 were removed and further converted to relative abundance divided by the total number of reads. A random forest classification model to classify case and control was constructed based on the estimated abundances of the HQMAGs in each dataset with leave-one-out cross-validation. High quality reads were de novo assembled for each sample using MEGAHIT ((-min-contig-len 500,-presets meta-large). The assembled contigs were binned using MetaBAT 2 and MaxBin 2. The quality of the bins was assessed using CheckM. Bins had completeness >95%, contamination <5% and strain heterogeneity <5% were retained as HQMAGs, which were further dereplicated by using dRep (Table 3). In the case and control groups, Fastspar with 1,000 permutations was used to calculate the co-abundance correlations based on the HQMAGs shared by more than 75% of the samples in both groups. Correlations with P≤0.001 were retained for further analysis Robust stable edges were defined as the unchanged positive/negative correlations between the same two genomes in both case and control groups. Same clustering analysis performed in the QD trial was conducted on stable network. DiTASiC was used estimate the abundance of HQMAGs in each TCG in each sample. A random forest classification model to classify case and control was constructed with leave-one-out cross-validation to test each TCG.

Case-Control Dataset Collection II (CCDC-II). Fifteen independent metagenomic datasets on AS, ASD, BD, COVID-19, CRC, GD, HT, MS, PC and PD were downloaded from SRA or ENA database (Table 4). The group information, i.e. case or control, was collected from the corresponding papers or form curatedMetagenomicData. Quality control of raw reads was conducted by KneadData. DiTASiC was used to recruit reads and estimate the abundance of HQMAGs in C-TCG and CC-TCG. A random forest classification model to classify case and control was constructed based on the estimated abundances of the HQMAGs in each dataset with leave-one-out cross-validation.

Treatment Dataset Collection (TDC). Eleven independent metagenomic datasets on pre-treatment samples related to four diseases, including IBD, rheumatoid arthritis (RA), advanced melanoma and B cell lymphoma, were download from SRA or ENA database (Table 5). The responder and non-responder categories of each sample were collected from the corresponding paper. Quality control of raw reads was conducted by KneadData. DiTASiC was used to recruit reads and estimate the abundance of HQMAGs in CC-TCG. A random forest classification model to predict responder and non-responder was constructed based on the estimated abundances of the HQMAGs in each dataset with leave-one-out cross-validation.

Gut microbiome functional analysis. Prokka⁶⁹ was used to annotate the HQMAGs. KEGG Orthologue (KO) IDs were assigned to the predicted protein sequences in each HQMAG by HMMSEARCH against KOfam using KofamKOALA with default parameters⁷⁰. KOs were further assigned to KEGG modules. Antibiotic resistance genes were predicted using ResFinder⁷¹ with default parameters. The identification of virulence factors was based on the core set of Virulence Factors of Pathogenic Bacteria Database (VFDB⁷²). The predicted proteins sequences were aligned to the reference sequence in VFDB using BLASTP (best hist with E-value <1e-5, identity >80% and query coverage >70%). Genes encoding carbohydrate-active enzymes (CAZys) were identified using dbCAN (releasee 6.0)⁷³, and the best-hit alignment was retained. Genes encoding formate-tetrahydrofolate ligase, propionyl-CoA: succinate-CoA transferase, propionate CoA-transferase, 4Hbt, AtoA, AtoD, Buk and But were identified as described previously¹¹.

### Statistical Analysis.

Statistical analysis was performed in the R environment (R version3.6.1). Friedman test followed by Nemenyi post-hoc test was used for intra-group comparisons for repeat measurements. Mann-Whitney test (two-sided) was used for comparisons between W and U at the same time point. Pearson Chi-square tests was performed to compare the differences of categorical data between groups or timepoints. PERMANOVA test (9,999 permutations) was used to compare the groups of gut microbiota structure.

Mann-Whitney test (two-sided) and Fisher's exact test (two sided) were used to compare the target functions between Guild 1 and Guild 2. Hierarchical clustering analysis based on Jaccard distance on the KO profiles was conducted to compare HQMAGs in CC-TCG.

Linear mixed effect model with subject id as random effect was applied to explore the associates between the abundance of guilds in QD-TCG and clinical parameters. For each HQMAG belonging to a guild, the robust clr-transformed abundance across each sample was first range-scaled. Subsequently, the guild abundance was then obtained as the mean of the range-scaled abundances of HQMAGs belonging to that guild. The M0 and M3 timepoints was used to train the linear mixed effect model, and M15 was used as testing.

The Random Forest with leave-one-out cross-validation was used to perform classification analysis based on HQMAGs of TCGs in each dataset of CCDC-I, CCDC-II and TDC. The Random Forest with 10-fold cross-validation was used to perform classification analysis based on HQMAGs in CC-TCG for universal model.

### Example 5—Identification of Combined Core Microbiome Signature from Combined Pool of Genomes

### 1. Combined Pool of Genomes

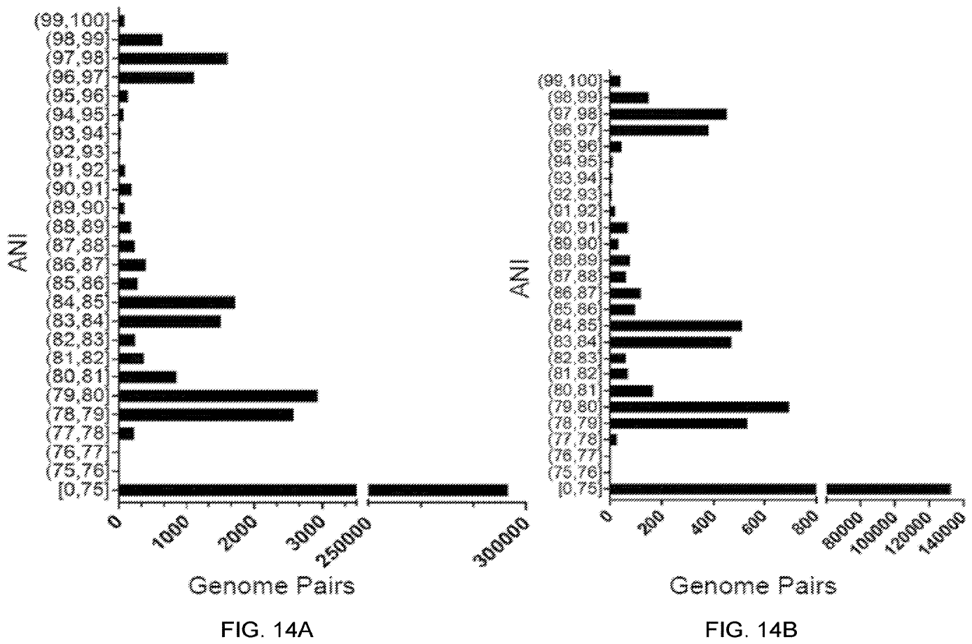

921 genomes (genomes in the two competing guilds found in QD and seven types of diseases including T2D, LC, SCZ, IBD, AS, ACVD, CRC) were further dereplicated by using dRep. Two genomes were collapsed into one if the average nucleotide identity, ANI, between them was >99%, 788 non-redundant genomes were obtained. The genome pairwise ANI comparison was performed for the 310,078 genome pairs among the 788 genomes. The ANI distribution is shown in FIG. 14A. The ANI comparison between the genomes assigned into two competing guilds: Guild 1 and Guild 2, was further studied. After removing genomes with inconsistent guild assignment from the 788 genomes, Guild 1 has 311 genomes, and Guild 2 has 440 genomes. The genome pairs between Guild 1 and Guild 2 were calculated by multiplying the total number of genomes in Guild 1, 440, by the total number of genomes of Guild 2, 331. The ANI distribution for the 136,840 genome pairs was shown in FIG. 14B.

DiTASiC, which applied kallisto for pseudo-alignment and a generalized linear model for resolving shared reads among genomes, was used to calculate the abundance of the genomes in each sample, estimated counts with P-value >0.05 were removed.

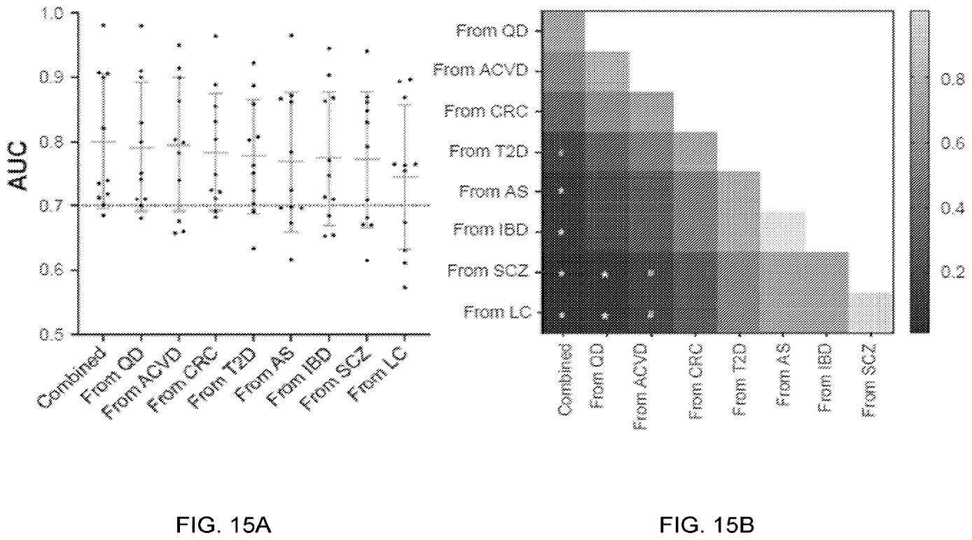

A machine learning classifier based on a Random Forest algorithm was trained to compare the capacity of the combined 788 genomes in classifying patients and control with the individual set of microbiome signature obtained from QD and various diseases cases including T2D, LC, SCZ, IBD, AS, ACVD, CRC. The area under the ROC curve (AUC) of the Random Forest classifier based on the combined pool or individual microbiome signature to classify control and patients in each dataset are shown in FIG. 15A. FIG. 15B shows the significance of intra-group comparison. Friedman test followed by Dunn's post hoc was performed for the analysis (#BH adjusted P<0.1, * BH adjusted P<0.05). Overall, Combined pool has the best capacity to classify case and control across different studies.

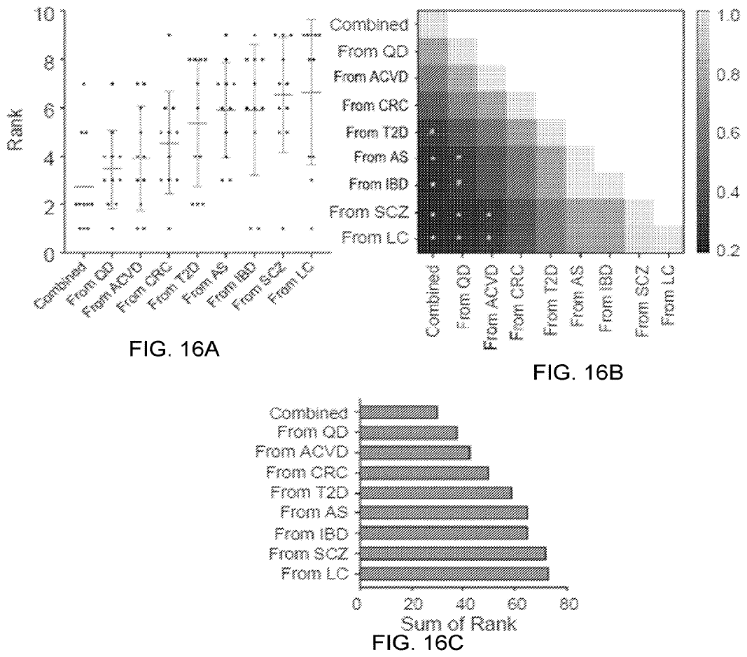

The classification performance of each model was further ranked. The nine sets of microbiome signature are ranked according to their performance in classifying case and control across 11 datasets. The rank values assigned to each set of signature microbiome are plotted FIG. 16A. FIG. 16B shows the significance of intra-group comparison. FIG. 16C shows the sum of the ranking values for each set of microbiome signatures. Kruskal-Wallis test followed by Dunn's post hoc was performed for the analysis (#BH adjusted P<0.1, * BH adjusted P<0.05). The results confirms that the microbiome signature obtained from the combined pool has the best performance to classify the healthy subjects vs. patients across 11 datasets.

### 2. Combined Core Pool of Genomes

The combined core pool of genomes from the combined 788 genomes was selected through the steps set out below. Random Forest classification based on a combined 788 genomes are performed for each dataset. Each of the 788 genome is ranked based on its importance for each dataset. A summed rank is obtained by adding up the value of ranks across 11 datasets and all 788 genomes are ranked again based on the summed value. The most important genome across 11 dataset gets the lowest summed rank value (Table 3).

TABLE 3
Ranking of Genome importance

| | | | | | | summed |
|---|---|---|---|---|---|---|
| Genome | Dataset 1 | Dataset 2 | Dataset 3 | . . . | Dataset 11 | rank |
| 1 | D1R1 | D2R1 | D3R1 | . . . | D11R1 | SR1 |
| 2 | D1R2 | D2R2 | D3R2 | . . . | D11R2 | SR2 |
| 3 | D1R3 | D2R3 | D3R3 | . . . | D11R3 | SR3 |
| . . . | . . . | . . . | . . . | . . . | . . . | . . . |
| 788 | D1R788 | D2R788 | D3R788 | . . . | D11R788 | SR788 |

Starting from the least important genome, every genome one by one is removed from each dataset based on order of importance. The classification performance (AUCs) is calculated for the remaining numbers of genomes after each round of removal by Random Forest model and all the genome numbers are ranked based on AUC values. The ranking values for each genome number across 11 datasets is summed (Table 4).

TABLE 4
Rank genome number based on AUC

| Genome | | | | | | summed |
|---|---|---|---|---|---|---|
| number | Dataset 1 | Dataset 2 | Dataset 3 | . . . | Dataset 11 | rank |
| 787 | D1R1_auc | D2R1_auc | D3R1_auc | D4R1_auc | D11R1_auc | SR1_auc |
| 786 | D1R2_auc | D2R2_auc | D3R2_auc | D4R2_auc | D11R2_auc | SR2_auc |
| 777 | D1R3_auc | D2R3_auc | D3R3_auc | D4R3_auc | D11R3_auc | SR3_acu |
| . . . | . . . | . . . | . . . | . . . | . . . | . . . |
| 2 | D1R786_auc | D2R786_auc | D3R786_auc | D4R786_auc | D11R786_auc | SR786_auc |

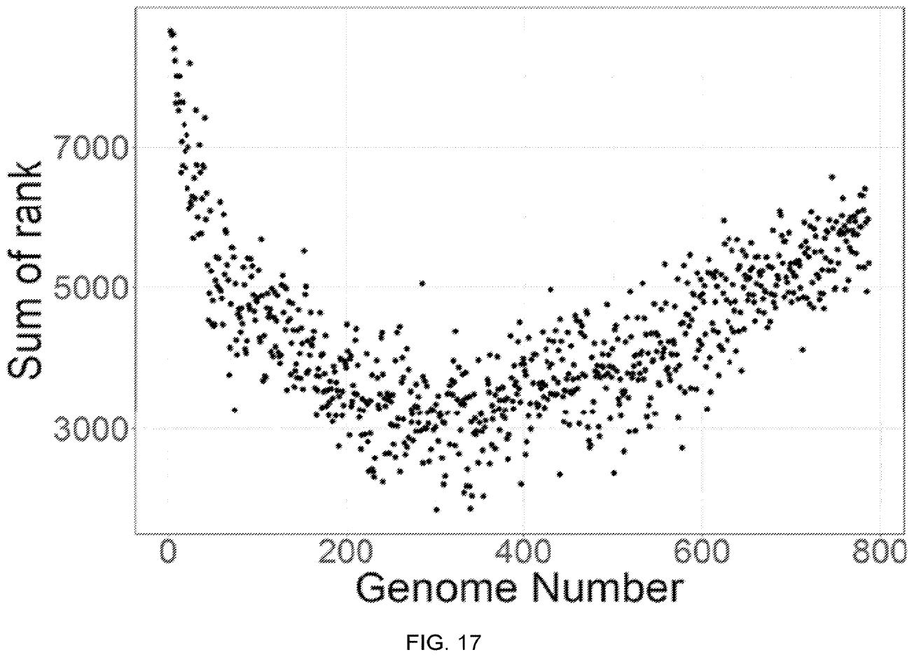

The sum of the ranking values for each genome number across 11 datasets is plotted in FIG. 17. 302 genomes achieved lowest summed AUC ranks. After removing 18 genomes which exhibit inconsistent C1A and C1B assignment, 284 genomes remained as the combined core pool genomes.

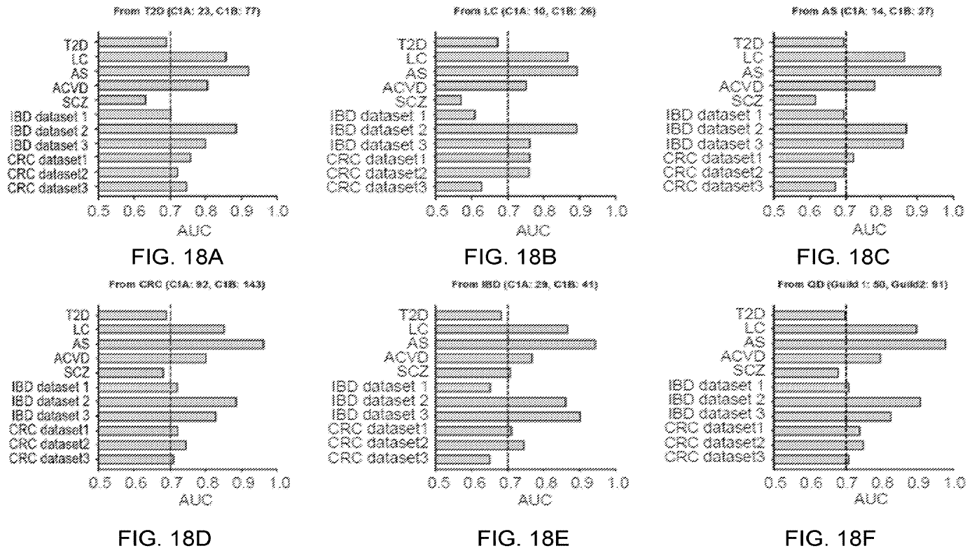

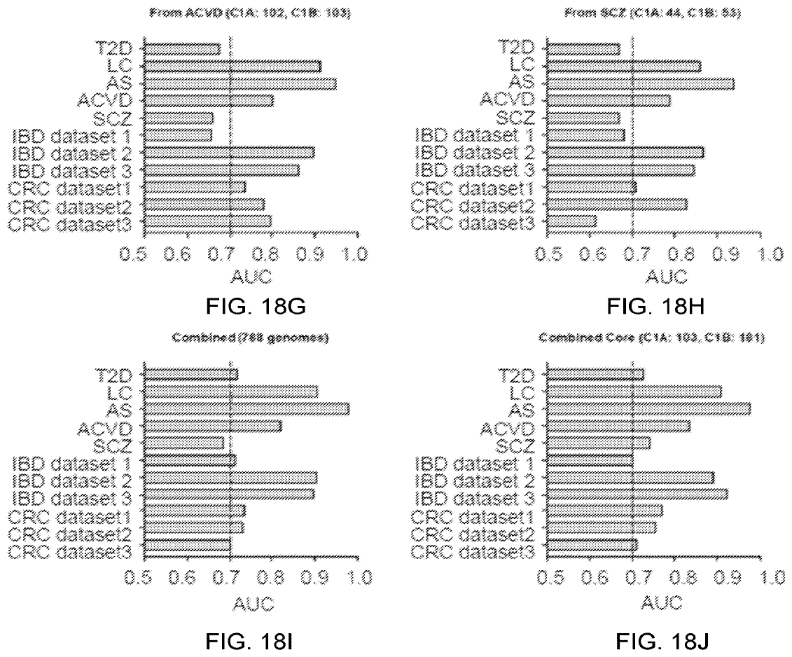

The classification capacity of the two competing guilds identified from: T2D (FIG. 18A), LC (FIG. 18B), AS (FIG. 18C), CRC (FIG. 18D), IBD (FIG. 18E), QD (FIG. 18F), AVCD (FIG. 18G), SCZ (FIG. 18H), combined pool (FIG. 18I), and the combined core pool (FIG. 18J) were compared to each other. The identified microbiome signature for each condition is utilized to classify control and patients in each dataset using Random Forest classifiers. FIG. 31 shows all microbiome signature have the capacity to classify case and control across different studies.

### Example 6—Universal Random Forest Classification Models Based on the 284 Core Genomes in the Seesaw Networked Two Competing Guilds

25 metagenomic datasets covering case-control studies on 15 different diseases (type-2 diabetes (T2D), hypertension (HT), schizophrenia (SCZ), atherosclerotic cardiovascular disease (ACVD), liver cirrhosis (LC), inflammatory bowel diseases (IBD), colorectal cancer (CRC), ankylosing spondylitis (AS), Parkinson's disease (PD), Multiple Sclerosis (MS), Gaucher disease type II (GDII), COVID-19 (COV), Behcet's disease (BD), autism spectrum disorder (ASD), and pancreatic cancer (PC)) were used to train random forest classification models for each dataset using the abundance of the same 284 core genomes present in our seesaw network of two competing guilds: the foundation guild and the pathogen guild. The models enabled us to distinguish between case and control in each of the 25 metagenomic datasets tested using leave-one-out cross-validation, with most AUCs above 0.7.

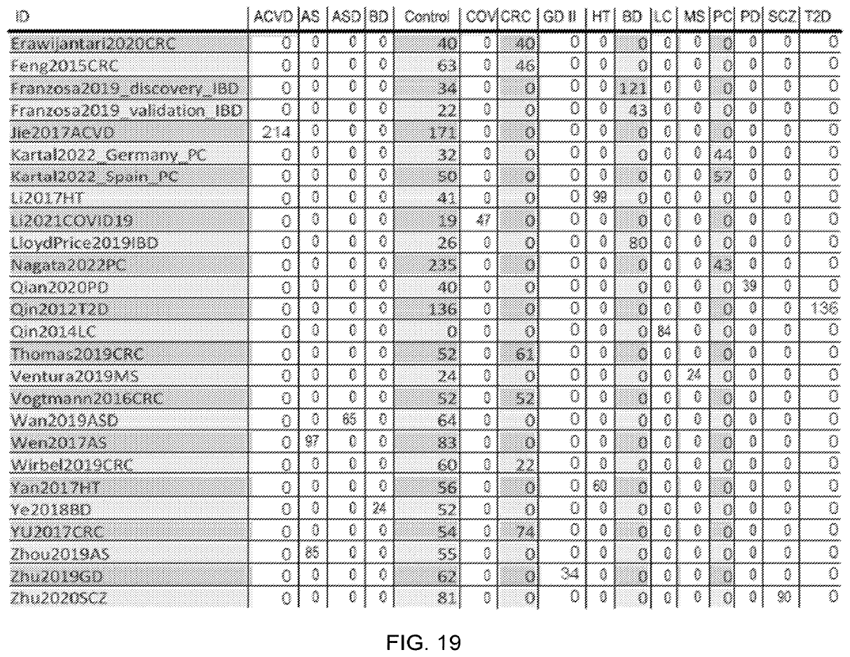

To classify case vs control, the case and control samples from the 25 datasets that corresponded to 15 various diseases (FIG. 19) were combined, considering patients with any disease as cases. All samples in the combined data set were split into two cohorts: 80% for training and 20% for testing. The 80% samples were used as training set to build Random Forest classification model based on the abundance of the 284 combined core genomes (control, n=1285; Case, n=1424;10-fold Cross Validation). The 20% samples were used as testing set to get probability score from the Random Forest classification model based on the abundance of the 284 combined core genomes (Control, n=319; Case, n=356).

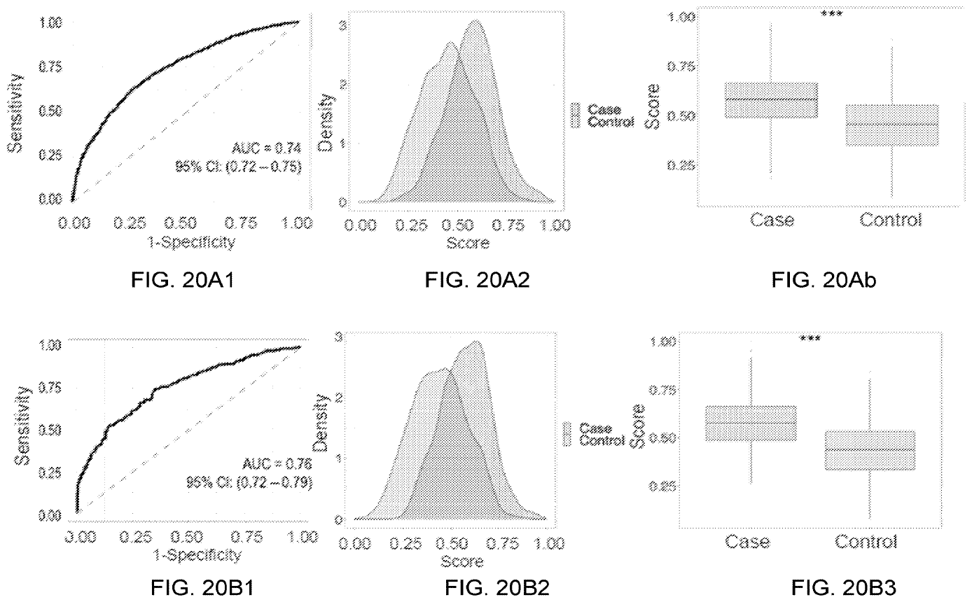

As shown in FIG. 20A1, training set resulted in an AUC of 0.74 to classify case vs. control. The best cutoff value is 0.5028, the specificity value is 0.7275, and the sensitivity value is 0.6374. As shown in FIG. 20B1, test set yielded an AUC of 0.76 to classify case vs. control. The best cutoff value is 0.531, the specificity value is 0.6489, and the sensitivity value is 0.7492. The model generated a significantly higher probability score for case than control, which were observed in both of the training set (FIG. 20A2, FIG. 20A3) and testing set (FIG. 20B2, FIG. 20B3). Accordingly, a universal model that differentiates between disease and control can be trained using the identified microorganism genomes described herein.

The success of Random Forest models based on the 284 core genomes in our seesaw networked two competing guilds suggests that the biological signals associated with these genomes are robustly detectable despite the variations introduced by all kinds of confounding factors, ranging from biological to technological. The further refinement and testing of our universal models will make a significant contribution to translational metagenomics.

### Example 7—Repeated Training for Universal Random Forest Classification Models Based on the 284 Core Genomes in the Seesaw Networked Two Competing Guilds

The 25 metagenomic datasets covering case-control studies on 15 different diseases were utilized to construct Random Forest classification models with randomly selected number of genomes out of the 284 core genomes.

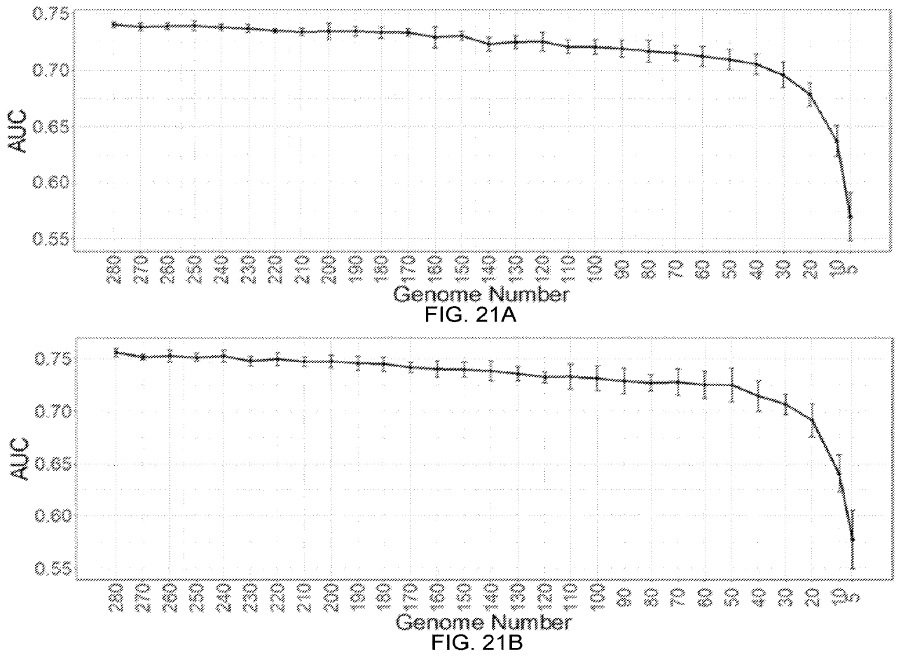

Briefly, multiple random forest classifiers were trained based on microbiota datasets obtained for diseased and healthy controls in at least one study of each of type-2 diabetes (T2D), atherosclerotic cardiovascular disease (ACVD), liver cirrhosis (LC), ankylosing spondylitis (AS), Parkinson's disease (PD), schizophrenia (SCZ), colorectal cancer (CRC), inflammatory bowel diseases (IBD), and hypertension. Specifically, datasets were randomly divided into 80% for training the RF model and 20% for testing. For each dataset, 10 classifiers were trained using randomly selected sets of 5, 10, 20, 30, 40, 50, 60, 70, 80, 90, 100, 110, 120, 130, 140, 150, 160, 170, 180, 190, 200, 210, 220, 230, 240, 250, 260, 270 and 280 genomes from the 284 genomes identified in Table 2 (290 total models per data set). The average AUC from ROC curves for each set of x randomly selected genomes was determined and plotted in FIG. 21. As shown in FIG. 21, fewer than all of the 284 genomes was required to adequately power a clinical model of disease state. In fact, in most, if not all cases, models trained with only 15-20 randomly selected genomes were adequately powered for clinical use (e.g., having an AUC of 0.65 or greater).

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown in FIG. 1, and/or as described in FIG. 2. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.
1 Zhao, L. The gut microbiota and obesity: from correlation to causality. Nat Rev Microbiol 11, 639-647, doi:10.1038/nrmicro3089 (2013).
2 Fan, Y. & Pedersen, O. Gut microbiota in human metabolic health and disease. Nat Rev Microbiol, doi:10.1038/s41579-020-0433-9 (2020).
3 Zhang, C. & Zhao, L. Strain-level dissection of the contribution of the gut microbiome to human metabolic disease. Genome Med 8, 41, doi:10.1186/s13073-016-0304-1 (2016).
4 Vacca, M. et al. The Controversial Role of Human Gut Lachnospiraceae. Microorganisms 8, doi:10.3390/microorganisms8040573 (2020).
5 Wu, G., Zhao, N., Zhang, C., Lam, Y. Y. & Zhao, L. Guild-based analysis for understanding gut microbiome in human health and diseases. Genome Medicine 13, 22, doi:10.1186/s13073-021-00840-y (2021).
6 Dominguez-Bello, M. G., Godoy-Vitorino, F., Knight, R. & Blaser, M. J. Role of the microbiome in human development. Gut 68, 1108-1114, doi:10.1136/gutjnl-2018-317503 (2019).
7 Kundu, P., Blacher, E., Elinav, E. & Pettersson, S. Our Gut Microbiome: The Evolving Inner Self. Cell 171, 1481-1493, doi:10.1016/j.cell.2017.11.024 (2017).
8 O'Hara, A. M. & Shanahan, F. The gut flora as a forgotten organ. Embo Rep 7, 688-693, doi:10.1038/sj.embor.7400731 (2006).
9 Koh, A. & Backhed, F. From Association to Causality: the Role of the Gut Microbiota and Its Functional Products on Host Metabolism. Mol Cell 78, 584-596, doi:10.1016/j.molcel.2020.03.005 (2020).
10 Sanna, S. et al. Causal relationships among the gut microbiome, short-chain fatty acids and metabolic diseases. Nat Genet 51, 600-+, doi:10.1038/s41588-019-0350-x (2019).
11 Meijnikman, A. S., Gerdes, V. E., Nieuwdorp, M. & Herrema, H. Evaluating Causality of Gut Microbiota in Obesity and Diabetes in Humans. Endocr Rev 39, 133-153, doi:10.1210/er.2017-00192 (2018).
12 Tierney, B. T., Tan, Y., Kostic, A. D. & Patel, C. J. Gene-level metagenomic architectures across diseases yield high-resolution microbiome diagnostic indicators. Nat Commun 12, 2907, doi:10.1038/s41467-021-23029-8 (2021).
13 Wang, J. & Jia, H. Metagenome-wide association studies: fine-mining the microbiome. Nat Rev Microbiol 14, 508-522, doi:10.1038/nrmicro.2016.83 (2016).
14 Duvallet, C., Gibbons, S. M., Gurry, T., Irizarry, R. A. & Alm, E. J. Meta-analysis of gut microbiome studies identifies disease-specific and shared responses. Nat Commun 8, 1784, doi:10.1038/s41467-017-01973-8 (2017).
15 Jackson, M. A. et al. Gut microbiota associations with common diseases and prescription medications in a population-based cohort. Nat Commun 9, 2655, doi:10.1038/s41467-018-05184-7 (2018).
16 Zhao, L. et al. Gut bacteria selectively promoted by dietary fibers alleviate type 2 diabetes. Science 359, 1151-1156, doi:10.1126/science.aao5774 (2018).
17 Zhang, C. H. et al. Dietary Modulation of Gut Microbiota Contributes to Alleviation of Both Genetic and Simple Obesity in Children. Ebiomedicine 2, 968-984, doi:10.1016/j.ebiom.2015.07.007 (2015).
18 Foster, K. R., Chluter, J. S., Oyte, K. Z. C. & Rakoff-Nahoum, S. The evolution of the host microbiome as an ecosystem on a leash. Nature 548, 43-51, doi:10.1038/nature23292 (2017).
19 Sommer, F., Anderson, J. M., Bharti, R., Raes, J. & Rosenstiel, P. The resilience of the intestinal microbiota influences health and disease. Nat Rev Microbiol 15, 630-638, doi:10.1038/nrmicro.2017.58 (2017).
20 Poisot, T. & Gravel, D. When is an ecological network complex? Connectance drives degree distribution and emerging network properties. PeerJ 2, e251, doi:10.7717/peerj.251 (2014).
21 Barabasi, A. L. Network science. Philos Trans A Math Phys Eng Sci 371, 20120375, doi:10.1098/rsta.2012.0375 (2013).
22 Qin, J. et al. A metagenome-wide association study of gut microbiota in type 2 diabetes. Nature 490, 55-60, doi:10.1038/nature11450 (2012).
23 Jie, Z. et al. The gut microbiome in atherosclerotic cardiovascular disease. Nat Commun 8, 845, doi:10.1038/s41467-017-00900-1 (2017).
24 Qin, N. et al. Alterations of the human gut microbiome in liver cirrhosis. Nature 513, 59-64, doi:10.1038/nature13568 (2014).
25 Wen, C. et al. Quantitative metagenomics reveals unique gut microbiome biomarkers in ankylosing spondylitis. Genome Biol 18, 142, doi:10.1186/s13059-017-1271-6 (2017).
26 Li, J. et al. Gut microbiota dysbiosis contributes to the development of hypertension. Microbiome 5, 14, doi:10.1186/s40168-016-0222-x (2017).
27 Lloyd-Price, J. et al. Multi-omics of the gut microbial ecosystem in inflammatory bowel diseases. Nature 569, 655-662, doi:10.1038/s41586-019-1237-9 (2019).
28 Franzosa, E. A. et al. Gut microbiome structure and metabolic activity in inflammatory bowel disease. Nat Microbiol 4, 293-305, doi:10.1038/s41564-018-0306-4 (2019).
29 Yu, J. et al. Metagenomic analysis of faecal microbiome as a tool towards targeted non-invasive biomarkers for colorectal cancer. Gut 66, 70-+, doi: DOI 10.1136/gutjnl-2015-309800 (2017).
30 Feng, Q. et al. Gut microbiome development along the colorectal adenoma-carcinoma sequence. Nat Commun 6, 6528, doi:10.1038/ncomms7528 (2015).
31 Qian, Y. W. et al. Gut metagenomics-derived genes as potential biomarkers of Parkinson's disease. Brain 143, 2474-2489 (2020).
32 Coyte, K. Z., Schluter, J. & Foster, K. R. The ecology of the microbiome: Networks, competition, and stability. Science 350, 663-666, doi:10.1126/science.aad2602 (2015).
33 Kamada, N., Seo, S. U., Chen, G. Y. & Nunez, G. Role of the gut microbiota in immunity and inflammatory disease. Nat Rev Immunol 13, 321-335, doi:10.1038/nri3430 (2013).
34 Vatanen, T. et al. Variation in Microbiome LPS Immunogenicity Contributes to Autoimmunity in Humans. Cell 165, 1551, doi:10.1016/j.cell.2016.05.056 (2016).
35 Bach, J. F. The hygiene hypothesis in autoimmunity: the role of pathogens and commensals. Nat Rev Immunol 18, 105-120, doi:10.1038/nri.2017.111 (2018).
36 Risely, A. Applying the core microbiome to understand host-microbe systems. Journal of Animal Ecology 89, 1549-1558 (2020).
37 Makki, K., Deehan, E. C., Walter, J. & Backhed, F. The Impact of Dietary Fiber on Gut Microbiota in Host Health and Disease. Cell Host Microbe 23, 705-715, doi:10.1016/j.chom.2018.05.012 (2018).
38 Reynolds, A. et al. Carbohydrate quality and human health: a series of systematic reviews and meta-analyses. The Lancet 393, 434-445 (2019).
39 Eaton, S. B. The ancestral human diet: what was it and should it be a paradigm for contemporary nutrition? P Nutr Soc 65, 1-6, doi:10.1079/Pns2005471 (2006).
40 Jew, S., AbuMweis, S. S. & Jones, P. J. Evolution of the human diet: linking our ancestral diet to modern functional foods as a means of chronic disease prevention. J Med Food 12, 925-934, doi:10.1089/jmf.2008.0268 (2009).
41 Spiller, G. A. & Amen, R. J. Topics in dietary fiber research. (Springer, 1978).
42 Thompson, H. J. & Brick, M. A. Perspective: Closing the dietary fiber gap: An ancient solution for a 21st century problem. Advances in Nutrition 7, 623-626 (2016).
43 Deehan, E. C. et al. Modulation of the gastrointestinal microbiome with nondigestible fermentable carbohydrates to improve human health. Microbiology spectrum 5, 5.5. 04 (2017).
44 Prevey, J. S., Germino, M. J. & Huntly, N. J. Loss of foundation species increases population growth of exotic forbs in sagebrush steppe. Ecol Appl 20, 1890-1902, doi:10.1890/09-0750.1 (2010).
45 Anderson, J. W. et al. Health benefits of dietary fiber. Nutr Rev 67, 188-205, doi:10.1111/j. 1753-4887.2009.00189.x (2009).
46 Kaczmarczyk, M. M., Miller, M. J. & Freund, G. G. The health benefits of dietary fiber: beyond the usual suspects of type 2 diabetes mellitus, cardiovascular disease and colon cancer. Metabolism 61, 1058-1066, doi:10.1016/j.metabol.2012.01.017 (2012).
47 Risely, A. Applying the core microbiome to understand host-microbe systems. J Anim Ecol 89, 1549-1558, doi:10.1111/1365-2656.13229 (2020).
48 Berg, G. et al. Microbiome definition re-visited: old concepts and new challenges. Microbiome 8, 103, doi:10.1186/s40168-020-00875-0 (2020).
49 Society, C. D. China guideline for type 2 diabetes (2013 edition). Chin J Diabetes 22 (2014).
50 yuexin, Y., guangya, W. & xingchang, P. China Food Composition (Book 1, Beijing Medical Univ. Press, ed. 2, 2009). (2009).
51 Ewing, D. & Clarke, B. Diagnosis and management of diabetic autonomic. British Medical Journal 285 (1982).
52 Feldman, E. L. et al. A Practical Two-Step Quantitative Clinical and Electrophysiological Assessment for the Diagnosis and Staging of Diabetic Neuropathy. Diabetes Care 17, 1281-1289 (1994).
53 Li, X., Zhou, Z. G., Qi, H. Y., Chen, X. Y. & Huang, G. Replacement of insulin by fasting C-peptide in modified homeostasis model assessment to evaluate insulin resistance and islet beta cell function. Zhong Nan Da Xue Xue Bao Yi Xue Ban 29, 419-423 (2004).
54 Ma, Y. C. et al. Modified glomerular filtration rate estimating equation for Chinese patients with chronic kidney disease. J Am Soc Nephrol 17, 2937-2944, doi:10.1681/ASN.2006040368 (2006).
55 Schmieder, R. & Edwards, R. Quality control and preprocessing of metagenomic datasets. Bioinformatics 27, 863-864, doi:10.1093/bioinformatics/btr026 (2011).
56 Langmead, B. & Salzberg, S. L. Fast gapped-read alignment with Bowtie 2. Nat Methods 9, 357-359, doi:10.1038/nmeth. 1923 (2012).
57 Peng, Y., Leung, H. C., Yiu, S. M. & Chin, F. Y. IDBA-UD: a de novo assembler for single-cell and metagenomic sequencing data with highly uneven depth. Bioinformatics 28, 1420-1428, doi:10.1093/bioinformatics/bts174 (2012).
58 Kang, D. D., Froula, J., Egan, R. & Wang, Z. MetaBAT, an efficient tool for accurately reconstructing single genomes from complex microbial communities. PeerJ 3, e1165, doi:10.7717/peerj. 1165 (2015).
59 Parks, D. H., Imelfort, M., Skennerton, C. T., Hugenholtz, P. & Tyson, G. W. CheckM: assessing the quality of microbial genomes recovered from isolates, single cells, and metagenomes. Genome Res 25, 1043-1055, doi:10.1101/gr.186072.114 (2015).
60 Olm, M. R., Brown, C. T., Brooks, B. & Banfield, J. F. dRep: a tool for fast and accurate genomic comparisons that enables improved genome recovery from metagenomes through de-replication. ISME J 11, 2864-2868, doi:10.1038/ismej.2017.126 (2017).
61 Fischer, M., Strauch, B. & Renard, B. Y. Abundance estimation and differential testing on strain level in metagenomics data. Bioinformatics 33, i124-i132, doi:10.1093/bioinformatics/btx237 (2017).
62 Chaumeil, P. A., Mussig, A. J., Hugenholtz, P. & Parks, D. H. GTDB-Tk: a toolkit to classify genomes with the Genome Taxonomy Database. Bioinformatics, doi:10.1093/bioinformatics/btz848 (2019).
63 Seemann, T. Prokka: rapid prokaryotic genome annotation. Bioinformatics 30, 2068-2069, doi:10.1093/bioinformatics/btu153 (2014).
64 Aramaki, T. et al. KofamKOALA: KEGG Ortholog assignment based on profile HMM and adaptive score threshold. Bioinformatics 36, 2251-2252, doi:10.1093/bioinformatics/btz859 (2020).
65 Zankari, E. et al. Identification of acquired antimicrobial resistance genes. J Antimicrob Chemother 67, 2640-2644, doi:10.1093/jac/dks261 (2012).
66 Liu, B., Zheng, D., Jin, Q., Chen, L. & Yang, J. VFDB 2019: a comparative pathogenomic platform with an interactive web interface. Nucleic Acids Res 47, D687-D692, doi:10.1093/nar/gkyl080 (2019).
67 Yin, Y. et al. dbCAN: a web resource for automated carbohydrate-active enzyme annotation. Nucleic Acids Res 40, W445-451, doi:10.1093/nar/gks479 (2012).
68 Bortolaia, V. et al. ResFinder 4.0 for predictions of phenotypes from genotypes. J Antimicrob Chemother 75, 3491-3500, doi:10.1093/jac/dkaa345 (2020).
69 Watts, S. C., Ritchie, S. C., Inouye, M. & Holt, K. E. FastSpar: rapid and scalable correlation estimation for compositional data. Bioinformatics 35, 1064-1066, doi:10.1093/bioinformatics/bty 734 (2019).
70 Shannon, P. et al. Cytoscape: a software environment for integrated models of biomolecular interaction networks. Genome Res 13, 2498-2504, doi:10.1101/gr.1239303 (2003).
71 Letunic, I. & Bork, P. Interactive Tree Of Life (iTOL) v4: recent updates and new developments. Nucleic Acids Res 47, W256-W259, doi:10.1093/nar/gkz239 (2019).

What is claimed is:

1. A method for predicting a subject's response to a therapy for a disorder, comprising:
at a computer system having one or more processors, and memory storing one or more programs for execution by the one or more processors:
A) obtaining, in electronic form, a plurality of genomic abundance values comprising, for each respective gut microorganism in a plurality of gut microorganisms selected from Table 1, Table 2, or FIG. 13A-13XX, a corresponding abundance value for the genome of the respective species of gut bacteria, in the plurality of gut microorganisms, in a biological sample from the subject; and
B) inputting the plurality of genomic abundance values into a model comprising a plurality of parameters, wherein the model applies the plurality of parameters to the plurality of genomic abundance values to generate as output from the model a prediction of the subject's response to the therapy.

2. The method of claim 1, wherein the obtaining A) comprises:
(i) obtaining, in electronic form, a plurality of at least 100,000 nucleic acid sequences for genomic DNA from the biological sample from the gut of the subject; and
(ii) determining, for each respective gut microorganism in the plurality of gut microorganisms, the corresponding value for the abundance of the genome of the respective gut microorganism from the plurality of at least 100,000 nucleic acid sequences.

3. The method of claim 2, wherein the determining A) (ii) comprises:
assembling, in electronic form, a corresponding plurality of gut microorganism genomes by metagenomic de novo sequence assembly from the plurality of at least 100,000 nucleic acid sequences, and
calculating, for each respective gut microorganism in the plurality of gut microorganisms, the corresponding value for the abundance of the genome of the respective gut microorganism based on the prevalence of respective nucleic acid sequences, in the plurality of at least 100,000 nucleic acid sequences, used to assemble a respective gut microorganism genome in the plurality of gut microorganism genomes corresponding to the respective gut microorganism.

4. The method of claim 2, wherein the determining A) (ii) comprises:
assigning each respective nucleic acid sequence in the plurality of at least 100,000 sequences to a respective gut microorganism in the plurality of gut microorganisms, thereby generating, for each respective gut microorganism in the plurality of gut microorganism, a corresponding count of respective nucleic acid sequences in the plurality of nucleic acid sequences assigned to the respective gut microorganism, and
determining, for each respective gut microorganism in the plurality of gut microorganisms, the corresponding genomic abundance value for the respective gut microorganism based on the corresponding count of respective nucleic acid sequences assigned to the respective gut microorganism.

5. The method according to any one of claims 2-4, further comprising sequencing genomic DNA from the biological sample from the gut of the subject, thereby obtaining the plurality of at least 100,000 nucleic acid sequences.

6. The method according to any one of claims 1-5, the method further comprising treating the subject by:
when the prediction of the subject's response to the therapy satisfies a threshold likelihood that the subject will respond favorably to the therapy, administering the therapy to the subject; and
when the prediction of the subject's response to the therapy does not satisfy the threshold likelihood that the subject will respond favorably to the therapy, administering one or more of the plurality of gut microorganisms to the subject.

7. The method of any one of claims 1-6, wherein the plurality of gut microorganisms comprises at least 20 microorganisms selected from those microorganisms in Table 1, Table 2, or FIG. 13A-13XX having a connectivity of at least 2.

8. The method according to any one of claims 1-7, wherein the biological sample from the gut of the subject is a fecal sample.

9. The method according to any one of claims 1-8, wherein the therapy is a biological therapy, an immunotherapy, a chemotherapy, a radiation therapy, a gene therapy, a hormone therapy, a photodynamic therapy, a targeted therapy, small molecules, antibodies, polynucleotide, natural compound, immune modulator, bone marrow therapy, stem cell therapy, surgery therapy, induction therapy, maintenance therapy, or a combination thereof.

10. The method of claim 1-9, wherein the disorder is selected from the group consisting of type-2 diabetes, hypertension, schizophrenia, atherosclerotic cardiovascular disease (ACVD), liver cirrhosis (LC), inflammatory bowel diseases (IBD), colorectal cancer (CRC), ankylosing spondylitis (AS), and Parkinson's disease (PD), inflammatory bowel disease (IBD), rheumatoid arthritis (RA), advanced melanoma and B cell lymphoma.

11. The method of claim 1-9, wherein the disorder is cancer.

12. The method of any one of claims 1-11, wherein the prediction of the subject's response is a class output of a respective response, in a plurality of possible responses, of the respective subject.

13. The method of any one of claims 1-12, wherein the prediction of the subject's response of the subject is a probability output for the respective subject's response.

14. The method of any one of claims 1-13, wherein the model is a neural network algorithm, a support vector machine algorithm, a Naïve Bayes algorithm, a nearest neighbor algorithm, a boosted trees algorithm, a Random Forest algorithm, a convolutional neural network algorithm, a decision tree algorithm, a regression algorithm, or a clustering algorithm.

15. The method of any one of claims 1-14, wherein the plurality of parameters is at least 1000, at least 10,000, at least 15,000, at least 50,000, at least 100,000, at least 250,000, at least 500,000, or at least 1,000,000 parameters.

16. The method of any one of claims 1-15, wherein the model applies the plurality of parameters to the information through at least 25,000, at least 50,000, at least 100,000, at least 250,000, at least 500,000, or at least 1,000,000 computations to obtain a corresponding output for the respective subject from the model.

17. A method of training a model for predicting a subject's response to a therapy for a disorder, comprising:
at a computer system having one or more processors, and memory storing one or more programs for execution by the one or more processors:
A) obtaining, in electronic form, for each respective training subject in a plurality of training subjects, wherein each respective training subject in the plurality of training subjects has received a therapy for a disorder:
(i) a corresponding plurality of genomic abundance values for the respective training subject at a time prior to receiving the therapy, wherein the corresponding plurality of genomic abundance values comprising, for each respective gut microorganism in a plurality of gut microorganisms, a corresponding value for the abundance of the genome of the respective gut microorganism in a corresponding biological sample from the gut of the respective training subject, and
(ii) an indication of the respective training subject's response to the therapy of the respective training subject;
B) inputting, for each respective training subject in the plurality of training subjects, information about the respective training subject into a model comprising a plurality of parameters, wherein the model applies the plurality of parameters to obtain a corresponding output for the respective training subject from the model, wherein:
the corresponding output comprises a prediction of the respective training subject's response to the therapy,
the information about the respective training subject comprises the corresponding genomic abundance value for each respective gut microorganism in the plurality of gut microorganisms, and
the plurality of gut microorganisms are selected from Table 1, Table 2, or FIG. 13A-13XX; and
C) adjusting the plurality of parameters based on, for each respective training subject in the plurality of training subjects, one or more differences between (i) the corresponding output from the model and (ii) the corresponding indication of the respective training subject's response to the therapy.

18. The method of claim 17, wherein the obtaining A) comprises, for each respective training subject in the plurality of training subjects:
(i) obtaining, in electronic form, a corresponding plurality of at least 100,000 nucleic acid sequences for genomic DNA from the corresponding biological sample from the gut of the respective training subject; and
(ii) determining, for each respective gut microorganism in the plurality of gut microorganisms, the corresponding value for the abundance of the genome of the respective gut microorganism from the corresponding plurality of at least 100,000 nucleic acid sequences.

19. The method of claim 18, wherein the determining A) (ii) comprises, for each respective training subject in the plurality of training subjects:
assembling, in electronic form, a corresponding plurality of gut microorganism genomes by metagenomic de novo sequence assembly from the corresponding plurality of at least 100,000 nucleic acid sequences, and
calculating, for each respective gut microorganism in the plurality of gut microorganisms, the corresponding value for the abundance of the genome of the respective gut microorganism based on the prevalence of respective nucleic acid sequences, in the plurality of at least 100,000 nucleic acid sequences, used to assemble a respective gut microorganism genome in the plurality of gut microorganism genomes corresponding to the respective gut microorganism.

20. The method of claim 18, wherein the determining A) (ii) comprises, for each respective subject in the plurality of training subjects:
assigning each respective nucleic acid sequence in the corresponding plurality of at least 100,000 sequences to a respective gut microorganism in the plurality of gut microorganisms, thereby generating, for each respective gut microorganism in the plurality of gut microorganism, a corresponding count of respective nucleic acid sequences in the corresponding plurality of nucleic acid sequences assigned to the respective gut microorganism, and
determining, for each respective gut microorganism in the plurality of gut microorganisms, the corresponding genomic abundance value for the respective gut microorganism based on the corresponding count of respective nucleic acid sequences assigned to the respective gut microorganism.

21. The method according to any one of claims 18-20, further comprising sequencing, for each respective subject in the plurality of training subjects, genomic DNA from the corresponding biological sample from the gut of the respective training subject, thereby obtaining the corresponding plurality of at least 100,000 nucleic acid sequences.

22. The method according to any one of claims 17-21, wherein the plurality of gut microorganisms comprises at least 20 gut microorganisms selected from Table 1, Table 2, or FIG. 13A-13XX.

23. The method of any one of claims 17-22, wherein the plurality of gut microorganisms comprises at least 20 microorganisms selected from those microorganisms in Table 1, Table 2, or FIG. 13A-13XX having a connectivity of at least 2.

24. The method according to any one of claims 17-23, wherein for each respective subject in the plurality of training subjects, the biological sample from the gut of the respective subject is a fecal sample from the respective training subject.

25. The method according to any one of claims 17-24, wherein the therapy is a biological therapy, an immunotherapy, a chemotherapy, a radiation therapy, a gene therapy, a hormone therapy, a photodynamic therapy, a targeted therapy, small molecules, antibodies, polynucleotide, natural compound, immune modulator, bone marrow therapy, stem cell therapy, surgery therapy, induction therapy, maintenance therapy, or a combination thereof.

26. The method of any one of claims 17-25, wherein the disorder is selected from the group consisting of type-2 diabetes, hypertension, schizophrenia, atherosclerotic cardiovascular disease (ACVD), liver cirrhosis (LC), inflammatory bowel diseases (IBD), colorectal cancer (CRC), ankylosing spondylitis (AS), and Parkinson's disease (PD), inflammatory bowel disease (IBD, rheumatoid arthritis (RA), or advanced melanoma and B cell lymphoma.

27. The method of any one of claims 17-26, wherein the disorder is cancer.

28. The method of any one of claims 17-27, wherein the prediction of the respective training subject's response is a class output of a respective response, in a plurality of possible responses, of the respective training subject.

29. The method of any one of claims 17-27, wherein the prediction of the respective training subject's response is a probability output for the respective training subject's response.

30. The method of any one of claims 17-29, wherein the model is a neural network algorithm, a support vector machine algorithm, a Naïve Bayes algorithm, a nearest neighbor algorithm, a boosted trees algorithm, a Random Forest algorithm, a convolutional neural network algorithm, a decision tree algorithm, a regression algorithm, or a clustering algorithm.

31. The method of any one of claims 17-30, wherein the plurality of parameters is at least 1000, at least 10,000, at least 15,000, at least 50,000, at least 100,000, at least 250,000, at least 500,000, or at least 1,000,000 parameters.

32. The method of any one of claims 17-31, wherein the model applies the plurality of parameters to the information through at least 25,000, at least 50,000, at least 100,000, at least 250,000, at least 500,000, or at least 1,000,000 computations to obtain a corresponding output for the respective training subject from the model.

33. A computer system, comprising:
one or more processors; and
a non-transitory computer-readable medium including computer-executable instructions that, when executed by the one or more processors, cause the processors to perform the method according to any one of claims 1-32.

34. A non-transitory computer-readable storage medium having stored thereon program code instructions that, when executed by a processor, cause the processor to perform the method according to any one of claims 1-32.

35. A pharmaceutical composition comprising a first gut microorganism selected from those microorganisms listed in FIG. 13A-13XX.

36. The pharmaceutical composition of claim 35, further comprising pharmaceutically acceptable excipient

37. The pharmaceutical composition of claim 35 or 36, wherein the first gut microorganism belongs to Guild 1, as identified in FIGS. 13A-13XX.

38. The pharmaceutical composition of claim 35 or 36, wherein the first gut microorganism belongs to Guild 2, as identified in FIGS. 13A-13XX.

39. The pharmaceutical composition of any one of claims 35-38, wherein the first gut microorganism has a genome having at least 99% sequence identity to a set of contigs for a microorganism listed in FIGS. 12A-12I.

40. The pharmaceutical composition of any one of claims 35-39, wherein the first gut microorganism comprises at least 50% of the total amount of gut microorganisms in the composition.

41. The pharmaceutical composition of claim 40, wherein the first gut microorganism comprises at least 75% of the total amount of gut microorganisms in the composition.

42. The pharmaceutical composition of claim 40, wherein the first gut microorganism comprises at least 90% of the total amount of gut microorganisms in the composition.

43. The pharmaceutical composition of claim 40, wherein the first gut microorganism comprises at least 95% of the total amount of gut microorganisms in the composition.

44. The pharmaceutical composition of claim 40, wherein the first gut microorganism comprises at least 99% of the total amount of gut microorganisms in the composition.

45. The pharmaceutical composition of claim 40, wherein the first gut microorganism comprises at least 99.5% of the total amount of gut microorganisms in the composition.

46. The pharmaceutical composition of claim 40, wherein the first gut microorganism comprises at least 99.9% of the total amount of gut microorganisms in the composition.

47. The pharmaceutical composition of any one of claims 35-46, further comprising a second gut microorganism selected from those microorganisms listed in FIG. 13A-13XX.

48. The pharmaceutical composition of claim 47, wherein the second gut microorganism belongs to the same Guild as the first gut microorganism, as identified in FIGS. 13A-13XX.

49. A method for treating a subject in need thereof, the method comprising administering to the subject a therapeutically effective amount of a pharmaceutical composition according to any one of claims 35-48.

50. The method of claim 49, wherein the administering is by fecal microbiome transplantation.

51. The method of claim 49, wherein the administering is by direct transplantation into the gut of the subject.

52. The method of claim 49, wherein the administering is by oral ingestion.

53. The method of any one of claims 49-52, wherein the subject has a condition selected from the group consisting of type-2 diabetes (T2D), hypertension (HT), schizophrenia (SCZ), atherosclerotic cardiovascular disease (ACVD), liver cirrhosis (LC), inflammatory bowel diseases (IBD), colorectal cancer (CRC), ankylosing spondylitis (AS), Parkinson's disease (PD), Multiple Sclerosis (MS), Gaucher disease type II (GDII), COVID-19 (COV), Behcet's disease (BD), autism spectrum disorder (ASD), and pancreatic cancer (PC).

54. The method of claim 49, wherein the subject has cancer.

55. The method of any one of claims 49-54, further comprising administering a second therapeutic agent to the subject.

56. A method for isolating a gut microorganism selected from those gut microorganisms listed in FIG. 41, the method comprising use of a sequence associated with the gut microorganism in FIG. 41 for isolation.

57. The method of claim 56, comprising isolating a set of one or more microorganism cultures grown from a single cell of a biological sample and determining, for each respective microorganism culture in the set of one or more microorganism cultures, whether genomic DNA isolated from the respective culture has sequence identity to one or more contig associated with a microorganism listed in FIG. 41.

58. A method for treating a subject, comprising:
obtaining, in electronic form by at least one processor, a plurality of genomic abundance values comprising, for each species of gut bacteria in a plurality of at least 20 species of gut bacteria, a corresponding abundance value for the genome of the respective species of gut bacteria, in the plurality of at least 20 gut species of gut bacteria, in a biological sample from the subject; and
inputting the plurality of genomic abundance values into a health detection model comprising a plurality of health detection model parameters, wherein the health detection model applies the plurality of health detection model parameters to the plurality of genomic abundance values to generate as output from the health detection model the indication of the health of the subject; and
administering to the subject at least one therapeutic agent comprising at least one gut microorganism transplant.

59. The method of claim 58, further comprising:
obtaining, in electronic form, for each respective training subject in a plurality of training subjects:
a corresponding plurality of genomic abundance values comprising, for each respective gut microorganism in a plurality of gut microorganisms, a corresponding value for the abundance of the genome of the respective gut microorganism in a corresponding biological sample from the gut of the respective training subject, and
a corresponding state of a biological characteristic of the respective training subject;
inputting, for each respective training subject in the plurality of training subjects, information about the respective training subject into a model comprising a plurality of parameters, wherein the model applies the plurality of parameters to the information to obtain a corresponding output for the respective training subject from the model, wherein:
the corresponding output comprises an indication of the corresponding state of the biological characteristic of the respective training subject, and
the information about the respective training subject comprises the corresponding genomic abundance value for each respective gut microorganism in the plurality of gut microorganisms; and
adjusting the plurality of parameters based on, for each respective training subject in the first plurality of training subjects, one or more differences between (i) the corresponding output from the model and (ii) the corresponding state of the biological characteristic of the respective training subject.

60. The method of claim 58, wherein the obtaining comprises:
obtaining, in electronic form, a plurality of at least 100,000 nucleic acid sequences for genomic DNA from the biological sample from the gut of the subject; and
determining, for each respective gut microorganism in the plurality of gut microorganisms, the corresponding value for the abundance of the genome of the respective gut microorganism from the plurality of at least 100,000 nucleic acid sequences.

61. The method of claim 60, wherein the determining comprises:
assembling, in electronic form, a corresponding plurality of gut microorganism genomes by metagenomic de novo sequence assembly from the plurality of at least 100,000 nucleic acid sequences, and
calculating, for each respective gut microorganism in the plurality of gut microorganisms, the corresponding value for the abundance of the genome of the respective gut microorganism based on the prevalence of respective nucleic acid sequences, in the plurality of at least 100,000 nucleic acid sequences, used to assemble a respective gut microorganism genome in the plurality of gut microorganism genomes corresponding to the respective gut microorganism.

62. The method of claim 60, wherein the determining comprises:
assigning each respective nucleic acid sequence in the plurality of at least 100,000 sequences to a respective gut microorganism in the plurality of gut microorganisms, thereby generating, for each respective gut microorganism in the plurality of gut microorganism, a corresponding count of respective nucleic acid sequences in the plurality of nucleic acid sequences assigned to the respective gut microorganism, and
determining, for each respective gut microorganism in the plurality of gut microorganisms, the corresponding genomic abundance value for the respective gut microorganism based on the corresponding count of respective nucleic acid sequences assigned to the respective gut microorganism.

63. The method according to any one of claims 60-62, further comprising sequencing genomic DNA from the biological sample from the gut of the subject, thereby obtaining the plurality of at least 100,000 nucleic acid sequences.

64. The method of any one of claims 60-63, wherein the plurality of gut microorganisms comprises at least 20 microorganisms having a connectivity of at least 2.

65. The method according to any one of claims 60-64, wherein the biological sample from the gut of the subject is a fecal sample.

66. The method according to any one of claims 60-65, wherein the indication of the health of the subject is an indication of a biological characteristic, wherein the biological characteristic is a disease or disorder, a therapy administered to the subject, or a diet of the subject.

67. The method of claim 66, wherein the disease or disorder is selected from the group consisting of type-2 diabetes, hypertension, schizophrenia, atherosclerotic cardiovascular disease (ACVD), liver cirrhosis (LC), inflammatory bowel diseases (IBD), colorectal cancer (CRC), ankylosing spondylitis (AS), and Parkinson's disease (PD).

68. The method of claim 66, wherein the disease or disorder is cancer.

69. The method of any one of claims 58-68, wherein the indication of the health of the subject is a class output of a respective state, in a plurality of possible states, of the health of the subject.

70. The method of any one of claims 58-68, wherein the indication of the health of the subject is a probability output for the corresponding state of the health of the subject.

71. The method of any one of claims 58-70, wherein the health detection model comprise at least one of a neural network algorithm, a support vector machine algorithm, a Naive Bayes algorithm, a nearest neighbor algorithm, a boosted trees algorithm, a Random Forest algorithm, a convolutional neural network algorithm, a decision tree algorithm, a regression algorithm, or a clustering algorithm.

72. The method of any one of claims 58-71, wherein the plurality of parameters is at least 1000, at least 10,000, at least 15,000, at least 50,000, at least 100,000, at least 250,000, at least 500,000, or at least 1,000,000 parameters.

73. The method of any one of claims 58-72, wherein the model applies the plurality of parameters to the information through at least 25,000, at least 50,000, at least 100,000, at least 250,000, at least 500,000, or at least 1,000,000 computations to obtain a corresponding output for the respective training subject from the model.
